(19) United States
(12) Patent Application Publication
(10) Pub. No.: US 2012/0023627 A1
(43) Pub. Date: Jan. 26, 2012

(54) PLANT GENE REGULATORY ELEMENTS

(75) Inventors: Srinivas Gampala, Zionsville, IN (US); Ramesh Nair, Manhattan, KS (US); Forrest Chumley, Manhattan, KS (US); Kirk Pappan, Abilene, KS (US); Prasanna Kankanala, Glendale, CA (US)

(73) Assignee: EDENSPACE SYSTEMS CORPORATION, Manhattan, KS (US)

(21) Appl. No.: 12/995,652

(22) PCT Filed: Jun. 4, 2009
(86) PCT No.: PCT/US09/46328

§ 371 Date: Aug. 12, 2011

Related U.S. Application Data
(60) Provisional application No. 61/058,907, filed on Jun. 4, 2008.

(51) Int. Cl. C12N 15/82; A01H 5/00; C12N 15/29

(52) U.S. Cl. 800/300; 536/23.6; 435/320.1; 800/298; 800/303; 800/302; 800/301; 800/320.1; 800/320; 800/320.3; 800/320.2; 800/317.3; 800/317.4; 800/317.2; 800/312; 800/322; 800/314

(57) ABSTRACT

Nucleic acids, vectors, and expression vectors comprising novel plant gene regulatory elements from sorghum. Novel transgenic plants expressing heterologous genes under the control of novel gene regulatory elements.

### RELATED APPLICATION INFORMATION

The present application claims priority to and benefit of U.S. provisional patent application 61/058,907, filed on Jun. 4, 2008, the contents of which are herein incorporated by reference in their entirety.

### BACKGROUND

Plant gene expression is highly regulated in a tissue-specific and developmental stage-specific manner. Plant gene expression is also regulated in response to many external factors, including biotic and abiotic stress. Nucleotide sequences upstream of gene coding sequences, commonly known as promoters, precisely regulate when and where any particular gene is expressed. Promoters also control the extent of foreign gene expression in transgenic plants and hence are crucial in determining the levels to which a desirable gene can be expressed.

Over the last three decades, plant biologists have isolated and characterized several plant promoters that can drive heterologous transgene expression. These well-characterized promoters include CaMV 35S promoter (Odell et al. (1985) Nature. 313:810-812), Opine promoters (U.S. Pat. No. 5,955,646), the rice actin promoter (McElroy et al. (1991) Mol Gen Genet. 231:150-160), the maize ubiquitin promoter (Christensen et al. (1992) Plant Mol Biol. 18:675-89.), the maize ADH1 promoter (U.S. Pat. No. 5,001,060) and the Rubisco promoter (Outchkourov et al. (2003) Planta 216:1003-1012).

Many of the dicot promoters do not perform satisfactorily in monocots such as maize and other cereal crops or grasses. In general, dicot promoters do not require intron sequences downstream of the transcription initiation site to enhance gene expression in transgenic dicot plants, whereas the first intron downstream of monocot promoters often enhances gene expression in transgenic monocot plants (McElroy et al. (1991) Mol Gen Genet. 231:150-160 and Christensen et al. (1992) Plant Mol Biol. 18:675-89).

Functional assays have demonstrated that differences in required promoter elements of dicot and monocot promoters may be one of the reasons why dicot promoters do not necessarily work well in monocots and vice versa.

### SUMMARY

The present invention encompasses the recognition that while transgenic monocot plants containing multiple transgenes (stacked traits) are desirable, the ability to create such plants is limited by the availability of suitable promoters for each transgene. The present invention further encompasses the recognition that a collection of novel monocot promoters, with divergent DNA sequences and an optimal range of functional characteristics, would, among other things, facilitate creating of transgenic monocot plants.

In various aspects, provided are a collection of novel monocot gene regulatory elements (including promoters), as well as nucleic acids and vectors (including gene expression vectors) comprising such novel gene regulatory elements. In one aspect, transgenic plants expressing a heterologous gene under the control of novel monocot gene regulatory elements are provided.

### BRIEF DESCRIPTION OF THE DRAWING

FIGS. 1A and 1B schematically illustrate particle bombardment expression vectors pUC18-GUSintron-NOS and pUC18-GUS-NOS. These vectors contain a multiple cloning site (MCS), a GUS reporter gene with the catalase intron (GUSintron; FIG. 1A) or without the catalase intron (GUS; FIG. 1B), and the nopaline synthase terminator (NOS).

FIGS. 2A and 2B schematically illustrate generic particle bombardment expression vectors pUC18-SbP-GUSintron-NOS and pUC18-SbP-GUS-NOS. These vectors contain various sorghum promoters (SbP), a GUS reporter gene with the catalase intron (GUSintron; FIG. 2A) or without the catalase intron (GUS; FIG. 2B), and the nopaline synthase terminator (NOS).

FIG. 3 shows GUS reporter gene expression driven by various sorghum promoters. (Expression is signified by blue spots). OsAct1, rice actin 1 promoter; SbActL1, sorghum actin like-1 promoter (SEQ ID NO: 1); SbActL5, sorghum actin like-5 promoter (SEQ ID NO: 5); SbActL6, sorghum actin like-6 promoter (SEQ ID NO: 6); SbUbiL3, sorghum ubiquitin like-3 promoter (SEQ ID NO: 10); SbUbiL4, sorghum ubiquitin like-4 promoter (SEQ ID NO: 11); SbC4HL2, sorghum cinnamate 4-hydroxylase like-2 promoter (SEQ ID NO: 43); SbPRP1L, sorghum proline rich protein 1-like promoter (SEQ ID NO: 45).

FIG. 4 shows the ubiquitous nature of the GUS reporter gene expression driven by the sorghum SbUbiL4 promoter in various tissues.

FIG. 5 shows tissue-preferred GUS reporter gene expression of sorghum promoter SbC4HL2.

FIG. 6 schematically illustrates results from structure-function analyses of sorghum promoters SbUbi3, SbUbiL4, and SbActL1. Ex, Exon; In, Intron; NE, No expression; NT, Not tested. Plus (+) indicates relative levels of GUS expression; Sizes are not to scale.

FIGS. 7A and 7B schematically illustrate plant transformation binary vectors pED-MCS-GOI-NOS and pED-SbP-GOI-NOS. These vectors contain a multiple cloning site (FIG. 3A) or various sorghum promoters (SbP) cloned into the MCS (FIG. 3B), a gene of interest (GOI), and the nopaline synthase terminator (NOS). LB, T-DNA left border sequence; RB, T-DNA right border sequence.

FIG. 8 shows the tobacco leaf infiltration activity assay results. C—, control extract; SbActL1, Sorghum Actin-like 1 promoter (SEQ ID NO. 1); 35S, Cauliflower Mosaic Virus 35S promoter.

### DEFINITIONS

Throughout the specification, several terms are employed that are defined in the following paragraphs.

As used herein, the terms “about” and “approximately”, in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the phrase “binary vector” refers to cloning vectors that are capable of replicating in both E. coli and Agrobacterium tumefaciens. In a binary vector system, two different plasmids are employed for generating transgenic plants. In many embodiments, the first plasmid is a small vector known as disarmed Ti plasmid has an origin of replication (ori) that permits the maintenance of the plasmid in a wide range of bacteria including E. coli and Agrobacterium. In many embodiments, the small vector contains foreign DNA in place of T-DNA, the left and right T-DNA borders (or at least the right T-border), markers for selection and maintenance in both E. coli and A. tumefaciens, and a selectable marker for plants. In many embodiments, the second plasmid is known as helper Ti plasmid, harbored in A. tumefaciens, which lacks the entire T-DNA region but contains an intact vir region essential for transfer of the T-DNA from Agrobacterium to plant cells.

As used herein, the phrase “cell wall-modifying enzyme polypeptide” refers to a polypeptide that modifies at least one component (e.g., xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof) or interaction (e.g., covalent linkage, ionic bond interaction, hydrogen bond interaction, and combinations thereof) in plant cell wall. In some embodiments, cell wall-modifying enzyme polypeptides have at least 50%, 60%, 70%, 80% or more overall sequence identity with a polypeptide whose amino acid sequence is set forth in Table 1 of co-pending U.S. patent application Ser. No. 12/476,247 (filed on Jun. 1, 2009), the contents of which are herein incorporated by reference in their entirety. Alternatively or additionally, in some embodiments, cell wall-modifying enzyme polypeptide shows at least 90%, 95%, 96%, 97%, 98%, 99%, or greater identity with at least one sequence element found in a polypeptide whose amino acid sequence is set forth in Table 1 of co-pending U.S. patent application Ser. No. 12/476,247, which sequence element is at least 4, 5, 6, 7, 8, 9, 10, 11, 12, 13, 14, 15, 16, 17, 18, 19, 20 or more amino acids long. In some embodiments, a provided cell wall-modifying enzyme polypeptide disrupts a linkage selected from the group consisting of hemicellulose-cellulose-lignin, hemicellulose-cellulose-pectin, hemicellulosediferululate-hemicellulose, hemicellulose-ferulate-lignin, mixed beta-D-glucan-cellulose, mixed-beta-D-glucan-hemicellulose, pectin-ferulate-lignin linkages, and combinations thereof.

As used herein, the term “construct”, when used in reference to a gene and/or nucleic acid, refers to a functional unit that allows expression of a gene of interest. Nucleic acid constructs typically comprise, in addition to the gene of interest (i.e., the heterologous gene whose expression is desired), a gene regulatory element capable of driving expression of the gene of interest (such as a promoter) and a terminator (also known as a stop signal), both of which are operably linked to the gene of interest. In some embodiments, constructs comprise additional sequences, e.g. marker genes that are also accompanied by a gene regulatory element (such as a promoter) and a terminator. In many embodiments, the sequences for each of the elements in the cnostruct do not exist in this combination and arrangement in nature and/or are arranged and/or combined by the hand of man.

As used herein, the phrase “externally applied”, when used to describe enzyme polypeptides used in the processing of biomass, refers to enzyme polypeptides that are not produced by the organism whose biomass is being processed. “Externally applied” enzyme polypeptides as used herein does not include enzyme polypeptides that are expressed (whether endogenously or transgenically) by the organism (e.g., plant) from which the biomass is obtained.

As used herein, the term “extract”, when used as noun, refers to a preparation from a biological material (such as lignocellulosic biomass) in which a substantial portion of proteins are in solution. In some embodiments of the invention, the extract is a crude extract, e.g., an extract that is prepared by disrupting cells such that proteins are solubilized and optionally removing debris, but not performing further purification steps. In some embodiments of the invention, the extract is further purified in that certain substances, molecules, or combinations thereof are removed.

As used herein, the term “gene” refers to a discrete nucleic acid sequence responsible for a discrete cellular product and/or performing one or more intracellular or extracellular functions. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. The gene and regulatory sequences may be derived from the same natural source, or may be heterologous to one another. The definition can also include nucleic acids that do not encode proteins but rather provide templates for transcription of functional RNA molecules such as tRNAs, rRNAs, etc. Alternatively, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA) or a protein produced by translation of an mRNA. Gene products also include RNAs that are modified by processes such as capping, polyadenylation, methylation, and editing, proteins post-translationally modified, and proteins modified by, for example, methylation, acetylation, phosphorylation, ubiquitination, ADP ribosylation, myristilation, and glycosylation.

The terms “genetically modified” and “transgenic” are used herein interchangeably. A transgenic or genetically modified organism is one that has a genetic background which is at least partially due to manipulation by the hand of man through the use of genetic engineering. For example, the term “transgenic cell”, as used herein, refers to a cell whose DNA contains an exogenous nucleic acid not originally present in the non-transgenic cell. A transgenic cell may be derived or regenerated from a transformed cell or derived from a transgenic cell. Exemplary transgenic cells in the context of the present invention include plant calli derived from a stably transformed plant cell and particular cells (such as leaf, root, stem, or reproductive cells) obtained from a transgenic plant. A “transgenic plant” is any plant in which one or more of the cells of the plant contain heterologous nucleic acid sequences introduced by way of human intervention. Transgenic plants typically express DNA sequences, which confer the plants with characters different from that of native, non-transgenic plants of the same strain. The progeny from such a plant or from crosses involving such a plant in the form of plants, seeds, tissue cultures and isolated tissue and cells, which carry at least part of the modification originally introduced by genetic engineering, are comprised by the definition.

As used herein, the term “genetic probe” refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acids from a sample (e.g., RNA from a plant extract) are hybridized. Genetic probes specifically bind (or specifically hybridize) to nucleic acid of complementary or substantially complementary sequence through one or more types of chemical bonds, usually through hydrogen bond formation.

As used herein, the term “gene regulatory element” means an element, typically within a nucleic acid, that has the ability to regulate genes, whether it is a by promoting, enhancing, or attenuating expression. In some embodiments, the gene regulatory element is a promoter. In some embodiments, the gene regulatory element is an enhancer. In some embodiments, gene regulatory elements are located at or near the 5′ end of the first exon of a gene. In some embodiment, gene regulatory elements are located within the region of a gene involved in transcriptional and translational initiation.

As used herein the term “heterologous”, when used in reference to genes, refers to genes that are not normally associated with other genetic elements with which they are nevertheless associated (e.g., in a nucleic acid construct) in such an arrangement in nature and/or refers to genes that are associated with such other elements by the hand of man. “Heterologous gene products” refers to products of heterologous genes.

As used herein, the term “lignocellulolytic enzyme polypeptide” refers to a polypeptide that disrupts or degrades lignocellulose, which comprises cellulose, hemicellulose, and lignin. The term “lignocelluloytic enzyme polypeptide” encompasses, but is not limited to cellobiohydrolases, endoglucanases, β-D-glucosidases, xylanases, arabinofuranosidases, acetyl xylan esterases, glucuronidases, mannanases, galactanases, arabinases, lignin peroxidases, manganese-dependent peroxidases, hybrid peroxidases, laccases, ferulic acid esterases and related polypeptides. In some embodiments, disruption or degradation of lignocellulose by a lignocellulolytic enzyme polypeptide leads to the formation of substances including monosaccharides, disaccharides, polysaccharides, and phenols. In some embodiments, a lignocellulolytic enzyme polypeptide shares at least 50%, 60%, 70%, 80% or more overall sequence identity with a polypeptide whose amino acid sequence is set forth in Table 1. Alternatively or additionally, in some embodiments, a lignocellulolytic enzyme polypeptide shows at least 90%, 95%, 96%, 97%, 98%, 99%, or greater identity with at least one sequence element found in a polypeptide whose amino acid sequence is set forth in Table 1, which sequence element is at least 4, 5, 6, 7, 8, 9, 10, 11, 12, 13, 14, 15, 16, 17, 18, 19, 20 or more amino acids long. It will be appreciated that the present invention describes use of lignocellulolytic enzyme polypeptides generally, but also of particular lignocellulolytic enzyme polypeptides (e.g., Acidothermus cellulolyticus E1 endo-1,4-β-glucanase polypeptide, Acidothermus cellulolyticus xylE polypeptide, Acidothermus cellulolyticus gux1 polypeptide, Acidothermus cellulolyticus aviIII polypeptide, and Talaromyces emersonii cbhE polypeptide).

As used herein, the term “mixed linkage glucans” refer to non-cellulosic glucans present in plants and often enriched in seed bran. β-D-glucan residues of mixed-linkage glucans are unbranched but contain both (1→3) and (1→4)-linkages. In some embodiments, enzymes that modify mixed-linkage glucans include laminarinase (E.C. 3.2.1.39), licheninase (E.C. 3.2.1.73/74). In some embodiments, some cellulases can hydrolyze certain (1→4)-linkages.

As used herein, the term “nucleic acid construct” refers to a polynucleotide or oligonucleotide comprising nucleic acid sequences not normally associated in nature. A nucleic acid construct of the present invention is prepared, isolated, or manipulated by the hand of man. The terms “nucleic acid”, “polynucleotide” and “oligonucleotide” are used herein interchangeably and refer to a deoxyribonucleotide (DNA) or ribonucleotide (RNA) polymer either in single- or double-stranded form. For the purposes of the present invention, these terms are not to be construed as limited with respect to the length of the polymer and should also be understood to encompass analogs of DNA or RNA polymers made from analogs of natural nucleotides and/or from nucleotides that are modified in the base, sugar and/or phosphate moieties.

As used herein, the term “operably linked” refers to a relationship between two nucleic acid sequences wherein the expression of one of the nucleic acid sequences is controlled by, regulated by or modulated by the other nucleic acid sequence. In some embodiments, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species.

As will be clear from the context, the term “plant”, as used herein, can refer to a whole plant, plant parts (e.g., cuttings, tubers, pollen), plant organs (e.g., leaves, stems, flowers, roots, fruits, branches, etc.), individual plant cells, groups of plant cells (e.g., cultured plant cells), protoplasts, plant extracts, seeds, and progeny thereof. The class of plants that may be used in the methods of the present invention is as broad as the class of higher plants amenable to transformation techniques, including both monocotyledonous and dicotyledonous plants, as well as certain lower plants such as algae. The term includes plants of a variety of a ploidy levels, including polyploid, diploid and haploid. In certain embodiments of the invention, plants are green field plants. In other embodiments, plants are grown specifically for “biomass energy”. For example, suitable plants include, but are not limited to, alfalfa, bamboo, barley, canola, corn, cotton, cottonwood (e.g. Populus deltoides), eucalyptus, miscanthus, poplar, pine (pinus sp.), potato, rape, rice, soy, sorghum, sugar beet, sugarcane, sunflower, sweetgum, switchgrass, tobacco, turf grass, wheat, and willow. Using transformation methods, genetically modified plants, plant cells, plant tissue, seeds, and the like can be obtained.

As used herein, “plant biomass” refers to biomass that includes a plurality of components found in plant, such as lignin, cellulose, hemicellulose, beta-glucans, homogalacturonans, and rhamnogalacturonans. Plant biomass may be obtained, for example, from a transgenic plant expressing at least one cell wall-modifying enzyme polypeptide as described herein. Plant biomass may be obtained from any part of a plant, including, but not limited to, leaves, stems, seeds, and combinations thereof.

As used herein, the term “polypeptide” generally has its art-recognized meaning of a polymer of at least three amino acids. However, the term is also used to refer to specific functional classes of polypeptides, such as, for example, lignocellulolytic enzyme polypeptides (including, for example, Acidothermus cellulolyticus E1 endo-1,4-β-glucanase polypeptide, Acidothermus cellulolyticus xylE polypeptide, Acidothermus cellulolyticus gux1 polypeptide, Acidothermus cellulolyticus aviIII polypeptide, and Talaromyces emersonii cbhE polypeptide). For each such class, the present specification provides specific examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented herein.

As used herein, the term “pretreatment” refers to a thermo-chemical process to remove lignin and hemicellulose bound to cellulose in plant biomass, thereby increasing accessibility of the cellulose to cellulases for hydrolysis. Common methods of pretreatment involve using dilute acid (such as, for example, sulfuric acid), ammonia fiber expansion (AFEX), steam explosion, lime, and combinations thereof.

As used herein, the terms “promoter” and “promoter element” refer to a polynucleotide that regulates expression of a selected polynucleotide sequence operably linked to the promoter, and which effects expression of the selected polynucleotide sequence in cells. The term “plant promoter”, as used herein, refers to a promoter that functions in a plant. In some embodiments of the invention, the promoter is a constitutive promoter, i.e., an unregulated promoter that allows continual expression of a gene associated with it. A constitutive promoter may in some embodiments allow expression of an associated gene throughout the life of the plant. Examples of constitutive plant promoters include, but are not limited to, rice act 1 promoter, Cauliflower mosaic virus (CaMV) 35S promoter, and nopaline synthase promoter from Agrobacterium tumefaciens. In some embodiments, the promoter is a promoter from sorghum. In some embodiments, the promoter comprises a polynucleotide having a sequence of at least one of SEQ ID NO: 1 to 48. In some embodiments of the invention, the promoter is a tissue-specific promoter that selectively functions in a part of a plant body, such as a flower. In some embodiments of the invention, the promoter is a developmentally specific promoter. In some embodiments of the invention, the promoter is an inducible promoter. In some embodiments of the invention, the promoter is a senescence promoter, i.e., a promoter that allows transcription to be initiated upon a certain event relating to the age of the organism.

As used herein, the term “protoplast” refers to an isolated plant cell without cell walls which has the potency for regeneration into cell culture or a whole plant.

As used herein, the term “regeneration” refers to the process of growing a plant from a plant cell (e.g., plant protoplast, plant callus or plant explant).

As used herein, the term “stably transformed”, when applied to a plant cell, callus or protoplast refers to a cell, callus or protoplast in which an inserted exogenous nucleic acid molecule is capable of replication either as an autonomously replicating plasmid or as part of the host chromosome. The stability is demonstrated by the ability of the transformed cells to establish cell lines or clones comprised of a population of daughter cells containing the exogenous nucleic acid molecule.

As used herein, the term “tempering” refers to a process to condition lignocellulosic biomass prior to pretreatment so as to favor improved yield from hydrolysis and/or allow use of less severe pretreatment conditions without sacrificing yield. In some embodiments, the lignocellulosic biomass transgenically expresses a lignocellulolytic enzyme polypeptide and tempering facilitates activation of the lignocellulolytic enzyme polypeptide. In some embodiments, tempering facilitates improved yield from subsequent hydrolysis as compared to yield obtained from processing without tempering. In some embodiments, tempering facilitates comparable or improved yield from subsequent hydrolysis using less severe pretreatment conditions than would be required without tempering. In some embodiments, tempering comprises a process selected from the group consisting of ensilement, grinding, pelleting, forming a warm water suspension and/or slurry, incubating at a specific temperature, incubating at a specific pH, and combinations thereof. In some embodiments, tempering comprises separating liquid from a slurry that contains soluble sugars and crude enzyme extracts and re-addition of the separated liquid back to the solid biomass after pretreatment. Specific conditions for tempering may depend on specific traits (such as, e.g., traits of the transgene) of the biomass.

As used herein, the term “tissue-preferred”, when used in reference to a gene regulatory element (such as a promoter) or an expression pattern, means characterized by expression preferences in certain tissues. For example, a tissue-preferred promoter can drive and/or facilitate expression that is high in certain tissues (eg. stem) but in low in others.

As used herein, the term “tissue-specific”, when used in reference to a gene regulatory element (such as a promoter) or an expression pattern, means characterized by expression only in certain tissues. For example, a tissue-specific promoter can drive and/or facilitate expression in some tissues but not others.

As used herein, the term “transformation” refers to a process by which an exogenous nucleic acid molecule (e.g., a vector or recombinant DNA molecule) is introduced into a recipient cell, callus or protoplast. The exogenous nucleic acid molecule may or may not be integrated into (i.e., covalently linked to) chromosomal DNA making up the genome of the host cell, callus or protoplast. For example, the exogenous polynucleotide may be maintained on an episomal element, such as a plasmid. Alternatively, the exogenous polynucleotide may become integrated into a chromosome so that it is inherited by daughter cells through chromosome replication. Methods for transformation include, but are not limited to, electroporation, magnetoporation, Ca2+ treatment, injection, particle bombardment, retroviral infection, and lipofection. In some circumstances, an exogenous nucleic acid is introduced in to a cell by mating with another cell. For example, in S. cerevisiae, cells mate with one another.

The term “transgene”, as used herein, refers to an exogenous gene which, when introduced into a host cell through the hand of man, for example, using a process such as transformation, electroporation, particle bombardment, and the like, is expressed by the host cell and integrated into the cell's DNA such that the trait or traits produced by the expression of the transgene is inherited by the progeny of the transformed cell. A transgene may be partly or entirely heterologous (i.e., foreign to the cell into which it is introduced). Alternatively, a transgene may be homologous to an endogenous gene of the cell into which it is introduced, but is designed to be inserted (or is inserted) into the cell's genome in such a way as to alter the genome of the cell (e.g., it is inserted at a location which differs from that of the natural gene or its insertion results in a knockout). A transgene can also be present in a cell in the form of an episome. A transgene can include one or more transcriptional regulatory sequences and other nucleic acids, such as introns. Alternatively or additionally, a transgene is one that is not naturally associated with the vector sequences with which it is associated according to the present invention.

### DETAILED DESCRIPTION OF CERTAIN EMBODIMENTS

In various embodiments, the present invention provides, among other things, novel nucleic acids and vectors comprising novel gene regulatory elements from sorghum that can be used to express a gene of interest in a variety of cells, including both monocot and dicot plants. Monocot and dicot transgenic plants expressing heterologous genes under the control of a novel gene regulatory element are also provided.

### I. Nucleic Acids

Nucleic acids of the present invention generally comprise a characteristic sequence corresponding to a novel gene regulatory element from sorghum.

Nucleotide sequences of certain provided sorghum gene regulatory elements are listed as SEQ ID NOs: 1 to 48 and presented in Table 5. In some embodiments, nucleotide sequences of provided nucleic acids comprise a sequence having at least 65%, at least 70%, at least 75%, at least 80%, at least 85%, at least 90%, at least 95%, at least 96%, at least 97%, at least 98%, at least 99%, or more identity to at least one of SEQ ID NO.: 1 to 48. In some embodiments, nucleotide sequences of provided nucleic acids comprise a sequence having at least 65%, at least 70%, at least 75%, at least 80%, at least 85%, at least 90%, at least 95%, at least 96%, at least 97%, at least 98%, at least 99%, or more identity to at least one of SEQ ID NO: 1, 5, 6, 10, 11, 43, and 45. (See, e.g., Examples 2, 3, 4, and 6.). In some embodiments, the nucleotide sequences of provided nucleic acids comprise a sequence having at least 65%, at least 70%, at least 75%, at least 80%, at least 85%, at least 90%, at least 95%, at least 96%, at least 97%, at least 98%, at least 99%, or more sequence identity to at least one of SEQ ID NO: 11 and 45.

In many embodiments, provided nucleic acids comprise gene regulatory elements from sorghum. In some such embodiments, the gene regulatory elements are promoters, that is, they can drive expression of an gene that is operably linked.

Nucleic acids of the invention may include, in addition to nucleotide sequences described above, sequences that can facilitate manipulations such as molecular cloning. For example, restriction enzyme recognition sites and/or recombinase recognition sites may be included in inventive nucleic acids.

Nucleic acids of the present invention included single stranded and double stranded nucleic acids. DNA, RNA, DNA:RNA heteroduplexes, RNA:RNA duplexes, and DNA-RNA hybrid molecules are contemplated and included. In some embodiments, nucleic acids of the present invention include unconventional nucleotides, chemically modified nucleotides, and/or labeled nucleotides (e.g., radiolabeled, fluorescently labeled, enzymatically labeled, etc.). For example, modifications, labels, and/or use of unconventional nucleotides may facilitate downstream manipulations and/or analyses.

### II. Vectors

Gene vectors of the present invention generally contain a nucleic acid construct that includes one or more expression cassettes for expression of a gene of interest (e.g., a heterologous gene) in a plant of interest. Nucleic acid constructs (also known as “gene constructs”) act as a functional unit that allows expression of a gene of interest. Nucleic acid constructs typically comprise, in addition to the gene of interest (e.g., a heterologous gene whose expression is desired), a gene regulatory element capable of driving expression of the gene of interest (such as a promoter) and a terminator (also known as a stop signal), both of which are operably linked to the gene of interest.

In many embodiments, the gene regulatory element regulates expression of the gene of interest (such as a heterologous gene).

In some embodiments, constructs comprise additional sequences, e.g. marker genes, which are also accompanied by a gene regulatory element (such as a promoter) and a terminator. In many embodiments, the sequences for each of the elements in the construct do not exist in this combination and arrangement in nature and/or are arranged and/or combined by the hand of man.

### A. Expression Cassettes

Expression cassettes generally include 5′ and 3′ regulatory sequences operably linked to a nucleotide sequence encoding a gene of interest.

Techniques used to isolate or clone a gene of interest are known in the art and include isolation from genomic DNA, preparation from cDNA, or a combination thereof. Cloning of a gene from such genomic DNA, can be effected, e.g., by using polymerase chain reaction (PCR) or antibody screening or expression libraries to detect cloned DNA fragments with shared structural features (Innis et al., “PCR: A Guide to Method and Application”, 1990, Academic Press: New York). Alternatively or additionally, other nucleic acid amplification procedures such as ligase chain reaction (LCR), ligated activated transcription (LAT) and nucleotide sequence-based amplification (NASBA) may be used.

Expression cassettes generally include the following elements (presented in the 5′-3′ direction of transcription): a transcriptional and translational initiation region, a coding sequence for a gene of interest, and a transcriptional and translational termination region functional in the organism where it is desired to express the gene of interest (such as a plant).

Other sequences that can be present in a nucleic acid construct include sequences that enhance gene expression (such as, for example, intron sequences and leader sequences). Examples of introns that have been reported to enhance expression include, but are not limited to, introns of the Maize Adh1 gene and introns of the Maize bronze1 gene (J. Callis et. al., Genes Develop. 1987, 1: 1183-1200). Examples of non-translated leader sequences that are known to enhance expression include, but are not limited to, leader sequences from Tobacco Mosaic Virus (TMV, the “omegasequence”), Maize Chlorotic Mottle Virus (MCMV), and Alfalfa Mosaic Virus (AlMV) (see, for example, D. R. Gallie et al., Nucl. Acids Res. 1987, 15: 8693-8711; J. M. Skuzeski et. al., Plant Mol. Biol. 1990, 15: 65-79).

Where appropriate, the gene(s) or polynucleotide sequence(s) encoding the enzyme(s) of interest may be modified to include codons that are optimized for expression in the transformed plant (Campbell and Gowri, Plant Physiol., 1990, 92: 1-11; Murray et al., Nucleic Acids Res., 1989, 17: 477-498; Wada et al., Nucl. Acids Res., 1990, 18: 2367, and U.S. Pat. Nos. 5,096,825; 5,380,831; 5,436,391; 5,625,136, 5,670,356 and 5,874,304). Codon optimized sequences are synthetic sequences, and preferably encode the identical polypeptide (or an enzymatically active fragment of a full length polypeptide which has substantially the same activity as the full length polypeptide) encoded by the non-codon optimized parent polynucleotide.

### 1. Transcriptional and Translational Initiation

Transcriptional initiation regions (also known as gene promoters, which may be said to comprise ‘promoter elements’) in nucleic acid constructs of the present invention can be native or analogous (i.e., found in the native organism such as a plant) and/or foreign or heterologous (i.e., not found in the native plant) to the plant host. Promoters can comprise a naturally occurring sequence and/or a synthetic sequence.

A given nucleic acid construct may contain more than one promoter, for example, in embodiments wherein expression of more than one heterologous gene is desired. In some embodiments, the two or more promoters include promoters that are the same. In the some embodiments, the two or more promoters are different from one another. In some embodiments that involve at least two different promoters, one promoter drives expression of a heterologous gene in cells of one species (such as a species bacterium) while one other promoter drives expression of a heterologous gene in cells of another species (such as a plant species). In some embodiments, the two or more promoters include at least two promoters that drive expression in cells of the same species.

As mentioned previously, the present invention provides in certain embodiments gene regulatory elements from sorghum, which include sorghum promoters capable of driving gene expression in plants, including sorghum and plants other than sorghum (including both monocotyledonous and dicotyledonous plants). In many embodiments, provided gene regulatory elements comprise isolated nucleic acids as described above. Nucleotide sequences of certain provided sorghum gene regulatory elements are listed as SEQ ID NOs: 1 to 48. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO.: 1 to 48. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO: 1, 5, 6, 10, 11, 43, and 45. (See, e.g., Examples 2, 3, 4, and 6.). In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more sequence identity to at least one of SEQ ID NO: 11 and 45.

Provided gene regulatory elements can be used alone, in combination with each other, and/or in combination with known promoters (such as known plant promoters) to drive and/or facilitate expression of a gene of interest (such as a heterologous gene). For example, in embodiments wherein two heterologous gene products are expressed in the same plant or other organism, expression of one heterologous gene product may be driven and/or facilitated by a gene regulatory element from sorghum provided herein, while expression of the other heterologous gene product may be driven and/or facilitated by another second gene regulatory element from sorghum provided herein. Alternatively or additionally, expression of one heterologous gene product may be driven and/or facilitated by a gene regulatory element from sorghum provided herein, while expression of the other heterologous gene product may be driven and/or facilitated by a known promoter such as a known plant promoter. Any number of heterologous gene products may be expressed with the aid of and/or under the control of any combinations of gene regulatory elements or promoters.

Provided gene regulatory elements include several types of plant promoters, such as constitutive plant promoters, tissue-specific promoters, and developmental-stage specific plant promoters.

In certain embodiments, at least one promoter in the nucleic acid construct is a constitutive plant promoter, i.e., an unregulated promoter that allows continual expression of a gene associated with it. Examples of known plant promoters that can be used in addition to provided gene regulatory elements include, but are not limited to, the 35S cauliflower mosaic virus (CaMV) promoter, a promoter of nopaline synthase, and a promoter of octopine synthase. Examples of other constitutive promoters used in plants are the 19S promoter and promoters from genes encoding actin and ubiquitin. Promoters may be obtained from genomic DNA by using polymerase chain reaction (PCR), and then cloned into the construct.

Constitutive promoters may allow expression of an associated gene throughout the life of an organism such as a plant. In some embodiments, the heterologous gene product is produced throughout the life of the organism. In some embodiments, the heterologous gene product is active throughout the life of the organism. Alternatively or additionally, a constitutive promoter may allow expression of an associated gene in all or a majority of tissues in the organism. In some embodiments, the heterologous gene product is present in all tissues during the life of the organism.

In certain embodiments, at least one promoter in the nucleic acid construct is a tissue-specific plant promoter, i.e., a promoter that allows expression of a gene in a specific tissue or tissues associated with it.

In certain embodiments, at least one promoter in the nucleic acid construct is a tissue-preferred plant promoter, i.e., a promoter that allows preferential expression in one or some tissues (e.g., higher in one or some tissues than in others). For example, a tissue-preferred plant promoter may allow a high level of expression in stem but a low level of expression in leaves and seed. Example 6 of the present application describes a tissue-preferred sorghum promoter (SBC4HL2) provided by the present invention.

### 2. Genes of Interest/Heterologous Genes

The gene of interest can be any gene whose expression is desired. In a nucleic acid construct (particularly expression constructs), genes of interest are generally heterologous, i.e., they are not normally associated with the other elements in the construct in such an arrangement in nature and/or they are associated with such other elements by the hand of man. In some embodiments, heterologous gene products (which may be polypeptides and/or RNA molecules) are expressed in cells, tissues, and/or organisms in which they are not expressed in nature; and/or are expressed at levels different than they are expressed in nature.

A given nucleic acid construct may have one or more than one heterologous gene.

a. Enzyme Polypeptides

In some embodiments, the heterologous gene encodes an enzyme polypeptide. A wide variety of enzyme polypeptides may be expressed under the control of, or facilitated by, sorghum gene regulatory elements provided by the present invention. A discussion of some classes of such enzyme polypeptides is presented below. The discussion below is not intended to be exhaustive; provided gene regulatory elements may be used to drive and/or facilitate expression of other enzyme polypeptides as well.

i. Lignocellulolytic Enzyme Polypeptides

In some embodiments, the heterologous gene is a lignocellulolytic enzyme polypeptide.

Plants generally comprise lignocellulosic biomass, a complex substrate in which crystalline cellulose is embedded within a matrix of hemicellulose and lignin. Lignocellulose represents approximately 90% of the dry weight of most plant material with cellulose making up between 30% to 50% of the dry weight of lignocellulose and hemicellulose making up between 20% and 50% of the dry weight of lignocellulose.

Disruption and degradation (e.g., hydrolysis) of lignocellulose by lignocellulolytic enzyme polypeptides leads to the formation of substances including monosaccharides, disaccharides, polysaccharides and phenols. In some embodiments, the lignocellulolytic enzyme polyeptide are characterized by and/or are employed under conditions and/or according to a protocol that achieves enhanced disruption and/or degradation of lignocellulose.

Lignocellulolytic enzyme polypeptides whose expression may be driven with gene regulatory elements of the invention include enzymes that are involved in the disruption and/or degradation of lignocellulose. Lignocellulolytic enzyme polypeptides include, but are not limited to, cellulases, hemicellulases and ligninases. Representative examples of lignocellulolytic enzyme polypeptides are presented in Table 1.

TABLE 1
Examples of lignocellulolytic enzyme polypeptides

| | | | GenBank |
|---|---|---|---|
| Gene | Microbial | Amino Acid Sequence of Exemplary | Accession |
| name | species | Lignocellulolytic Enzyme Polypeptide | Number |
| E1 | Acidothermus | AGGGYWHTSGREILDANNVPVRIAGINWFGFETCNYVVHGLWSRDYRS | AAA75477 |
| | cellulolyticus | MLDQIKSLGYNTIRLPYSDDILKPGTMPNSINFYQMNQDLQGLTSLQV | |
| | | MDKIVAYAGQIGLRIILDRHRPDCSGQSALWYTSSVSEATWISDLQAL | |
| | | AQRYKGNPTVVGFDLHNEPHDPACWGCGDPSIDWRLAAERAGNAVLSV | |
| | | NPNLLIFVEGVQSYNGDSYWWGGNLQGAGQYPVVLNVPNRLVYSAHDY | |
| | | ATSVYPQTWFSDPTFPNNMPGIWNKNWGYLFNQNIAPVWLGEFGTTLQ | |
| | | STTDQTWLKTLVQYLRPTAQYGADSFQWTFWSWNPDSGDTGGILKDDW | |
| | | QTVDTVKDGYLAPIKSSIFDPVG | |
| gux1 | Acidothermus | MGAPGLRRRLRAGIVSAAALGSLVSGLVAVAPVAHAAVTLKAQYKNND | ABK52390.1 |
| | cellulolyticus | SAPSDNQIKPGLQLVNTGSSSVDLSTVTVRYWFTRDGGSSTLVYNCDW | |
| | | AAMGCGNIRASFGSVNPATPTADTYLQLSFTGGTLAAGGSTGEIQNRV | |
| | | NKSDWSNFDETNDYSYGTNTTFQDWTKVTVYVNGVLVWGTEPSGATAS | |
| | | PSASATPSPSSSPTTSPSSSPSPSSSPTPTPSSSSPPPSSNDPYIQRF | |
| | | LTMYNKIHDPANGYFSPQGIPYHSVETLIVEAPDYGHETTSEAYSFWL | |
| | | WLEATYGAVTGNWTPFNNAWTTMETYMIPQHADQPNNASYNPNSPASY | |
| | | APEEPLPSMYPVAIDSSVPVGHDPLAAELQSTYGTPDIYGMHWLADVD | |
| | | NIYGYGDSPGGGCELGPSAKGVSYINTFQRGSQESVWETVTQPTCDNG | |
| | | KYGGAHGYVDLFIQGSTPPQWKYTDAPDADARAVQAAYWAYTWASAQG | |
| | | KASAIAPTIAKAAKLGDYLRYSLFDKYFKQVGNCYPASSCPGATGRQS | |
| | | ETYLIGWYYAWGGSSQGWAWRIGDGAAHFGYQNPLAAWAMSNVTPLIP | |
| | | LSPTAKSDWAASLQRQLEFYQWLQSAEGAIAGGATNSWNGNYGTPPAG | |
| | | DSTFYGMAYDWEPVYHDPPSNNWFGFQAWSMERVAEYYYVTGDPKAKA | |
| | | LLDKWVAWVKPNVTTGASWSIPSNLSWSGQPDTWNPSNPGTNANLHVT | |
| | | ITSSGQDVGVAAALAKTLEYYAAKSGDTASRDLAKGLLDSIWNNDQDS | |
| | | LGVSTPETRTDYSRFTQVYDPTTGDGLYIPSGWTGTMPNGDQIKPGAT | |
| | | FLSIRSWYTKDPQWSKVQAYLNGGPAPTFNYHRFWAESDFAMANADFG | |
| | | MLFPSGSPSPTPSPTPTSSPSPTPSSSPTPSPSPSPTGDTTPPSVPTG | |
| | | LQVTGTTTSSVSLSWTASTDNVGVAHYNVYRNGTLVGQPTATSFTDTG | |
| | | LAAGTSYTYTVAAVDAAGNTSAQSSPVTATTASPSPSPSPSPTPTSSP | |
| | | SPTPSPTPSPTSTSGASCTATYVVNSDWGSGFTTTVTVTNTGTRATSG | |
| | | WTVTWSFAGNQTVTNYWNTALTQSGKSVTAKNLSYNNVIQPGQSTTFG | |
| | | FNGSYSGTNTAPTLSCTASZ | |
| XylE | Acidothermus | MGHHAMRRMVTSASVVGVATLAAATVLITGGIAHAASTLKQGAEANGR | ABK51955.1 |
| | cellulolyticus | YFGVSASVNTLNNSAAANLVATQFDMLTPENEMKWDTVESSRGSFNFG | |
| | | PGDQIVAFATAHNMRVRGHNLVWHSQLPGWVSSLPLSQVQSAMESHIT | |
| | | AEVTHYKGKIYAWDVVNEPFDDSGNLRTDVFYQAMGAGYIADALRTAH | |
| | | AADPNAKLYLNDYNIEGINAKSDAMYNLIKQLKSQGVPIDGVGFESHF | |
| | | IVGQVPSTLQQNMQRFADLGVDVAITELDDRMPTPPSQQNLNQQATDD | |
| | | ANVVKACLAVARCVGITQWDVSDADSWVPGTFSGQGAATMFDSNLQPK | |
| | | PAFTAVLNALSASASVSPSPSPSPSPSPSPSPSPSPSPSPSPSPSPSP | |
| | | SSSPVSGGVKVQYKNNDSAPGDNQIKPGLQVVNTGSSSVDLSTVTVRY | |
| | | WFTRDGGSSTLVYNCDWAVMGCGNIRASFGSVNPATPTADTYLQLSFT | |
| | | GGTLPAGGSTGEIQSRVNKSDWSNFTETNDYSYGTNTTFQDWSKVTVY | |
| | | VNGRLVWGTEPSGTSPSPTPSPSPTPSPSPSPSPSPSPSPSPSPSPSP | |
| | | SSSPSSGCVASMRVDSSWPGGFTATVTVSNTGGVSTSGWQVGWSWPSG | |
| | | DSLVNAWNAVVSVTGTSVRAVNASYNGVIPAGGSTTFGFQANGTPGTP | |
| | | TFTCTTSADLZ | |
| aviIII | Acidothermus | MAATTQPYTWSNVAIGGGGFVDGIVFNEGAPGILYVRTDIGGMYRWDA | ABK52391.1 |
| | cellulolyticus | ANGRWIPLLDWVGWNNWGYNGVVSIAADPINTNKVWAAVGMYTNSWDP | |
| | | NDGAILRSSDQGATWQITPLPFKLGGNMPGRGMGERLAVDPNNDNILY | |
| | | FGAPSGKGLWRSTDSGATWSQMTNFPDVGTYIANPTDTTGYQSDIQGV | |
| | | VWVAFDKSSSSLGQASKTIFVGVADPNNPVFWSRDGGATWQAVPGAPT | |
| | | GFIPHKGVFDPVNHVLYIATSNTGGPYDGSSGDVWKFSVTSGTWTRIS | |
| | | PVPSTDTANDYFGYSGLTIDRQHPNTIMVATQISWWPDTIIFRSTDGG | |
| | | ATWTRIWDWTSYPNRSLRYVLDISAEPWLTFGVQPNPPVPSPKLGWMD | |
| | | EAMAIDPFNSDRMLYGTGATLYATNDLTKWDSGGQIHIAPMVKGLEET | |
| | | AVNDLISPPSGAPLISALGDLGGFTHADVTAVPSTIFTSPVFTTGTSV | |
| | | DYAELNPSIIVRAGSFDPSSQPNDRHVAFSTDGGKNWFQGSEPGGVTT | |
| | | GGTVAASADGSRFVWAPGDPGQPVVYAVGFGNSWAASQGVPANAQIRS | |
| | | DRVNPKTFYALSNGTFYRSTDGGVTFQPVAAGLPSSGAVGVMFHAVPG | |
| | | KEGDLWLAASSGLYHSTNGGSSWSAITGVSSAVNVGFGKSAPGSSYPA | |
| | | VFVVGTIGGVTGAYRSDDGGTTWVRINDDQHQYGNWGQAITGDPRIYG | |
| | | RVYIGTNGRGIVYGDIAGAPSGSPSPSVSPSASPSLSPSPSPSSSPSP | |
| | | SPSPSSSPSSSPSPSPSPSPSPSRSPSPSASPSPSSSPSPSSSPSSSP | |
| | | SPTPSSSPVSGGVKVQYKNNDSAPGDNQIKPGLQVVNTGSSSVDLSTV | |
| | | TVRYWFTRDGGSSTLVYNCDWAAIGCGNIRASFGSVNPATPTADTYLQ | |
| | | LSFTGGTLAAGGSTGEIQNRVNKSDWSNFTETNDYSYGTNTVFQDWSK | |
| | | VTVYVNGRLVWGTEPSGTSPSPTPSPSPTPSPSPSPSPGGDVTPPSVP | |
| | | TGVVVTGVSGSSVSLAWNASTDNVGVAHYNVYRNGVLVGQPTVTSFTD | |
| | | TGLAAGTAYTYTVAAVDAAGNTSAPSTPVTATTTSPSPSPSPTPSPTP | |
| | | SPTPSPSPSPSLSPSPSPSPSPSPSPSLSPSPSTSPSPSPSPTPSPSS | |
| | | SGVGCRATYVVNSDWGSGFTATVTVTNTGSRATSGWTVAWSFGGNQTV | |
| | | TNYWNTLLTQSGASVTATNLSYNNVIQPGQSTTFGFNATYAGTNTPPT | |
| | | PTCTTNSD | |
| XylE | Acidothermus | MGHHAMRRMVTSASVVGVATLAAATVLITGGIAHAASTLKQGAEANGR | ABK51955.1 |
| | cellulolyticus | YFGVSASVNTLNNSAAANLVATQFDMLTPENEMKWDTVESSRGSFNFG | |
| | | PGDQIVAFATAHNMRVRGHNLVWHSQLPGWVSSLPLSQVQSAMESHIT | |
| | | AEVTHYKGKIYAWDVVNEPFDDSGNLRTDVFYQAMGAGYIADALRTAH | |
| | | AADPNAKLYLNDYNIEGINAKSDAMYNLIKQLKSQGVPIDGVGFESHF | |
| | | IVGQVPSTLQQNMQRFADLGVDVAITELDDRMPTPPSQQNLNQQATDD | |
| | | ANVVKACLAVARCVGITQWDVSDADSWVPGTFSGQGAATMFDSNLQPK | |
| | | PAFTAVLNALSASASVSPSPSPSPSPSPSPSPSPSPSPSPSPSPSPSP | |
| | | SSSPVSGGVKVQYKNNDSAPGDNQIKPGLQVVNTGSSSVDLSTVTVRY | |
| | | WFTRDGGSSTLVYNCDWAVMGCGNIRASFGSVNPATPTADTYLQLSFT | |
| | | GGTLPAGGSTGEIQSRVNKSDWSNFTETNDYSYGTNTTFQDWSKVTVY | |
| | | VNGRLVWGTEPSGTSPSPTPSPSPTPSPSPSPSPSPSPSPSPSPSPSP | |
| | | SSSPSSGCVASMRVDSSWPGGFTATVTVSNTGGVSTSGWQVGWSWPSG | |
| | | DSLVNAWNAVVSVTGTSVRAVNASYNGVIPAGGSTTFGFQANGTPGTP | |
| | | TFTCTTSADLZ | |
| aviIII | Acidothermus | MAATTQPYTWSNVAIGGGGFVDGIVFNEGAPGILYVRTDIGGMYRWDA | ABK52391.1 |
| | cellulolyticus | ANGRWIPLLDWVGWNNWGYNGVVSIAADPINTNKVWAAVGMYTNSWDP | |
| | | NDGAILRSSDQGATWQITPLPFKLGGNMPGRGMGERLAVDPNNDNILY | |
| | | FGAPSGKGLWRSTDSGATWSQMTNFPDVGTYIANPTDTTGYQSDIQGV | |
| | | VWVAFDKSSSSLGQASKTIFVGVADPNNPVFWSRDGGATWQAVPGAPT | |
| | | GFIPHKGVFDPVNHVLYIATSNTGGPYDGSSGDVWKFSVTSGTWTRIS | |
| | | PVPSTDTANDYFGYSGLTIDRQHPNTIMVATQISWWPDTIIFRSTDGG | |
| | | ATWTRIWDWTSYPNRSLRYVLDISAEPWLTFGVQPNPPVPSPKLGWMD | |
| | | EAMAIDPFNSDRMLYGTGATLYATNDLTKWDSGGQIHIAPMVKGLEET | |
| | | AVNDLISPPSGAPLISALGDLGGFTHADVTAVPSTIFTSPVFTTGTSV | |
| | | DYAELNPSIIVRAGSFDPSSQPNDRHVAFSTDGGKNWFQGSEPGGVTT | |
| | | GGTVAASADGSRFVWAPGDPGQPVVYAVGFGNSWAASQGVPANAQIRS | |
| | | DRVNPKTFYALSNGTFYRSTDGGVTFQPVAAGLPSSGAVGVMFHAVPG | |
| | | KEGDLWLAASSGLYHSTNGGSSWSAITGVSSAVNVGFGKSAPGSSYPA | |
| | | VFVVGTIGGVTGAYRSDDGGTTWVRINDDQHQYGNWGQAITGDPRIYG | |
| | | RVYIGTNGRGIVYGDIAGAPSGSPSPSVSPSASPSLSPSPSPSSSPSP | |
| | | SPSPSSSPSSSPSPSPSPSPSPSRSPSPSASPSPSSSPSPSSSPSSSP | |
| | | SPTPSSSPVSGGVKVQYKNNDSAPGDNQIKPGLQVVNTGSSSVDLSTV | |
| | | TVRYWFTRDGGSSTLVYNCDWAAIGCGNIRASFGSVNPATPTADTYLQ | |
| | | LSFTGGTLAAGGSTGEIQNRVNKSDWSNFTETNDYSYGTNTVFQDWSK | |
| | | VTVYVNGRLVWGTEPSGTSPSPTPSPSPTPSPSPSPSPGGDVTPPSVP | |
| | | TGVVVTGVSGSSVSLAWNASTDNVGVAHYNVYRNGVLVGQPTVTSFTD | |
| | | TGLAAGTAYTYTVAAVDAAGNTSAPSTPVTATTTSPSPSPSPTPSPTP | |
| | | SPTPSPSPSPSLSPSPSPSPSPSPSPSLSPSPSTSPSPSPSPTPSPSS | |
| | | SGVGCRATYVVNSDWGSGFTATVTVTNTGSRATSGWTVAWSFGGNQTV | |
| | | TNYWNTLLTQSGASVTATNLSYNNVIQPGQSTTFGFNATYAGTNTPPT | |
| | | PTCTTNSD | |
| cbhE | Talaromyces | MDPQQAGTATAENHPPLTWQECTAPGSCTTQNGAVVLDANWRWVHDVN | AAL33602.2 |
| | emersonii | GYTNCYTGNTWDPTYCPDDETCAQNCALDGADYEGTYGVTSSGSSLKL | |
| | | NFVTGSNVGSRLYLLQDDSTYQIFKLLNREFSFDVDVSNLPCGLNGAL | |
| | | YFVAMDADGGVSKYPNNKAGAKYGTGYCDSQCPRDLKFIDGEANVEGW | |
| | | QPSSNNANTGIGDHGSCCAEMDVWEANSISNAVTPHPCDTPGQTMCSG | |
| | | DDCGGTYSNDRYAGTCDPDGCDFNPYRMGNTSFYGPGKIIDTTKPFTV | |
| | | VTQFLTDDGTDTGTLSEIKRFYIQNSNVIPQPNSDISGVTGNSITTEF | |
| | | CTAQKQAFGDTDDFSQHGGLAKMGAAMQQGMVLVMSLDDYAAQMLWLD | |
| | | SDYPTDADPTTPGIARGTCPTDSGVPSDVESQSPNSYVTYSNIKFGPI | |
| | | NSTFTASGD | |

A—Cellulases

Cellulases are lignocellulolytic enzyme polypeptides involved in cellulose degradation. Cellulase enzyme polypeptides are classified on the basis of their mode of action. There are two basic kinds of cellulases: the endocellulases, which cleave the polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrate, yielding mainly higher oligomers. Cellulobiohydrolases are exocellulases which hydrolyze crystalline cellulose and release cellobiose (glucose dimer). Both types of enzymes hydrolyze β-1,4-glycosidic bonds. β-D glucosidases or cellulobiase converts oligosaccharides and cellubiose to glucose. Beta-glucan glucohydrolase hydrolyzes oligosaccharides to glucose.

According to the present invention, the heterologous gene may encode a cellulase enzyme polypeptide. Transgenic plants of the invention may be engineered to comprise one or more than one gene encoding a cellulase enzyme polypeptide. For example, plants may be engineered to comprise one or more genes encoding a cellulase of the cellubiohydrolase class, one or more genes encoding a cellulase of the endoglucanase class, and/or one or more genes encoding a cellulase of the β-D glucosidase class.

Examples of endoglucanase genes that can be used in the present invention include those that can be obtained from Aspergillus aculeatus (U.S. Pat. No. 6,623,949; WO 94/14953), Aspergillus kawachii (U.S. Pat. No. 6,623,949), Aspergillus oryzae (Kitamoto et al., Appl. Microbiol. Biotechnol., 1996, 46: 538-544; U.S. Pat. No. 6,635,465), Aspergillus nidulans (Lockington et al., Fungal Genet. Biol., 2002, 37: 190-196), Cellulomonas fimi (Wong et al., Gene, 1986, 44: 315-324), Bacillus subtilis (MacKay et al., Nucleic Acids Res., 1986, 14: 9159-9170), Cellulomonas pachnodae (Cazemier et al., Appl. Microbiol. Biotechnol., 1999, 52: 232-239), Fusarium equiseti (Goedegebuur et al., Curr. Genet., 2002, 41: 89-98), Fusarium oxysporum (Hagen et al., Gene, 1994, 150: 163-167; Sheppard et al., Gene, 1994, 150: 163-167), Humicola insolens (U.S. Pat. No. 5,912,157; Davies et al., Biochem J., 2000, 348: 201-207), Hypocrea jecorina (Penttila et al., Gene, 1986, 45: 253-263), Humicola grisea (Goedegebuur et al., Curr. Genet., 2002, 41: 89-98), Micromonospora cellulolyticum (Lin et al., J. Ind. Microbiol., 1994, 13: 344-350), Myceliophthora thermophile (U.S. Pat. No. 5,912,157), Rhizopus oryzae (Moriya et al., J. Bacteriol., 2003, 185: 1749-1756), Trichoderma reesei (Saloheimo et al., Mol. Microbiol., 1994, 13: 219-228), and Trichoderma viride (Kwon et al., Biosci. Biotechnol. Biochem., 1999, 63: 1714-1720; Goedegebuur et al., Curr. Genet., 2002, 41: 89-98).

In certain embodiments, the heterologous gene encodes the endo-1,4-β-glucanase E1 gene (GenBank Accession No. U33212, See Table 1). This gene was isolated from the thermophilic bacterium Acidothermus cellulolyticus. Acidothermus cellulolyticus has been characterized with the ability to hydrolyze and degrade plant cellulose. The cellulase complex produced by A. cellulolyticus is known to contain several different thermostable cellulase enzymes with maximal activities at temperatures of 75° C. to 83° C. These cellulases are resistant to inhibition from cellobiose, an end product of the reactions catalyzed by endo- and exo-cellulases.

The E1 endo-1,4-β-glucanase is described in detail in U.S. Pat. No. 5,275,944. This endoglucanase demonstrates a temperature optimum of 83° C. and a specific activity of 40 μmol glucose release from carboxymethylcellulose/min/mg protein. This E1 endoglucanase was further identified as having an isoelectric pH of 6.7 and a molecular weight of 81,000 Daltons by SDS polyacrylamide gel electrophoresis. It is synthesized as a precursor with a signal peptide that directs it to the export pathway in bacteria. The mature enzyme polypeptide is 521 amino acids (aa) in length. The crystal structure of the catalytic domain of about 40 kD (358 aa) has been described (J. Sakon et al., Biochem., 1996, 35: 10648-10660). Its pro/thr/ser-rich linker is 60 aa, and the cellulose binding domain (CBD) is 104 aa. The properties of the cellulose binding domain that confer its function are not well-characterized. Plant expression of the E1 gene has been reported (see for example, M. T. Ziegler et al., Mol. Breeding, 2000, 6: 37-46; Z. Dai et al., Mol. Breeding, 2000, 6: 277-285; Z. Dai et al., Transg. Res., 2000, 9: 43-54; and T. Ziegelhoffer et al., Mol. Breeding, 2001, 8: 147-158).

Examples of cellobiohydrolase genes that can be used in the present invention can be obtained from Acidothermus cellulolyticus, Acremonium cellulolyticus (U.S. Pat. No. 6,127,160), Agaricus bisporus (Chow et al., Appl. Environ. Microbiol., 1994, 60: 2779-2785), Aspergillus aculeatus (Takada et al., J. Ferment. Bioeng., 1998, 85: 1-9), Aspergillus niger (Gielkens et al., Appl. Environ. Microbiol., 65: 1999, 4340-4345), Aspergillus oryzae (Kitamoto et al., Appl. Microbiol. Biotechnol., 1996, 46: 538-544), Athelia rolfsii (EMBL accession No. AB103461), Chaetomium thermophilum (EMBL accession Nos. AX657571 and CQ838150), Cullulomonas fimi (Meinke et al., Mol. Microbiol., 1994, 12: 413-422), Emericella nidulans (Lockington et al., Fungal Genet. Biol., 2002, 37: 190-196), Fusarium oxysporum (Hagen et al., Gene, 1994, 150: 163-167), Geotrichum sp. 128 (EMBL accession No. AB089343), Humicola grisea (de Oliviera and Radford, Nucleic Acids Res., 1990, 18: 668; Takashima et al., J. Biochem., 1998, 124: 717-725), Humicola nigrescens (EMBL accession No. AX657571), Hypocrea koningii (Teeri et al., Gene, 1987, 51: 43-52), Mycelioptera thermophila (EMBL accession No. AX657599), Neocallimastix patriciarum (Denman et al., Appl. Environ. Microbiol., 1996, 62: 1889-1896), Phanerochaete chrysosporium (Tempelaars et al., Appl. Environ. Microbiol., 1994, 60: 4387-4393), Thermobifida fusca (Zhang, Biochemistry, 1995, 34: 3386-3395), Trichoderma reesei (Terri et al., BioTechnology, 1983, 1: 696-699; Chen et al., BioTechnology, 1987, 5: 274-278), and Trichoderma viride (EMBL accession Nos. A4368686 and A4368688).

Examples of β-D-glucosidase genes that can be used in the present invention can be obtained from Aspergillus aculeatus (Kawaguchi et al., Gene, 1996, 173: 287-288), Aspergillus kawachi (Iwashita et al., Appl. Environ. Microbiol., 1999, 65: 5546-5553), Aspergillus oryzae (WO 2002/095014), Cellulomonas biazotea (Wong et al., Gene, 1998, 207: 79-86), Penicillium funiculosum (WO 200478919), Saccharomycopsis fibuligera (Machida et al., Appl. Environ. Microbiol., 1988, 54: 3147-3155), Schizosaccharomyces pombe (Wood et al., Nature, 2002, 415: 871-880), and Trichoderma reesei (Barnett et al., BioTechnology, 1991, 9: 562-567).

Other examples of cellulases that can be used in accordance with the present invention include family 48 glycoside hydrolases such as gux1 from Acidothermus cellulolyticus, avicelases such as aviIII from Acidothermus cellulolyticus, and cbhE from Talaromyces emersonii. (See Table 1.)

Transgene expression of cellulases in plants for the conversion of cellulose to glucose has been reported (see, for example, Y. Jin Cai et al., Appl. Environ. Microbiol., 1999, 65: 553-559; C. R. Sanchez et al., Revista de Microbiologica, 1999, 30: 310-314; R. Cohen et al., Appl. Environ., 2995, 71: 2412-2417; Z. Dai et al., Transg. Res., 2005, 14: 627-543).

B—Hemicellulases

Hemicellulases are lignocellulolytic enzyme polypeptides that are involved in hemicellulose degradation. Hemicellulases include xylanases, arabinofuranosidases, acetyl xylan esterases, ferulic acid esterases, xyloglucanases, β-glucanases, β-xylosidases, glucuronidases, mannanases, galactanases, and arabinases. Similar to cellulase enzyme polypeptides, hemicellulases are classified on the basis of their mode of action: the endo-acting hemicellulases attack internal bonds within the polysaccharide chain; the exo-acting hemicellulases act progressively from either the reducing or non-reducing end of polysaccharide chains.

According to the present invention, heterologous genes may encode a hemicellulase enzyme polypeptide. Transgenic plants of the invention may be engineered to comprise one or more than one gene encoding a hemicellulase enzyme polypeptide. For example, plants may be engineered to comprise one or more genes encoding a hemicellulase of the xylanase class, one or more genes encoding a hemicellulase of the arabinofuranosidase class, one or more genes encoding a hemicellulase of the acetyl xylan esterase class, one or more genes encoding a hemicellulase of the glucuronidase class, one or more genes encoding a hemicellulase of the mannanase class, one or more genes encoding a hemicellulase of the galactanase class, and/or one or more genes encoding a hemicellulase of the arabinase class.

Examples of endo-acting hemicellulases include endoarabinanase, endoarabinogalactanase, endoglucanase, endomannanase, endoxylanase, and feraxan endoxylanase. Examples of exo-acting hemicellulases include α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-mannobiohydrolase, exo-mannanase, exo-xylanase, xylan α-glucuronidase, and coniferin β-glucosidase.

Hemicellulase genes can be obtained from any suitable source, including fungal and bacterial organisms, such as Aspergillus, Disporotrichum, Penicillium, Neurospora, Fusarium, Trichoderma, Humicola, Thermomyces, and Bacillus. Examples of hemicellulases that can be used in the present invention can be obtained from Acidothermus cellulolyticus, Acidobacterium capsulatum (Inagaki et al., Biosci. Biotechnol. Biochem., 1998, 62: 1061-1067), Agaricus bisporus (De Groot et al., J. Mol. Biol., 1998, 277: 273-284), Aspergillus aculeatus (U.S. Pat. No. 6,197,564; U.S. Pat. No. 5,693,518), Aspergillus kawachii (Ito et al., Biosci. Biotechnol. Biochem., 1992, 56: 906-912), Aspergillus niger (EMBL accession No. AF108944), Magnaporthe grisea (Wu et al., Mol. Plant Microbe Interact., 1995, 8: 506-514), Penicillium chrysogenum (Haas et al., Gene, 1993, 126: 237-242), Talaromyces emersonii (WO 02/24926), and Trichoderma reesei (EMBL accession Nos. X69573, X69574, and AY281369).

In certain embodiments, the heterologous gene comprises the A. cellulolyticus endoxylanase xylE.

C—Ligninases

Ligninases are lignocellulolytic enzyme polypeptides that are involved in the degradation of lignin. Lignin-degrading enzyme polypeptides include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as co-substrate by the peroxidases, can be generated by glucose oxidase, aryl alcohol oxidase, and/or lignin peroxidase-activated glyoxal oxidase.

According to the present invention, heterologous genes may encode a ligninase enzyme polypeptide. Transgenic plants of the invention may be engineered to comprise one or more than one gene encoding a ligninase enzyme polypeptide. For example, plants may be engineered to comprise one or more genes encoding a ligninase of the lignin peroxidase class, one or more genes encoding a ligninase of the manganese-dependent peroxidase class, one or more genes encoding a ligninase of the hybrid peroxidase class, and/or one or more genes encoding a ligninase of the laccase class.

Lignin-degrading genes may be obtained from Acidothermus cellulolyticus, Bjerkandera adusta, Ceriporiopsis subvermispora (see WO 02/079400), Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride.

Examples of genes encoding ligninases that can be used in the invention can be obtained from Bjerkandera adusta (WO 2001/098469), Ceriporiopsis subvermispora (Conesa et al., J. Biotechnol., 2002, 93: 143-158), Cantharellus cibariusi (Ng et al., Biochem. and Biophys. Res. Comm., 2004, 313: 37-41), Coprinus cinereus (WO 97/008325; Conesa et al., J. Biotechnol., 2002, 93: 143-158), Lentinula edodes (Nagai et al., Applied Microbiol. and Biotechnol., 2002, 60: 327-335, 2002), Melanocarpus albomyces (Kiiskinen et al., FEBS Letters, 2004, 576: 251-255, 2004), Myceliophthora thermophile (WO 95/006815), Phanerochaete chrysosporium (Conesa et al., J. Biotechnol., 2002, 93: 143-158; Martinez, Enz, Microb, Technol, 2002, 30: 425-444), Phlebia radiata (Conesa et al., J. Biotechnol., 2002, 93: 143-158), Pleurotus eryngii (Conesa et al., J. Biotechnol., 2002, 93: 143-158), Polyporus pinsitus (WO 96/000290), Rigidoporus lignosus (Garavaglia et al., J. of Mol. Biol., 2004, 342: 1519-1531), Rhizoctonia solani (WO 96/007988), Scytalidium thermophilum (WO 95/033837), Tricholoma giganteum (Wang et al., Biochem. Biophys. Res. Comm., 2004, 315: 450-454), and Trametes versicolor (Conesa et al., J. Biotechnol., 2002, 93: 143-158).

For example, transgenic plants of the invention may be engineered to comprise one or more lignin peroxidases. Genes encoding lignin peroxidases may be obtained from Phanerochaete chrysosporium or Phlebia radiata. Lignin-peroxidases are glycosylated heme proteins (MW 38 to 46 kDa) which are dependent on hydrogen peroxide for activity and catalyze the oxidative cleavage of lignin polymer. At least six (6) heme proteins (H1, H2, H6, H7, H8 and H10) with lignin peroxidase activity have been identified Phanerochaete chrysosporium in strain BKMF-1767. In certain embodiments, plants are engineered to comprise the white rot filamentous Phanerochaete chrysosporium ligninase (CGL5) (H. A. de Boer et al., Gene, 1988, 69(2): 369) (see the Examples section).

D—Other Lignocellulolytic Enzyme Polypeptides

In addition to cellulases, hemicellulases and ligninases, lignocellulolytic enzyme polypeptides that can be used in the practice of the present invention also include enzymes that degrade pectic substances or phenolic acids such as ferulic acid. Pectic substances are composed of homogalacturonan (or pectin), rhamno-galacturonan, and xylogalacturonan. Enzymes that degrade homogalacturonan include pectate lyase, pectin lyase, polygalacturonase, pectin acetyl esterase, and pectin methyl esterase. Enzymes that degrade rhamnogalacturonan include alpha-arabinofuranosidase, beta-galactosidase, galactanase, arabinanase, alpha-arabinofuranosidase, rhamnogalacturonase, rhamnogalacturonan lyase, and rhamnogalacturonan acetyl esterase. Enzymes that degrade xylogalacturonan include xylogalacturonosidase, xylogalacturonase, and rhamnogalacturonan lyase.

Phenolic acids include ferulic acid, which functions in the plant cell wall to cross-link cell wall components together. For example, ferulic acid may cross-link lignin to hemicellulose, cellulose to lignin, and/or hemicellulose polymers to each other. Ferulic acid esterases cleave ferulic acid, disrupting the cross linkages.

Other enzymes that may enhance or promote lignocellulose disruption and/or degradation may be expressed under the control of a gene regulatory element provided in the present disclosure and include, but are not limited to, amylases (e.g., alpha amylase and glucoamylase), esterases, lipases, phospholipases, phytases, proteases, and peroxidases.

E—Combinations of Lignocellulolytic Enzyme Polypeptides

According to the present invention, heterologous genes may encode a lignocellulolytic enzyme polypeptide, e.g., a cellulase enzyme polypeptide, a hemicellulase enzyme polypeptide, or a ligninase enzyme polypeptide. Transgenic plants of the invention may be engineered to comprise one or more than one gene encoding lignocellulolytic enzyme polypeptides, e.g., enzymes from different classes of cellulases, enzymes from different classes of hemicellulases, enzymes from different classes of ligninases, or any combinations thereof. For example, combinations of genes may be selected to provide efficient degradation of one component of lignocellulose (e.g., cellulose, hemicellulose, or lignin). Alternatively, combinations of genes may be selected to provide efficient degradation of the lignocellulosic material.

In certain embodiments, genes are optimized for the substrate (e.g., cellulose, hemicellulase, lignin or whole lignocellulosic material) in a particular plant (e.g., corn, tobacco, switchgrass). Tissue from one plant species is likely to be physically and/or chemically different from tissue from another plant species. Selection of genes or combinations of genes to achieve efficient degradation of a given plant tissue is within the skill of artisans in the art.

In some embodiments, combinations of genes are selected to provide for synergistic enzyme activity (i.e., genes are selected such that the interaction between distinguishable enzyme polypeptides or enzyme activities results in the total activity of the enzymes taken together being greater than the sum of the effects of the individual activities).

Efficient lignocellulolytic activity may be achieved by production of two or more enzyme polypeptides in a single transgenic plant. As mentioned above, plants may be transformed to express more than one enzyme polypeptide, for example, by employing the use of multiple gene constructs encoding each of the selected enzymes or a single construct comprising multiple nucleotide sequences encoding each of the selected enzymes. Alternatively, individual transgenic plants, each stably transformed to express a given enzyme, may be crossed by methods known in the art (e.g., pollination, hand detassling, cytoplasmic male sterility, and the like) to obtain a resulting plant that can produce all the enzymes of the individual starting plants.

Alternatively or additionally, efficient lignocellulolytic activity may be achieved by production of two or more lignocellulolytic enzyme polypeptides in separate plants. For example, three separate lines of plants (e.g., corn), one expressing one or more enzymes of the cellulase class, another expressing one or more enzymes of the hemicellulase class and the third one expressing one or more enzymes of the ligninase class, may be developed and grown simultaneously. The desired “blend” of enzymes produced may be achieved by simply changing the seed ratio, taking into account farm climate and soil type, which are expected to influence enzyme yields in plants.

Other advantages of this approach include, but are not limited to, increased plant health (which is known to be adversely affected as the number of introduced genes increases), simpler transformations procedures and great flexibility in incorporating the desired traits in commercial plant varieties for large-scale production.

G—Thermophilic and Thermostable Enzyme Polypeptides

It may be sometimes desirable to expressing thermophilic and/or thermostable enzyme polypeptides. Gene regulatory elements provided by the presnt invention may be used to drive and/or facilitate expresion of genes ecncoding such polypeptides as well. For example, enzyme polypeptides whose optimal range of temperature for activity (thermophilic enzyme polypeptides) may be expressed in transgenic plants in accordance with the invention. Without wishing to be bound by any particular theory, the limited activity or absence of activity during growth of the plant (at moderate or low temperatures, at which the enzyme polypeptide is less active) may be beneficial to the health of the plant. Alternatively or additionally, and without wishing to be bound by any particular theory, such enzyme polypeptides may facilitate increased hydrolysis because of their high activity at high temperature conditions commonly used in the processing of cellulosic biomass.

In some embodiments, the present invention provides a transgenic plant, the genome of which is augmented with a recombinant polynucleotide encoding at least one lignocellulolytic enzyme polypeptide that exhibits low activity at a temperature below about 60° C., below about 50° C., below about 40° C., or below about 30° C. In some embodiments, the present invention provides a transgenic plant, the genome of which is augmented with a recombinant polynucleotide encoding at least one lignocellulolytic enzyme polypeptide that exhibits high activity at a temperature above about 50° C., above about 60° C., above about 70° C., above about 80° C., or above about 90° C.

In some embodiments, the present invention provides a transgenic plant, the genome of which is augmented with a recombinant polynucleotide encoding at least one lignocellulolytic enzyme polypeptide that is or is homologous to a lignocellulolytic enzyme polypeptide found in a thermophilic microorganism (e.g., bacterium, fungus, etc.). In some such embodiments, the thermophilic organism is a bacterium that is a member of a genus selected from the group consisting of Aeropyrum, Acidilobus, Acidothermus, Aciduliprofundum, Anaerocellum, Archaeoglobus, Aspergillus, Bacillus, Caldibacillus, Caldicellulosiruptor, Caldithrix, Cellulomonas, Chaetomium, Chloroflexus, Clostridium, Cyanidium, Deferribacter, Desulfotomaculum, Desulfurella, Desulfurococcus, Fervidobacterium, Geobacillus, Geothermobacterium, Humicola, Ignicoccus, Marinitoga, Methanocaldococcus, Methanococcus, Methanopyrus, Methanosarcina, Methanothermobacter, Nautilia, Pyrobaculum, Pyrococcus, Pyrodictium, Rhizomucor, Rhodothermus, Staphylothermus, Scylatidium, Spirochaeta, Sulfolobus, Talaromyces, Thermoascus, Thermobifida, Thermococcus, Thermodesulfobacterium, Thermodesulfovibrio, Thermomicrobium, Thermoplasma, Thermoproteus, Thermothrix, Thermotoga, Thermus, and Thiobacillus; in some such embodiments, the thermophilic microorganism is a bacterium that is a member of a species selected from the group consisting of Acidothermus cellulolyticus, Pyrococcus furiosus, and Talaromyces emersonii.

ii. Cell Wall-Modifying Enzyme Polypeptides

In some embodiments, the heterologous gene (whose expression is driven by a provided gene regulatory element) encodes a cell wall-modifying enzyme polypeptide described in U.S. patent application Ser. No. 12/476,247 (filed on Jun. 1, 2009), the contents of which are herein incorporated by reference in their entirety. In some embodiemnts, cell wall-modifying enzyme polypeptides are lignocelluloytic enzyme polypeptides

Cell wall-modifying enzyme polypeptides useful in accordance with the present invention include those having at least 50%, 60%, 70%, 80% or more overall sequence identity with a polypeptide whose amino acid sequence is set forth in Table 1 of U.S. patent application Ser. No. 12/476,247. Alternatively or additionally, in some embodiments, cell wall-modifying enzyme polypeptide shows at least 90%, 95%, 96%, 97%, 98%, 99%, or greater identity with at least one sequence element found in a polypeptide whose amino acid sequence is set forth in Table 1 of U.S. patent application Ser. No. 12/476,247, which sequence element is at least 4, 5, 6, 7, 8, 9, 10, 11, 12, 13, 14, 15, 16, 17, 18, 19, 20 or more amino acids long.

A variety of organisms produce cell wall-modifying enzyme polypeptides. Cell wall-modifying enzyme polypeptides may have, for example, archael, fungal, insect, animal, or plant origins.

In some embodiments, the cell wall-modifying enzyme polypeptide has cellulase activity. In some embodmients, the cell wall-modifying enzyme polypeptide has an activity selected from the group consisting of feruloyl esterase (also known as ferulic acid esterase), xylanase, alpha-L-arabinofuranosidase, endogalactanase, acetylxylan esterase, beta-xylosidase, xyloglucanase, glucuronoyl esterase, endo-1,5-alpha-L-arabinosidase, pectin methylesterase, endopolygalacturonase, exopolygalacturonase, pectin lyase, pectate lyase, rhamnogalacturonan lyase, pectin acetylesterase, alpha-L-rhamnosidase, mannanase, exoglucanase, glucan glycohydrolase, licheninase, laminarinase, beta-(1,3)-(1,4)-glucanase and beta-glucosidase activity. Such activities may be similar to that of other enzyme polypeptides, including those known in the art that are classified by an EC class and/or listed in enzyme databases (such as CaZY, www.cazy.org, which lists carbohydrate-active enzymes).

In some embodiments, the cell wall-modifying enzyme polypeptide modifies a plant cell wall component. In many such embodiments, the cell wall-modifying enzyme polypeptide modifies the plant cell wall component in such a way that the plant biomass is more amenable to processing steps (e.g., enzymatic digestion). For example, cell wall-modifying enzyme polypeptides may modify plant cell wall components in such a way as to allow increased digestability, increased hydrolysis, and/or increased sugar yields.

In some embodiments, modifying comprises cleavage and/or hydrolysis of the plant cell wall component. Examples of plant cell wall components that may be modified include, but are not limited to, xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof.

In some embodiments, the cell wall-modifying enzyme polypeptide disrupts an interaction in the plant biomass such as a covalent linkage, an ionic bonding interaction, a hydrogen bonding interaction, or a combination thereof. Examples of linkages that may be disrupted include, but are not limited to, hemicellulose-cellulose-lignin, hemicellulose-cellulose-pectin, hemicellulose-diferululate-hemicellulose, hemicellulose-ferulate-lignin, mixed beta-D-glucan-cellulose, mixed-beta-D-glucan-hemicellulose, pectin-ferulate-lignin linkages, and combinations thereof. In some embodiments, disrupting comprises hydrolyzing a linkage, such as a feruloyl ester linkage.

b. Heterologous Gene Products Conferring Resistance to Pests, Disease, and Environmental Stress

Heterologous genes may express products that confer benefit(s) to the transgenic plant such as herbicide resistance, insect resistance, disease resistance, resistance against parasites, and/or increased tolerance to environmental stress (e.g., drought).

Herbicide Resistance

A number of gene products are known in the art that can confer resistance to herbicides. For example, glyphosate (N-(phosphonomethyl)glycine) is a broad-spectrum systemic herbicide and the active ingredient of ROUNDUP™ formulations. Glyphosate acts by inhibiting 5-enolpyruvoyl-shikimate-3-phosphate synthetase (EPSPS) (encoded in some organisms by the aroA gene), starving the affected cells for aromatic amino acids. Some micro-organisms have a mutant form of EPSPS that is resistant to glyphosate inhibition, and this form of the enzyme can be used to impart glyphosate resistance.

As a further example, the herbicide bromoxynil (marketed as Buctril) is applied post-emergence to kill broadleaf weeds, and works by inhibiting photosynthesis in plants. Bromoxynil nitrilase (BXN), a gene from the bacterium Klebsiella pneumoniae, detoxifies bromoxynil in genetically engineered plants and therefore can confer resistance to herbicides.

The L-isomer of phosphinothricin (PPT, glufosinate ammonium) is the active ingredient of several commercial broad spectrum herbicide formulation. An analogue of L-glutamic acid, PPT, is a competitive inhibitor of glutamine synthetase, the only enzyme that can catalyze assimilation of ammonia into glutamic acid into plants Inhibition of glutamine synthetase ultimately results in the accumulation of toxic ammonia levels, resulting in plant cell death. Phosphosphinothricin acetyltransferase, which is encoded by the bar gene from Streptomyces hygroscopicus, confers resistance to herbicides that contain PPT.

Dalapon is an herbicide used to control grasses in a wide variety of crops. Dalapon dehalogenase is capable of degrading high concentrations of the herbicide dalapon.

Additional non-limiting examples of genes that provide resistance to herbicides include, but are not limited to, mutant genes that confer resistance to imidazalinone or sulfonylurea, such as genes encoding mutant form of acetohydroxyacid synthase (AHAS), also known as acetolactate synthase (ALS) (Lee at al., EMBO J., 1988, 7: 1241; Miki et al., Theor. Appl. Genet., 1990, 80: 449; and U.S. Pat. No. 5,773,702); and genes that confer resistance to phenoxy propionic acids and cyclohexones such as the ACCAse inhibitor-encoding genes (Marshall et al., Theor. Appl. Genet., 1992, 83: 435).

Resistance to Pests and/or Diseases

Genes that confer resistance to pests and/or disease include, but are not limited to, genes whose products confer resistance to infestation from an organism selected from the group consisting of insects, bacteria, fungi, and nematodes. Heterologous genes whose products confer resistance to viruses may also be expressed using gene regulatory elements of the present invention.

Gene products that can confer resistance to insects and/or insect disease include, but are not limited to, Bt (Bacillus thuringiensis) proteins (such as delta-endotoxin (U.S. Pat. No. 6,100,456)); vitamin-binding proteins such as avidin and avidin homologs (which can be used as larvicides against insect pests); insect-specific hormones or pheromones such as ecdysteroid and juvenile hormone, and variants thereof, mimetics based thereon, or an antagonists or agonists thereof; insect-specific peptides or neuropeptides which, upon expression, disrupts the physiology of the pest; insect-specific venom such as that produced by a wasp, snake, etc.; enzyme polypeptides responsible for the accumulation of monoterpenes, sesquiterpenes, asteroid, hydroxamic acid, phenylpropanoid derivative or other non-protein molecule with insecticidal activity; insect-specific antibodies or antitoxins (Tavladoraki et al., Nature, 1993, 366: 469); and TcdA protein (Liu et al., 2003 Nature Biotechnology 21: 1222-1228).

Gene products that can confer resistance to bacteria and/or bacterial diseases include, but are not limited to, nucleotide-binding-sequence LRR (also known as ‘NBS-leucine rich repeat’) proteins (Van Der Biezen and Jones, 1998 Trends in Biochemical Sciences 23: 454-456).

Gene products that can confer resistance to fungi and/or fungal diseases include, but are not limited to, Pi-ta (U.S. Pat. No. 6,743,969), Pathogenesis-related (PR) proteins, chitinases and β-1,3-glucanases, ribosome-inactivating proteins (RIPs), thionins, hydrophobic moment peptides (such as derivatives of Tachyplesin which inhibit fungal pathogens), and antifungal peptides such as LCI.

Gene products that can confer resistance to viruses and/or viral diseases include, but are not limited to, nucleotide-binding site-leucine-rich repeat (NBS-LRR proteins), virus-specific antibodies and antitoxins (Tavladoraki et al., Nature, 1993, 366: 469), viral invasive proteins or complex toxins derived therefrom (Beachy et al., Ann. Rev. Phytopathol., 1990, 28: 451), PR proteins, and Rx proteins (genetically engineered cross protection is conferred by expressing viral coat protein genes in the plant genome).

Gene products that can confer resistance to nematodes and/or nematode diseases include, but are not limited to, peroxidases, chitinases, lipoxygenases, proteinase inhibitors, Mi proteins, Gro, Gpa and Cre proteins.

Other gene products that can confer resistances to diseases or pests include, but are not limited to, lectins (Van Damme et al., Plant Mol. Biol., 1994, 24: 825); protease or amylase inhibitors, such as the rice cysteine proteinase inhibitor (Abe et al., J. Biol. Chem., 1987, 262: 16793) and the tobacco proteinase inhibitor I (Hubb et al., Plant Mol. Biol., 1993, 21: 985); enzyme polypeptides involved in the modification of a biologically active molecule (U.S. Pat. No. 5,539,095); peptides that stimulate signal transduction; membrane permeases (channel formers or channel blockers) (Jaynes et al., Plant Sci., 1993, 89: 43); and developmental-arrestive proteins produced by a plant, pathogen or parasite that prevents disease.

Resistance to Stress

Gene products that confer resistance to environmental stress include both biotic and abiotic stress proteins.

Biotic stress in plants can be caused by bacteria, fungi, viruses, insects and nematodes. Non-limiting examples of proteins that can provide biotic stress resistance/tolerance in plants include those that confer resistance to diseases and pests mentioned above, as well as DREB transcription factors (Agarwal et al., 2006 Plant Cell Reports 25: 1263-1274) and MAP Kinases (U.S. Pat. No. 7,345,219).

Abiotic stress in plants can be caused by a variety of factors, including, but not limited to, nutrient imbalances, light (high light, UV, darkness), water imbalances (deficit, desiccation, flooding), temperature imbalances (frost, cold, heat), oxidation stress, hypoxia, physical factors (such as wind and touch), salt, and heavy metals. Examples of gene products that can provide abiotic stress resistance/tolerance in plants include HSFs, LEAs, CORs, CBFs and ABFs (Vinocur and Altman, 2005 Current Opinion in Biotechnology 16:123-132).

Examples of genes whose products confer resistance to environmental stress include, but are not limited to, mtld and HVA1 (which confer resistance to environmental stress factors); and rd29A and rd19B (Arabidopsis thaliana genes that encode hydrophilic proteins induced in response to dehydration, low temperature, salt stress, and/or exposure to abscisic acid and enable the plant to tolerate the stress (Yamaguchi-Shinozaki et al., Plant Cell, 1994, 6: 251-264)). Other such genes contemplated can be found in U.S. Pat. Nos. 5,296,462 and 5,356,816.

c. Other Heterologous Gene Products

Gene regulatory elements provided by the present invention may also be used to drive and/or facilitate other heterologous gene products that confer advantages to the plants that express them.

For example, nutrient utilization polypeptides can be expressed in transgenic plants. Such polypeptides can maximize utilization of nutrients by plants and may lead to increased yields. Nutrients whose utilization maximization may be desired include, but are not limited to, nitrogen, phosphorous, potassium, iron, zinc etc.

It may be desirable to trnasgenically express anthranilate synthase, which catalyzes the conversion of chorismate into anthranilate. Anthranilate is the biosynthetic precursor of both tryptophan and numerous secondary metabolites, including inducible plant defense compounds

It may be desirable to express mycotoxin reduction polypeptides in plants. Mycotoxins are toxic and carcinogenic chemicals produced by fungi in plants during growth or storage of grains and are major concern for growers. Bt proteins, when expressed in plants reduce mycotoxin content (Wu et al., 2004 Toxin Reviews 23: 397-424).

Male sterility polypeptides may also be expressed in transgenic plants using gene regulatory elements of the present invention. Male sterility in plants can be induced by expressing several types of polypeptides such as RNase/Barnase (Mariani et al., 1990 Nature 347: 737-741).

Heterologous gene products that affect grain composition or quality (e.g., by altering key components of grain, such as starch, protein, bran, etc.) may also be expressed. Desired changes in composition may include, for example, relative proportions of starch fractions such amylose and amylopectin; decreased amounts of undesirable components such as phytic acid; and/or improved amino acid content conferred, for example, by modified seed storage proteins that have been. For example, corn zeins modified to contain more lysine can be expressed.

Polypeptides having therapeutic value can also be expressed in plants using provided gene regulatory elements. Such polypeptides can be harvested from plants transgenically expressing them and then purifed for downstream applications. Such polypeptides include, but are not limited to, antibodies, blood products, cytokines, growth factors, hormones, recombinant enzymes, and vaccines that would have a variety of applications in human and animal health. For example, lactoferrin and lysozyme has been produced in rice grains (Ventria Bioscience).

Heterologous gene products that may be expressed also include RNA molecules, for example, those that regulate a plant gene.

### 3. Transcriptional and Translational Termination

The transcriptional and translational termination region generally comprises a sequence that encodes a “terminator” (the “terminator sequence”). The transcriptional and translational termination region can be native with the transcription initiation region, can be native with the operably linked polynucleotide sequence of interest, and/or can be derived from another source. Convenient termination regions are available from the T1-plasmid of A. tumefaciens, such as the octopine synthase and nopaline synthase termination regions (An et al., Plant Cell, 1989, 1: 115-122; Guerineau et al., Mol. Gen. Genet. 1991, 262: 141-144; Proudfoot, Cell, 1991, 64: 671-674; Sanfacon et al., Genes Dev. 1991, 5: 141-149; Mogen et al., Plant Cell, 1990, 2:1261-1272; Munroe et al., Gene, 1990, 91:151-158; Ballas et al., Nucleic Acids Res., 1989, 17: 7891-7903; and Joshi et al., Nucleic Acid Res., 1987, 15: 9627-9639).

### 4. Marker Genes

In some embodiments, nucleic acid constructs include one or more marker genes. Marker genes are genes that impart a distinct phenotype to cells expressing the marker gene and thus allow transformed cells to be distinguished from cells that do not have the marker. Such genes may encode, for example, a selectable and/or screenable marker. In some embodiments, nucleic acid constructs comprise a marker that allows selecting and/or screening in a transformed cell.

In some embodiments, the transformed cell is grown in culture medium under conditions that select for cells that either have (positive selection) or do not have (negative selection) the marker. In some embodiments, a combination of postive and negative selection is used.

In some so-called positive selection schemes, most cells in a population are unable to divide and because they lack the ability to use a nutrient (such as, for example, a carbon source) present in the selection medium. In these schemes, the selectable marker confers an ability to use the nutrent. Thus, cells that have the selectable marker gain an advantage over other cells in the population and therefore can be selected.

In some so-called negative screening/selection schemes, most cells in a population are unable to divide because of the effects of a toxic agent (such as, for example, an antibiotic present in the selection medium). In these schemes, the selectable marker confers an ability to overcome the toxicity (for example, by blocking uptake or by chemically modifying the toxic agent). Thus, cells that have the selectable marker gain an advantage over other cells in the population and therefore can be selected.

In some embodiments, the transformed cell undergoing selection is a prokaryotic cell, such as E. coli and Agrobacterium. In some embodiments, the transformed cell undergoing selection is a eukaryotic cell, such as a yeast (for example, S. cerevisiae), mammalian, insect, or plant cell.

In some embodiments, the characteristic phenotype allows the identification of cells, groups of cells, tissues, organs, plant parts or whole plants containing the construct.

Many examples of suitable marker genes are known in the art and can be used in screening and/or selection schemes. Reagents such as appropriate components of selection media are also known in the art. Examples of such marker genes include, but are not limited to, phosphomannose isomerase, phosphinothricin, neomycin phosphotransferase, hygromyci phosphotransferase, enolpyruvoyl-shikimate-3-phosphate synthetase, etc.

For example, phosphomannose isomerase (PMI) catalyses the interconversion of mannose 6-phosphate and fructose 6-phosphate in prokaryotic and eukaryotic cells. After uptake, mannose is phosphorylated by endogenous hexokinases to mannose-6-phosphate. Accumulation of mannose-6-phosphate leads to a block in glycolysis by inhibition of phosphoglucose-isomerase, resulting in severe growth inhibition. Phosphomannose-isomerase is encoded by the manA gene from Escherichia coli and catalyzes the conversion of mannose-6-phosphate to fructose-6-phosphate, an intermediate of glycolysis. On media containing mannose, manA expression in transformed plant cells relieves the growth inhibiting effect of mannose-6-phosphate accumulation and permits utilization of mannose as a source of carbon and energy, allowing transformed cells to grow.

Reporter proteins (such as GUS (β-glucuronidase), green fluorescent protein and derivatives thereof, and luciferase). Reporter genes may allow easy visual detection of transformed cells by visual screening and may also be used as marker genes. Non-limiting examples of eporter proteins include GUS (a β-glucuronidase), green fluorescent protein and derivatives thereof, and luciferase.

In some embodiments, the marker confers benefit(s) to the transgenic plant such as herbicide resistance, insect resistance, disease resistance, and increased tolerance to environmental stress (e.g., drought). (See, for example, the section on genes of interest above for an expanded discussion of some of these genes.)

Alternatively or additionally, a marker gene can provide some other visibly reactive response (e.g., may cause a distinctive appearance such as color or growth pattern relative to plants or plant cells not expressing the selectable marker gene in the presence of some substance, either as applied directly to the plant or plant cells or as present in the plant or plant cell growth media). It is now well known in the art that transcriptional activators of anthocyanin biosynthesis, operably linked to a suitable promoter in a construct, have widespread utility as non-phytotoxic markers for plant cell transformation.

B. Tissue-Specific and/or Tissue-Preferred Expression

In certain embodiments, heterologous gene product(s) is/are targeted to specific tissues of the transgenic plant such that the heterologous gene product(s) is/are present in only some plant tissues during the life of the plant. For example, tissue specific expression may be performed to preferentially express polypeptides encoded by heterologous genes in leaves and stems rather than grain or seed (which can reduce concerns about human consumption of genetically modified organism (GMOs)). Tissue-specific expression has other benefits including targeted expression of enzyme polypeptide(s) to the appropriate substrate.

In certain embodiments, heterologous gene product(s) is/are preferentiallly expressed certain tissues of the transgenic plant such that the heterologous gene product(s) is/are present at higher levels in some plant tissues than in others during the life of the plant.

Tissue-specific and/or tissue-preferred expression may be functionally accomplished by using one or more tissue-specific and/or tissue-preferred gene regulatory elements, such as some of the sorghum promoters disclosed herein (see, for example, Example 5). A number of known tissue-specific promoters may be used in combination with gene regulatory elements disclosed herein. For example, in embodiments wherein two heterologous gene products are expressed in the same plant or other organism, expression of one heterologous gene product may be driven by a gene regulatory element from sorghum as disclosed herein, while expression of the other heterologous gene product may be driven by a gene regulatory element that is known, such as a known tissue-specific promoter. Several tissue-specific regulated genes and/or promoters have been reported in plants. Some reported tissue-specific genes include without limitation genes encoding seed storage proteins (such as napin, cruciferin, β-conglycinin, and phaseolin), genes encoding zein or oil body proteins (such as oleosin), genes involved in fatty acid biosynthesis (including acyl carrier protein, stearoyl-ACP desaturase, and fatty acid desaturases (fad 2-1)), and other genes expressed during embryo development (such as Bce4 (Kridl et al., Seed Science Research, 1991, 1: 209)). Examples of tissue-specific promoters that have been described in the art include the lectin (Vodkin, Prog. Clin. Biol. Res., 1983, 138: 87; Lindstrom et al., Der. Genet., 1990, 11: 160), corn alcohol dehydrogenase 1 (Dennis et al., Nucleic Acids Res., 1984, 12: 983), corn light harvesting complex (Bansal et al., Proc. Natl. Acad. Sci. USA, 1992, 89: 3654), corn heat shock protein, pea small subunit RuBP carboxylase, Ti plasmid mannopine synthase, Ti plasmid nopaline synthase, petunia chalcone isomerase (van Tunen et al., EMBO J., 1988, 7:125), bean glycine rich protein 1 (Keller et al., Genes Dev., 1989, 3: 1639), truncated CaMV 35S (Odell et al., Nature, 1985, 313: 810), potato patatin (Wenzler et al., Plant Mol. Biol., 1989, 13: 347), root cell (Yamamoto et al., Nucleic Acids Res., 1990, 18: 7449), maize zein (Reina et al., Nucleic Acids Res., 1990, 18: 6425; Kriz et al., Mol. Gen. Genet., 1987, 207: 90; Wandelt et al., Nucleic Acids Res., 1989, 17 2354), PEPCase, R gene complex-associated promoters (Chandler et al., Plant Cell, 1989, 1: 1175), and chalcone synthase promoters (Franken et al., EMBO J., 1991, 10: 2605). Particularly useful for seed-specific expression is the pea vicilin promoter (Czako et al., Mol. Gen. Genet., 1992, 235: 33).

Tissue-specific and/or tissue-preferred expression may also be functionally accomplished by introducing a constitutively expressed gene in combination with an antisense gene that is expressed only in those tissues where the gene product is not desired, or where it is desired that the gene be expressed at lower levels. For example, a gene encoding an heterologous or homologous polypeptide may be expressed in all tissues under the control of a constitutive promoter such as constitutive sorghum promoters disclosed herein and/or a known constitutive promoter such as the 35S promoter from Cauliflower Mosaic Virus. Expression of an antisense transcript of the gene in a particular tissue, using for example tissue-specific promoter or tissue-preferred promoter, would prevent accumulation of the enzyme polypeptide in that tissue. A tissue-specific and tissue-preferred sorghum promoter disclosed herein and/or a known tissue-specific or tissue-preferred promoter may be used to drive expression of the antinsense transcript. For example, an antisense transcript of the gene for which tissue-specific or tissue-preferred expression is desired may be expressed in maize kernel using a zein promoter, thereby preventing accumulation of the gene product in seed. Hence the polypeptide encoded by the heterologous gene would be present in all tissues except the kernel.

### C. Subcellular-Specific Expression

In certain embodiments, heterologous gene product(s) is/are targeted to specific cellular compartments or organelles, such as, for example, the cytosol, the vacuole, the nucleus, the endoplasmic reticulum, the cell wall, the mitochondria, the apoplast, the peroxisomes, plastids, or combinations thereof. In some embodiments of the invention, the heterologous gene is expressed in one or more subcellular compartments or organelles, for example, the cell wall and/or endoplasmic reticulum, during the life of the plant.

In some embodiments, directing the product (e.g., a polypeptide and/or RNA molecule) of the heterologous gene to a specific cell compartment or organelle allows the product to be localized such that it will not come into contact with another molecule until desired. For example, if the product is an enzyme polypeptide, it may be possible to prevent the enzyme polypeptide from coming into contact with its substrate during plant growth. Thus, the enzyme polypeptide would not act until it is allowed to contact its substrate, e.g., following physical disruption of cell integrity by milling.

As another example, targeting expression of a cell wall-modifying and/or lignocellulolytic enzyme polypeptide to the cell wall (as in the apoplast) can help overcome the difficulty of mixing hydrophobic cellulose and hydrophilic enzymes that make it hard to achieve efficient hydrolysis with external enzymes.

In some embodiments, gene products are targeted to more than one subcellular compartments or organelles. Such targeting may allow one to increase the total amount of heterologous gene product in the plant. In some embodiments, targeting to one or more subcellular compartments or organelles is achieved using a gene regulatory element (such as a promoter) that drives expression specifically or preferentially in one or more subcellular compartments or organelles. Thus, for example, using an apoplast promoter with the E1 endo-1,4-β-glucanase gene and a chloroplast promoter with the E1 gene in a plant would increase total production of E1 compared to a single promoter/E1 construct in the plant.

Furthermore, in the case of expression of enzyme polypeptides that modify the cell wall (e.g., cell wall-modifying enzyme polypeptides and/or lignocellulolytic enzyme polypeptides)) one can minimize in vivo (pre-processing) deconstruction of the cell wall that occurs when multiple synergistic enzymes are present in a cell by using promoters targeted to different locations in the plant. For example, combining an endoglucanase with an apoplast promoter, a hemicellulase with a vacuole promoter, and an exoglucanase with a chloroplast promoter, sequesters each enzyme in a different part of the cell and achieves the advantages listed above. This method circumvents the limit on polypeptide or other heterologous gene product mass that can be expressed in a single organelle or location of the cell.

Localization of a nuclear-encoded protein (e.g., enzyme polypeptide) within the cell is known to be determined by the amino acid sequence of the protein. Protein localization can be altered, for example, by modifying the nucleotide sequence that encodes the protein in such a manner as to alter the protein's amino acid sequence. Polynucleotide sequences encoding polypeptides can be altered to redirect cellular localization of the encoded polypeptides by any suitable method (see, e.g., Dai et al., Trans. Res., 2005, 14: 627, the entire contents of which are herein incorporated by reference). In some embodiments of the invention, polypeptide localization is altered by fusing a sequence encoding a signal peptide to the sequence encoding the polypeptide. Signal peptides that may be used in accordance with the invention include without limitation a secretion signal from sea anemone equistatin (which allows localization to apoplasts) and secretion signals comprising the KDEL motif (which allows localization to endoplasmic reticulum).

### D. Expression Vectors

Generally, any vector that can be used constructed to express a product (e.g., polypeptide or RNA molecule) of a gene after introduction of such a vector in a host cell is considered an “expression vector.” Expression vectors typically contain nucleic acid constructs such as expression cassettes described above inserted into a vector. Expression vectors can be designed for expressing a gene product in any of a variety of host cells, including both prokaryotic (e.g., bacteria such as E. coli and Agrobacterium) and eukaryotic (e.g. insect, yeast (such as S. cerevisiae), and mammalian cells) host cells.

Nucleic acid constructs according to the present invention may be cloned into any of a variety of vectors, such as binary vectors, viral vectors, phage, phagemids, cosmids, and plasmids. Vectors suitable for transforming plant cells include, but are not limited to, Ti plasmids from Agrobacterium tumefaciens (J. Darnell, H. F. Lodish and D. Baltimore, “Molecular Cell Biology”, 2nd Ed., 1990, Scientific American Books: New York); plasmid containing a β glucuronidase gene and a cauliflower mosaic virus (CaMV) promoter plus a leader sequence from alfalfa mosaic virus (J. C. Sanford et al., Plant Mol. Biol. 1993, 22: 751-765); and plasmids containing a bar gene cloned downstream from a CaMV 35S promoter and a tobacco mosaic virus (TMV) leader. Other plasmids may additionally contain introns, such as that derived from alcohol dehydrogenase (Adh1) and/or other DNA sequences. The size of the vector is not a limiting factor.

For constructs that are intended be used in Agrobacterium-mediated transformation, the plasmid may contain an origin of replication that allows it to replicate in Agrobacterium and a high copy number origin of replication functional in E. coli. This permits facile production and testing of transgenes in E. coli prior to transfer to Agrobacterium for subsequent introduction in plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

Methods of preparation of nucleic acid constructs and expression vectors are well known in the art and can be found described in several textbooks such as, for example, J. Sambrook, E. F. Fritsch and T. Maniatis, “Molecular Cloning: A Laboratory Manual”, 1989, Cold Spring Harbor Laboratory: Cold Spring Harbor, and T. J. Silhavy, M. L. Berman, and L. W. Enquist, “Experiments with Gene Fusions”, 1984, Cold Spring Harbor Laboratory: Cold Spring Harbor; F. M. Ausubel et al., “Current Protocols in Molecular Biology”, 1989, John Wiley & Sons: New York.

### II. Transgenic Plants

In one aspect, the present invention provides novel transgenic plants that express one or more polypeptides or RNA molecules under the control of a gene regulatory element provided by the present disclosure. The polypeptides or RNA molecules may be any polypeptide or RNA molecule for which expression in a plant is desired, including, but not limited to, those described herein.

In certain embodiments, provided are transgenic plants, the genomes of which are augmented with a recombinant polynucleotide comprising a gene regulatory element from sorghum as described herein. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO: 1 to 48. In some embodiments, the nucleotide sequence of the gene regulatory element is one of SEQ ID NO: 1 to 48. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO: 1, 5, 6, 10, 11, 43, and 45. (See, e.g., Examples 2, 3, 4, and 6.). In some embodiments, the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO: 11 and 45.

In some embodiments, the transgenic plant further comprises a heterologous gene operably linked to the gene regulatory element. In some such embodiments, the gene regulatory element regulates expression of the heterologous gene.

The heterologous gene may encode any polypeptide or RNA molecule for which expression in a plant is desired, including, but not limited to, those described herein. In some embodiments, the recombinant polynucleotide further comprises a gene terminator sequence that is operably linked to the heterologous gene.

Nucleic acid constructs, such as those described above, can be used to transform any plant. In some embodiments, plants are green field plants. In some embodiments, plants are grown specifically for “biomass energy” and/or phytoremediation.

In some embodiments, the plants are monocotyledonous plants. Examples of monocotyledonous plants that may be transformed in accordance with the practice of the present invention include, but are not limited to, bamboo, barley, maize (corn), sorghum, switchgrass, miscanthus, wheat, rice, rye, turfgrass, millet, and sugarcane.

In some embodiments, the plants are dicotyledonous plants. Examples of dicotyledonous plants that may be transformed in accordance with the practice of the present invention include, but are not limited to, Arabidopsis, cottonwood (e.g., Populus deltoides), eucalyptus, tobacco, tomato, potato, rape, soybean, canola, sugar beet, sunflower, sweetgum, alfalfa, cotton, willow, and poplar.

In some embodiments, the plants a pine trees (pinus sp.)

In some embodiments, the transgenic plant is fertile. In some embodiments, the transgenic plant is not fertile (i.e., sterile).

Using transformation methods, genetically modified plants, plant cells, plant tissue, seeds, and the like can be obtained.

Transformation according to the present invention may be performed by any suitable method. In certain embodiments, transformation comprises steps of introducing a nucleic acid construct, as described above, into a plant cell or protoplast to obtain a stably transformed plant cell or protoplast; and regenerating a whole plant from the stably transformed plant cell or protoplast.

Cell Transformation

Delivery or introduction of a nucleic acid construct into eukaryotic cells may be accomplished using any of a variety of methods. The choice of a particular method used for the transformation is not critical to the instant invention. Suitable techniques include, but are not limited to, non-biological methods, such as microinjection, microprojectile bombardment, electroporation, induced uptake, and aerosol beam injection, as well as biological methods such as direct DNA uptake, liposome-mediated transfection, polyethylene glycol-mediated transfection, and Agrobacterium-mediated transformation. Any combinations of the above methods that provide for efficient transformation of plant cells or protoplasts may also be used in the practice of the invention.

Methods of introduction of nucleic acid constructs into plant cells or protoplasts have been described. See, for example, “Methods for Plant Molecular Biology”, Weissbach and Weissbach (Eds.), 1989, Academic Press, Inc; “Plant Cell, Tissue and Organ Culture: Fundamental Methods”, 1995, Springer-Verlag: Berlin, Germany; and U.S. Pat. Nos. 4,945,050; 5,036,006; 5,100,792; 5,240,855; 5,302,523; 5,322,783; 5,324,646; 5,384,253; 5,464,765; 5,538,877; 5,538,880; 5,550,318; 5,563,055; and 5,591,616).

In particular, electroporation has frequently been used to transform plant cells (see, for example, U.S. Pat. No. 5,384,253). This method is generally performed using friable tissues (such as a suspension culture of cells or embryogenic callus) or target recipient cells from immature embryos or other organized tissue that have been rendered more susceptible to transformation by electroporation by exposing them to pectin-degrading enzymes or by mechanically wounding them in a controlled manner. Intact cells of maize (see, for example, K. D'Halluin et al., Plant cell, 1992, 4: 1495-1505; C. A. Rhodes et al., Methods Mol. Biol. 1995, 55: 121-131; and U.S. Pat. No. 5,384,253), wheat, tomato, soybean, and tobacco have been transformed by electroporation. As reviewed, for example, by G. W. Bates (Methods Mol. Biol. 1999, 111: 359-366), electroporation can also be used to transform protoplasts.

Another method of transformation is microprojectile bombardment (e.g., through use of a “gene gun”) (see, for example, U.S. Pat. Nos. 5,538,880; 5,550,318; and 5,610,042; and WO 94/09699). In this method, nucleic acids are delivered to living cells by coating or precipitating the nucleic acids onto a particle or microprojectile (for example tungsten, platinum or gold), and propelling the coated microprojectile into the living cell. Microprojectile bombardment techniques are widely applicable, and may be used to transform virtually any monocotyledonous or dicotyledonous plant species (see, for example, U.S. Pat. Nos. 5,036,006; 5,302,523; 5,322,783 and 5,563,055; WO 95/06128; A. Ritala et al., Plant Mol. Biol. 1994, 24: 317-325; L. A. Hengens et al., Plant Mol. Biol. 1993, 23: 643-669; L. A. Hengens et al., Plant Mol. Biol. 1993, 22: 1101-1127; C. M. Buising and R. M. Benbow, Mol. Gen. Genet. 1994, 243: 71-81; C. Singsit et al., Transgenic Res. 1997, 6: 169-176).

The use of Agrobacterium-mediated transformation of plant cells is well known in the art (see, for example, U.S. Pat. No. 5,563,055). This method has long been used in the transformation of dicotyledonous plants, including Arabidopsis and tobacco, and has recently also become applicable to monocotyledonous plants, such as rice, wheat, barley and maize (see, for example, U.S. Pat. No. 5,591,616). In plant strains where Agrobacterium-mediated transformation is efficient, it is often the method of choice because of the facile and defined nature of the gene transfer. In some embodiments, Agrobacterium-mediated transformation of plant cells is carried out in two phases. First, the steps of cloning and DNA modifications are performed in E. coli, and then the plasmid containing the gene construct of interest is transferred by heat shock treatment into Agrobacterium, and the resulting Agrobacterium strain is used to transform plant cells. In some embodiments, Agrobacterium infiltrates plant leaves. In some embodiments, the bacterial strain Agrobacterium tumefaciens is used to transform plant cells.

Transformation of plant protoplasts can be achieved using methods based on calcium phosphate precipitation, polyethylene glycol treatment, electroporation, and combinations of these treatments (see, e.g., I. Potrykus et al., Mol. Gen. Genet. 1985, 199: 169-177; M. E. Fromm et al., Nature, 1986, 31: 791-793; J. Callis et al., Genes Dev. 1987, 1: 1183-1200; S. Omirulleh et al., Plant Mol. Biol. 1993, 21: 415-428).

Alternative methods of plant cell transformation, that have been reviewed, for example, by M. Rakoczy-Trojanowska (Cell Mol. Biol. Lett. 2002, 7: 849-858; the contents of which are herein incorporated by reference in their entirety), can also be used in the practice of the present invention.

In some embodiments, successful delivery of the nucleic acid construct into the host plant cell or protoplast is preliminarily evaluated visually. Selection of stably transformed plant cells can be performed, for example, by introducing into the cell a nucleic acid construct comprising a marker gene which confers resistance to some normally inhibitory agent, such as an antibiotic or herbicide. Examples of antibiotics that may be used include aminoglycoside antibiotics (such as neomycin, kanamycin, and paromomycin) and the antibiotic hygromycin. Several aminoglycoside phosphotransferases confer resistance to aminoglycoside antibiotics, and inclide aminoglycoside phosphotransferase I (aph-I) enzyme and aminoglycoside (or neomycin) phosphotransferase II (APH-II or NPTII), which, though unrelated, both have ability to inactivate the antibiotic G418. The hygromycin phosphotransferase (denoted hpt, hph or aphIV) gene was originally derived from Escherichia coli. Hygromycin phosphotransferase (HPT) detoxifies the aminocyclitol antibiotic hygromycin B. As is known in the art, plants have been transformed with the hpt gene, and hygromycin B has proved very effective in the selection of a wide range of plants

Examples of herbicides that may be used include phosphinothricin and glyphosate. Potentially transformed cells then are exposed to the selective agent. Cells where the resistance-conferring gene has been integrated and expressed at sufficient levels to permit cell survival will generally be present in the population of surviving cells.

Alternatively or additionally, host cells comprising a nucleic acid sequence of the invention and expressing a gene product encoding by inventive nucleic acids may be identified and selected by a variety of procedures, including, but not limited to, DNA-DNA or DNA-RNA hybridization and protein bioassay or immunoassay techniques such as membrane, solution, or chip-based technologies for the detection and/or quantification of nucleic acids or proteins.

Plant cells are available from a wide range of sources including the American Type Culture Collection (Rockland, Md.), or from any of a number of seed companies including, for example, A. Atlee Burpee Seed Co. (Warminster, Pa.), Park Seed Co. (Greenwood, S.C.), Johnny Seed Co. (Albion, Me.), or Northrup King Seeds (Hartsville, S.C.). Descriptions and sources of useful host cells can be found in I. K. Vasil, “Cell Culture and Somatic Cell Genetics of Plants”, Vol. I, II and II; 1984, Laboratory Procedures and Their Applications Academic Press: New York; R. A. Dixon et al., “Plant Cell Culture—A Practical Approach”, 1985, IRL Press: Oxford University; and Green et al., “Plant Tissue and Cell Culture”, 1987, Academic Press: New York.

Plant cells or protoplasts stably transformed according to the present invention are provided herein.

Plant Regeneration

In plants, every cell is capable of regenerating into a mature plant and contributing to the germ line such that subsequent generations of the plant will contain the transgene of interest. Stably transformed cells may be grown into plants according to conventional ways (see, for example, McCormick et al., Plant Cell Reports, 1986, 5: 81-84). Plant regeneration from cultured protoplasts has been described, for example by Evans et al., “Handbook of Plant Cell Cultures”, Vol. 1, 1983, MacMilan Publishing Co: New York; and I.R. Vasil (Ed.), “Cell Culture and Somatic Cell Genetics of Plants”, Vol. I (1984) and Vol. II (1986), Acad. Press: Orlando.

Means for regeneration vary from species to species of plants, but generally a suspension of transformed protoplasts or a Petri plate containing transformed explants is first provided. Callus tissue is formed and shoots may be induced from callus and subsequently roots. Alternatively, somatic embryo formation can be induced in the callus tissue. These somatic embryos germinate as natural embryos to form plants. The culture media will generally contain various amino acids and plant hormones, such as auxin and cytokinins Glutamic acid and proline may also be added to the medium. Efficient regeneration generally depends on the medium, on the genotype, and on the history of the culture.

Regeneration from transformed individual cells to obtain transgenic whole plants has been shown to be possible for a large number of plants. For example, regeneration has been demonstrated for dicots (such as apple; Malus pumila; blackberry, Rubus; Blackberry/raspberry hybrid, Rubus; red raspberry, Rubus; carrot; Daucus carota; cauliflower; Brassica oleracea; celery; Apium graveolens; cucumber; Cucumis sativus; eggplant; Solanum melongena; lettuce; Lactuca sativa; potato; Solanum tuberosum; rape; Brassica napus; soybean (wild); Glycine canescens; strawberry; Fragaria×ananassa; tomato; Lycopersicon esculentum; walnut; Juglans regia; melon; Cucumis melo; grape; Vitis vinifera; and mango; Mangifera indica) as well as for monocots (such as rice; Oryza sativa; rye, Secale cereale; and Maize).

Primary transgenic plants may then be grown using conventional methods. Various techniques for plant cultivation are well known in the art. Plants can be grown in soil, or alternatively can be grown hydroponically (see, for example, U.S. Pat. Nos. 5,364,451; 5,393,426; and 5,785,735). Primary transgenic plants may be either pollinated with the same transformed strain or with a different strain and the resulting hybrid having the desired phenotypic characteristics identified and selected. Two or more generations may be grown to ensure that the subject phenotypic characteristics is stably maintained and inherited and then seeds are harvested to ensure that the desired phenotype or other property has been achieved.

As is well known in the art, plants may be grown in different media such as soil, growth solution or water.

Selection of plants that have been transformed with the construct may be performed by any suitable method, for example, with northern blot, Southern blot, herbicide resistance screening, antibiotic resistance screening or any combinations of these or other methods. The Southern blot and northern blot techniques, which test for the presence (in a tissue such as a plant tissue) of a nucleic acid sequence of interest and of its corresponding RNA, respectively, are standard methods (see, for example, Sambrook & Russell, “Molecular Cloning”, 2001, Cold Spring Harbor Laboratory Press: Cold Spring Harbor).

### III. Uses of Inventive Transgenic Plants

Transgenic plants and plant parts disclosed herein may be used advantageously in a variety of applications. In many embodiments, transgenic plants of the present invention express polypeptides that confer desirable traits to the plant and/or plant biomass (e.g., resistance to herbicides, resistance to environmental stress, resistance to pests and diseases). In some embodiments, expression of such polypeptides results in downstream process innovations and/or improvements in a variety of applications including ethanol production, phytoremediation and hydrogen production.

### A. Ethanol Production

In some embodiments, plants transformed according to the present invention provide a means of increasing ethanol yields, reducing pretreatment costs by reducing acid/heat pretreatment requirements for saccharification of biomass; and/or reducing other plant production and processing costs, such as by allowing multi-applications and isolation of commercially valuable by-products. For example, a gene regulatory element provided by the present disclosure may drive expression of one or more lignocellulolytic enzyme polypeptide(s) and/or cell wall modifying enzyme polypeptide(s) in a transgenic plant and such enzyme polypeptides may allow biomass from the transgenic plant to be processed to produce more easily and/or cost effectively.

Plant Culture

Farmers can grow different transgenic plants of the present invention (e.g., different variety of transgenic corn, each expressing a transgenic polypeptide or RNA) simultaneously, achieving the desired “blend” of gene products produced by changing the seed ratio.

Plant Harvest

Transgenic plants of the present invention can be harvested as known in the art. For example, current techniques may cut corn stover at the same time as the grain is harvested, but leave the stover lying in the field for later collection. However, dirt collected by the stover can interfere with ethanol production from lignocellulosic material. The present invention provides a method in which transgenic plants are cut, collected, stored, and transported so as to minimize soil contact. In addition to minimizing interference from dirt with ethanol production, this method can result in reduction in harvest and transportation costs.

Tempering

In some embodiments, provided transgenic plants undergo a tempering phase that conditions the biomass for pretreatment and hydrolysis. Tempering may facilitate reducing severity of pretreatment conditions to achieve a desired glucan conversion yield and/or improving hydrolysis and glucan conversion after treatment. For example, a typical yield from biomass that has been pretreated under standard pretreatment conditions (e.g., 1% sulfuric acid, 170° C., for 10 minutes) is at least 80% glucan conversion. When tempered as described herein, the same typical yield may be achieved under less severe pretreatment conditions and/or with reduced amounts of externally applied enzymes. Less severe pretreatment conditions may comprise, for example, reduced acid concentrations, lower incubation temperatures, and/or shorter pretreatment times.

In some embodiments, when tempered as described herein and using the same pretreatment conditions, typical yield may be increased above at least 80% glucan conversion.

Without wishing to be bound by any particular theory, tempering may facilitate such improvements by, for example, allowing activation of endoplant enzyme polypeptides after harvest, increasing susceptibility of lignin and hemicellulose to traditional pretreatment, and/or increasing accessibility of polysaccharides (e.g., cellulose).

A variety of techniques for tempering may be used. In some embodiments, tempering comprises increasing the temperature of the biomass to activate thermophilic enzymes. Increasing the temperature to activate thermophilic enzymes may be achieved, for example, by one or more of ensilement, grinding, pelleting, and warm water suspension/slurries. In some embodiments, tempering comprises disrupting cell walls. Cell wall disruption may be achieved, for example, by sonication and/or liquid extraction to release enzyme polypeptides from sequestered locations in the plant (which may allow further activation and/or extraction to be added back after pretreatment). In some embodiments, tempering comprises adding accessory enzyme polypeptides during an incubation period before pretreatment. Such accessory enzyme polypeptides may weaken cross linking and improve accessibilty of the biomass to embedded glucanases or xylanases. In some embodiments, tempering comprises incubating the biomass in a particular set of conditions (e.g., a particular temperature, particular pH, and/or particular moisture conditions). Such incubations may in some embodiments increase susceptibility to various glucanases and/or accessory enzyme polypeptides present in the plant tissues or added to the sample. For example, samples may be tempered as a liquid slurry (e.g., comprising about 10% to about 30% total solids) under conditions favorable to activate cell wall-modifying enzymes. In some embodiments, samples are tempered as a liquid slurry for about 1 to about 48 hours. In some embodiments, conditions favorable to activate cell wall-modifying enzymes comprise a pH of about 4 to about 7 and a temperature of about 25° C. to about 100° C. Alternatively or additionally, samples may be tempered as a lower moisture ensilement (e.g., about 40% to about 60% total solids) under anaerobic conditions. In some embodiments, samples are ensiled for about 21 days to several months.

In some embodiments, tempering is integrated with other processes such as one or more of harvest, storage, and transportation of biomass. For example, biomass can be ensiled under conditions that condition the biomass for subsequent pretreatment and hydrolysis; that is, storage and tempering are combined. In some embodiments, during ensilement of biomass, temperatures are increased in the ensiled material such that thermally active embedded enzymes are activated. Ensilement conditions may allow preservation of biomass while providing sufficient time for enzyme polypeptides to affect characteristics of the biomass (such as, for example, amenability to pretreatment and improvement of subsequent hydrolysis).

In some embodiments, the tempering phase precedes entirely the pretreatment phase. In some embodiments, the tempering phase overlaps with the pretreatment phase.

In some embodiments as described herein, transgenic plants express more than one cell wall-modifying enzyme polypeptide. In some such embodiments, it may be desirable to activate enzyme polypeptides sequentially. It may be desirable to do so, for example, if the efficiency of endoplant enzymes is a function of the sequence in which they are activated. For example, beta-glucosidases may be most efficient after endo- and exoglucanases have cleaved cellulose into dimers, and cellulases and hemicellulases may be more efficient when accessory enzymes have reduced cross-linkages between cellulose, hemicellulose, and lignin. Accordingly, in some embodiments, cellulases might be activated after ferulic acid esterases (FAEs) have had the opportunity to cleave ferulate-polysaccharide-lignin complexes, or after other accessory enzymes have had the opportunity to cleave cellulose-hemicellulose cross linkages.

Sequential activation could be attained, for example, by using enzymes with different peak temperature and/or pH optima. Increasing temperature continually or stepwise (e.g., during a tempering step), could thereby allow activation of enzyme polypeptides with lower temperature optima first. For example, a wound-induced promoter could be used to produce a non-thermostable enzyme polypeptide after harvesting that breaks lingin cross-links and leads to cell death, before increasing temperature during tempering to activate a thermostable cellulase in the biomass.

In some embodiments as described herein, cell wall-modifying enzyme polypeptides are specifically targeted to organelles and/or plant parts. In some embodiments, cell wall-modifying enzyme polypeptides are specifically targeted to seeds. Cell wall hydrolyzing enzymes in the grain could improve yields of fermentable sugars by targeting the cellulose and hemicelluolose in the grain bran and fiber, or could loosen or weaken the outer layers of the grain kernel, making it easier to mill. Starch in corn grain is often processed to produce ethanol, but significant quantitiues of cellulose and hemicellulose from the bran and fiber are not used. In some embodiments, incorporating a tempering step prior to starch hydrolysis (e.g., of transgenic corn grain), endogenous enzymes can act on the fiber and bran and increase the yield of fermentable sugars. In some embodiments, dry seed (e.g., dry wheat) is tempered by soaking in water at a slightly elevated temperature for several hours before further processing. Such a tempering step may decrease the energy required for milling and increase the quality and eventual yield. Endogenous enzymes in the grain may also provide additional benefits.

In some embodiments, tempering comprises externally applying an amount of at least one cell wall-modifying enzyme polypeptide. External application of cell wall-modifying enzyme polypeptides is discussed in more detail in the “Saccharification” section.

In some embodiments, the seed or grain of a transgenic plant is tempered.

Pretreatment

Conventional methods for processing plant biomass include physical, chemical, and/or biological pretreatments. For example, physical pretreatment techniques can include one or more of various types of milling, crushing, irradiation, steaming/steam explosion, and hydrothermolysis. Chemical pretreatment techniques can include acid, alkaline, organic solvent, ammonia, sulfur dioxide, carbon dioxide, and pH-controlled hydrothermolysis. Biological pretreatment techniques can involve applying lignin-solubilizing microorganisms (T.-A. Hsu, “Handbook on Bioethanol: Production and Utilization”, C. E. Wyman (Ed.), 1996, Taylor & Francis: Washington, D.C., 179-212; P. Ghosh and A. Singh, A., Adv. Appl. Microbiol., 1993, 39: 295-333; J. D. McMillan, in “Enzymatic Conversion of Biomass for Fuels Production”, M. Himmel et al., (Eds.), 1994, Chapter 15, ACS Symposium Series 566, American Chemical Society: B. Hahn-Hagerdal, Enz. Microb. Tech., 1996, 18: 312-331; and L. Vallander and K. E. L. Eriksson, Adv. Biochem. Eng./Biotechnol., 1990, 42: 63-95). The purpose of the pretreatment step is to break down the lignin and carbohydrate structure to make the cellulose fraction accessible to cellulolytic enzymes.

Simultaneous use of transgenic plants that express one or more enzyme polypeptides (e.g., lignocellulolytic enzyme polypeptides and/or cell wall-modifying enzyme polypeptides) according to the present invention may reduce or eliminate expensive grinding of the biomass and/or reduce or eliminate the need for heat and strong acid required to strip lignin and hemicellulose away from cellulose before hydrolyzing the cellulose.

In some embodiments, lignocellulosic biomass of plant parts obtained from inventive transgenic plants is more easily hydrolyzable than that of non-transgenic plants. Thus, the extent and/or severity of pretreatment required to achieve a particular level of hydrolysis is reduced. Therefore, the present invention in some embodiments provides improvements over existing pretreatment methods. Such improvements may include one or more of: reduction of biomass grinding, elimination of biomass grinding, reduction of the pretreatment temperature, elimination of heat in the pretreatment, reduction of the strength of acid in the pretreatment step, elimination of acid in the pretreatment step, and any combination thereof.

In some embodiments, lower temperatures of pretreatment may be used to achieve a desired level of hydrolysis. In some embodiments, pretreating is performed at temperatures below about 175° C., below about 145° C., or below about 115° C. For example, under some conditions, the yield of hydrolysis products from lignocellulosic biomass from transgenic plant parts pretreated at about 140° C. is comparable to the yield of hydrolysis products from non-transgenic plant parts pretreated at about 170° C. Under some conditions, the yield of hydrolysis products from lignocellulosic biomass from transgenic plant parts pretreated at about 170° C. is above about 60%, above about 70%, above about 80%, or above about 90% of theoretical yields. Under some conditions, the yield of hydrolysis products from lignocellulosic biomass from transgenic plant parts pretreated at about 140° C. is above about 60%, above about 70%, or above about 80% of theoretical yields. Under some conditions, the yield of hydrolysis products from lignocellulosic biomass from transgenic plant parts pretreated at about 110° C. is above about 40%, above about 50%, or above about 60% of theoretical yields. Such yields from transgenic plant parts can represent an increase of up to about 20% of yields from non-transgenic plant parts.

In some embodiments, such improvements are observed in inventive transgenic plants expressing an enzyme polypeptide (e.g., a cell wall-modifying enzyme polypeptide and/or lignocellulolytic enzyme polyeptide) at a level less than about 0.5%, less than about 0.4%, less than about 0.3%, less than about 0.2%, or less than about 0.1% of total soluble protein. Without wishing to be bound by any particular theory, the inventors propose that low levels of enzyme expression may facilitate modifying the cell wall, possibly by nicking cellulose or hemicellulose strands. Such modification of the cell wall may make the biomass more susceptible to pretreatment. Thus, biomass from inventive transgenic plants expressing low levels of cell wall-modifying enzymes may require less pretreatment, and/or pretreatment in less severe conditions.

In certain embodiments, the pretreated material is used for saccharification without further manipulation. In other embodiments, it is desired to process the plant tissue so as to produce an extract comprising the cell wall-modifying enzyme polypeptide(s). In this case, the extraction is carried out in the presence of components known in the art to favor extraction of active enzymes from plant tissue and/or to enhance the degradation of cell-wall polysaccharides in the lignocellulosic biomass. Such components include, but are not limited to, salts, chelators, detergents, antioxidants, polyvinylpyrrolidone (PVP), and polyvinylpolypyrrolidone (PVPP). The remaining plant tissue may then be submitted to a pretreatment process.

Saccharification

In saccharification (or enzymatic hydrolysis), lignocellulose is converted into fermentable sugars (i.e., glucose monomers) by enzyme polypeptides present in the pretreated material. If desired, externally applied cellulolytic enzyme polypeptides (i.e., enzymes not produced by the transgenic plants being processed) may be added to this mixture. Extracts comprising transgenically expressed enzyme polypeptides obtained as described above can be added back to the lignocellulosic biomass before saccharification. Here again, externally applied cellulolytic enzyme polypeptides may be added to the saccharification reaction mixture.

In some embodiments, the amount of externally applied enzyme polypeptide that is required to achieve a particular level of hydrolysis of lignocellulosic biomass from inventive transgenic plants is reduced as compared to the amount required to achieve a similar level of hydrolysis of lignocellulosic biomass from non-transgenic plants. For example, in some embodiments, processing transgenic lignocellulosic biomass in the presence of as low as 15 mg externally applied cellulase per gram of biomass (15 mg/g) yields a similar level of hydrolysis as processing non-transgenic lignocellulosic biomass in the presence of 100 mg/g cellulase. This represents a reduction of almost 90% of cellulases needed for hydrolysis can be achieved when processing biomass from inventive transgenic plants. Such a reduction in externally applied cellulases used can represent significant cost savings.

In some embodiments, a mixture of enzyme polypeptides each having different enzyme activities (e.g., exoglucanase, endoglucanase, hemi-cellulase, beta-glucosidase, and combinations thereof), and/or an enzyme polypeptide having more than one enzyme activity (e.g., exoglucanase, endoglucanase, hemi-cellulase, beta-glucosidase, and combinations thereof) is added during a “treatment” step to promote saccharification. Without wishing to be bound by any particular theory, such combinations of enzyme activity, whether through the activity of an enzyme complex or other mixture of enzymes, may allow a greater degree of hydrolysis than can be achieved with a single enzyme activity alone. Commercially available enzyme complexes that can be employed in the practice of the invention include, but are not limited to, Accellerase™ 1000 (Genencor), which contains multiple enzyme activities, mainly exoglucanase, endoglucanase, hemi-cellulase, and beta-glucosidase.

Saccharification is generally performed in stirred-tank reactors or fermentors under controlled pH, temperature, and mixing conditions. A saccharification step may last up to 200 hours. Saccharification may be carried out at temperatures from about 30° C. to about 65° C., in particular around 50° C., and at a pH in the range of between about 4 and about 5, in particular, around pH 4.5. Saccharification can be performed on the whole pretreated material.

The present Applicants had previously shown that adding cellulases to plants transgenically expressing E1, an endoglucanse (EC 3.2.1.4) increases total glucose production compared to adding cellulases to non-transgenic plants, which suggests that simply using transgenic E1 plants with current external cellulase techniques can substantially increase ethanol yields. The experiment also indicates that adding cellulases to E1 plants increases total glucose production compared to adding cellulases to non-transgenic plants. This is an important result since it suggests that simply using transgenic E1 plants with current external cellulase techniques can substantially increase ethanol yields in the presence or absence of pretreatment processes.

Fermentation

In the fermentation step, sugars, released from the lignocellulose as a result of the pretreatment and enzymatic hydrolysis steps, are fermented to one or more organic substances, e.g., ethanol, by a fermenting microorganism, such as yeasts and/or bacteria. The fermentation can also be carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation or SSF.

Fermenting microorganisms and methods for their use in ethanol production are known in the art (Sheehan, “The Road to Bioethanol: A Strategic Perspective of the US Department of Energy's National Ethanol Program” In: “Glycosyl Hydrolases For Biomass Conversion”, ACS Symposium Series 769, 2001, American Chemical Society: Washington, D.C.). Existing ethanol production methods that utilize corn grain as the biomass typically involve the use of yeast, particularly strains of Saccharomyces cerevisiae. Such strains can be utilized in the methods of the invention. While such strains may be preferred for the production of ethanol from glucose that is derived from the degradation of cellulose and/or starch, the methods of the present invention do not depend on the use of a particular microorganism, or of a strain thereof, or of any particular combination of said microorganisms and said strains.

Yeast or other microorganisms are typically added to the hydrolysate and the fermentation is allowed to proceed for 24-96 hours, such as 35-60 hours. The temperature of fermentation is typically between 26-40° C., such as 32° C., and at a pH between 3 and 6, such as about pH 4-5.

A fermentation stimulator may be used to further improve the fermentation process, in particular, the performance of the fermenting microorganism, such as, rate enhancement and ethanol yield. Fermentation stimulators for growth include vitamins and minerals. Examples of vitamins include multivitamin, biotin, pantothenate, nicotinic acid, meso-inositol, thiamine, pyridoxine, para-aminobenzoic acid, folic acid, riboflavin, and vitamins A, B, C, D, and E (Alfenore et al., “Improving ethanol production and viability of Saccharomyces cerevisiae by a vitamin feeding strategy during fed-batch process”, 2002, Springer-Verlag). Examples of minerals include minerals and mineral salts that can supply nutrients comprising phosphate, potassium, manganese, sulfur, calcium, iron, zinc, magnesium and copper.

Recovery

Following fermentation (or SSF), the mash is distilled to extract the ethanol. Ethanol with a purity greater than 96 vol. % can be obtained.

By-Products

The hydrolysis process of lignocellulosic raw material also releases by-products such as weak acids, furans, and phenolic compounds, which are inhibitory to the fermentation process. Removing such by-products may enhance fermentation.

In some embodiments, processing of provided transgenic plants comprise removing, from the hydrolysate, products of the enzymatic process that cannot be fermented. Such products comprise, but are not limited to, lignin, lignin breakdown products, phenols, and furans. In certain embodiments, products of the enzymatic process that cannot be fermented are separated and used subsequently. For example, products can be burned to provide heat required in some steps of the ethanol production such as saccharification, fermentation, and ethanol distillation, thereby reducing costs by reducing the need for current external energy sources such as natural gas. Alternatively or additionally, such by-products may have commercial value. For example, phenols can find applications as chemical intermediates for a wide variety of applications, ranging from plastics to pharmaceuticals and agricultural chemicals. Phenol condensed to with aldehydes (e.g., methanol) make resinous compounds, which are the basis of plastics which are used in electrical equipment and as bonding agents in manufacturing wood products such as plywood and medium density fiberboard (MDF).

Separation of by-products from the hydrolysate can be done using a variety of chemical and physical techniques that rely on the different chemical and physical properties of the by-products (e.g., lignin and phenols). Such techniques include, but are not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, distillation, or extraction.

Some of the hydrolysis by-products, such as phenols, or fermentation/processing products, such as methanol, can be used as ethanol denaturants. Currently about 5% gasoline is added immediately to distilled ethanol as a denaturant under the Bureau of Alcohol, Tobacco and Firearms regulations, to prevent unauthorized non-fuel use. This requires shipping gasoline to the ethanol production plant, then shipping the gas back with the ethanol to the refinery. The gas also impedes the use of ethanol-optimized engines that make use of ethanol's higher compression ratio and higher octane to improve performance. Using transgenic plant derived phenols and/or methanol as denaturants in lieu of gasoline can reduce costs and increase automotive engine design alternatives.

Reducing Lignin Content

Another way of reducing lignin and lignin breakdown products that are not fermentable in hydrolysate is to reduce lignin content in a transgenic plant of the present invention. Such methods have been developed and can be used to modify the inventive plants (see, for example, U.S. Pat. Nos. 6,441,272 and 6,969,784, U.S. Pat. Appln. No. 2003-0172395, US and PCT publication No. WO 00/71670).

Combined Starch Hydrolysis and Cellulolytic Material Hydrolysis

Transgenic plants and plant parts disclosed herein can be used in methods involving combined hydrolysis of starch and of cellulosic material for increased ethanol yields. In addition to providing enhanced yields of ethanol, these methods can be performed in existing starch-based ethanol processing facilities.

Starch is a glucose polymer that is easily hydrolyzed to individual glucose molecules for fermentation. Starch hydrolysis may be performed in the presence of an amylolytic microorganism or enzymes such as amylase enzymes. In certain embodiments of the invention, starch hydrolysis is performed in the presence of at least one amylase enzyme. Examples of suitable amylase enzymes include α-amylase (which randomly cleaves the α(1-4)glycosidic linkages of amylose to yield dextrin, maltose or glucose molecules) and glucoamylase (which cleaves the α(1-4) and α(1-6)glycosidic linkages of amylose and amylopectin to yield glucose).

Hydrolysis of starch and hydrolysis of cellulosic material from provided transgenic plants can be performed simultaneously (i.e., at the same time) under identical conditions (e.g., under conditions commonly used for starch hydrolysis). Alternatively, the hydrolytic reactions can be performed sequentially (e.g., hydrolysis of lignocellulose can be performed prior to hydrolysis of starch). When starch and cellulosic material are hydrolyzed simultaneously, the conditions are preferably selected to promote starch degradation and to activate cell wall-modifying enzyme polypeptide(s) for the degradation of lignocellulose. Factors that can be varied to optimize such conditions include physical processing of the plants or plant parts, and reaction conditions such as pH, temperature, viscosity, processing times, and addition of amylase enzymes for starch hydrolysis.

Provided transgenic plants (or plant parts) may be used alone or in a mixture with non-transgenic plants (or plant parts). Suitable plants include any plants that can be employed in starch-based ethanol production (e.g., corn, wheat, potato, cassava, etc.). For example, the present inventive methods may be used to increase ethanol yields from corn grains.

### EXAMPLES

The following examples describe some of the preferred modes of making and practicing the present invention. However, it should be understood that these examples are for illustrative purposes only and are not meant to limit the scope of the invention.

### Example 1

### Identification and Isolation of Sorghum Promoters

Promoters of sorghum genes were identified by searching for gene sequences similar to that of genes having or suspected of having desirable expression patterns in other plants. Nucleic acids containing identified promoters were isolated by polymerase chain reaction (PCR)-based amplification. These promoters may be useful, for example, in driving expression of genes in transgenic plants.

### Materials and Methods

### Identification of Sorghum Promoters

Genes in rice and maize having desirable expression patterns (such as tissue-specific and developmental stage-specific expression) and/or likely to have desirable expression patterns (such as high expression levels in many tissues) due to their functions (such as genes involved in cell structure and function or intermediary metabolism) were identified. Using TBLASTN, predicted protein products from the sorghum genome (annotated and available at www.phytozome.net) were searched for sequences similar to amino acid sequences of products from the rice and maize genes that had been identified.

### Isolation and Cloning of Sorghum Promoters

Oligonucleotide primers for PCR-based amplification of some identified sorghum promoters were designed and synthesized. (See Table 2.) Primers were engineered to include recognition sites for appropriate restriction enzymes in order to facilitate subsequent cloning steps. Nucleic acids containing sorghum promoters were amplified with high-fidelity Phusion Taq Polymerase (New England Biolabs, MA) using genomic DNA isolated from two-week old sorghum leaves (Sorghum bicolor, cultivar BTx623) as template. Gradient PCR was performed using a dual block thermal cycler (BioRad, CA) for optimum amplification of PCR products.

TABLE 2

Sequence-specific oligonucleotide primers for
amplifying various sorghum promoters

| SEQ | | | |
|---|---|---|---|
| ID | Regulatory/ | Primer | Sequence |
| NO. | Gene sequence | Name | (listed in 5′ to 3′ direction) |
| 49 | GUS-NOS | ES190 | CGCGGATCCATGGTAGATCTGAGGGTAAATTTC |
| 50 | GUS-NOS | ES191 | CGCGGATCCATGGTAGATCTGAGGGTAAATTTC |
| 51 | SbUbiL4-1 | ES274 | GAGAGGCGCGCCAGCAACCACGGTGCTAGAAGCTAT |
| 52 | SbUbiL4-1 | ES275 | GAGAGGATCCCTGCAGAGAAACCAAACA |
| 53 | SbUbiL4-2 | ES358 | GAGAAAGCTTTCGCTTCAAGGTACGGCGAT |
| 54 | SbUbiL4-2 | ES275 | GAGAGGATCCCTGCAGAGAAACCAAACA |
| 55 | SbUbiL3-1 | ES272 | GAGAGGCGCGCCCTGTTTGGCTATTCCAAGTGGTTC |
| 56 | SbUbiL3-1 | ES273 | GAGAGGATCCCTGTAGAAGAAAAAACAAGCAAC |
| 57 | SbUbiL3-2 | ES370 | GAGAAAGCTTGACTCCCTTAGGGTCCATTCGTTT |
| 58 | SbUbiL3-2 | ES273 | GAGAGGATCCCTGTAGAAGAAAAAACAAGCAAC |
| 59 | SbUbiL3-3 | ES370 | GAGAAAGCTTGACTCCCTTAGGGTCCATTCGTTT |
| 60 | SbUbiL3-3 | ES372 | GAGAGGATCCCTTAGAAGCGGGTGATGGATTGA |
| 61 | SbUbiL3-4 | ES438 | GCGAAGCTTATTTAATGCTCCATGCATGTG |
| 62 | SbUbiL3-4 | ES372 | GAGAGGATCCCTTAGAAGCGGGTGATGGATTGA |
| 63 | SbActL1-1 | ES264 | GAGAGGCGCGCCAGTCGGTAGTACATGTATATG |
| 64 | SbActL1-1 | ES265 | GCGAGTTAACTTGCTACAGATTCTGGAACA |
| 65 | SbActL1-2 | ES436 | GCGAAGCTTATTGGGCGAATAGTTTTACTAG |
| 66 | SbActL1-2 | ES265 | GCGAGTTAACTTGCTACAGATTCTGGAACA |
| 67 | SbActL5 | ES650 | GAGACTAGTAGTGCTGAAAGCACCGACGATGTA |
| 68 | SbActL5 | ES652 | GAGGGATCCTCCTCAAAGTGTTCTGCAGC |
| 69 | SbActL6 | ES654 | GAGAAGCTTACACGATTAGGTCAGCAGTGC |
| 70 | SbActL6 | ES655 | GAGGGATCCTCTCAACTATTCTGTAACAG |
| 71 | SbPRPL1 | ES581 | GAGAAGCTTTACTGAGAGCGTTGTGGATG |
| 72 | SbPRPL1 | ES555 | GAGGGATCCGGCTGCTTCGCTGCTCCTGC |
| 73 | SbC4HL2 | ES637 | GAGAAGCTTACTAATTGCGCAGTTTGGTCA |
| 74 | SbC4HL2 | ES639 | GAGGGATCCGCTGGAGGAGCGTGGAGC |

### Results

TBLASTN amino acid sequence comparison analyses resulted in identification of putative homologous proteins from sorghum. Genomic DNA sequences that encode these putative proteins were determined, and corresponding upstream promoter sequences were subsequently identified for several classes of genes.

Identified promoters included consititutive, tissue-specific, and developmental stage-specific promoters and their sequences are listed as SEQ ID NO: 1 through SEQ ID NO: 48 in the Sequence Listing.

Sorghum promoters were cloned by PCR-amplification from DNA isolated from sorghum leaves, gel purification of PCR products, and cloned into appropriate base expression vectors described in Example 2.

### Example 2

### Expression Vectors Comprising Sorghum Promoters

Promoters from sorghum that were identified and isolated in Example 1 were cloned into gene expression vectors containing a reporter gene. These expression vectors are useful, for example, for characterizing patterns of gene expression driven by each promoter from sorghum. (See Examples 4, 5 and 7.) They are also designed to accommodate another gene, which can be cloned into the expression vector and expressed as part of a fusion with the reporter gene. Thus, these expression vectors can be used to generate transgenic cells and/or organisms (such as plants) that express genes under the control of a sorghum promoter.

### Construction of Base Expression Vectors to Generate Fusion Polypeptides Containing a Reporter Gene (GUS, a β-Glucuronidase)

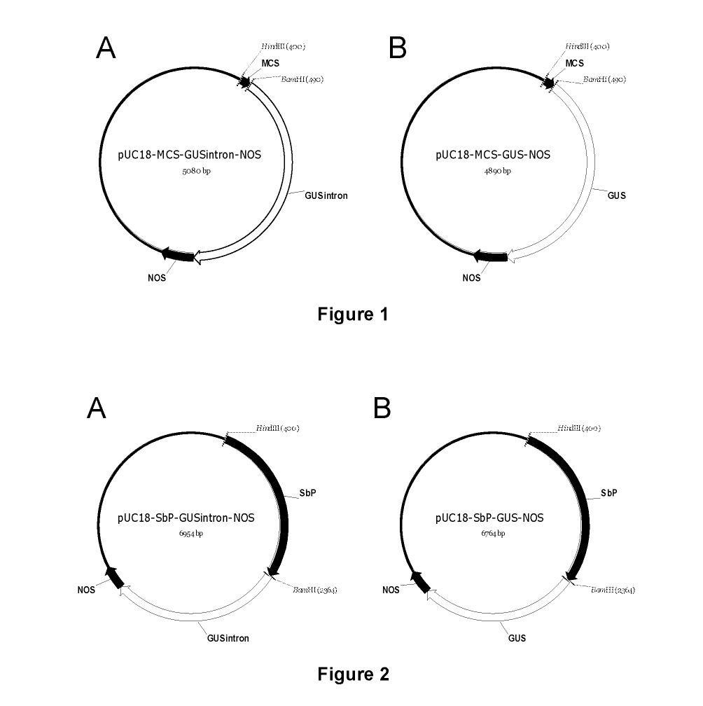

A high-copy number cloning vector pUC18 (Invitrogen, CA) was used to create base vectors containing a reporter gene. First, a region comprising the coding sequences of β-glucuronidase (GUS) gene with or without an intron from catalase (“GUSintron” and “GUS” respectively in plasmid names in FIGS. 1 and 2) and the nopaline synthase (NOS) terminator was amplified by PCR using pCAMBIA1301 plasmid DNA as template. pCAMBIA1301 contains GUS cDNA, the catalase intron, and a NOS terminator and is available from CAMBIA (www.cambia.org). Catalase intron present within the GUS gene is spliced out during transcription in plant cells. As with other prokaryotes, bacteria (including E. coli and Agrobacteria) do not have the splicing mechanism for introns and will not be able to express the GUS reporter gene, though they can still carry the vector.

Restriction enzyme recognition sites BamHI-KpnI were engineered into PCR primers ES190 and ES191 (see Table 2). PCR-amplified GUSintron-NOS and GUS-NOS fragments were digested with BamHI-KpnI enzymes and cloned into pUC18 vectors to create the pUC18-GUSintron-NOS and pUC18-GUS-NOS vectors. A multiple cloning site (MCS) cassette comprising HindIII-AscI-PstI-SalI-PacI-NotI-XhoI-SpeI-HpaI-XbaI-BamHI restriction enzyme recognition sites was PCR amplified, digested with HindIII-BamHI enzymes and cloned into pUC18-GUSintron-NOS and pUC18-GUS-NOS to create pUC18-MCS-GUSintron-NOS (FIG. 1A) and pUC18-MCS-GUS-NOS (FIG. 1B) constructs respectively.

Cloning of Sorghum Promoters into the Expression Vectors

Sorghum promoters were generally classified into one of two categories depending upon the presence or absence of the first intron located within the promoter region. Since the first intron had been previously shown to enhance gene expression in monocots, efforts were made to retain the first intron in the tested sorghum promoters. Sorghum promoters without the first intron were cloned into pUC18-MCS-GUSintron-NOS vector and promoters with the first intron were cloned into pUC18-MCS-GUS-NOS vector. PCR-amplified sorghum promoters (SbP) were digested with appropriate restriction enzymes and were cloned into above described vectors (whose maps are depicted in FIGS. 1A and 1B) to create pUC18-SbP-GUSintron-NOS (FIG. 2A) and pUC18-SbP-GUS-NOS (FIG. 2B) vectors.

### Example 3

### Transformation of Corn by Particle Bombardment

The present Example demonstrates successful generation of transgenic corn plants expressing a gene under the control of sorghum promoters isolated as described in Example 1. Corn leaves were transfected with expression vectors (generated as described in Example 2) encoding a reporter gene under the control of a sorghum promoter. Reporter gene expression was also analyzed and demonstrated that sorghum promoters SbUbiL4 and SBPRP1L can drive high levels of heterologous gene expression in monocot plants.

### Materials and Methods

### Transfection of Corn by Particle Bombardment of Hi-II Corn Leaves

M10 Tungsten particles (Sylvania, Mass.) were used for microprojectile bombardment experiments. Gene expression vectors used in transfection experiments were generated as described in Example 2. These vectors encode a GUS reporter gene under the control of a sorghum promoter (either SbUbiL4 (sorghum ubiquitin-like-4 promoter; SEQ ID NO: 11), SbPRP1L (sorghum proline rich protein 1-like promoter; SEQ ID NO: 45), SbActL1 (sorghum actin like-1 promoter; SEQ ID NO: 1), SbUbiL3 (sorghum ubiquitin like-3 promoter; SEQ ID NO: 10), SbC4HL2 (sorghum cinnamate 4-hydroxylase like-2 promoter; SEQ ID NO: 43), SbActL5 (sorghum actin like-5 promote; SEQ ID NO: 5), or SbActL6) or of a control promoter in monocots (OsAct1; rice actin promoter that is known in the art. See U.S. Pat. No. 5,641,876, the contents of which are herein incorporated by reference in their entirety).

Stock solution for transfections was prepared by washing 50 mg of tungsten particles in 500 μl 95% ethanol, followed by washing in water 4-6 times. Particles were then suspended in 500 μl ddH2O. The stock solution was used for a maximum of 12 hours after resuspension. 25 μl of resuspended tungsten particles were mixed with 5 μl of DNA (200 to 500 ng/μl) in a microcentrifuge tube and vortexed for a few seconds. The mixture was allowed to sit at room temperature (RT) for 1 minute. DNA was precipitated by adding 25 μl of 2.5 M CaCl₂ and 10 μl of 100 mM Spermidine and leaving the mixture on ice for 4 minutes. Fifty microliters of the supernatant was discarded, the remaining coated particles were kept on ice, and 2 μl were used per shot within 15 minutes. Mixtures were discarded 15 minutes after preparation and, if needed, freshly coated particles were prepared for additional transfections.

Leaves from 2 to 3 week old corn seedlings were used for the experiments. The youngest leaf was trimmed into ˜7 cm pieces and placed in a petri dish with wet filter paper. Coated particles were bombarded against leaves at pressures of 60 psi and 28 mm Hg. After particle bombardment, leaf tissue samples were kept in Petri plates under moist conditions for a 24 hr period.

### Analysis of Histochemical GUS Expression in Plant Tissues

In order to analyze GUS expressions pattern in plants transformed with each expression vector, bombarded corn leaves were incubated with 5-bromo-4-chloro-3-indolyl glucuronide (X-Gluc) for 24-48 hr as previously described (Jefferson et al. (1987) EMBO J. 6:3901-3907.). Tissue samples were cleared using 70% ethanol repeatedly until the most of the chlorophyll is removed. Samples were observed for GUS expression (seen as blue spots) and images were taken using a Leica stereo microscope (Leica, N.J.). Blue colored GUS spots were counted from all the experiments and are presented in Table 3.

### Results

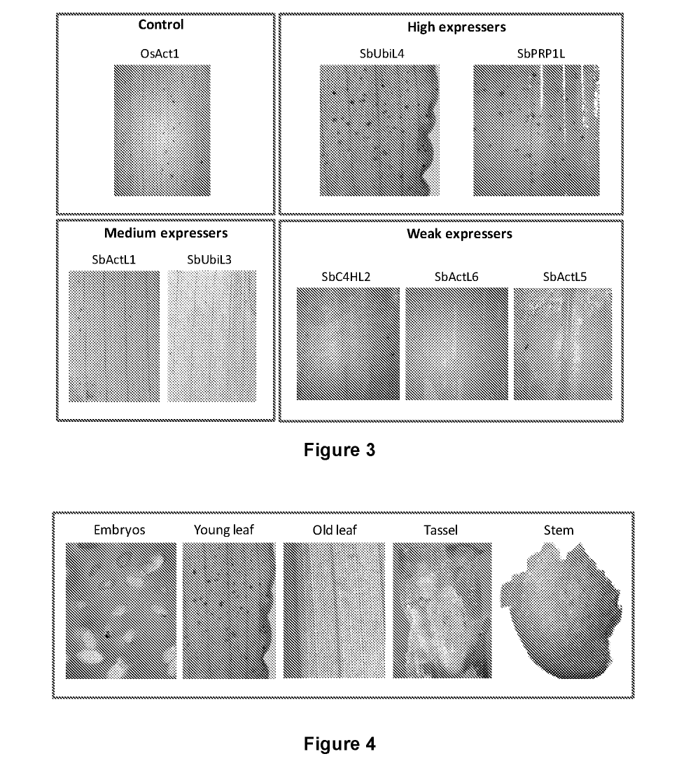

As shown in FIG. 3, expression of GUS was successfully driven by a variety of sorghum promoters. Based on the strength of the histochemical GUS expression and/or on counts of GUS spots, sorghum promoters were classified into high expressers (SbUbiL4 and SbPRP1L), medium expressers (SbActL1 and SbUbiL3) and the weak expressers (SbC4HL2, SbActL5 and SbActL6).

TABLE 3
Quantification of GUS reporter gene expression
driven by various sorghum promoters

| | SEQ | No. of GUS | Tissue |
|---|---|---|---|
| Promoter | ID | spots | type |
| OsAct1¹ | — | ~60 spots | Leaves |
| SbUbiL4 | 11 | ~250 spots | Leaves |
| SbPRP1L | 45 | ~250 spots | Leaves |
| SbActL1 | 1 | ~58 spots | Leaves |
| SbUbiL3 | 10 | ~96 spots | Leaves |
| SbActL5 | 5 | ~4 spots | Leaves |
| SbActL6 | 6 | ~16 spots | Leaves |
| SbC4HL2 | 43 | ~12 spots | Leaves |
| SbC4HL2 | 43 | 20 to 30% of the | Stem |
| | | sections | |

¹rice actin 1 promoter

As shown in FIG. 3 and Table 3, sorghum promoters (SbUbiL4 and SbPRP1L) can drive high levels of heterologous gene expression in a monocot plant.

### Example 4

### Sorghum Promoter SbUbiL4 can Drive Gene Expression in Multiple Tissues

To determine if sorghum promoters can drive reporter gene expression in tissues other than leaves, a sorghum promoter (SbUbiL4; SEQ ID NO: 11) that was characterized as a “high expresser” as demonstrated by experiments described in Example 3 was characterized further. Expression plasmids containing a reporter gene under the control of SbUbiL4 were transfected into other tissues in corn plants. Results from these experiments demonstrated successful expression of transgenes in multiple plant tissues using SbUbiL4.

### Materials and Methods

Expression of SbUbi4L:GUSintron:NOS in corn (Hi-II genotype) was tested in a variety of corn tissue: embryos, young leaves, old leaves, stems, and reproductive organs such as tassels. Tissues were bombarded with tungsten particles coated with plasmid DNA of sorghum promoter SbUbiL4 driving the GUS reporter gene using materials and methods as described in Example 3.

### Results

GUS expression was detected in a variety of tissues, and was especially notable in embryos and young leaf (see FIG. 4). These results show that the SbUbiL4 promoter can successfully drive heterologous gene expression in multiple tissues and demonstrate the ubiquitous nature of the expression of SbUbiL4 promoter.

### Example 5

### Analysis of Gene Expression Pattern of Sorghum Gene SbUbiL4

Results described in Example 4 demonstrated that the SbUbiL4 promoter from sorghum can drive expression of a transgene in multiple plant tissues. To further characterize the pattern of activity driven by the SbUBiL4 promoter, the expression pattern of the SbUBiL4 gene was studied by searching Expression Sequence Tag databases with SbUbiL4 coding sequences.

Full length coding sequence of SbUbiL4 was searched using the BLASTn program against publicly available EST databases (http://fungen.org/Sorghum.htm) generated using 26 sorghum tissue libraries. EST results (n=517) from the BLASTn search were sorted in decreasing order for the relative abundance of transcripts in each EST library. Consistent with the GUS reporter gene expression data presented in FIG. 4, the EST profile of SbUbiL4 showed expression of SbUbiL4 in multiple tissues (Table 4), suggesting a ubiquitous nature of expression of the SbUbiL4 promoter.

TABLE 4
Expression Sequence Tag (EST) profile of sorghum gene SbUbiL4
across 26 sorghum EST libraries

| EST Library | # ESTs |
|---|---|
| Oxidatively-stressed leaves and roots | 95 |
| GA- or brassinolide-treated seedlings | 63 |
| Callus culture/cell suspension | 44 |
| Wounded leaves | 43 |
| Acid- and alkaline-treated roots | 33 |
| Pollen | 31 |
| Abscisic acid-treated seedlings | 30 |
| Anaerobic roots | 30 |
| Heat-shocked seedlings | 25 |
| Nitrogen-deficient seedlings | 24 |
| Ethylene-treated seedlings | 21 |
| Salt-stressed seedlings | 18 |
| Pathogen-induced: compatible | 15 |
| Phosphorous-deficient seedlings | 10 |
| Dark-grown seedlings | 9 |
| Embryos | 9 |
| Light-grown seedlings | 6 |
| Pathogen-induced: incompatible | 4 |
| Immature panicles | 2 |
| Drought-stressed | 2 |
| Iron-deficient seedlings | 1 |
| Ovaries | 1 |
| Salicylic acid-treated seedlings | 1 |

### Example 6

### Tissue-Preferred Expression of SBC4HL2

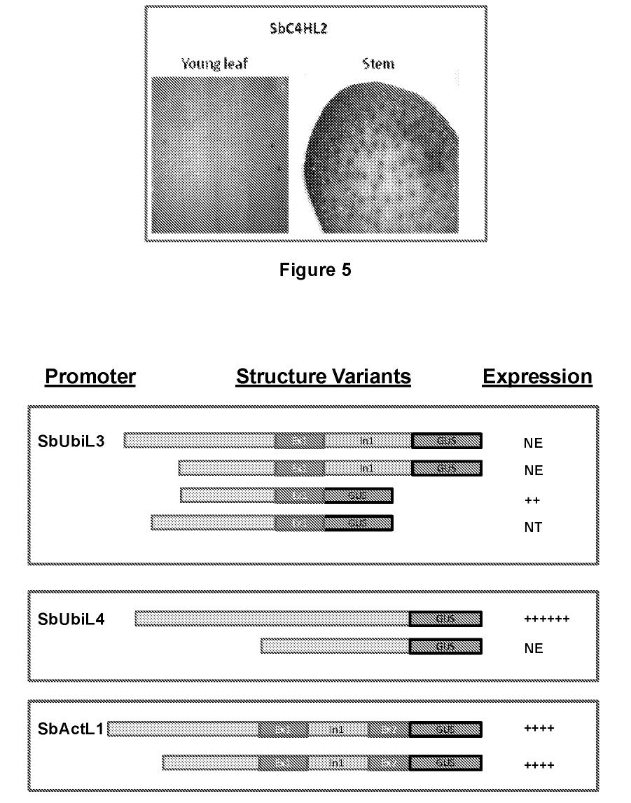

Tissue-specific and tissue-preferred promoters play an important role in driving heterologous transgene expression to the appropriate levels in the desirable tissues. In order to test the expression levels of sorghum promoter we bombarded corn leaves and stems with tungsten particles coated with plasmid DNA containing sorghum promoter SbC4HL2 positioned to drive a GUS reporter gene. As shown in FIG. 5, the SbC4HL2 promoter is highly expressed in the stem tissues as compared to young leaf, demonstrating tissue-preference.

These results show that tissue-preferred expression can be achieved using a sorghum promoter.

### Example 7

### Structure-Function Analysis of Sorghum Promoters

Analyses described in this Example are directed to understand structural requirements of promoters for driving transgene expression in plants. Structure-function analysis of promoters should help identify the optimum size and the sequence of promoter that can drive high levels of gene expression in transgenic plants.

Monocot promoters typically contain introns in their regulatory regions and the first introns have been shown to control and enhance the gene expression in transgenic monocot plants. In addition, promoters contain regulatory elements such as binding sites for transcriptional activators or repressors that are implicated in controlling gene expression levels throughout plant growth and development.

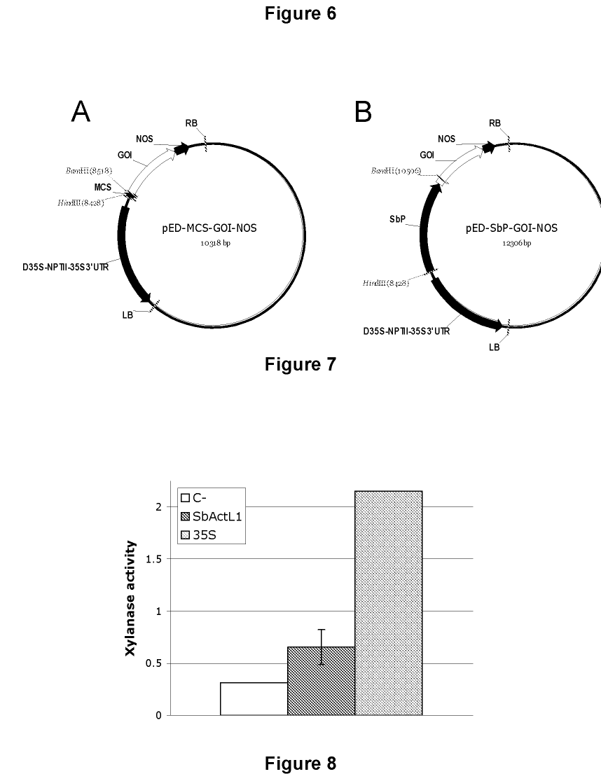

To determine which gene regulatory regions are beneficial to and/or required for gene expression, systematic deletions were carried out in the regulatory regions of sorghum promoters SbUbiL3, SbUbi4L4 and SbActL1 using primers listed in Table 2. Different structural variants were cloned into expression vectors to drive a GUS reporter gene. These structural variants were tested in corn leaves using particle bombardment. As summarized in FIG. 6, results indicated that shorter versions of both SbUbi4L4 and SbActL1 promoters are functionally more active when compared to their respective full-length parent versions. In case of SbUbiL3, the longer parent version had no activity, whereas a shorter version without the first intron was functional.

These results provide some clues as the structural requirements of some sorghum promoters and demonstrate that systematic analysis of promoters can facilitate optimization of the promoter activity.

### Example 8

### Use of Sorghum Promoters to Drive Gene Expression in Transgenic Plants

Sorghum promoters provided by the present disclosure may be used, among other things, to direct expression of a gene that encodes a particular protein or polypeptide in plants. The choice of the particular selected genes (gene of interest; “GOI”) includes but, is not limited to, cell wall modifying enzymes and agronomically important traits as described herein.

To facilitate expression of a gene of interest in plants, a plant transformation binary vector pED-MCS-GOI-NOS was created that will allow cloning of different sorghum promoters to drive the gene of interest (FIG. 7A). This vector uses the kanamycin selection (NPTII) as a selectable marker for identifying and isolating the transgenic plant cells. Sorghum promoters provided in the present disclsoure will be cloned into this vector to develop pED-SbP-GOI-NOS, as shown in FIG. 7B.

Polypeptides encoded by genes of interest can be, if desired, targeted to various subcellular compartments for the optimum expression. These expression vectors will be transformed into plant cells to generate transgenic plants using standard plant transformation methods (such as, for example, agrobacterium-mediated transformation, particle bombardment, and electroporation).

### Example 9

### Sorghum Promoters can Drive Expression of Genes in Dicot Plants

Examples 3, 4, and 6 show that sorghum promoters can be used to drive gene expression in monocotyledonous plants. Results described in the present Example demonstrate that sorghum promoters provided in the present disclosure can also be useful in driving expression of a gene in dicotyledonous plants.

The SbActL1 promoter was cloned into a plant binary transformation vector upstream of a microbial xylanase gene that encodes an enzyme that catalyzes the hydrolysis of xylan substrates such as remazol brilliant blue-xylan (Biely et al., 1988, Methods in Enzy. 160: 536-541.). The SbActL1:Xyl construct was transiently expressed in tobacco leaves using agrobacterium infiltration, along with a xylanase construct under the control of the 35S Cauliflower Mosaic Virus promoter. Infiltration media alone was used as a negative control. Total protein extracts were prepared from the infiltrated leaf tissue and assayed on RBB-xylan to measure xylanase activity spectrophotometrically at 595 nm. Activity of extracts from SbActL1:Xyl leaves was significantly greater than that of the control (C—) extracts (FIG. 8), though lower than extracts from 35S:Xyl leaves.

These results demonstrate that sorghum promoters could be used to produce transgenic dicotyledonous plants.

TABLE 5
Sequences of novel gene regulatory elements

SEQ ID NO: 1
Sequence Length: 2765
Sequence Type: DNA
Organism: Sorghum sp.
GTACACCATTGATCCCCAGCATATAAAACTTTAATAAAGTCGGTAGTACATGTATATGGGCT
CACTAAATCCGTATCAGCACGCGTGTGCCACTACCACTAGAGATGTGTGCTCAGCTGGAGTA
CTCTAGTTTATTATTATTATTATAGTTCCAGGTCATATGATCCTGGACCCAAATCGCATTAA
ATTTGTTGCAACTGCATGCAAGGTGTTGCTCTTTAAAAGCAATTATATATATATATATATAT
AGTAATAAAAAAAGGGGAAAAATAAGGATCTGGAAGCCGGCCCAGGCGCAAAAGGACCGGTC
CAGCGAGGAATGGGTTGGGCTTGCTGGGTGCACCTCCACGCTAGTCCAGCCGCACAAATGGG
CCCGCCGCCATCTCGCTCCATCGGACGGGTCAGCTTGCTCCACGTAGCCCATCGGAAGGGAA
GGCCCTTTCCTTTTTTTTTTTCCCTTGCCAGTGCCAGGTATGCTGCTTCATATTATACCCCT
GCGCCCAATTTGGAATCTTGGCCAATCGATCGATAATAACAAGGACAGATGATTCGTGACCC
ACGCTTCTTCCTTGATTGTTTGGTTGCTTTAGTTGAAGGCATCATCATCAACTAGCTGTGCT
GTGCAGCTCGTCGGCTCTAGCTAGATGCCATGTGGGTTATGCATGAGTTTGTTTCGTGTTGC
ACATTTTAGCCTATATGTTTGCTGGTTTCATCGACTTCAAGTTAATCTTTGAAAGAGAGCAT
ACAACGTAAAATATTTTTCTAGAAACCGGAGCCATTTTGTGAAGAAAAACTTCTCCCAAATG
TGACGCCTTACTATTACATTACACCTCTTTAGTAGAGCTTTGGCCAGTCCTAGCTACTAGGC
CCTATGTGTACTATAGCAATGAATTATTTGTATCTCTTTTGTCAAACCGAACTAAGATTGGG
CGAATAGTTTTACTAGCTCTGACTTCTCGATCTAAATATATATTACTATTTTGTTTGAAATT
GTAAGTCATTTTGACTTTTATAAATTTAGAGTTTTTACTATATATCTAGACATAGTATGTAT
GTATGTATGTATGTATGTATGTATGTATGTATGTATGTATCTATGTGTATAGCTAAATCTAT
TATGGATCTAGAAAGGCTAAAATGACTTAGAACTTGAAATGGATGAAATAATTCATTACAAA
ATAGTTGCTTCATCCTATTGCAAACCTTGATTGGCCTTGTTTGGATCCACATATATTGATCC
ATAATGCACATATATTAGAGTTGATTGAAATGAAACTTAGTTTAATTTCACTTCAACATATG
TGGATTGAGGTACATACACATACATGTAAACAAGATCTTATATAATTTATCTTACAACTCTT
CTCTATGCTTTAGATCGATATAACTCATCACAAAAATATGTACGCGTCTTTGGTCATTAATT
CCTTGCATCTATTTAATGAAAGAGACCAATCTTATCTTGTAAATAAATAAAGGCATTATTTG
ATGGAGCTCCAAAAAAGTTAGGTATTTGTACCTTCATGAAAAGTTGCATTTAGAGTTAAGGA
CTTAAGGTACAACGAGGGCATTTAGATAATTCATTTTCCTACTGGGTACAATTATTTAAATA
TCTAGATCTAGAGCATAGGCATCAATCACCGCTCGATGTACTAAACAAATGGCATCAAAAGT
TTTTCTTAAAAAAATGGCCTCAAAAGTAACCACACAAAAAGTTTCAGGAGTAGTTAGTGGTG
ACATTCATAGATAAAATCATCTCAATCACTTCTAATTTTCCTAGATATAGCCTAACATAAAC
AAGAACACAAAAAAGATCGTTTAAGGAAAAAAAGTACACCTGCCTATACAAATAAAAAAAAC
TGTACCATTTAAACCATTTTGCAATCAGAATTCAGAACTAGGCAGAAACTACTCCTTTTTTT
TTGTACTAATGTTTTTTAAATTAATTTTCTCCCACCCGGATGCGCATATAAAAACCGCCGAA
ACCCTTGGCTCTCCTCACTTGACCACCGCAACCACTTCTCCCCTGTTTCCTCTCGTGCATTC
TCCGTGGGAAGCGAGGAGGACTCGCGGCCGGCGGCAGGTTCTGCTAGATCTCCGGGTAAGTG
TTGAAAGAAAAGAAAAATTGTTGGAGGAATTAATTCCGAATCTTTCTCCATTGCGATTTTGG
TGTCTAGTCTGAAACGCGCAGTTCATCCCATTCCTGCCCACAGTAGATCAGATCCGATTTGC
TCCTGCGGCAGGCATTGCGATTGCTGGGCCCTGGTTGCCGATTAGTTATCGACATAAACCGC
GGCCAAGCCCCCCAAGAAGTTCTTGCGGGGTGACAAGAAGAAGATAAACAGTTTGGCCATCT
CGCCTCGCAAGGATAACCGCCCGTTAATGCATTTTTGTTTCTTGATTTCTGAATAAACATAG
GTATTATTCGATTTCTGGATAATAGAGTGCCATTAACTGGTCCCTGCATAGCGTGGTATTGG
TTACAGAAACTGTGCCGGTTCTGTAGATTTAGATGAATCACAGTGTCAAGAGAGACAGGATT
CACCTGAATCCTTCTGTTAAATTAGAAAATAGACATGATCCAAGGTCCTGTGATTTCTCAGT
GAGTAGTCGTGTAGAGTTTATTTATTTTGGTCCTGTTTCCTTTGCTGAAAATGCAGTTAATA
CCAAGTTTTCTGCTGTTTCCTATTAAGATAGATACTCTGTTACTGATTTTTACCCTTTGGCT
CCTCCTTGTGTTTTTGTTCCAGAATCTGTAGCAAATG
SEQ ID NO: 2
Sequence Length: 2720
Sequence Type: DNA
Organism: Sorghum sp.
ATCCGCCGGATCGTCGGCCCTCGGGCGCTCAAACACGCCAGAGCAAATATTCCTGCTCGAGC
CGCGCAAGTCGCACAATATTTCCATCATCCTCAAGTCCCTCACGGTGGGGCGGGATGAAATC
GACGCCCTCCGGGATGGGCACACAGACAGAACTCAGCACTGGGAGGTCCTGGAGAAGCTTTC
GCGGCTCAACATCTCTAAAGAGGAAGAGTCCACCATCTTGAAGTTCTGTGGGAACCCCGACA
GGCTTGCCCCAACGGAGGCCTTCCTCCTCCGTCTCCTCCTTGATGTGCCAGGGGGTTGTGAA
GGCTGCAGAACAGGAGCTGAAGGCACTAAGAAGGGAGCAGGAAAGAGTACTTGAGCTGGTCC
AGAAGACAACAGAGTACAACCATGCTGGTGCCGCCAAGGAACGGAACGCACATCCCCTCCAG
CTGTTCATCGTAGTGAGGGACTTCCTGGGTATGGTTGATCAGGCATGTGTTGACATCAAGAG
GAAAGTGCAACAGAAGAAACCAGCACCATCGTCATCGCAGCCAAACGCAGCAGCTGCTGCCC
CCACGGTGGCGGCTGCGGCTGCGGCCACAACAGCGGTGACAGCGTCAGTGACAAAGGAAGCG
ACCGATGGTCAAGCAGCACCAACTCACAAACCACCCGAAGAGGCAGATAGTAAAAGGAAGAG
GGTCATGCCAAGATTTCCAAACCTACCAGCGCACTTCATGAAGGAAGTTCAGATTCTGATTC
AAGTAGTGACGAGGAATAGATTGAACGGGTGGCTGTCAATGATTGTTTACATTGTTTTGAAT
TGGTTTTGTAGAGGTATAGGATAGCTGCAGACTGTACATAAAAGCAATTTTTTACATTGGTT
CTTTTGTCCATTTCTTCAATCAAGATCCATATCGAGAGCACCGAATAGAAATATAGAATTCA
GAAATTTGTGAAAAAAAATGATGTGAAGATCTCAATTGCTTAGAAATGATATCTTTGTTTGA
GGGGAAAGCCCCTACTGTCGGTAGCTATATAAAGAAAATGTGAAGGCATTGTACAAATACGA
AGGATAAAAAGTTAACAAAAAGGAGAGAAGACCTGGACATAGAGAGACAATTAGGAAAGATA
TCCCAGCCATGTCAGAAGATATGTTGGGTCTCTTTTTTAAAAAAATGGGTCCATTCCGTTGC
TTCCAAATTTTTCAGACTACTATTATAAAAAAATCCCCATGAATGAAGAAAGGTAACTGTAA
ATATTTTTTTCCAGCTTGCGTCAATCATGATGAGAAAAGGTAGAGGCATGATCCAGGTGAGA
CCAACCACATGCCAACAGGTCTACTGAATTGACATCCAAAGAAGAGATGCAGCCTTGTTTAG
TTCCCAAAAAGTTTTCAAAATTTTTTAATTTCTCGTCACCTCGAATCTTGTGGCACATGCAT
AGAGCATTAAATATATATAAAAATAATAACTTATTGCGCAGTTTGTCTGTAATTTACATATA
TATACTCAAATGTGTGATAGGATGGCAGAGAGATGCGGCGGAACTTATTTATAGATTTACTA
GCGCACGAAAGAAATGGATATCGAAGATTTCTTCCTACCGATATTAAACATTATCTTGTCGT
ATCACGGAAAGGTCTATAAAGTAGAATTTGTGAGCCTATAACGTCGTGCTCTCCGTGACCGT
GTTTGATTTCATGAACTTTGTTTTCTTTAAGTTAAATGGAACTTTATTTGGTATTTAAGTTT
TATTATAATATTATTATCTTACTGTGCGATCCATGTATTTTAAATAAAAAATGTTAGCTATC
CCGTAGCAACGCACGGGCACGCTACCTAGTTTAACTAGTAAAATCATCGTAAAAATAACTTT
GAACCCTTTCAATTTTCGGATTGATAGTATAGCCTCAAGGGGCTTTTGGTGCGGTTTTGAGA
GCTATTTTCTTTAAAAAAAGATTTATAATTTTTTTTAAATCCGGGAGTCGTGAGGAGTCAAG
GAGACCACGTTCTCCTCACGTTCTCCTCCTCCTCCTCTCCTTCACACCACAGACAGCCCCCG
ACTGCCACAAGTCTCTCTCCTCTCCTCTCTCTCTCTCTCTCTCTCTCCTCGCGACGACTGGG
CGAGACCGCCGCCGCCCTTCGCCAGGTGCCCAGGTCTCCGCCGCTTCCTTCGCCGGAGGTCA
CCAGGTTCGCCTGCCCCCTTCCTTTCCCTTGGTGCTGGGCGAAACCGATCTCCCAAACCGTA
TCTTAGGCTTCCCATTTGTGTACTCGCATCCAGATCTGATCTAGTTACACGTATAGCATGCT
TGCACCCGGATCTAATCTAGTTAGAAGCGTAGTGAACTTGCATTCGGATCTGATCTACAGTT
GCATCCGGATCTGGCGTAAAAGAGTTTGCTAGTTTTCTTTTACGAATTGGTCTAAGCTAACT
GGATGCTTGTTGTTGGTGTGATTCCAGTGAAGAGCAGTTGGTCTTTCGTCCGGAAGTAGACT
TCCACCACGCATATTAGCTTACTGGAATATGATGTTGCAAATTTCAGATGCTATAAGTCATG
AATAAATTGTTTTCTTTGTGTGACCTTTTTTCTGTACGAAGAATGTGATTTACTGCTCAATT
GGCACGTGTATCCGAATCACAGTGTTCCTTTCAATCAATCATATATTGAAAATAAAAAAAAT
TAATCACTGATGGATTCTCTTTATATGCGAACCTGCAGCAGTTCTGTAGAAATG
SEQ ID NO: 3
Sequence Length: 2348
Sequence Type: DNA
Organism: Sorghum sp.
CGGGCGCGCACGTACTGCTACTGCCGCGTGGGCTGGGCCCCACGCTGGGTGATCGGACGACT
CGGAGCTCCTCCGCGTCATCTATCGTGCGGTCCAGCTTGGCCGAGCATCTCCAGCGCATTCC
AGCCTCAGGAACTCAGGATCTTCGGCACTTTTCTTTCCCCCTTTTTTTTTCAAAAAAACGCT
TGATTTGATTTTTTCTCTGTTTCCATTTTCTCTGGCTGTGGACCACTAGTTATTGTGCCTGA
TGTGGAAGGAATTATTTCTCACACCATATATATTATTTATTTTCATTTTTTTAATATATAAA
AGTGTGTCCTTCGATGTCACGCTTTCAAGCCCATGACAACTGGAGTTCGGGAGATGCATAAC
TGTATGTGAGATTTCTTTTTCATTTTTTTTTCGTTTGTATTGCATACTACTTACAGTTGGCA
CTGGCCAGGACGGTCTGGAAGATTCTAGAGATGTTGGTTAACAATCAAGTGTCCTCCACATT
TTCATGGAAAAAAATAAACCGCATTGGAAAAATATAATATGGGTTCCTAAAAAAAGGAACAC
GAGTGATGATATTTATAGTATTTTTTCAACAAAGTAGCCTTACTTATAATTTTTGCTAAAAA
TGATGGCTCTGTGTCATTCAGAAATAATACTATTGTTTTCATAAAAGCAAAAGTTTTTTCTT
ATCCCTAATGGTATTGAAATATTAGAGTTCATTAAAAATGGTAAAAAGGGAATGATGGATGA
AGGAAGTTTGTTTTTAACTCCCCTACAACATCTAAACCTATTTATGGTTAGGGGTCTAAATA
AAATAAAATGAACTGGAAATTAGCTAAGCAAAGATAGTTCTAGCAGACGAAACAACTGCAAC
ACTCTTTCGCCAGCAAAACAAGAACAAGAACACAATTAACCAAGGCACAATCAAACAGGCAT
CTACCATGTTTTTGTTGGTAGTAAAATGAACATGAAACCAACAAACACATGATGGTGCCTAG
CACACAAACACATCCACCACACTCTTTTCACATCAAAATGAATTAGACTACAATAAAACCAG
GTGTGACAACCAAAATTATGGCCACAGAGCAAACTTTGGATAAAGTGAATGCAAATATTAAC
AGGAGAATAGATGGCACTTGGACAAAAAAACCGGATCCCATCATAGATAGAACAAATGAAAA
GTAACATTATTAAGGCCTTGTTTAGTTCCCACCAAAATCCAAAAAGTTTTCAAGATTTTCCG
TCACATCAAATCTTGCGGCACATGCATAAAACACTAAATATAGACGAAAACAAAAACTAATT
ACACAGTTTTTCTGTAAATCGCGAGACGAATCTTTTGACTCTAGTTAGTCTATGGTTGGACA
ATATTTGCCACAAACAAACGAAAAGTGCTACAGTAGCGAAATCCAAAAAAAATTCGCATCTA
AACAAGGCCTAAATTACAGCATGATCAAATCACACCAAAAAATACACCAACCAAACAACATC
TAAGTGAGTGTGGAGCACTAAACATATGACACCATCTATTTAAAAACAAAATCAACACAACT
GAAAAATAGTTACACGTGTACTTAAAAAAATCTCTATCGCAGAGAGAGAGAGAAACAATAAA
TATATACTATGAAAACATTATCATGGTTGACGACGATCCTTGCTCGAAATACAGGCTGAATA
TTTGAAACCGGTTTTGAGGGAGAAAAAAAAAACCCGAAAATGTACACCTCGTGTTTGGAACC
GTATCCCCGTGGGGTCCCTTCCATCCCTTCCCCCGGCTGCCTCCCTCGTATAAAACTCCACC
ACCCCAGTCCTGGGCGGCGAGCACGCCGCCATCCAGGTCCAGCCGACCTGCCTCCCGCCGCC
GCGACCCCACACCGCCTCCCTTTGCCGGCGGCCCCGTCCCGCGGATCGGTTGGCGTCTCCCC
TTGCTGCTGGTATGCAAGCCCTGCCTTCCTCTGTCTGTTTTGTTTGTTTTTTCCCCCCTTCT
TTCGTGCTGTTCAGCGGTGGATCTCACCCTTTGCCGTGGGTGGTGAGCGCTCGAACCCGACC
GAATCCGCTGGTACGCGCGCTCCGATCCGTCTAGTTCGTCGCGGATTCATTCGCTTAAACGC
GGGCGGAGGTTTGTAGCTGGGAGCGGTTGATTTCCCGAACTTTGTGTTAAAAAAAATTATGG
GGAGTTTAAGTGCGACAGCAAACTGCAGCAGGATTTGTAAGAATTTCCTGCGGAATTTGCCC
AGTAGGACTGCCCTTGATGGGCTGTGTGTGCTGGACACAGATTCTGCTATAGTGATTATTAG
TAGGAGTAGCCCTCTTATTATGCTTGAATCCGTGGCAGAATCACTGACCAGATG
SEQ ID NO: 4
Sequence Length: 3552
Sequence Type: DNA
Organism: Sorghum sp.
CCGAGGGCAGCCTGCCTGCACTGCAGAATGTGCTTGGTCACGAAAAGGTGACTTGCAGACAG
ACAGGATCACAGGAGGTGGACTGGCGAGGCGTCGAGGGGAGGGGGGGCAGAGCGAAGCAAGC
AGGTGCCGTAGAGCGACGGCGAGAAATGTTGGCACGGTGGAGGCGTTGCCGTAAAGCGACCA
AAACGAAGCCAAAAAAAAGGCGGTCCGGAAAAGGCCGCCGCCCACGGTCCATCTTTTGCCCT
GTTCCGTGGTTGGCCCCGGCGCGGCACCGTCCCCCCCTGGCCGGCCCCCATTCCACTTTCGT
GTCATGTGCTCATTTTTTCTTCTCTTCCAATCCTACTGTCAAGTAGTAGTACCAACCAAAGC
ACCGACAGCGCAAGGCGTAGGACGAAAAGGAAAACAAGGAAATGGTGTGCTGGGATGATTGG
AAAAGGTGATATGAGATGGGTGATATGGACTCCTAGACAACGGCAGCTAGCTTTGCCAACAA
AGTAAAGTTGCTATAAGGCGTATGAGATTATATGTATGGCTCTTTTTTTTTTTGTTAAGCGT
ACGAAGTGCTCACTGTACAGAGATTTAGGAAGCCAATTGCTCCGGATTATGACTCTGTTTCT
AAGCAACTGATAAAGAGGTTGTGGTTTTTCATGTGACCCACCCCTTGTTGCCATCTTGTGAG
ATAAATGTTGCCAAGAGGAGTGCTATCCACCAGGCTTCAGTCTGGCCGAGTTTACGACGGAC
GCTGGCCTGCTCAGTACGTCGCTACAGGGCGACCATCCGGTTTGTGGTCAGTGACAGTTCCG
CCACACCTCAACAATTTTACCAAGCGAGTGCTGAAGAAGGTGGACGCCTCGCTAATTCGTTA
ACCTCCCAAGCTGATGTCACCTACCAACCCGGTGCTACAGCTGTGGAGCAAACAGGGTGGCT
CACAGTCTTTCTCGGTGCCTGATTCTAAACAAGATGAGATCCTAATCATGCAACATATGGGC
TTGACCAAAGGTCTATCAGCACCTAGTCTATCAGGAAGGAATGCCTATGTTGAACTCTTCGA
GGCTTGGCGAAATATGTCTAACGTCAAAGCACTGCACATGCTCTTCCCAAATGGGAATAGGT
CGCACAAGCAGTAGCAAAGACGCAAAGCCATTTCTTAGGCTGCATCACTGCTCTTATCCCGA
TTGTCTATCTTTTATGTAATATCAAAACTTGAGTCTCCAGGACGATCCAACCTTTGACTTTG
TACATTGTGAAAATCTTTTTGCCAAACTATTCACTCTTACTTTTTTTGTTACACATAGATGT
ACACATTTACTATTTGGCTAATACTTTATCTATTTTAAATTATAAGATATTTTAGTTTTTAT
AGTTACATTACTTTTGTTATGTATTTAGTCTAAATACATAGCAAAATCGATTTATCTAGAAA
AATCGAAACATCTTATGATTGAGACTCAAGGGAGTATATGTTACTTTTGCTCTTACGGTGTT
ATATTCCTAGGAATCTTGTGGACAACTTTGACTCGTGACATGTGGGTAAAAGGAAAAGATTT
GTGCTGCCTTTGATGGGTAAGAAGATTTTAACCTTGAGGACGGTGACATTATATAGCGCAGA
GGAAGGTGACATTATCTAACTACTTTAATAATACTTTTTAGTGTATAAATAATGTTTTTCTC
TTATAATATTTCAACATAAGTATTAGCATAAAACAAATTTTAGCAAAACGAACAATTAAAGA
AGCAGCTAAGATTGAATCAAAAGACAACTTAGAAAAAAAATTATATTAAAAGAAAATTCGAA
CTTACTGCTCCTTCTGACTTCGGTCTGCATACACGGAAAATGAAATTTGAAACAAAAAAGAA
AAGAAAACGAAAACGAAAACACGATGCATCAATCAATCATATAAAACCGAAAGAGAAAAGAA
ATGTGATTCTGTATGGTGTGCCCCAGCCCAGGTGGGCAAATTATTCCGCGTTGGCACGGCCG
CCCTCCCCCAACTTTCCAGTGCGGCACCGGCCACCTTTACACCTCATTCCCACCGCCGCCAC
CACCACCACCTCCACCTCCACGCTCCACCGTTGCCACCCCAGCACGCTCTCGCCGTCGCCAC
GCCCTATATCTCGCGCCTCTCGCCTCCCACTCTTTCTCCATCCGCCCGCTCGCCTCCCTGCT
ACGCTTCGTGCCGCCGCCGCAACCTCCTCCTTCCCGTCCCGTTCCAGGTGAGCGAATCGAGG
GCCCCTTGCCGTACTGCTTTAATGCTGCTGTTTCTTGATGCTCTAGAGGACTGGAGTCTGGG
TGATAGGATGCACTGGGGTCTGGGGGTTCGTTGGTTGTATTATGTTCTGGTTGGCTGTTAGT
GCTGGATCCGTAGTAGGAGTAAGGTTCGTCAAAGTTGCTGGGACTTTATTGGTGACCTGTGG
GCTGTGGATGCTTCGATCCTGTGTTTATGTTAAGGTGGCTACTAGTATTACACTAGATCTCT
TTGTAAATTTTCGGTATTAATATTAGCCTGTGGTAATGGATCCATGGTTCTTCTGTGCAAGC
TCTTTGTGATTAGAATTTATAGAAAGGAAATTTGTGATCCACGTTTAGAGTCGTTTAATGGA
TCCATTGTCCTCATTTGAAACTTCGGAAACTATGGCCTTTTTTATTGGTTTTATAACGCCAT
CTGGCTATAGAAATCTTGGCGAAGTTTGTGCTTCCGTAGAGCAGTATAGGCTTATAGCCAAA
TGTTGCTTCAAGATTTCTTGTTCAAAAATATTTCCACCTGAAAGGGGAAGAGTCTATGAAGT
TTGTGTACCAGACATCGATGCATCGAATGCCTTCTGATCAGAAAATGCTAAATTCTTCAGAT
CCTCACTTGTGAGGAATCATAACTGGGCACGATCAGAAGTACTGGCTGCAGCAGCACTACTC
AGTAAAACCTATGGAAATCAAGCAGCATCAGCCGCTGCAGCTGTGGTAGAAGTGCAGCCAAG
CAGAGCAACTTTGTTTTCAATAATTGACCATGTGTTTGATTAAATCTTCAGGGCCTGGTGTC
ATTCCTTGTGCTATTGCACAAAGCGTTTGTATGTATTGGAGTTTAGATGCTCCAGAGTCCAG
AACAGCCTGACTTTTTTTTGGGTATATTTACAACTAAAAACTAATTGTTTAATATAGTTGGA
AAATTATAAATTAATATTTAATTGCACAAAGGGTTTTATTTTTGTAAGAATAGTGGTTTCTG
ATTACACTTAATTGTTTTTTGCTTGATTTTTCACAGTTACCGTTGCTTGTGTCTTTGGTTTC
GTGTTTGATAAATGTTAGTACTCAATGGGAATATATTAGCCTCTTTTTCTGAGTCAACAGAT
AATAGCTTGTTAAGATGTGACTCATGTCTTCTGTTGGGAGTAGGCACATCAGCCTTTTTTTC
TCTTACTGAAGTAATTAGCACATATAAGTCTACGGTCTTTTCTTTTAGGGGAATATTAGTCT
ACAGCCTTGGTAAATGCTTCTCCTGATAAATTGTTTTCATTCATGCAGAATTGCAGATTACA
GGTCATTTAACATAAATG
SEQ ID NO: 5
Sequence Length: 3351
Sequence Type: DNA
Organism: Sorghum sp.
ACCTCCGAATGGTGATGCTGCAACCGTTTCATGAAAAAAAAAGTCAGACTTACATACGATTT
TTTTACTCATAATACCATGTGCTATCTTCCATCGTTTGCTTGTAAACCATACCATCCCTCCT
CGGTGCAGAACTTTGCTTGGTTTAGCGCTGTTCATCGCAATAAACCGCTGAAATAATTGCGG
AAGCATGAAGCACGCTTTTGACTCGGACCGTTTGGAAAATGGAAACTTCCCCCCGCCCGTCC
AGAGTGGGAGCTGCTGGCTGGGCCGTCGCCCGTCGGCGAAATGAAATGGAATTATTGCCCCG
CGTAATGGCTTCTCCCCGGGCCTTCCACTAGCGGAAGCATCAGGCGGGCGTGTGGCTCGACG
CAGTTGCAGGACGCCAATAATGCAGCCACTGTTGGTGAAAGCACCGGGCTTTTTGCCCATGC
TGGTGTGGTGAGGCGTCACCGCGTCAGGCGTAGTATTTGCGTATCGCCTTATCATGCGCGCG
GTCGAGTGCTGAAAGCACCGACGATGTATCACCCCCCTTGTTCACCGGTGCATGCGCGTTGC
TGTTCCGGCGTCAGAGGAAGGAAGGGAAGGAAAAGATGATGGAATCGCGTCAGATCTCCCGG
AAAAGAACGTGCGAATTATTGCCACTTTGAGCAGCTGGAAAAAGTCAATCATTGGACTTGTT
AACTCCCGGGGAGTTTGGTTTCGCGCTTTTGCGGTTTCTAAACGAGAGTGATTCGGCGGTGG
TGCGGGCAGTTTCGGCTCACAGATAATATTGCCGTCGATCCCACGCTCTCAAACTATCGCGT
TTGATAAGTACCAGGGCTCCAATTGCTGCGCTCCTTGTTGCCATCCCGAGTCACGAGGCATG
AATGAAACGGTTTACCACTCGTCGGTAGTAGTAGGCCTTGATGTGAGGGTAAAATTGACGTC
GTAGTAGCTAGCTTACCTCGTAGCTAGCCGTTTTTTAGGGCACAGGCAGCAGACACGGGTGC
TGGAAATCGCGATCATCCGATGAGGATTTGTTCTCATGAGCAAATGGTCGACGTTGAACATG
CCTGTCTCGTGGCCTCTGCTAGCTGACTCTGCTCTGCTGTGAAAGATCTTTGAGCTCAGATC
GCACTTGTGGTAATAAAGGGTGAAAAGAGTTATCTCGAAAGTGGAACGTGATACACGGGTTC
CTCCATAAACCTTCGAAAATATCGCATGTGGAATCTGCTAGAAAAAAAGGACAGTGCGACCT
TGTACCGGTGCAGGAACAAGTACCTGTTACACCCCGACTACCTGGACTGCCTGGAGTCGAAA
GCGAATACCGAGACTGCATTACTATTACTAATTCTCGTAGGTTTTAGGACACATATACCAAT
CACAGGGATGCTAGGAATCGCGATCATCTGATGAACATGGTCGGAGGTGAGTAATGGCTGAC
GTCGAACTTGCCTGTCTTGTCGCTTTGCTCTGAATTTGTAATACGTAGATCTTTGAGCTCAG
ATCGCACTTGATAAAGGGTGAGCAGATGGAACTCGAGCGTGGATGGAATGTGATACGGGCTC
CTCTGAAAAGTCTTCAAAGACATTCCATGTGATTCTGCTCGAAAAACGAACAGTGCGATCCT
GTTGTGCTCTCCGCGAGTATTTTTGACTGGCCGGGGGCGAAAGAAAATAATACAGTAATAAC
AATCGGCCCAGCTCGGTCGCGATCCTAGAGCCTTTGGGCCAAAGGAAACAAATCTGTGGGCC
TTTCGACAAGTCTAGAAGTCTCGAATCTTATTCTCGGGCGGTTGGCGACGGTGACGATGAGT
GGGCCGGACCGGTCGCGTTAGTTGGCGCCTGCTGCTTCTGCCAGCGACGGCTGGGTCCACCA
GCCCACGGAGCACGGCCGCCGCCCGTGCCCCACCACCCGCCCATATCATCCGACGAGGACGT
GCGCCGGATTCGCACCGAAAGCTTCGCGGTCCGGCCGCCGTCCGCCTGCGCAGATTTTTGTT
TCATCTGGCGGCCGCTGCGTCTCCATTGACCCGGCAGCCGGCACGGGCGAGGTCAGAGAGAG
CGAGCTGTTGGCCGGGTCGTCCCTGCGCTCGCCAGCGCTGCTGTCATGCCGTGCCGAGACAC
GGTGGCAGGCGGCTTCCCAACGACGCTGGGCATCCGCAGCCAGCGCGTGCAGCAGCAGACCA
GTCCAACCAGGCGGGCTCGTATAAAGAGGTTCCCCTGTTCCCCAACTTTGGCTGCCGCTCTC
CCATTTGTCTCGTCTCGCTCTCACGCTCGCGTCACCGGAGCTCTCCAGAAGCGAGCCCCAAC
TGCCCAAGGGCGAGCGATCCGATCCCCTTCGCAGCCTCGTCAACGACGCCGAGGTATACCCG
TGTTTCCCCCTTGCTCTCGCACCGATTTTATCCGAGGAAGCGATCGGCTGTAGACAGTCGGT
TCGATTGGTCCCCGCTCGAGCTTCCCGTCGGTGTTGACTTCGGTTTTTCATGTCGATTTTGT
TGTTTTTGTCATGTTTGTTTCGGCTCTAGAATTCGGATAGGCGGTCTGATATGGTTCAAAGT
GATCCAGCATATAATAATGAGCAAAACTACCGCATATGGGTAGTTTGAGTGATCCAGCATAT
AACGAGCAAAAACTGCCGGCAAAAACTGCCGGTCTGTGGTGTTTGATTGGATTCCCTGTTTG
TGTGATGGAACTTAATGTCCAGTTACTCAGTCAGTTCGTAGGTGTATATATGATTCAGCAAT
CAGCATGTGCTAAGATATGTGCTATGCTGATCCATAACAGAGTAGCAGAACCCGCAAATTGC
TTTCAGATCACTTTGTGAAGAACTGGAGGTGATCTCAAGCAGGTGAAACCACACTGTTTCTG
TCGATGATTCAGATGAAATGTGACTCAAATATTCAGTTAATAATTTTCCTCAACCAAATTGG
CCCCCGTCATCCTCTTACCCAAATAGGAGTGTGGTTTCAGCCAATTCGGTACATACGTGTGA
TGCTTGCTCACCATAAAAAAATGAAACATGCATGGTATAATGGTAGCAGAGAAATTTGTGGC
CGATCAAGTATCAAGATAATGAATAATTTGTATTATCAGCTGGCACCAAAGTCTGGTCGTCT
CTGCGAATTGCTTGAAAGCTTATACTTGTATAAGTCAAGTATCCGCTTTTTTAACGCAAAGT
AAAGTATCAGTTTCTTAAAAAATGAACAGTTGTGCACATTAGTTTTGACTGATGATTGCCAT
ATTGCAATTTGATATTTTTTTGTGGGGTTTACCTGACTCCATGGACACTTTAGTGATTAACA
CCGCTGCAGATAAACTCAAATACATTCTGAATATAGCATTCTGCTGCAGAACACTTTGAGGA
ATG
SEQ ID NO: 6
Sequence Length: 3273
Sequence Type: DNA
Organism: Sorghum sp.
CTGGCATAGATATATTAGATCTAAAAATTAAGAGTATCTCCATAAAAAGAACTTTTTAAAAT
TTGCCCTCTAAAACATAATTTGAGAAGTCATTTGAATAAAATTCGCTTTCTATATCTTTGTA
CCTTGAATAACTTTTCTATATCTTGTGAACACTCTAGAGAGCCATCCTTGCTCTCCATTTTT
TGCTACGAAAAATCCAAAATAGATTATGTTTTTGGAGGGTATTTTTTCACCTGAAATATTTA
TTCCAAGAAATCAGAAAAAAATATAAAGGGGTATTTTGGAGTTGCTCTAAATTTATATGGCG
GTGCTATTTAGGTAGGTCCCAAAACTTATCACATGAGCCATGCAAAATGTCATTTAGGTTAC
GCTAGTCTCGGTGATGGATATGAAAGTTTTATTTACATTAAATAGTCTAGCACATATGTATT
TTTTATGACATGATAATGTTTTAATGAAGAGAGAGAGAGAGAGAGAGAGAGAGAAAATAGGT
TTTGAGGGACAGAATTTTCTTGACACGATTAGGTCAGCAGTGCCATATTTCAATGCACTGTT
TTCAAAACAAATCCGCTCACAGGGCATCCATGAAACGAATAATGAAACAACCTCCACAATGC
ATGAGTTTCATCTTGACGTTTCCTAGGCTGGGCAAAGCATTTAATTACTGCAAAATGATTGG
ATCACATGCAAGATGGTGAAACGATTTAGCCCTCAGTGAGAATTTCATCTCGTTTCACCGCG
TGGGAAACAACGCCCGCGGGGTTTCACCATGGTGAAACTACTTCCTTCTCTCTCCTCTTCGT
TTCATGAAAAAAATACAGTTTTGCTGACATGACGCACTAATAAATGTGCATGATATCCTGGT
GAAACCCCCACTGAGACCGGCCTTACTAACTTTCTATTAGTCTAAGAATTTAATGTCTATGA
AACCATAGAACGAAACTTTGCATTAAAAGTAAATGTTTCATCTAAGTTTTATTTTATTGTAT
ATGACATGTCTTTGAAACAACGCAATCTCCACTGAGACTGGTCTTAGACTTGCAATTTAGTG
GGCCAGTGTGCTCATCGCTGGAGGTCCGAAAACTGTCATTTGCATAGGTCCAAAACTTATTA
CACGATCCAAACTTATTCCAAAGAAGTCAAAGCTCTTTAAATTCATGCAAAAATATAATTCC
AGCTCGCCTTCGGCATAGCTAGTTTTAGAAATCGTTTTTTTTTTCACGGGATGGACTTCCTG
AAGGCATAATTTGCTCGTTTGGATTCTAAATTTAGCTCTCTTTTTTTAAAAGAAAAATGTAA
ATTTAACTTTCTATGTATTCATTTCGAACGTAACCAGTGCAATGCAATTCACTCCAATGCAC
TGCGCACCGCGGTAATTTGTGCTTGGACATGTGTTGGGCCCATAATCTGTGATCATTGATCC
AAGTGATGTTTGGCCCTTTCACCAACCGAGTGAGCCCAGCAGAAAGTCCACTTCTTCATCGG
GCCATCAGACAAGCCCAATCCTCCATGCCGTGCGAAAAGTCCATACCGTGCAGGGTGCACGG
TTGCGGGTAGCGTCAGCCGGCACCCGGCAGCGACGGACCTAGCCGGCGGCCGCTAGGCGCCG
GGATTGGGGCGAGGCTCGCCAAACTCTACGCTGGCGCCGACGTGGCCCTGCCACTTTGGGCA
CCGAAAGTTCCCTGCGACGGCGACGGCGACGGCTAGGCCGGAACCCGGTGGAGCCTTTCGGT
TTCGGGCGCTTTCTCTGCCGTCCCCCGGAATCATCACCAGGCCGGACCGCGGGGCCCAGATA
CATACAGAGACAAGTCCTGCACGTCTCAACCGGCGGGGACGCTCCGACGTGTCGATCGAGAA
CAACACGTGCCGATTCGCAGTACAAGCGGCGTGGGAGCCAAAGGTACGGGACCAGTCCCGCG
CGGCCCGTCAGGCCGTCGTCACGCTCGCAGCACCGGCCGGTTTCTACGGGCGGTGCAGTGGA
CGCACTAGTCTCTTCGAAGACGGCGGCGGCGTGTGGTATAAACCCGGGGCCGCCCGTCACGC
CGCCCCGTCCGTGCGTTTCCTTTTGTTTCTTGCTTTGCTTCTTTTCGAGTGCTTGCCGTCCG
TCACTGCTCGCCGATCGAGTTTTCTCCGGATTCCAGCAGAGAGGCCCCGAACGAAGCGATCA
TCGCCGCACCTCTCTTCGAGCAGGTGATAATCCCTCTTGCTCTTCTGTTCCATCTCAAATTC
TCTGCGGAATCGTACTAGTTTTTATCCCCCCCGACAGATCGCCTCGCTCACCAGTCGCCAGG
CTTCCGATTGGTGGTGTCTGGTAACTTTTTTTTGTGGTTTCTGCGTCTGGCGCTGCTGATTC
CCCGTGGTTTCGGTTGGTTTGTGGCGATTTCTTTCAAGGGCAAAGGAATTCCCTGGGATAGT
AAGGCGTGCTGTAGGCTATGGTAGGATTATGCTACGGCGATGGCGCCGTGCCGCCGTCAGTA
CTGAAACACTACTAGTATAGGTCACCCGCTGTCAGTGGCGTTGGTGAGTTTTTTTTTTTTGT
TTTTGTTTTTTTTTGGGTAAAACAGCGAGCTACGAATTAACTGGTTGCTGAAATACGTGTCT
CGTATCTCTTCGTATGGTTACTCCGCGCCAAACAAAGAAATTAGGATTGACTTTCAGATTGT
AGAGGCTGCTGCTATATATGGAGCTCTTCGGTTCCCTGTTGGTCTCTAGTTGCCTGGGCAGG
GCAGGGGTAGTAGTAGTAGTAGTAATTTTTATTTTTATTTGAAATGCCCGTCAAGAGTTCTG
CCAAGCATATATTGATAGAGCAGGGATAATACTAATTTTATTGCCGTAGTGCACAGCAGCAG
CAGCAAGGCCAGATTGTGTAGAGACAGTTCTTGCTGTTTTGGCAAATACCCCCAGAAGTTAG
TTCCTGTGACCAGTGGTGATATATGATTTACTTTAGATAACTCAGCTCATCGTGGAGCATAA
TTTGTCTGACACAGATCTACATTTATACGCGAATACTGCGTATAGAAGTATCTTAGGCTGTT
GTACTCCCGTAGTAGCTGTGTAGAAGTCACAAGTAAAGGAAAGACAATTACAGAAACAGAAG
GCTTTTCCTTGAACCATGAGCAGAAAGTGTGTTTTGATTCCCCTGACCAGGCTTGATGACTA
TTAACTAGTACTCAATTACTGCAGTTTTCTCTGAATGTGTACTGAACGCTTTCTCTGAATGT
GTACTGAACGCTGAACATTGATCTCTCTGTTACAGAATAGTTGAGAATG
SEQ ID NO: 7
Sequence Length: 3003
Sequence Type: DNA
Organism: Sorghum sp.
TATGTCACTCTTGGCCTCTTGCGCAGAATAGAATCATCTCCTTCCGCTCATGTGGTATTAAC
TTTGATCAACGAAGAACAAAACTTGGTTCTCACTTCATTTCAAATTTTAGGCGTAGTATAAC
ACTAAGATCCATGCGCATCTTATAAAAAGAAACCAAATGAGAGTGAGGGTACTGCAAAATCT
GGAGCCAATCTCTCCAACCCCATCAGGCTGATTACTGGAAGAAAAAAATATTTTAAAATTAT
TATAACAATGGCTACAGCGAAGAAAAGACACAACTGAAGTAATAAGGTATATGAAGATACAA
CTGCAACGAAGGAGAGTAACCACCAATCTCCAGCACAGAGATTGGCTGGTACATTTGCTGGA
CACAAAATTGTTATCTATGGACAAACAGCTAGCAACACCGCAGCACCTTTTATATCTTACAC
TGCCCCTATCCTATTCGCCTATTAGAAGGTGCCCGACTATCTCTCAATTAGCGATAATATAG
GATTATTCAACCATCATCTCTTCTTAATATTTGAATATCAGGCACTAGTTTTGTGATGCCAA
CCAGCAGGAAACCTTGTGATTCATAGGCGTTCAATGTTAACACACCAGAGTAGGCTTAACTG
TCGAAAGGATAAACTTATATCGCACAAATAAGGAACTCAAAGCTGAAATGGCAATGTAGATG
GCTGAAGCCCTGGAACAACATGTGTTTGCACATCTTGCTGGCCCTGTCGAAACTAAGACTGC
TGCAGAAACAACCAGGGTTGCAAAATTTTCTGAAGAATGCCTGGCTCTTCAATTCAGGCAAT
GTTTTATTCAAATAAGAAAATTCAGGCAATGCCGCGCAACCAGGGAATGTGATGGTTGAACT
TCAGCCCATAGCCATAACCAAAAGTACCTCCGCCAGACCAAACTGAAGAACTAGATCAAACT
CATGAATCCTAACAAATTACCTGGAATAAAGCAACCTTTGCAGAATGATTTGTCAGGATAAA
GCAACCTTTGAGAGGTTCAACCTTCCCTCATGAATCTTGAACTACCAATATGCCACAATTAT
GTCCATGACAGTTGAAACATGACTTTTGTGTTCCAAGCATCAAATGCCACAACTATGTCCAT
GGCACTACCTCTATCAACTATGTCATACCACTCTGAAAATCCATGCACCTTCTCATTTGGTT
ATCTCCAGCACACAAACCTCCTGCTCATGGCACCATCAATCCATTCTCCATCAGAAGTGCGC
AAGCACCGTTGAAAATGGCTTACTGACATGATTTCCCATGAAGTTGACAACATCCTTCTACT
GTTACTTCTCCGATACATCCAACAAGCAATCCTCATGCTTAACAAAATCATCACCACTCCCT
CTGGCATCAGCAGTGGCACCAGAAGCCATGTTGGCACTTGAATTAGCATTCCCAAGACCACG
TCGACGCAAGAGCATGAGCTCGACGAGTTTGACCCTTGCCTCAGAGACATCATTCGACTCAA
CAAGCTTTCCACGATCATACATCTCCCTCAGAAACACCGTGTGCTGCTTTCGTTTCGCCGAG
AGGTAGAATATCCCAGGGTGATCCAAGAACACGTCCCGGACATTGACCTCGATCCCAAACCA
CTTCCTGAACTGACTGAACTTCTCCACCTCCACCATCTTCTCCACGGTCAGGCTGAGGAACT
CGTGGGCAATCCCTACCGCCCTCTTCTCCATCTTCCTCCTTGCCAACTTCGACACCCTCTTG
CTCCCTCCACCCCTCGGACTGACGACCTGATACGGGCCGGTGTAAGGCAGCAGCTGCCACTC
CTTCACCTTTTTCCGATACTCCTTGGTCAGCCTGAACCCCGGCGGAAACTGCAGCTTGAAGG
CGTACCTGTCCGGCCGGGTCTTCTCCACCGCCGGGGTGAATTCCTCAGTCGCCGGATCGGCG
ACGAGGTGGAGAATGTGGGTGTTGGGCTCGTCGGGATTGGGGGCAAGGCGGAAGAGGTGCGG
GTGCCCCTCGACGACGGAGTCCTCGAAGTCGTCGGGGAGGGCGAGCTCGCGCCAGACGCGGA
AGACAGCGCGGAGGGGCAAGGAGCGGCTGACCGACATGGCGAGGAGGCGGTGGAGCGTCCGC
GCCGCGTCGGCGGGGGAGCTGGCGACTGCGAGGAGGCCCGCGGCGGCGGGGGTCAGGGACAG
CGAGAGCGGGAGCGGGGCGCGGAGGTGGAAGGGTCATGAAGTCTGTGTATTGGACATCGATG
CGCTTGTGCTGGATTCCTTGAGCTGTCTGCTGTCTGATGCATCGAATGCCTTCTGATCAGAA
AATGCTAAATTCTTCAGATCCTCACTTGTGAGGAATCATAGCTGGCCGCGATCAGAAGTGCT
GGCTGCAGCAGCACTACTCAGTAAAACCTATAGAAATCAAGCAGCATCAGCCGCCACAGCTG
TGGTAGAAGTGCAGCCCTGTTTTCAGTTGTGAGGAAATCACAACTTTTGTTTTCAATAATTG
ACCATGTGTTTGACTAAATCTTCAGGGACTGGTGTCATTTCTTGTGCTATTGCACAAAGCGT
TTGCATGTATTGGAGTTTAGATTTTCCAGAGTCCAGAACAGCCTGACTTTTTTTTTGTTGTA
TATTTACAACTAAAAACTAATTGTTTAATATAGCTGGAAAATTATAAATTAATATTTAATTC
CACAAAGGGTTTTATTTTTGTAAGAATAGTGGTTTCTGATTACACTTAATTTGTTTTTGCTT
GATTTTTCACAGTTACCATTGCTTGTGTCTTTGGTTTCGTGTTTGATAAATGTTAGTACTCA
ATGGGAATATATTAGCCTGTTAAGATGTGACTCATGTCTTCTGTTGGGAGTAGGTGCATCAG
CCTTTTTTTCTCTTACTGAAGTAATTAGCACATATAAGTCTACGGTCTTTTCTTTTAGGGGA
ATATTAGTCTACAGTCTTGGTAAACGCTTCTCCTGATAAATTGTTTCATTCATGCAGAATTG
CAGATTACAGGTCATTTAACATAAATG
SEQ ID NO: 8
Sequence Length: 3324
Sequence Type: DNA
Organism: Sorghum sp.
AGCTCAAAGGAAATGCATTTGCAGCTGTCTGTCCCAATCAATCCACTAGCAGACTCATATTA
TTGATGGAGGAAATTAAATTCAGTCTTTGACGTAGATGCAACAACTGCACATGATACGTTTT
GAGAAAATTAAACCAGCTTTGACCAACACGAAATGAGCGCCTTACGTTTGGCACGTACTCCG
GCACGGCAAGTTAGACTCTGTATGTAGTGGTAGAGCCGGCCTCCTTACGTTGGGCACAGTTT
TAGTTGAGCCCGGCATGGCAGGTTAGACCAGAGTGTGAGCCGGCCACCACAAGTTATTATTT
ATAACATATATATAGGAGCAAGTGCACATAACAAAATAATTAGCATGTTCGCTTGAGCTTAT
CAGCCGAATCTGTCAATCATTTAGCAGTGTTTTTCTTTTTTAAAAAATCAGCCAACAATACT
TCTGTCATGGCTTCCAAACAAACAAGCGAATGTGAGCAAACTATATGAATTGTCACGTCATA
TTTATGTTGAGATGAAGAAGAGAAATAAATGGCATGTAAAATTATAGCCAGTGATAGACGAG
CACAAGGCCTTCTATTCTTAAATCAGACTTTGAAAGAAAAAAAAAGGACTTGAATGGGAGAC
ACGAGTAAGGCCATTTTTTTTGTAAGAATGGGTTCTTAAAAAAATTTTAAAAATTTTCAAGA
TTTTCAGTCACATAGAATTTTTGGACATATTCATAGAGCACTAATATAGATAAAAAAATAAC
TAATTACACAGTTTGTCTGTAATTTGCGAGATAAATCTTTTGAGCCTAGTAAGTCCATGATC
ACACAAAAATTATCAAATACAAATGAAAGTGCTACAGTAGCTAAACCTAATTTTTTTTGACC
GACTAAACAAGGCCCAAAATTGTTAAATTTACTCAGGTGACACGGCATTAACGATAGTAGGT
AGCTAAATTAATAGTCATACTCTAACAGCTATAGCCGAGAAGGCTAAACAACTATAACCGTC
TGGCTAGCTAATGGTCGAGTGAGGCCCGTATAGATGTAGTTAAATAGCTAAAATTTTTGGAG
AAATAAGCATTTTTTTGGAAGAATATATTTAAACATGGGCTTGTAAAACTTGGCTGTAAAGA
TTTGGAATTTAGGATCTTGGAGCCCCAAAACTGTATAAACTTGCTTAGGGACCCGTGTCTTG
TGTGTTGCAGACCAAAAAATTTAGAAAGCATCTAAACACCTATTTGAATGTAAAGTTTACAG
CCAAAAGTTTTAGGATGTAAAGATTTGGGATCTAAAAGTAGTCATTAGGAAATAACACGTTA
GAGAGAGAGAGTAGATCTTCTTATTGGTTTCTCATGCACTAATCGAACCAATCACTGGACCA
CTTGAACCAAACTTTATCACATTGAACTTTGTCAGTTCAGTTCGAACGCAGGACTGGAGCTG
CCCTTAAGGCCAATTGCTCAAGATTCATTCAACAATTGAAACATCTCCCATGATTAAATCAG
TATAAGGTTGCTATGGTCTTGCTTGACAAAGTTTTTTTTTTGAGGGAATTTCAACTAAATTT
TTGAGTGAAACTATCAAATACTGATTTTAAAAATTTTTTATAAAAGGAAGCGCAGAGATAAA
AGGCCATCTATGCTACAAAAGTACCCAAAAATGTAATCCTAAAGTATGAATTGCATTTTTTT
TGTTTGGACGAAAGGAAAGGAGTATTACCACAAGAATGATATCATCTTCATATTTAGATCTT
TTTTGGGTAAAGCTTGAGATTCTCTAAATATAGAGAAATCAGAAGAAAAAAAAACCGTGTTT
TGGTGGTTTTGATTTCTAGCCTCCACAATAACTTTGACGGCGTCGACAAGTCTAACGGACAC
CAAGCAGCGAACCACCAGCGCCGAGCCAAGCGAAGCAGACGGCCGAGACGTTGACACCTTCG
GCGCGGCATCTCTCGAGAGTTCCGCTCCGGCGCTCCACCTCCACCGCTGGCGGTTTCTTATT
CCGTTCCGTTCCGCCTCCTGCTCTGCTCCTCTCCACACCACACGGCACGAAACCGTTACGGC
ACCGGCAGCACCCAGCACGGGAGAGGGGATTCCTTTCCCACCGTTCCTTCCCTTTCCGCCCC
GCCGCTATAAATAGCCAGCCCCATCCCCAGCTTTTTTCCCCAATCTCATCTCCTCTCTCCTG
TTGTTCGGAGCACACGCACAATCCGATCGATCCCCAAATCCCCTTCGTCTCTCCTCGCGAGC
CTCGTGGATCCCAGCTTCAAGGTACGGCGATCGATCATCCCCCCTCCTTCTCTCTACCTTCT
TTTCTCTAGACTACATCGGATGGCGATCCATGGTTAGGGCCTGCTAGTTTCCCTTCCTGTTT
TGTCGATGGCTGCGAGGCACAATAGATCTGATGGCGTTATGACGGCTAACTTGTCATGTTGT
TGCGATTTATAGTCCCTTTAGGAGATCAGTTTAATTTCTCGGATGGTTCGAGATCGGTGGTC
CATGGTTAGTACCCTAAGATCCGCGCTGTTAGGGTTCGTAGATGGAGGCGACCTGTTCTGAT
TGTTAACTTGTCAGTACCTGGGAAATCCTGGGATGGTTCTAGCTCGTCCGCAGATGAGATCG
ATTTCATGATCCTCTGTATCTTGTTTCGTTGCCTAGGTTCCGTCTAATCTATCCGTGGTATG
ATGTAGATGTTTTGATCGTGCTAACTACGTCTTGTAAAGTTAATTGTCAGGTCATAATTTTT
AGCATGCCTTTTTTTTTGTTTGGTTTTGTCTAATTGGGCTGTCGTTCTAGATCAGAGTAGAA
GACTGTTCCAAACTACCTGCTGGATTTATTGAACTTGGATCTGTATGTGTGTCACATATCTT
CATAAATTCATGATTAAGATGGATTGAAATATCTTTTATCTTTTTGGTATGGATAGTTCTAT
ATGTTGGTGTGGCTTTGTTAGATGTATACATGCTTAGATACATGAAGCAACGTGCTGCTACT
GTTTAGTAATTGCTGTTCATTTGTCTAATAAACAGATAAGGATAGGTATTTATGTTGCTGTT
GGTTTTGCTGGTACTTTGTTGGATACAAATGCTTCAATACAGAAAACAGCATGCTGCTACGA
TTTACCATTTATCTAATCTTATCATATGTCTAATCTAATAAACAAACATGCTTTTAAATTAT
CTTCATATGCTTGGATGATGGCATACACAGCGGCTATGTGTGGTTTTTTAAATACCCAGCAT
CATGGGCATGCATGACACTGCTTTAATATGCTTTTTATTTGCTTGAGACTGTTTCTTTTGTT
TATACTGACCCTTTAGTTCGGTGACTCTTCTGCAGATG
SEQ ID NO: 9
Sequence Length: 3704
Sequence Type: DNA
Organism: Sorghum sp.
TCGGGGGTACTATCGCGCCGACTTGCATGCAAAGCTCAATGACAGCAGGCGCATGGCCATAA
TAATCCCTCCCGCCATCCACAAGGACCGGTGGATCATGAGGCCGCCGATGCCACACAATCTT
CTGATGAACTAGGTCATCCACTCCGGCCTTGTCCCACCTGTGCATCCCTTTCCAGCCCTCAA
ACTTGAATACTCCACACATTCTGGCCTTGTGCCCTGATTGCCCAATATGCACTTCAGAGCAA
TGCTTACAAACTTTGGATGGGTACACCAAGAGGAGTTTTGTGACACCTAATCTCAGGCTTTC
CCATGCATCTAGTGTTCTCTGACCAATATATCTGAGTTCATCTGGCAAGATTGGAGAAGATT
GCTGATTGTTGGTTTTGAGTGTGGTAGGTATTTGTTCACTTTTGTATAGAATCTCATCAGGT
ATGTTTGCACCTGCATGATGGCAAAGCTCAAGTACAGCTGGGACACGAGTGAAGTCGAACCG
CTGGTTATGCTTTATCTCAGTCTCAAACATGTTTTTCTGGTGGAAAGCCTGAACAGGGACAA
GGATGTCATTCAAGTTGCTCGGATCCCATTCATGTGGTCGGTCCTTGATCATACGCTTGAAA
CCATAGCATGTTTTCATTTGATGACCTGTGGCTCCAATATGAACTTCAGGACAAAACCTTCA
GTGCAAATCAATGAAGAAAGTGTTAAACAGACTCATAAAATGCAGAAATATTCAATTCAGGA
CAAGAATCAATAAACTGTAATCAGGCATAAATATCACAGAGAACGCTTAGCTACAAGAATGA
TTGGATTGTGTTTATAAAAACCTAAAATGGTGAAGATACAGTAGAAAAACAGTAACCTATCC
ATTCATAGTATCTCTACTTTATTCTCCATTTCTTCAATCCTTTTGATACTTGCCTAATTCAA
GATGCTATAGTTTCTTTTTTTTTTTAAAAAAAAAGCACTTCCAGCAAATCATACTGGGTTTA
CTATATGTAATACTGCCACACTGGGTTTACTATCGTTTTACAGTTAGGACAAGAACAGACTA
ATTTCCCATTTATTTTCCTTACTTGCATGACTGAACAGGAACAACTTTGAGCAGTCTGGAGA
CACCCTCATAGACAACCTCCCTTGCCCTGACCACCTCCTCTGCGACCGGGATCATCCGCTTG
ATTGGGTATTCCTTTGTCCGGTTCTTAATCCTCTTAAGAATCATTGGCCGCAGCTGCTTCCA
ATCCACCCTCGAAGTGGAGTAGCGCCTCTGGTTCAGAGTCGTGCCACACAGCTCCGAGCAGA
TGTAGCTCCCGGCTCTTCGCCACAGAGTCGCCATTGTAGCACATATACAACAAGCCTACAAG
TTCATAATTCGAATAAGCGGGTCACCAGCAAACAATGCGTTCTAAATTCCCCCCGTTTAACA
GTTGATTAGAACTGAGAAATCATACTGGATCAGAAGAACTTACTAGTCGGGGAGATCGATCT
GGCGAACCGCGCCCGACCCCTGATCCGGCACTTCCACGACTCTACTAACAGCGCCTCCCGCT
CGCCGATAGCGAAACAGGGGGCGTTCCCTCGTCGGCGCCGGAGAGCAGCCGTGGAAGTGCCG
ACGCCTGAGCGCGGCGAGACACAGCAGCGCCTGGAGCGAGAGGATGCGGAGTGCGGTGGACC
GATAGGACGGGACCTGCCCCGGGGGAGAGGCGACGACGCCGGGGCCGGACGCGCAGAGATGA
CTGGGCCGGCGGCAGCGCGGCCGCAGGCGGAGCCGCCGTGCGGGAAAGGTGGATGCGCGGCG
TGGACGGCGTCTCACTCTCTCCATGCTTTGCCTGGGCCTCAATTGGGCCATGAATCTGGGCT
CTTGGCCCATAAATATCTCCCAGCTTCAGCCTTTTTTTTTCAGCCCAAACATGCCGATTCTT
TAACCGCACCGGATATCTCTCTCTTCGCACCGCCTTACCGGGGCTATAAAAGGAGAGCCCCG
CACACGGGCTCCTCTCATTGATCGCCGCCATCACCATTTAATCCCCAAGGAAATACCTCATC
AGACGCTAATCTTCTCCTCATCAAGGTAAAAAAAAAATCTCATCTCATTGTCAGTTCTTCGC
CAAGTCAGGGGTTAGTTAGTGCGGTCGGCGGATTCATGGTTCGTTTCGTCGGCCGCAGTGTT
AGGGGTTATGGTTCGTGGGGTGACGTTTGATCTAAGTGGGTTTCTAGTCAAAATCATGTCTA
GTTCATTTGAGTTGGCAAATTATCTCAAAATTGCGTGTGCTGGTTTCTACCGATTTTGGCGA
AAAATAGGTATACTTTGTTAGTTCTAGATTAGATTCTAATTTGCCTTTGACTGGGTAAATAC
TTTTATATGGAACTGATTAGTTACATTTGGATTTGTATGAGATTGATAAATACTTGTGGATT
GCTGTTAATTAGATTCTCTTGTGTTTATAGAACTCTGATCTGATTATTTTCAGTAGTCTAGT
CTGGCCCTCAGCAGCCGGGGTACGGGAACATAGGTACGTTCCTCGTTCCGGGAACTTCGTTC
CGAAACGTGGAACGGGAACATCATGGAACATCGTTCCCAAATGCGGTGGAACACGAATCATA
TATAGGAGCAATCATAGGAACGTCAGTCCCAAGCGACTATGGAACTTCGTGGTGTGTGTGCA
TACTGCGCCGTGCCCGTCTATGAGCGGTCGGCGAAAAATCTAGATGGTGATTGACTCATTCG
CCTACACATGCAATGAGCCAAAAGGTTGGAGTTGGACAGCCCTAAATGAAACAAAAATACAA
TAGTAAACTATATCACGCTAGAAATCGAACTCATTTGGTTCAAAACAACATATAAAACTATC
TGAGCCAACCACTACGTCCCAAACACGTTCCTTTAGAAAGTGGAACGCGTTCCGCAACTTAA
AGGAACGAGACGAAGCTATATACCCCCTACGTTCCATAGTTTATGAGAATGTCGTACCGCGT
ACCACGTTCCCGTACCACGTACCCTATGTTCCGGGAACTTGGCTGCTAAGGTCTGGCCCTTT
TGAGATTCCTATTAATTGACTGTGATTGGGATTATTGTCGGTCTGGCCTTTTGGGATTCCTA
ATTGTTTGTATGTTTAATATCGTTTATTTAGTAATTTATTCAGTTTTGATGTTTTTATTGTT
CATCTGTTAATTAATACTGTACCTATACATGTCATATGTAGAACTCTTTAGTAGTGAAAAGA
TTTATTTGTACATTGGTGGTTTATGATGAATCTGTTTAGTTTCCACTGTTGTCATCCTACCT
ATTTGTTAACCCAGGTTTGTTTCTATATGTAGCCTATGCATGTGATTAATAGCTCTAGCTGA
GGTGCAGCTTGTGTGGATCCAATCATTTCATTTTTTCTGAACAGCCAGTAATCGCCGAGGCT
AGCTATAATAATCTCTCAGATTTTCTTGAAATGGCTAGGGCCTCTCCCTCTTAGTTGGTAGT
CTCTCCTTTTATCTGTTCTCTCGCCGGGCTTCAGTCGGCTACAACTGCTTTGCAGTTCTGTA
CTGTAATTTGCTTACTGTAATTACGAGCCGTTTTGGGCTAAAATAAAGTTTTTACAGGTGGG
GAACGCCTCCCCCCGTTGACCCTTAAAAAAAAAATCTCTCAGATATCATTGCACATTCTGTA
AGATATGAATTCGTCATGTTTCCATACCTTCTGTCCATTTCAGATG
SEQ ID NO: 10
Sequence Length: 3123
Sequence Type: DNA
Organism: Sorghum sp.
CAACAAATATAATCTGTTTGGCTATTCCAAGTGGTTCTTTTTTTTCTGGGTAAAGAAGAACT
TGTCATTTTGAATTCTCGGGTTCAGGAATTTTTCTATAACTTAAATGATTCCATAAAAGCTT
TCTTTTATTCTTTTAGTTACTGATTTTTTTGTTGGATTTCACTGCACCTAACAGTGTTTGGA
AGGGACTAGGGAAACGTGGAATGGCGAGATAAATTGTTTCTATACTTTGATGACTTAGGCCT
TGTTTAGTTCACGAAAAGTTTTGGTTTTGACTACTGTAGCATTTTCGTTTGTATTTGACAAA
TACTATTTAATTATGGACTAATTAGGTTCAAAAGATTCGTTTCGCAAATTACAGGCAAACTG
CGTAATTAGTTTTTATTTTCGTCTGTATTTAATGCTCCATGCATGTGTTAAAGATTCTATGT
AATGAGGAATCTTAAAAACTTTTTGTTCTTGGGAGAAACTAAACAAGGCCTTACGTTTTCTA
GGATGGTGAGTTTCGGTTCATGGATCTTGCTGTCTATATTTATCTATACACGTTGTTGTACT
TATAATTGAAAAAATATTGTGTAAGTTCTTGCATCGCATTCACTGCCACTGCAATGCACCAG
ATGGTGCCACAGCCTTGCATTCTGATGTCGAGCGGACCATGGATGATACTAATTGGTTTGGT
AATGATGAATTCAGTCCTTCGTCATCAAGTAAACGACTCTACTTTCAGTGCATCACCAGAAG
AAGCACATATATACCAGATTCCTCAATTTTAAAGACCTTTGTTCCAGAGCAGTTCCTAGAAA
CTCAGTTACACTTTCTAGCCATCTCAGAAAAAAAAAAGTGATCCACTAGAGGAGACTCCCTT
AGGGTCCATTCGTTTAGCTCTGGTTCCGGATGAATTCATTTCAGATGATCAAAAATAACATA
AATTTACACAACATTCTTGACTGGAATCATTCCAGGCATCCATTCCATAAGAAACGAACAGA
GCCTTAGGATATGGCAACACTAAGTAGATGTCGCGCTTCAAACCGGGGCCGACCAGGGGCTT
CAACGATCCCTGGAATTCAACGTTCTAACCGGTTGCATCGTGATAAACTTAGCTTCTGGCCA
TCTCCAGAGACAGTGAGTTGATGCTTGATGCTAGACGAGGGGAAAAAAAGCAGAAAATCAGC
CATACTAAATCAACTAATGATTTCAAAGAGAGGTACCTAATGCTCAAAAAGGAAGAGATTGG
GCGATTTGCGACTAAAGAAGAGAATAAAATAGATTTTTTATAGCGTTAAGAAGTGTGTCACA
GCTCTTACAGGAATGCTTGATCTACAAATGGAATAGATGATAATGGCAGCGGATATGGACGG
ATCGGGGTTGTTTAGTTCCTAATTTTTTAAAAAGTTTTTCGTCACATCGAATCTTATAACAC
ATGTGTGAGTATTAAATATAGATAAAAAATAACTAATTACATAATTTATTTGTAGTTTGCTA
GATAAAATTTTTGAGCCTAGGGTTAGTTTATGATTGAATAATAATTATCACGAAAGTACTAC
AGTAGTTAAATTTAAAATTGTTCGCAAACTAAACAAGGCCATGGTGTGTTTTTATTTTACTC
TCTAAAAATCTGCACAAAGGTTTTCTGACTCATGGGCCACACGTCTCAGTGTCGGTAAACAC
GGACGGAATCACGGGAGAAGGCATTAACAGCGTCGGGTCTAACGGCCACAAACCAGCGACGA
ACGAAACAGACGTTCTGACGTCTCCGTGTCCACTCCGTCACTGGTTCCTTCTGGAGAGCTCT
GACCTCCTCCGTCTCTATCTACGGCCGGCTCGCCTTCCGTTCCGCGTTCGCGTTGGACTCTT
TGCGCTGGCGTGTTCCTGGAATTGCGTGGCGGAGACGAGGCGGATTTCTCTCGCACGGAACG
GAACCGCCACGGGCCCAAAGGCACGGTGATTCCTTCTCCACCAACATAAATAGCCAGGACCC
CTCCTCGCCTTTCCCCAATCTCATCTCGCATTGTGTTGTTCGGAGCAAGGAGAACCCAGCCC
CCCATCGCTCTCAATCCCAATCGATCTTCTTCTCGTGAGCCTCGTCAATCCATCACCCGCTT
CTAAGGTACGGCTCCCCCTCTAATCTTCTCTTCCCATCTCAGATTGGCGAGTTTATGTGATT
AGATTAGATGCTTCTCATCTAGATTGCGAGTTTCTGTTCGTAGATGGCTGGCTTGTAAGCGG
TTCCTAGGTGGGTTTCTGTTCGTAGATGACTGGCTTGTAAGCGGTTCCTAGGTGGGATCGTT
CTGATGATTTCTTTGGCTGCTGCGTAGAGATAGATCTGGTCCTGCTTTTCTTAATTCTTGGT
GCAGATTTTGTGACCTGGTTCTATGTTCTTGTTCCTGCTTTGTAGCTCAAATAGTTGTCTTA
ACTAGCTGGGCTTATTATTTGATTTGTACCTGCATGTATTATCACCAAATACAATTACTGTG
AAGGAGTCAATATACCCTGCTCTGTACCTTTTACCTGACGAGCCATACTATCATTTTGATTC
GTGTCATATGCATGCCAGATACGGAAATTATATGCTGCTACTTGCGTTATTATCATGCTGAT
TTGTTTCATATGCACGCCTAGATAGATGGAAATTATATGCTACTGCTGAGCGTTATTATCAT
GCTGATTCGTTTCATATGCATGCCTAGATAGTTGGAAGTTTTGTTGTTTGCTGAGTGTTACT
ATCATGTTGATTTGTAATCATATGCATGCCTAGATAGATGAAGATACATGAATGTTATTCGT
TTCAGATAGATGGAATATGCTGCTACTGAGCGTTACTATCATGTTGATTTGTTTCATATACA
CGCCTAGATAGATGAAGAGATGGATGTTGATTTGTTTCATATGCATGCCTAATAGATGAAGA
TATATGCTGCTACTGATGATTACTTACTACTTCGTGCCCATGCATGCTCTTTGGTTTACTTG
GATGGTGACATGCTGATGCAGTTTTGCTGGTTCTATAGTACCTATGTGCTTAGCATGTATAT
CTGTTTCTTGTTGCTGACTGTTTCTTTCCCTCCTTAGTCTACCGCCGTATACTTATCATGTT
GCTTGTTTTTTCTTCTACAGATG
SEQ ID NO: 11
Sequence Length: 3003
Sequence Type: DNA
Organism: Sorghum sp.
GTTTTTGTAAGAGAACTAAATAGGAGCTGAATATATATAGAAATATACTAGTATTTTTTTAA
CGTAATGGTGACAAGATCTCTGTCTTCAATTAAGATATATTAGTATTTATTATGTGTATAAT
AACTACTGTAAATTGAGCAAAAATTATATTTTTAGAATAAATATATTTGAAATTATGAATAT
AGATATTTATTTCATAAATTAATTAAACTTGAATTCTTTTACTCATCCGAAGGGAATTGAGA
TTTACGTTTTTTCTTTACGAAGGGAGTAACATCCCGATGAGAAAAAGATAAATGGATATCGG
GTAAGAGTTTCCGAGAAACACCGCATAAGCGTTTGGCTGGCATTATTTTATAGAAGAGGGAT
CAAACTTATTTTTAGGGTGTTTGGAACTGCATGCTTTAAAACTTTACTTCATAAACTTCACT
ATAGATATACTATTATTTTTACGGTGTTTGGAACTGCATGCTTTAAAACAAAATTAGTTTAA
AGCACCTCTACAATTTTACTCTTTTTCTCAAACAAAGTACGTTAAAATCCTTTTGCTCAAAA
ATATAGAAGGGAACTAATTCCAAACACCTTAATTCTATTTCTATACTCGTATATTAGAAAAA
AAAAATTCTTCCAAGCGGCAGGCCACATCCATCAGCGTCATTGAGCATAGAGATATTTGGCG
TCGCGTCGACCGATCAACTACCGCCATCCAACAGAAAGAGAAAAAAACGATTCTAAGGCCTT
GTTTAGTTCGCAAAATTTTTTATTTTTGGCTACTGTAGCATTTCGTTTTATTTGACAAACAT
TGTCCAATCACGGAGTAACTAGGCTCAAAAGATTCATCTCACAAATTACAAGTAAACTGTGC
AATTAGTTTTTATTTTCATCTATATTTAATGCTCCATGCATACGACCAAAGATTCGATGTGA
CGGAGAATCTTGAAATTTTTTACGAACTAAACAAGGCCTAAGGAATAAAAAAAAAGGAAAAA
TTGTGCAAACTCTTCGTCAGTGCTGATGACAGAAGCAGCTGCCCTTACTCTAGCAACCACGG
TGCTAGAAGCTATGTACATGATTGATTCCACTATTTTAACAGATAATCAATAGTTAGTACTC
TTTCTAAACGGGTCTTAGTTTGATCATCATCCTGATGGAGAATTAAATCCTACATTCAAATT
ACCAGCTCCAAGATTCATGGTACAACTATAGCGATTCGCAAGATTACCAGAATCATATGGCT
GATCAACTAGCTAGATAGGCTCTGAGTGAATTAGTTTGCAATCAAATCTCTCTTAATAGTGC
TTGTTGTCATTCTGCTCATGAGCAAAAGTGTCCTTTACTTTCGACACTCTCAAATATAACTA
TTAACTCTATAATGGTCCTAACCGTAACACGCTGTTAATCATATAGGCCTTGTTCAGTTGGC
AAAAATTTTGGGTTTTAACACTGTAGCATTTTTGTTTTTATTTGATAAACATTGTCAGATGA
ACTGTGTAATTAGTTTTTATTTTTATGTATATTTAATGCACCATACATCTGCCGTAAAATTT
GATGGGATGGAAAATCTTGAAAATTTTTGAAACTAAACAAGGCCATAGTTTCATTGTAAAAA
AAAAAACAGCTAAGCAAGATGGCCGAGAGAGCCGTTGACGCAGAGCATTGAACGGCATCTCT
CTCGGCTGCTCTCGAATGCGCTGCCTGCCGGCATCCCGGAAATTGCGTGGCGGAGCGGAGCC
GAGGCGGGCTGGTCTCACACGGCACGAAACCGTCCCGGCACACGGCACCACGATTTTTCCTT
CCCCTCCCCCTGCCCTTCTTTTTCCTCATAAATAGCCACCCCCTCCTCGCCTCTTTCCCCCC
AACTCGTCTTCGTCCCTCGTGTTGTTCGGCGTCCACGGACACAGCCCGATCCCAATCCCTCT
TCTCCGAGCCTCGTCGATCGCCCCCTTCCCTCGCTTCAAGGTACGGCGATCGTCCTCCCGCT
TTCGCTTCTCCCCTCCCCTCCTCTCGATTATGGGTTATTGGGGCTGCGAGTCATCTTTCTGG
CGATTTATTATGGTCTCGATCTGGTGGTAACTGTGGCGATTTATTATGGGAGCCCTCGATCT
AGAAGTCGAGTACTCTCTCTGGTAACTGTAGCGATTTGTTATGGGGGCTCTCGATCTAGAAG
CCGAGTACTCTCTGGTAACTGTGGGACCCTTGTAGGGTTGGGTTGTTATGATTATTTGGGCT
TGTGATTAGGTTGTATCTGATGCAGAATGATGTATTGATCGTCCTATTAGATTAGATGGAAA
CAAGTAGGGTGACTCTGATTTATTTATCCTTGATCTCGTTTGATGTCCCTAGCTAGGCCTGT
GCGTCTGGTTCGTCATACTAGTTTTGTTGTTTTTGGTGCTGGTTCTGATGCCCGTCCAGATC
AAGTCATATGAACCAGCTGCTGTCTTATTAAATTTGGATCTGCCTGTTTTAACATATATGTT
CATATAGAATTGATATGAGCTAGTATGAACTAGCTGCTTGTCTTATTAAATTTGGATCTGCA
TGTGTTATATGATGGATGAAATATGTGCTTAAGATATATGCTGCGGTTTTCTGCCGAGGCTG
TAGCTTTTGTCTGATTAAAGTGCATCATGCTTATTCGTTGAACTCTGTGGCTGTCTTAATAA
GAATTCATGTTTGCCTGATGTTGGAGAAAACATACATAAGAATTCATGTTTGCCTGATGTTC
GAGAAAATATGCATCGACCTACTTAGCTATTACTTGATGCGCATGCTTTGTCCTGTTTTGTT
TGATATGCATGCTTAGAAAGATTAAAATATATGTGGCTGCTGTTTGATTCGATAATTCTTTA
GCATCTACCTGATGAGCATGCATGCTCTTGTTATTCACTGCTACTGTTCCTTGATTCTGTGC
CACCTACATGTTACATGTTTATGGTTGCTTCTTTTTCTACTTGGTGTACTACTATATGCTTA
CCCTTTTGTTTGGTTTCTCTGCAGATG
SEQ ID NO: 12
Sequence Length: 1121
Sequence Type: DNA
Organism: Sorghum sp.
AAAACTGACGATGTTGCCCCTGGTAGCTTCATGTTCATGGGGTTTCCTACTCTTCCTGCAGT
TTACACCTCAGTACCTCACTGTCCAGCCAGCATAACAGGCTAACAGCACTCAAAAATGCATG
AAGCCCCTCGTTTTTGAGGAAACATAATGCCCCTCGTTGTCGTTGTTCGATCAGATATCAGA
AGATTTGGGACCCTAATTAGTGGCAGTCCAGTCATTTGTTTGCACAATGAGTAGTCATCAAG
ATTGACGAAACAAGTATTTCTTCAAGAGATCATCAGCATGGCAGCACCCGGCCCCCTGTTTG
TGCTCATTGGTGTGGTGGCGTGGCCATCCTAATGGCGTCCTGTCATGGATGACCATGACGGC
AGCGTGGTGCTGGTTATGATGACGGCACTTGATTTGGAATGCATGGTGAAAACGAAGTGCGG
TCATTTTTTACTACCAAATATCGTGGTTGGTGTCCTACTATCCTGACTCCTGAGAGGAACAA
CATTCGACATAGACCTTTGGACGAGTACACGAAAGAAAACCCAAACAGGCAAGGATGCAACT
CATGTGTCAGACAGAATGTGATCTTTTCCCCAACAAGGATATGCGACAATACAGATTTCTCG
TAAACAGTCATTTTCCACATAAACGAAAAAAAAGAACCCAGCACCACAAAAACGTGGAATTC
TTGCACTTTTTAACCCTGTCGCAGCAAAAAGCTAATGACAAGATTGCCAGGCAAATAATTCC
AGCACTGCTGCCAGATTGCCACCATAGCATAGCAGACAGATTGGAGTAGACGATCATCATCT
CCAGCGGCCCTATATAGTAGCCATCGCAGGAGTATTGATTTTTGTCCGGGTCGCTTTCCGTC
CGACGTGTGTAGTGTAGCGCAATCCATCGGTCGCTGTCTGCTTTCTGAAGAACGTTCCCGTT
GACGCCGCTACGCTGCCTTGTCCTCTTTTCTTCCCTTCTACCCTGCCGCACGCCCCCTTCTT
CCGTGTGCAGTGGTGCAGGCCTTTTCGGCCCTCAGGCTTCTTCCCTTCCTTTCCCTGCTTCG
GAACTCCCGAGGCTGCATAACCTGATTCAGAGGCAGAGCGAGAGAGCGTGAGGAAGGGAGGG
AGATG
SEQ ID NO: 13
Sequence Length: 3003
Sequence Type: DNA
Organism: Sorghum sp.
GTTAATTCCCCCGCTTTGTCACTGGGTTATTAAAAATGTCGTTATAATGTATGGTTGTCTAC
ATTGTTGCTTCTTGATAATTAAGATGCTGTCGTCATGTCATCACTCGATGAGTAGTTGTCGG
AGCTTTGTGCCTCTGGAGCTTTGTTGCAACCTTCAAGGCCAGTCAAGCTTCAGGGTATTACT
ACCCCAAGCATGCTCCGGTAAGCGTCAAACTTACATTCGTCTTGGGCATGCACCAGAGTGGC
AGGTCTACCCTCTTCTTGCTTGAATGCTTGACCATTTTGCCACGCAAAACAAGCGGATGGTC
TTCCCATCCTCGCGCAAAACAAGCGGTACGTTCCATTAGCGGCTGAGTTTGGTTTGGGTTTA
GAGAATCGCACCGGAAAACGGTAATCATGTGCCGGCTTTCCCTTTTGTAACGGAATAACATT
TCCAGGCAACGATACGCACAACACACCGTCACAGCGTTGGTGTTCGGTAGAGTATCTAGTTC
TCTTCATAAGAAGAAAATTTCCTATTTGACAGCTGAGAAAAGTTGTGCTCTTTTATTTGACA
TCCAAACTCAATCTTTCTTATTTGTTTACTCTTTCAATTCTTCTTTCCTATATAACATTCCC
GTCAATTAGTTGGTGTAACAGTGTTAAATCCCATGTAAAAAGATTATTTTGCCCCTAGTCAG
TTTTTTGGTCATGATGTTGCACTCTCAAGATGGATATTGCAGGTTGTCTTCCTCATACGTTG
GTTGTTGCTGTCTTTCGTGACAATTTTTTTTAAAAAAATGAACATTTTTTTGTTGTACAAAT
TAATAGATCTCATTAATTAAAAGCACCAGCATCACCTTATAATATGATTCAATTTTGTGTAT
AAAAAAGACAAAAACATGAGATGGCAACAACATCTAGGTTTCACATGTAACATGAGATGACC
AATGAGATGTGTCTGAGCCTAACTGCTTTGTCCCCAACGCGTCGCTATGCTGCTACGCCCCT
GTGTCGGGCGACCGCACCCTTGTGTTGGCAGCCTCCACACTCCGCCTCTCCATGACCAATTA
GGGATTTGGTTGAGAACCAGTCGATTCCCCTCTTCTATGAGTACTTATTTTGGTCTTTGCAG
AAGCATCCATGAGGGCAACACATGGTGAAGTCTATGGACATGACAGAAATATCATATATGAA
TGAAAGAAAAATTGAGAGAGTGGACAGTTAAGAAATATTTTGAGTCGAGTGTCGAATAAGGA
AGCATTACTTTTTTCAGTGTAGAATAGGGAATTTTCTTCTTCATCATCTGCACAAGTTTATA
TGACACACTATTACGCTTTTTCTAGTCAGAGTAACCTCCCGATGCTAGCTCGCCGTTGCGCT
GGCTCTAGTATCTGGGATGTCCCCTAGCGATGGTGGCCACACGAGTCCACATCAATCATCAA
GCTCCACACGCTGTCGGCAAGGGAACTCTCGCCGCCGCGGGTCATGAGGCCTTGTTTAGTTC
GCAAAAATTTTCAAAATTCTCCGTCACATCGAATTTTTGGTCGCATGCATAAAACATTAAAT
ATAAACAAAAATAAAAACTAACTGTATAGTTTATCTGTAATTTGTGAGATAAATCTTTTGAT
CCTAGTTACTCTGTGATTGGACAATGTTTGTCAAATAAAAACGAAATGCTACAGTAGCAAAA
AAACAAAATTTTTTTACCAATCTGAATTCATCTCACCGCCGCCACCTAGGCACCCTCAGCTC
CGCCATCACTAGGGCCTTGTTTAGTTCCCAAAAAATTTTGCAAAATTTTTCAGATTTCCCGT
CACATCGAATCTTTAGACACATGCATGAAGTATTAAATGTAATAAAAATAAAAACTAATTAC
ACAGTTTAGTCGAAATTGACGAGACGAATCTTTTAAGCCTAGTTTATCAAATACAAACGAAA
AAGCTACAATATCGATTTTGTAAAAAATATTTTGGAACTAAACAAGGCCTAGGATCTCTTGA
CTCCACTGCCACCAGGAGACCCTCGGCTCGACCGCCACCATGGAACTCTCGACTCTGCCGCC
ACTAGGGTGAAGGCACGAGTCTTTATTTTCAACGGTTTTGGTCAAATCCATTCGTAAAAATA
GTAGGTTCACTGGATATCCGAACAGCAAGTTTGGATCTGGGTTGAAAAAATTGACAGGCCTG
CAATTTATAAGCGTTTCGGCCCAATTACCGGGCATCAAACAGGCCAAACTCTATATATTTGT
TTGGCCAACGGCCCAATGGCCAGCGCAGCGTCAGGCAGCCAGCACTCCCGCTCCCCCATTTC
AAAATTTGAAAATATCAGCCCGGCACCGACGAAGCGGAACCACCCGTCCAAATCGACACCAA
CGTCGAGCGTCTCACCTCACTCACCTATAAACGGGCCCCACCATATTCCTGGCCCACAGACA
GGTAAGATGTCGCTGCTTGTGCTGTCTTCCCGCACCGAACGTTAGGTGACCCGAGAATAGAT
CACCAACTGGTCTTTTTTAGAGCCACACACCTTGGGAGTACACACAGTCGTGGACACGCACA
AAATTGGAGTACAAGAAGGTCTGTGGGGCCCATTTAACAGACAAATAATAGGCAGCGGTGGG
TCGCGGGCTAGTCGCAACTCAGGTACCAATAAAACGCGAGTAGTTTTGAAATATTTTGCTCC
TAAGCCCCTGTAAGGTTTTTCTTTTTATGTCAGCTTGATCCAGAGTCCCAGATTAGCCGCCT
TCCGACCGTTGAGGAGCCCGAACCGTGGAATTAATCAAAACTCTAGGGGCTGAAACGCAAAA
AACCGTCTCCTCGCTTTCGCTTCGCCGGCAATCCATCGTGGCCCAGGCTGTCGTTCCGTTCT
ATAAAGCGAGCCGAGTGGGAGGAGCCGGAACCCTAGCCGAGCACCGCAGAGACAGGCGTCTT
CGTACTCGCCTATCTCCGCGACTCAAAGCTTCTCCCCCTTCTCCCATTTCCCACCGCCGCCG
CCGTTCCACCCTTCCGACGACACCATG
SEQ ID NO: 14
Sequence Length: 3162
Sequence Type: DNA
Organism: Sorghum sp.
AAAACTAGCAAGTAAAGGACAAAACATATTATATTAGTTATTAATATCGAGAGGGGATAAAA
GACTACAAATTTTAAACTATTCGCAGATGAGGTTTTATGGGACACAATAAGATGGGTAAATA
TTAAAACTAAAAGAAAAAATATATTATATACAGTTAACTTTTAAAGGGGCAAAAAACAGAGA
ATAAAGAAATGCAAAACGGAAAAATGAAAACAAAATTGCAGTTGCAAGGGTTTGAACTAGCG
ACCTCTTGGTTAAAGAGCAAAAGTCTTACCATTGGGTTAGCAAGCCATGTAGATAATTAAAG
TTGCAAATTCTTATATGTAGATTAATACATTGTTAATTAAGTAAAAGGCTTACCCACAGGGT
ATGCCGTGGCCCACCTGACATACCCCATGGGTCCGCCCCTGCTAGGGCAAGTAGGCACCTAG
CGGCGCATAGGCACCGACATGTGATCCTTGCCTTGTCGTCGCTTGATGCATGATGCTCCAGC
AATGGCTGCCCCCATGCGCGACCCGTTGGCGTATGACGCACGGGCTGCCACCACGTGTGTGG
AGGTGAGCCCATCCTCATCCTCTTTGTTCCTTTCCCCTTTTTATTTGCATCTCTCTCTCTCT
CTCACACACACATGAATCTAGTAGCGGTGTCAAGGGGGTCAAGTAGATGGCGATGGGTTGGA
GAGAGGAGTCAACACCACTACTACAAAATAACCTAACCACGACCTTTTTGAAACCCCCTCGA
AGGCGGGCACAATTTGGAACTGCCTTAGTTAATAACCTAAAATCCATTATCCGAGGCGTGAT
GTAATATAACCGCCTTGGTTAATGCCTATTAACCAAGGCGGACATTATAACGCAACCGCCTT
AGTAAATCATTAACTGAGGCGGTTATATTACATTGCCCGCCTCCTAAAATCTAGCCCAACCC
ACTACAAATTCTCAGCCCAATATCAGAGACGAGGCCCGATATGAGCGAGTAGTACATAAACA
TAAGTTAGGGTTTCCTAGCTAACCGATCCCTTCACTCCCTCCCTCCCCAGCCACCTCACTCC
CCAACCGAGAGCGGTTGCTCTCCTAGCCATCCTTTGGTGGCGTCGTGGGGGGTGTGGCCCTC
CCCACTCCGGCGGCAGCGCATGGGAACACGACCCCCTCCTCTAGCGGTGACGTGGCGCGGGT
GGTGCGGCCTCTCCTCCGATGGCGTCCTGAGACCCTACTTCGGCTCCTCACCAGCCCCATAG
CCCTGGCCACCCCGACACTCTCCAGCAGCGGCGTTGCACGAGAACCGACGTGAGGCTATGAG
GATGGCGGCGTCGGAGCCCGTGCGTCAGCAGCAGGGTGGCGACAATGGTGGACTGGGCTTGG
TGGGCCCGTCGATGATTTAAATGGGCTCGTCGATGGGCTTTTTTTAATTATTTTTTTTCTGA
TTTATTTATCGAGACGGGTAAGCAATCGCCTCCGTTAATGCTTGATTAACCGTGACCTTTTG
TTGGAGACATTTATCTTGCCCGTATCGGAAAATCCTTTTTGCCCGCTTCAGATAAGGATGGA
GATGACCTTACGAGTGGCCCGTGGTTTTTCTCTAGTATAACTAAATGAACTAAATAAAAAAA
TAAGGGAAGAAGTTGTTTATTTAGTTCATTAAATTAGACTAGAGAAAAACCACACGTACTGG
AGCAGCGTCCCCATAGCTTCAGAAAAAGAATTTTGTAGTAGTGCACAATGCAGATTCAAATA
GCGATGGCATGAGCAACACAGATTCTCGACAAATAGCAAGAAGCACCGACACACGACTCTCT
AGCATCACTCTCTGGATGTTTGATCGAATGAGAAAATAGGATCAAGATCAATGTGGTTGCAA
AAGATATTCGATTTCTCACCGGTCTATAGCGGAAACAACCATCAACGATTGCAGACCTAACT
TATGAGCTGTCTTGCATGGACTATCAGAAATCGAACAAAAAGAATGGAGCTGCGTGTGAGAA
AGACAAGCGGAATTTAGTTATTTCACTTTGTTTTCTTTTTATCATGTCACATATGGGCAGCT
AGTGATGCCTTCGCATCACAGCACTTGAAGTGAGATTCTATTTTGTTTTTGTTACCATGGGA
CCTGATTTTCTTTTGGCTCCCACACTCTAGGGGCTTGTTTAGTTCCTAAAATATTTTGCAAA
TTTTTTCACATTTCTCGTCACATCGAATCTTGTGACACATGCATGAAGCACTAAATATAGAT
AAAAGAAATAACTAATTACACAGTTTACTTGTAATTTGCGAGACGAATCTTTAAGCCTAGTT
AGTCTATGATTAAATAGTATTTGTTAAATACAAACGAAAGTAACACTATTTATATTTTGTAA
TTTTTTTTAAAGTAAACAAGGCCTAGAATCAGACACTTGGCCGTTACGGTTGCAACTGACCG
GCCATTCCATAGGGGCCGAGTCAGCAGGTCCAAGCGCCCAAGGGTAACCCTGTACTTTCCCG
CGACGGTACGATACAAAGTTTCAAATTTCAAAATTTGAAACGGCTGGCCAACAGAACCCGCC
GGCGGCCGCTCCCCTCCATTCCCCTGACGTCGTCCCATAGGCTCCCCAGCCTCACACATACT
ACAAATCTCACCCGCATCAATGCTCCAGGGGGCTCAAATATTTGTGCCCATCAGTTGGTCCC
ACATGTCCGTGTCACAACATCCACGACCGGGTAAATGTCGCCGAGACCCCGAGCGCGCCGGC
TCCGCGGGACCCGCCCGCCACAGCTCATTCCCACCGTTGCCGGCCGCCGATCACGCAAGCCT
CAGAGCCGTTCGAATCCAAACGGTCGTTAACCCCTCGTTGCCTCCGCCCCGCCCACCACCCA
GAGACTGATCCGTGGGCCACACCATCACACCGTCAGTCCCGAACCAGACGGCGGCTAGGTCT
ACCGCGCCGCGCCACACCATCACGGGCCGGCCGCGGCCGCCTCTCCACTCTGCCTATAAAAG
CCGCCGCGGGGCTGGGCGGCATTTATCGTTCACCTCGGCGTCTTCACAAACGCCGGCGCTTC
CACTCTCGATCGATCGATCCTCGACCATTCCCCATTCCGTCCTCCCCCGATCGATCCTCGAC
CATTCCCCTTCCCGTCCTCCCCCGATCGACGAGCGGTTGTCTGAGAGAAGAGGAGGAAGATG
SEQ ID NO: 15
Sequence Length: 3131
Sequence Type: DNA
Organism: Sorghum sp.
GCCACTTTACCAGACTGCTTCAACAAATTTGAGCAGCCAAATTATGAGTTGTGCTTGCAACT
GAACGTCTGGACCTGTTCAAGCTTTTGAGCAAAATGTCTATTCTAAATGCGATTCAAATTTA
AAAGGCTTTGATTCAACACTCAAGGAGCCTTAATTTGAATGTTTGAGAAGCCACTAATCCTC
TGTCAGTCTGCAATATGTTTTACTCCTTCCATTCTGAATTATAAGACGTTTGACTTTTTTGA
CTCTAAATTTGACCACTTGTCTTATTAAAAAATTTACACAAGCATAATCAAATTTAAGTTAT
TATTGAAGAACCTTTATTAATAAACCAGGCCACGATAAAAGAAATGATATCTTAGACAATTT
TTTGAATAAAACGAATAGTTAAACTTGGTGTTAAAAAAAATCAAATATCTTGTAATTTGAAA
TGAATTGCGTACTATATTATTGTCATGAGTCTGTTTCTTTGCCGTATAACTCGTATAAAAGA
GCAGATTTGTTGTTCCCTTTTTGAATTCTAGTAGCTTTGATGTTCTGCTATCTCAATTTTTA
TTCTCACCTCTCGTGCTCGCGTCTCCCAGAGATCCATGGTAGCAGTTTAGCCACGTAAGACC
TTGTTTGGATGTTGTCGGATTTACTTCAATCCATGTGTGTTGGTGTGGATTAAGATGGAATT
TAGTTCAAGTTCTACTCCAATCCACGTCGACACATGTGGATTGGATTGATGTGAATCCGACT
ACATCTAAACAAAGTGTGAGCAGGACTGTTGACCGATCGCTATGTTACACCATTCAGGACCG
GCGCTGCCCCAAGTCATGTACGATAACAATAACAAGCATTTCCCCTGACTAATCAACGAAGA
ATCGGGGCGAGGACAAGAGTGGTTAGCGTTGCTGTTGACCATCCTACCTGGCAGCAATGTTC
AACTCGAAGCTAGTGTACCCATATATAGCGTGATAGCAATGATGTACTGGCATCGAAGCAGT
CAATAAAATAAGACCCCACTGTTTTTGTTAACCATGATTGGATCGATGTATCGCTAGGGGCT
GTTAGGATCACTTGGCTATCTAAACGGGTACTGCGTCCTTAGCTTTCCTCCACCGTTGAACC
TAGGTATCGTATTGCCGTGTGGCAAGCGAAAAAATAGCGGTTCTTTTCTCTTGTCTCATGAT
AAAGTTTTAGTACGTGTTTAGCTTGTACTTTCAGCTAGTCCGTCCCCAGCCATGCATGCATT
ATAGTTGGTGCCACTGCCACCCTCTCTGTGTTCCCTACCTGAACATGAGCTGATCAGCAACA
TGCTGGTAATTGGTCCTTGTCATTCCTCTGACTAAGCAACAGCTACGTCTGTTGACACGCAG
GGTCCAGGCTTTGCTTGCTTGCATTACCTACCGGCCAAGCGTCGCTATCCGCTGTCTAAAAT
AAATAGCCGCCATAATCACACATCTAATCATTAGGGCACTTACAAGACTCTATCACAGAGTC
CAAGAGAATTAATTACATACTATTTATGATATTTTGCTGATGTAGCAGCATATTTATTGAAG
AAAGTGGTAAAAAAATAAGACTCCAAGTCTTATTTAGACTCTAAGTCCACATTGTTCAAGAT
AATAAATAACTTTAGACTTTATGATAGAGTCTGCATTGTGAGTGCCCTTAGGACAGTCCCAA
TGGAAGAAACCACGACAGTTTCTATAGCATAGGATACTGTACCAAGAAACTATCTTTTCCAT
TGCATTATTTTGTACTAGTGTCTAGATAAAAATGTTCCCAATCATTCACTTTATTTCTCTCT
CACATCTTTGGATCCTCTGTGCATTTGGTTTACTCTTTCTTACTCTATCATCCTCTGCTGCC
GGCGTCCAAATAGCGAACGAGCTTCGTCTCTATCGGATAACTAGCTAGCAGCTCCGTCACCT
GGAAGGAAGGATTGTTTGCCTGCTCATCATTCAAGGCTGCCGGGCTAGCAGGCGGCAGAGCC
TTTTCTACTACTTTGCTGAGGCACAGCAGAATGCCGCCAAGCCGCTCACATTGTGGCTTAAT
GGAGGTTTGTTGCTACTGCTCGCACCTCATCCATGGCGTGAGCATGTGCGGACCGGCGAGCC
CTCGTCCATGGCGTGAGCATGCACAGACCAACGAGCCAGCAAGCTCCATCGCGTCGTGCGGC
ACGATCGGGAGATTGGGCTGCGGCCGGAGCTCCATGGCGGCGCATGTGCTGCCAGGCTAGAG
TTCCACGCCGCTGGCCCTTGGCGACGGGAGCGACAACACAATTTGGAATAGGAGATCCGAAC
GGAGTTGAATGGAAACGACCAGTTTCTCCCCAACGCAGATCACGTAGTTTCCTCACGCATCG
GTGTGAGAGACGACTTCGTTTCCTCCATAGGAAACGGTTTCTCTAATTTTTCTTCTCTCTCC
CTAATAAATCTATTTTCACATCACCAATTTGCTTAGTTGGCAAGTTAATTAATAACGATAAA
AACTACCATCAACACATCATTGGGACTGCCCTTACATGCTCAAGACGAGAAGAGAGCACCAC
AGGCTACAACAGGCTACAGCTCGGGTAAGCTTGTCTTTTTGGGCCCGCCGTTCGGTTACGAC
AGGTCGACAGCCTGCCACGTGGGCCCACGCCCAAACCTGGACCCAAAAGTCGCCAACGCCAA
TACCAACGCCAACAGAAAGAGCCCAACCAACAAATCGACACAAACTTCCCTTTTTTTAAAAA
AAAAACACAAAAGAAATCCAGGAAACGGGCCCTCTAGCCGTCCGATCAACAAACGCACGGTG
GAGATGGACCAGCTCCACCGCCTCAACGCGTCGGCGCCTGGGCCCCTACCGCGGCGGCCGGG
TCTCTCTCCAGTCTCCACTCTCCACCCACCGGGCACGGGCCGCCACAGCACAAGAAGGTCCA
CAACCCCCTCCTCCAGCTCGAAGGCTCTCGGTGGAAGTCGCACGGGGGCAACAGCATAGAGC
AGCATTTCAAATCCGTCCTCACCTATAGACAAGACCGCAAGCCCACAGCACCCGAGAGAGGT
CGAGACCGTGCGCCGCTCCCCGCCCGCCTTTTCCCCGCCCGCGTCCGACCTCGACCCCAGCC
CCGGCGAGCCAGCAGGCAGGCGTCAGCCATG
SEQ ID NO: 16
Sequence Length: 2680
Sequence Type: DNA
Organism: Sorghum sp.
AGCTATCTGATAGCAAGGCTGTTGTGGACCTCTTTTTTTTTAAGAAGTTATCTATTTTACAA
AATAGCTAAACATTAGAATTTGACTATTAATTTTAGCCAAGCTCTTGGAGATGCTCTAAGGT
GTTAAGCCTGGCTTGATTAGGAAGCCAAGGGGGTTTGGACCCTGGTTTATGGGAAACGGCTA
GGGCGAGCATTTTCTTGGATCTAGGTTAGGGCTATGCGACCACCATCTCTGCTCATCACTGC
TTGTGCTATCACCTACTCCCTTCGTCCCAAAAAAAGTGACGCTTTTGACTTTCAAACATCGT
GTTTGACCGTTTATCTTATTCAAAAAATTTATGTAAATTATAAAATAAATAAATCATTAGTA
AGTATCTATAATGATAAAATAATTCTTAACAAAATATATAATATTTATGTAAAAAATTTGAA
TAAGACGAATAGTTAAATAAAATGTCTAAAATTCTAAAACCACATTCTTTTTAGGATGAAGG
GAGTATTATGCCGTTGTCAAGCTGCCAAGCACTTGCGTCACTTGTGTTGCCACCCTTCGTCC
CGTTTGCACACACCGACTAAAGTCTCATCTGCAATGGCTTCGACTTCGCGGTGGGTGCAGAC
TGCCACTACGCTTCGAGGAAGCCGTCGCCGGTGATTGCATTTGCATTTGAAGGGATTTTTTT
TTCATAAACCTAACTTTGTGATTTTTTCATGTCAGAGGCATTATGATTTTTTATTGTCCACT
TAATACCGTTAAATGCTAGAAACGAACGGAAAGCCATAGAAGGAACGAAAGTTGGTTTAAAG
AAAGAGAGTTCAAAAAAAAGAACTATACAAAAAAGAGGTATTTTTTGAGGGCCAAGTAAGGG
CATGTTTAGATTGGAGATGAAAAATTTTTGGATGTCACATCGGATATGTCGGAAGGATGTCG
AGAGGGGTTTTTAAAAACTAATAAAAAAACAAATTACATATCTCGACTGGAAACTGAAAGAC
AAATCTATTAAGTATAATTAATCTGTCATTAGCACATGTGGGTTAATGTAGCACTTAAGGCT
AATCATAGACTAACTAGGCTTAAAAGATTCGTCTCGCGATTTTCAACCAAACTGTGTAATTA
GTTTATTTTTTATCTACATTTAATATTCCATGCATGTGTTCAAATATTTGATAGGATGGGTG
AAAAAATTTTAGGCTGTAAACTAAACAGGGCCTAAATCCTTAGCATAACACTCTTGGCACGA
TGTACAGAGACCAAAATCCAGTCGAATTTCAAATTTGGATAAACAAATACTCCTGACCTGAT
GTACGCAAACCAATAAGGCCTTGTTTAGTTCCGAAAACTGAAAAGTTTTTGGAACTGTAGCA
CTTTCGGTTATTTGTGTCAAATATTGTCCAATTATAGACTAAATAGGATCAAAAGATTCGTC
TCGCGATTTACAGATAAACTGTGCAATTAATTTTTATTTTCGTCTATATTTAGTGCTCTATG
CATATGCCACAAGATTTGATGTGACAGAGAATGTTGAAAAGTTTTTGGTTTTCGGAGTGAAC
TAAACAAGGCCTAAAATAAAATAAAAAGATTTGCCATGTACGCAAAACGAGACAGTCAGACA
GCCCATCCTGGGCCGACGCCGGCAAACCAGAAGCAAACAAACGGCGAGACGCGCCCGGGGCA
GTAGCGTCACACCGCAACAACCTGTTCCGTTCCGCGCCGGGGGGGGGGGGGGGGGGTGGGGT
GGGGTGGGGTGGCGCCGGGGCAACACCGTCATTTCCGCTGACACGGAAGCGGACACCCGAAA
AATTTCAAAATCCAAGCGCCCAACGGGCCGTTTTCGAACCCGACGCAGCCGCCCGTCCGATG
GGAACGATCGGACGGCCTCCGGCGGTCGACGGCGGCGTTGGAGGGAACGCGACTGGGCCGCC
TGATCCGGTGCCCTAGCCCCCCGCGCCCACTATAAAATCCGCCCCCTTTCTGGCCACTCGCT
CATTTCATTTACCACACCCTCCCCCTTCCCCTCCCCGCTCCCCCCTCATCTGGACGGCCGAC
TCGCTTCTTCTTCTGTGAGGTAATGCGGCGGAATCCTTGTGCCATATTACGATTTTGGGTTT
TGTTTTCGTGTTCCCTCCGGGATTTATGTCTGGTAGTAGCAGATTTGGGGACTTTTTTTTGG
TTTCGTTTTGTGAGGTTTGAATTTTGGGGCTAGATTTGGGTGGATGTTGCGGTGTCCTTCGC
TGCTGGTGCGGCTATGTTTTTTTATTAGATCTGCACCGCTCCAAATTTTGTTTAGGCGTTTG
ATTGTCAGATCATCAGTCATCTTTCGCTGCTTCTGGATTCTACATGTTCTCGGTTCTTATAT
TGGGATTTGAGATTTGGCTTTGTTCATAGGTGACGCGCTTCCGTGAGGTATTCTCATAGAAT
TTCAGGTAGATCTCAAGGGGCTCCTCACTTCCCTTGTGGTGCTACAGCCAGTATTTTAAGTT
TTCTGCAGTCCTCTCTCTTTTTTTAACTGCACTTTTTCCTTTATTCCCGGATCTGATTGATT
TCGTGCCGGAGCTTGTTATTCCTCCATAGATCTGGTTCTCCACTCCCTTTCGGAGTAATGTC
TCCATCATTTTCACGCTACTAACCGCCCTTCTGCTCCCCTCCCACCTGCAGCTACCAACCTT
GAGATCAAGCCATG
SEQ ID NO: 17
Sequence Length: 3081
Sequence Type: DNA
Organism: Sorghum sp.
TTATAGTCCAATAGTCTTTTGCATCTTCAGACAAAAGCCTAAGATCAACAAACATCACTTTG
CATAGCATTATCATCGTCACAGAGATAAGTGATAGGATGTTGTAACAAATTTTATGAGTCCT
TGATATATTTCAAGTTCTCATGGTAGAACTACAAATATCTAAAATTAACATGAGAGCTATTC
ATAGCAATTCACTTTGCTATCTAAGAAATCAATTTCAAACTATGACATAATTAATTTTTTCG
CACAAAAACTGTAAGCATATATGTGTGCCATGAAAGCTAATAGGTTACATGTTTGATTGGCA
AATTGGTAATGGCAACGACAAATTGCGGAGGGGGATCAATGACGAGTACACTTACAAGACTT
TGTTTGGTTGGACAACACGAGATTGAGAGTTTGAGTATATTTAGATAACACCTCGAGGTGAG
AAATTGGCATCGCTTGAACTTATCAGTCAAAATCAGCTATACTTTTTCAATCATAGAATGGT
GTTGTTTTTTTCTCACAGCGAATTAGCATCAGTCACAGAAATGAGAAACATCAAGTAACGTG
AAGTGATCATGTTGTTAATCATCGCAGGGGAAAAGCACTGAACCAAATAACATGTTAGTGTT
CCTGCTTTTTGTTTCAAGCCCAATTATGGCTTACCCTCCTTTGAAGCCCCTTTATATTTCAT
TAAGATGATTTAAAAATATCAACTAAGCTATAAAAAAACTAGTTGCCACACGGAATTGCAAT
TGCCTACTTTTGTACGTACTTTTATGACCCCCCCTTATTGTGACATTAGCATTTTGAAAGAT
ACCAAAATAATTTTGACAATAAAACTTGACAAAAATTGCATGCATTTCAACTTTGATCAAAC
TCTGACAAACACTATTTTAAAAAGTACGTAAGTGCATAGATAAAATTACAAACTCACTAATA
ATTCTTCTACCAATTCTCTAGATGTTTTCCCTTTTTTTAACTCTGTTATTTGAACTCCAACC
AGCACAATTAAAAATAGGGAAAGCAGTTGTTCGGGGTGTAAAAGAAAGGACAAAATCACAAA
CTTAGACACAAAAAGTTAGGCCTTGTTTAGTTCCCAAAAAATTTTGCAAAATTTTTCAGATT
TCCCGTCACATCGAATCTTTAGACGTATGCATTAAGTATTAAATATAGACGAAAATAAAAAC
TAATTACACAGTTTGGTCGGAATTGACGAGACGAATCTTTTGAGTCTAGTTAGTCTATGATT
GGACAATATTTGTCAAATACAAACAAAATTGGTACTATTCACATTTTGCAAAATATTTTGGA
ACTAAACAATCTTCACAACAAGGGAAAGGACGCCATTATCATCTCTCAAAAACTTTTATGAA
GCTAAATTGAGATCTAGATCTCCTAGATCATTTATCCTGAAGTGATACTTGCATAGTTTACT
TATCTCAATAGAAGTGATTCTTTCTCCAAAATCAAATTAGAAAGTTGAGGCTAAACTTAACT
ATCATGTTGCTTCAAATTCGAAACAAACTTTTCATTCTCCAAAAACATGGGCCATGAAACGT
ACTCTCTCCACATACCAAAACAAGTGCACGTATTGCTTATCGAAGAACCAACCATTTTTTAA
AAGTTTAACTAATAAATATATAAAAAACACTATCAATATTTATATCTTTAAATAAATTTATA
ATAAAATTATATTCCACTATTAATCCAATAACATTCTACAACTAGAGTTGGGCCTTGTTTAG
TTCACTCCGAAAACCAAAAACTTTTCAAGATTATTCGTCATATCAAATCTTCGAGCACATAC
ATAAAGCATTAGATATAGACGAAAATAAAAACTAATTGCACAGTTTGCCTGTAAATTACGAG
ATGAATCTTTTGAGTCTAGTTAGTCTATAATTGGATAATATTTGTCAAATAAAAACGAAAAT
TATATAGTGCCGAAATCCGAATTTTTTTCGAAACTAAACAAGGCCTTGCTTGTTTTTGAAAC
TAATAAAACACACAATTATCCAGGTCTTGTTTAGATGCGAAAAGATTTTGGATTTCGCTACT
GTAGCACTTTCGTTTGTTTGCGGCAAACATTATCCAATTATGGACTAATTAGAATTAAAAGA
TTCATCTCATATAATTAGTTTTTATTTTTATTCATATTTAATGCTTCATACATGTAGCGAAA
GATTCGCTTGAAAATTTTTGTAAGGCCTTGTTTAGTTTCGAAAAAATTTCGGATTTCGCTAC
TGTAGCACTTTCGTTTTTATTTGATAAATATTGTCCAATCATGAACTAACTAGGATTAAAAG
ATTCGTCTCGTGATTTACAGACAAATTGTGTAATTAGTTTTTGTTTTTGTCTATATTTAATG
CTTCATGCATGTGCCGTAAGATTCGATGTGACGGAGACTCTCGAAAACTTTTTGGATTTCGG
TTGAGGCCTTGTTTAGTTCCGAAAAATTTTGGGAAATGGACACTGTAGCGCTTTCGTTTGTA
TTTGATAAATATTGTCCAATCATGGACTAACTAGACTCAAAAGATTCGTCTCGTCAATTTCG
ACCAAACTGTGCAATTAGTTTTTATTTTCGTCTATATTTAATACTCCATGCATGCGTCTAAA
AATTCGATGTGACGGGGAATGTGAAAAATTTTGCAAAATTTTCTGGGAAGTAAACAAGGCCT
GAACAAGGCCTTGGTTTCGCGCTCGTGCTGTCATGCAGTACCCGCAGAGGCGCAGAGCAACA
GAGGAATTCTCGCTCACGTGACAATGACGTCACCCGCGTGCGCGACGAAAACCATTTCCCTC
CGTTTCTTCCCGCGCACACTTTGGCCATGTCATCGATCCGCTCCAGAACGCATCTCAGCCGT
CCAAGCCAAGAAGCACCAACGCCTCGCGCGCCTTCCACGCCAGCGATCCGCGGCATCCACCC
TTCCACCAACCAGCGCGCACTACATTTCCGCTTCCGCTATAAAGTAACCGCCGCCCCACATC
CCTTTTCTCCACCGCAATTCCTCCGCAACTTCACAACACAGATCATCGTCTTCCAATCGAGC
AAACCCTCCTTCGGTTTAGAGAATCCGAGCGGCGGCATCGATG
SEQ ID NO: 18
Sequence Length: 3062
Sequence Type: DNA
Organism: Sorghum sp.
CAAAAGATAACGCATATATTTTTACTGGCACAAAAAGAATAGAGTGGATGGAAAGACGGTCA
TGCAGAGGGTGTATAGTTCACCTTTTATTTAAAAAAAGAAAAAGTCTAAATAGCCCCCTCAA
CTATACGCGGTGGACTACTTCACCCTCTGAACTATAAAACCGAATTTTCTACTCCCTGATCT
TTCCAAAACTGGTTAAATAACCTCGCAAGGGTTTTAGACCGTAGTTTTACTATAGTGATAAT
GGTTTTGTCTTTTAAAAAAAAATATTTTCGTTGAATCTTTGAAAAATCATAATAAATTACAA
AATAAAAAATCTAGTCTTTTTAGGCTCCACATGAGTAGATCTAATATGATATATTTTACTAC
AATTTTTTTGTTGTAACTTTAGAGCTATGAATTATTCCAATTAATTAAGCATAGATCTAAAG
CTGCAGTGAAAACTTATACTAAAGTATACCATATTATATGTTTACTATGCATATCTAGGAGT
CCAATAATTTATTTTATAATTTTTAAATATTTAGCAAAAATAAATAAAAAAGAAAAAAACAA
AACCACCATTAAAACCGGCTGAGGGGGCTTATCTGACTGGTTTTGGAAAGGTTAGGGGTCTA
GAAAATCTGATTTTATAGTTGAGAGGGTGAAGTAGTTCACTATGTATAGTCGAGGGGGTTAT
ATAGACTATTTTCCCCCAAAAAATTGGGTTTTGATTCATCATTTTGCAAAATAGAACTAAAC
ATTATGCATTTTTTTAGGAAAAAAATGGTTATTCTCCATTTTGGATTTTGACCTCAAGTGGC
TTTTACGAGAGCAATAAATTCTACATTTTGGATGAAACTAAATATGACCCTGAAAATTTCAG
CTTTTTACGTTCCATTATTCCAAAGTAGTTGGTATTTTATATTTTATATTTTTATTTTAACT
AAACACCCCCATAGATTTTCATTGGCACAAATGTTTGCATCCCCTTAGGGCCTGTTTAGATT
GGAGATGGAAATTTTTTAGATGTCACATCGAATGTGTCGGAAGGATGTCGGGAAGAGTTTTT
ATAAACTAATAAAAAAACAAATTACATAGCTCGTCTGAAAACTGTAAGACAAATCTATTAAG
CATAATTAATCTGTCATTAGCACATGTGGGTTACTATAACACTTAAGGCTAATCATGGACTA
ACTAAGCTTAAAAGATTCGTCTCGCGATTTGTCGGTGTTTTTCCCCCGGGGGGGGGTCACAC
CAACGAGTAAATTTGTATGCGTGCTCCCCTTTCCGGATGGTGATGCAAGAAGACACAGAGAT
TTATCCTGGTTCGGGCAAGAGAAGGCCCTACGTCCAGCGGGGGGAGAGAGTTTGTATTATCT
TGCACCTAAGTGCTTGTACAGGGGTGAATACAAGCGTGGTATGAAGTGTGTAGCTCTACTAT
GTGTGTGTGTTCTTGTGTTGTGATCTCTCTCCCTTCTATTCCTGAGTCTCTCCTTTTATAGC
TCCAAGGAGAGACACAGGGTACATGCGTAGATGTTAGAGTAGGGATCGATAGCCACATGGAG
CGCTGACCTACTCGAGGCTTCCGTACGGCATGGCCTCGAGCCGTCCCATCTTGATAGCCTGG
TGATGATTACGCGTGCTCCTGCGTGCTGCCCTGCCTGCCGCCTTGTGCTGGTTCTGAGGTCG
CATGCTCGTATGGTATGGTGGCGGATCCGGCGGGGCAGCTGTGGTGTTGTCAGTCGACGCCC
AACTTGTCTCTGGGAAGGGACCTTGTTCGGTCGAGGGTCGGGCGCCGCGTAATATGCTGATA
TCTGGAGCGCTGACCTTGGGTGTCCCGAGGGGGTCCCGATTAGACGTTCCATCCTTGTTTCC
TGCGTCCTGACACGCCCTGGGTCGTTCGGTGGGAAGACTGCAAAAAGAACGATGGGACAGAA
GCTTGTTCCCTATCACGCCTTCCCAAACTGTGTAATTACTTTATTTTTCATCTACATTTAAT
GTTTCATGCATGTGTCCAAATATTCGATGGGATGGATGAAAAATTTTTAGGTTGGGAACTAA
GGCCTTGTTTAGTTCCTCAAAAAATTTGCAAAATTTTTTAGATTCTCCGTTGTATCGAATCT
TTAGACGTATGTATGGAGTATTAAATATAGATGAAAATAAAAACTAATTGCACAGTTTGGTC
GGAATTGATAAGACGAATCTTTTGAGCGTAGTTAGTCCATAATTAGACAATATTTGTCAAAT
ACAAACGAAAGTGCTACTATTCTTATTTTACAAAATTTTTTGAAGTAAGGCCTTATTTAGTT
TCGAAAAGTGAAAAGTTTTCAGTACTGTAGCACTTTTGTTTGTTTGTGACAAATATTATCCA
ATTATGGACTAATTAGGATCAAAAGATTCGTCTCGTGATTTTCATGCATGTGCCATAAAATT
CGATGTGACGGAAAATCTTGAAAATTTTTTGATTTTGAGGGTGAACCAAACAAGCTTAGCGC
ACTGACTGTTGGGCCTGACCGAGACCGACGCTCCGACGCCAAGGCCTTGTTTGGTTCAAAAA
GTTTTGCAAAATTTTTCAGATTCTCTGTCACATCGAATCTTTAAACATATGTATAAAGTATT
AAATACAGACAAAAATAAAAACTAATTACACAGTTTGGTCGAAATTGACGAAACGAATCTTT
TAAGCCTAGTTAGTCTATGATTGGATAATATTTGTCAAATACAAACGAAAAAACTACAATAT
CAATTTTGCAAAATATTTTGGAACTAAACGAGACCCAAAACCAACCGCCAGCGCGCCGAAAC
GCACAGTTCCCTCCGGCTCCTCCCGGCTACACACGTCAGCAATCCGCGTCAATACCCATCTC
TGCCGTTCTGCGATGGCACCAACGCATCGCGCCCCTCCACGCCACCGATCCGCGGCACCGAC
CCCTCCGCCAATCAGAGACCGCTGCTCCATTCCATAAATAAAACCGCACCCCACGCCTCTCC
TCGCAGCAATCGAAATTCCCCGTCCTCAAATCGACCTAGCTAGCGAATCCCTCCGTCCCCGC
AGCCTCACCCCCACAGCATCGATG
SEQ ID NO: 19
Sequence Length: 3076
Sequence Type: DNA
Organism: Sorghum sp.
ACCGATCCGCGGCACCGACCCCTCCGCCAATCAGAGACCGCTGCTCCATTCCATAAATAAAA
CCGCACCCCACGCCTCTCCTCGCAGCAATCGAAATTCCCCGTCCTCAAATCGACCTAGCTAG
CGAATCCCTCCGTCCCCGCAGCCTCACCCCCACAGCATCGATGGCGCCCAAGGCGGAGAAGA
AGCCGGCGGCGAAGAAGCCCGCGGAGGAGGAGCCCGCGGCCGAGAAGGCCCCGGCGGGGAAG
AAGCCCAAGGCGGAGAAGCGCCTCCCCGCGGGCAAGTCTGCCGGCAAGGAGGGCGGCGACAA
GAAGGGCAAGAAGAAGGCCAAGAAGTCGGTGGAGACCTACAAGATCTACATCTTCAAGGTGC
TCAAGCAGGTGCACCCCGACATCGGCATCTCCTCCAAGGCCATGTCCATCATGAACTCCTTC
ATCAACGACATCTTCGAGAAGCTCGCCGGCGAGGCCGCCAAGCTCGCCCGCTACAACAAGAA
GCCCACCATCACCTCCAGGGAGATCCAGACGTCGGTGCGCCTCGTCCTCCCAGGCGAGCTCG
CCAAGCACGCCGTGTCCGAGGGCACCAAGGCCGTTACCAAGTTCACCTCATCTTAGATTGGA
TGGTGTAGGTAGATGTGGCTCGGTTCGGTTTATGTGATATTGCTACCTGTAGTAGTAGCTGG
TGGGGGTTCGAAATGGTTGGATGTTGATCTATGTGTAGATGGATTGTGGTAAGAATTATGGT
GGTGCTTTTGGAACCCTGTTTCATGATCCAGAATAGTCACAGTGCTTGTTCTATTTTTGATT
TGTCAGGATGGATGCTCTTAATGTGTAGTTATCATGTTGCTGACAGTGAACTGATGATTCCA
TGTGCAAAGCTTTATGTCAAGTCTGGAGCAAGCGTGTTGTGCATTTGATGGTTGCTAGCTAA
AGTATCTCAGTGTGTTGAGGTGGGAAATGTTATCCAAGTGTCGTAAAGTTGGATATCATATT
AAGGTTTGTTGACACATTTGCCAGGAGGGAATGAACATGCACAGGCAATTTAGGCGTCATTT
CCTCTCTGGAAGCTTGATAGTGTAGGAAGTTGTGATCTATGGACAATGTCATGGCAATTGCT
GTTCTGCTAATCTGAGTTCTGAGCTTCTGAGCTTCAAATTCTGATATCAATGGTAAATCTAC
TTGATATTTAGAATATTTCTGTTGTCATTGAGGAACATGTAGAAAAGATATGCTGTTTTTTT
GGGTTGCAAGTTGGCTAGCACTAGAACCCATGTATAGGCTGGGCATCCCTACTTGTTTGGCT
CCTGTTATCTCAGGTTCATATTCGAGCAGCATGTTTGTTGGTCATTCTTCTGAATCCCAGCT
GCATGGAGCCTTCCATTTCTTGCAAGCTATCCTTAAAAAAAACAAGATAGCTGGTAAGTTAT
CATCCTGTCCAAGTCCAACCTTCAGCATTGATGTTCATTGTTATTCATTTTGCAGAGATGTC
TTTCCAGCCACTGATGTTCTTCTATCAGCTAAATTCCAGGAGTACTATTTTTTGTGTTATTC
ATCGTGACTGTGTCATGCACTGACTAATGCTTCATCTGGGGTGTTAGGCCATGTTTAGTTCG
GGTTGGAAAAAATTTCGTGACACTGTAGCATTTTCGTTTGTTTGTGGTAAATATTGTCCAAC
TACAGACTAACTAGGCTCAAAAGATTTGTCTCGTAAATTTCGACCAAACTGTGCAATTAGTT
TTTATTTTCATCTATATTTAATACTTCATGTATGTGTCTAAAGATTCGATGTCACAGGGAAT
CTTGAAAAATTTTGAGTGTTGGGGTGGAAGTAAACAAGGCCTTACTTGGAGTTGGAGCATGA
TGAGCAAGCCCAATGACATGATCCAAATATTTCTAAATTATTATTGGTGCAATCCTTGGATT
ATGCGCTTATCCATATTGCCATATTGTTGGTTTTGAACTCTGGATGTTACCTGTTTTGATAT
TGTTGATAAAATTTCTGTGGTTACTTTGTTTTTTGGTTAAGCTTTTAAGTGGTTGATTGGAA
CTTGTGGTCATTAGTTAGAAATATAGTGTGCCTGTTATGTTGAAGCATGGTGAAATGTGTTT
ACTTCTGGAACTGTGTAAGTTCTGGAGTAAGAGTAATTATGGTTCCTACGGCTTACATTTAT
ATAGTTACTACTTGCAACGGAATGATTTTATCTGGACCTCAAATATATCTTCTGATTTTTTT
TGGCCATCGCACTGCTTTGTGGAATTGAAGCTGAAATTGAAGCTTTCAAGGATAGAGAGAGT
ACGATGTACTTACTGTTCGCTAAATCGTTTTTGTGGCCAATGTTGATTTGTTACAAGAGAAA
AACGTTGTTCTGTGCCTAGAAAAGTATGGTTGATTCTGGCTAATAAGTTCAAATGTTATTTT
AGTAAATGCTGAGAATGTATATTGCAGTAATATTATTAGTATATTACACATTTACACTAGAC
AGTTACTGGCTGTTTTCTTTTATATAAAAGTTGCATAATGGTTGTATAATAAAGTACTATTC
CCTTCGTTCTAATTGATAAGATATTTTATTTTTAAGATTTATATTATTTTTATTATGCATCT
AAATATAGTTTATATCTAATTGCATAGCAAAAGCCAAAACAGTTAAGCAAAAAAAACTATAT
ATAATTTTTGAAATGGAGGAGGTGTATTCTGTAGGAGTATCACATTAGACAGTTGGACCAGG
CCGAAACCAACTGCTAAGAGAAAGGCCGACCGGCCCACCCCACTCTGCGCGCTGAAAGCCAG
TTCCCTCCGTCTCCTCCCGCCCTATGCTCTGACCACCTCAACTATCCGCGCCAAAACCCATC
TCCACCGTCCATTTGCGACAGGATCAACACATCGCAGCCATCCACGTCAGCCATCCGCGGCA
CCGGCCCTTCCACCAATCACCACCAGCTGCTCCGTCCCGTTAAATTCGCCGCACCCCTCTCC
TCTTTCTCCATCGAAATCGACCGAGCGAAAGCGAATCCCTCCCCGCCGCAGCCTCACATCGC
ACGCCACCGCGAAACCCCAGCAGCCGCATCCATCCATG
SEQ ID NO: 20
Sequence Length: 3003
Sequence Type: DNA
Organism: Sorghum sp.
AGATTTTGAATTAGGGGTTCAAATTGAAAAGGGGGCAATTTGTAAAAATCTGTATTTTCAAA
ATTACTTTGGATTTTGCATTGAAACTTCAAAAACTCAAAACACCAAAGTTGTACACCTTAAC
AAGATCTACAACTTTGCTTTTGAACTCATCCCCAAATTTTGCTTAGTTTTTAAGTTACAGAA
AAGGGGGTAGAAACTGAGGTTGAAATTAGGGTTTTTCTTAACTATTTCCTTACAACTCTCCT
TAACTAGGGATTAAACCACCATCACAAGCATCACTTCACAAAATAAACACACTTTATCTTCC
TAAGCACAATCATCAAAAATAAACTTATTTTAAGTTGATGCATCATGATGTGCTTAACAAAC
ATGTTTTGCAATGCTTATGATGACATGATCAAGTTTTAATATTCGTAACATCAGGGATGTTA
CATGAACTCTTTGGTACAACCCTTAGACTCATCATACAAAGGCTCTTCTGCTGCCTTCTTCA
AGGCCTTCATCCCTTTCATTAATAACATTGATGGTTTAGTATGACGACGCAACATTGCCTTC
AAGAATTCTACATCTTCCGCAGTCGTATCATTTGGTAAGACATGAGTTCTGGCACCATCATT
TCCCCCAGCAAATCCACCAACGGTAACATTTGGCACAACATGTTCACTCTGGAGTGTGTCGT
GGAAAAACTGATCAACGGGCATGTCTAGAGCATCGGTGTCGATGTTTGCTGCGTCCCCAACT
GGATAAGGCACCGAGCTACCCTCTCCATGCTATGTCCAAATCAAGTAGTCCTTTATAAATCC
TCGGTAGACTAGATGAGAAATGATTACTTCAGTATCTTCAAATAGAACAATATTTTTGCAGT
CGTAGCATGGACAATATATGTGCTTCGTCTTTGTTCTCAAAGCATGGTTCTTAGCGGCATCA
ACAAACCTATGCACCTCGGGTATATATGATGGATCTAGTCTTGATAAGTTATACATCCATAA
GGACCTCTCCATCATGAGCTGTTTAAAATTTAGTAAATTAATTGAAATATTATCTAGGAATA
TTTGTTATCAAAAATAAAGAACACCTAACAATTAAATCAAATTGAAAAAAATAAAACAAGTA
TTAAAAATAAAGATAAAACCCTAAGTAATAATTAAAAAAGAACACCTAACAACTAAATCAAA
TTGACAAGGTAGAAAAACACCGTTGATGGAAGCTAAATATATATCTTTATTTCACTAAAATA
AATTTGAGAAGGAAACATGGAAATGGTGAGAGGAGAGACAACATCTTAAGCAACCTCATACA
TGAAAAATGCTTATTGCATAATTAAATCATATTTACATAAAAAATAACATGCTAAACAATAT
AATTTAAAAAACTAAAATGAAAACTTATCTTTCTCTCTCTTCCCAAGCATGAAAACACCATT
CATGGAAACCACTACATATATATTTCTTGGATTCACTAATTAGAGAAGAAAACATAGAAAAG
AAGAGAGGAGAGACATCATCTAACCATCTTAAGCAACCTCATTTGAGCAAATATAGTCCATA
CCTAGCTATTCTACTCTCTCCCTCTCATGGTGAAACCCTAGATCCAATTAAAAAGTACCTCA
AAATGAGCAAGAGGGCACAAAAAGAGGCATTGGAGGCATCAACTAACCTTTCTTAGCCACGT
CCTTCTAAGAAATGAAGATCAAAACCTCCCCTTGTATTTTGAAAAATATGGACCTCCAAGAT
AGGCTGCAATGGAAGGTGGCTGCGAGCTACAGTTCTGTTCGAGGAGGAAGAAGAGGGGCTGG
GGGTATTTATAGGAAGAATAAGCACATGCGGTTTGCTTAAGGAACCGCTTGTGTAAATCTAT
TAACACAGGCGGTTCACATAGCAGAACCGCCTGTGTAAATGGACTATTTACACAGACGGTTC
AGTTATGTAAACCTTCTGTGCTAATAGATTTGCACAGGAGGTTCATATAACTGAACCGCCTG
TGTAAATGATCCATTTACACAGGCGGTTTTCTTACAATAACCGTCTATAGTGACTCTTTTAT
ACAAACGGTTTTTAATTCTGGCCGTACAAATTTACAACACAGATGCATTATAAGTAAAACCG
TCTGTGCAAAGTTTTTGCCCCCGCCGACTTAGAGCATCGTACTAGTGGAAGAAGCCAATAGA
CTCTAATGAATGTAGTAATGGATGTAGTAGATTGCGATTAGATTGAGATTTAGATTAGAAAT
GTAGTAACTGCAAATTGTGGACTAATGGGAACGACTTTGTACAAATATCATAGATTAGAAGA
TTTTTACAATGGCACGGTTACATCCTAAACTTCTTTCTACTCCCTCCGTTCTATGATGTGAA
AACATTTATAAGACCAAATTAATGCACTATAATTAAAACATATATTAGTGAAAGTGATTTTA
TCTTATAAAACTATAGACGACGTTTTATGCATTTTCTACAAATTTAGTATAAAGAATTAGTG
AAGTATCTAAAAATTAGCTAAGTATCTAGGACAAAACTAAAAAATCGTAAACAAAATCTTGA
AGGCGTGATTCGAACAAAACAGAGAGCCCGCGCGCGCGTGGGACGAGTTCCTCAAAATTCAA
AACTGGTCGTCTTCTCGATCTCTCGGCTCTTTCTGCTCTTATCCTGTGTGTGTGTCTCCTAT
GGGTCTGGACAGGCATGGAGTGGTCATGAGAGGACGACACGAGCACGACCATGCAGCCGCGT
CTGTCTGCGGCCGGTCTGCCTGTTCACCGAGCCCCCCAGGCGGTCCAAGTTACCACGAGTTA
CCACGTCGCCCGTCCAGGCAGCCGCGTGTCCATCCACCCGCTTCTCACGTGTCCCTCCCGTC
CTCTTCTGCTCTGCATAAAGCGTGGAGCCTCCGCTCCACCCATGCTTCGTACTACCTGTCCC
TCGACGCGCGCGACCCCTCCATTTCTTCATTTCTTCACGCTACTCACCGTGTAGTTTGTTGC

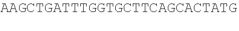

SEQ ID NO: 21
Sequence Length: 3069
Sequence Type: DNA
Organism: Sorghum sp.
GCTGTTGCTATGGCGAGTGGGTCACACGAAGATGAATCTCGTATCCCTTCCCTGCCTTTTTG
GTTCTTTGTGCTTAAACTCTTGTATGTTTGTACTTAATCAACCGTAGCATTTCTTTGGTTCT
CGCGGTGACAACCGCACCGCTAAGAACCTTAGTGATGTCTTAGTGCATTTAGTGCACCTAGG
TTGTGGCGCCCTACAAGTAGTTTGAGCACTCGTGTCCTTGGTGTGTCACTCCCTCTTATGCC
CTGTCTTTTGCCGTGGCATGAGTATTGGAAGGAGTAGACCACTTCCCTTCTTCTTCTCTCTT
TATTCCACCCTCTCTTGGCTCTCTCCAACTACAATGGCGTATGGGTTGAGAGAGACCGGAAC
TTCTCGTGCTCATAGTCTTTACGATTCCTGTCGATCTCTATGACACTTGGATCGAAAGACCG
TAAGCTGTGGTGTTGCTTAGAATGAGTTAGAGTCTAAGCCATTTATTAAAGCCGTACAGGTC
GACACGATCGACCCGGGAAGTACCGGCTAGGCTAAGACTCAAGCTTGTACTTGGTGAACAAC
CATTTCCGTTTCTTTTAGCCCAGGGATCGGACACCCACCGAGAGGGCTAAGCCGTTTTCCTT
TCGGTGCTCTTTCAGTGTAGTTGTCCTTCAGTGTTTTGTCGCCTCTTTTCACAGTCTTTCTA
GGACTAGTGGATTGATTGTTTCGCCTTGTTTGTCGTTTACGAGGTAGTTGCTTCGGGTAGCG
TTGATCGAATCGGAACCAGTTGAAGGAAGGACATGCAGATAGGAGAAAGACCTTGGATGAGT
ACAACTACAAGTGAACTGAGGATCTTGGAAATGTCACTCGACAGGTGCCACGTCCCACCCAA
CCTCGTAGAATCCTATTAGACATACAATATGTAGATGCTAGTGCTTTACTTTTATGCAAATG
AATTGAGATAGGTTGCATGGTAGAAATGCTTGTGAGCCATTGCCTTGTTGCAACCTATAACC
CTCGCACACCCGCTGTTAGGTTAGACGCTTGCAAACTACTTGCTACTGCTTCTACTACGCAT
TATATCTGTGATGTGATGCATTGTGGAGGATTGGATGTGAGTGGATCAGGCACGTGGTGCCG
ATAACTGGTTAAGAAATGGATAATGGATTTGGGGAGATCTTGGCGTGTGTCTTGGGTGTGTG
GTGAGGGTCGAGTCGACCGAGCAGGATCTACGACGAGTCTTGGGACAAGTCTTGCCGGAGGA
CGCTACCTGGGCGTTCTCCACGAGAGATACCTGTGGCGGGTACATGTGACAGGGAGAGGTCC
CGGAGTGGAGTGTCTTCGTGGGACGAAGCACCGGGATGGGAGGTGCTGTTTAGCACGGGGTA
ATCGGATGTCCCGTCGAGCGGGGCATCGGTTGGCCCCTCGTGAAGATGTCCTGTTCGGTCAC
CCTAAGGACTGAGATGTCCTGAGAACCGGTTCGTAGGAAGCCTTGCATTCCCACTCGCCTTA
GCCATGGAACGGGACGGATGTACGACCAGCTAGGGCGGTGCCACTACTACTAGGTTGTTAGC
GGAAAGTGTAGGGAGGTACGGGCCTGGGACCCACACCCTCCTAAGACAGCGTAGTGACCTTG
GGGGCCCGGTACTACGTCTCACAGTCTCAGCATGCCGGTGGTACTCCGGCATGGCCCCAGTC
CTGAGTGGTAGGGTGGCATCGTGTTTAGTTGGAAGGCAGCCCGGTATCAGCCTAGACGATGT
ACAGCGTCGATGATGGTGATCTTGTGGGTAGTGCAAACCTCTGCAGAGTTTCTGGTTGATCG
ATCGATACATATGCCGTTTACGGCTATGGACCTTTCCTATGTTTCCGCTTCACTTGACTAGT
GAGAGGAGTCCTTTCTACCTTCCCCTGGGTTTGTGTTGGATCCGGCGTTGGCCGATGAGGCA
AGGCACGAGCGGGAGTCGTACTTGCCGCCTAGAGAGTGAGAGTGTGGTGAGATGTGTGTGAT
GGGATGGATGGATGGATGTGTGGAAGAGATGGAATAAAACTTGATGAATTATTACTATATAA
TATTGATGAACTTACATAGGAAAAACTACAGCCATATATATAGGCCTCTTGAATCACCCTTG
CATTCCACTTACCACAAAGCTTACGCAAAAGCATAGGGTGGGAGCCAGTGGCCAGTACAAAT
CGTACTAAAAATTGTTTAGCAGGTTTTGAACGTGGTCCATGACGATGACTACGGAGAATAGA
AGGATTAGGTGGTCTTGTTCCTGCGCTCAAGTTTGGTTCGGAGATGAAGGCTACGCCCGCTG
ATAAACTACGCCGACTCTGATGATTGCCTGTGAAGGAGGAGCCTTCACCGCTGACGCGCTAC
ATCAACTTTGATATAGACCCGTGTGTGTTTCCGCTAGAAAAACGATGTAATAGGCTGGTTGA
CCAAGAGTTGTAAAGTAAATGTGATGTAATCTTGTTTTTCACGATGTATGACTATGATAACA
GCTGATATATGATAATGTGATGGATCAATTTTTGAATTATCACATTATAATTCGAATCTGAG
GATTTTTCCCTTTGTGGAAAAAATCTAGGTCGTTTCAGAGGAGGGCATTGTAATTTGAAACG
GAGGGAGTACATTGCATATTTGCATGGTCCAAGATGCGGAGGTTTTCAAATTCCAACTGCAC
AAATGTTTACGTAACTGAGACTGACTAGTAGGTCCAGGAGTGGGCCTGGCCAGAGCTGGACC
GACTCCAAAATCAACCGCCAAAAGAGCCTGGACGGGCCCACCGTTGCGCGCCGAAACCCAGT
TCCATCCGTCTCCTCGTAGGGCCCACACTCCAACGACGTCAGTAATCCCCGGCAAAAACCCA
TCGCCACCGTCTACTTGCGATGGCACCAACGCATCCCACCCGTCCACGTCGGCGATCCGCGG
CACATGCCGCTCCGCCAATCAGCGCCCGCTGCTCCGTTCTATAAATACACCGCAGCCCTCCC
CTTTTCTTCCTCACAGCCAACGAAATCTCCCGTCCCCAAATCGACCGAGCGAATTCACCACA
GCCTCACCGTCCCGAATCCGCACCACCGATG
SEQ ID NO: 22
Sequence Length: 3089
Sequence Type: DNA
Organism: Sorghum sp.
AGAGCTCTCTTGCCCATTTGAACACCTGAGAAACTTGTTGTGGAGCAAGAGAACAGCAAGAG
CCTAGAGAGGATTGAGATTTGAGTGATTTCTTGAGAGAATCCTTCTCTAGTAAGTTCCAAGA
GTCAAGTGTGCATCCACCACTCTCTAGAGCCTTGTTTTGGCCAAGTGAGAGTTCTTTGCTTG
TTACTCTTGGTGATCGCCATTTTCTAGACGGTTCGGTGGTGATTGGAGGCACGAAGACCGCC
CGGAGTTCTTGTGGGTGGCTCGTGTCAAGCTTGTGAGCGGTTTTGGGCGATTCACCGCGACA
GAGTGTCGAAGAATCAGCCCGTAGAGAGCACTTGGTCCTTGCGCGGACCAAGGGGGAGCAAG
GCCCTTGCGCGGGTGCTCCAACGAGGACTAGTGGAGAGTGGCGACTCTTCGATACCTCGGCA
AAACATCGCCGAGCACTTTCTTCCACTACTCCTTTACATTCTAGCATTTACTTTGTGTTTTT
ACATTCTTAGAATTGCCTTGCTAGAATAGGATTGGAACTAGGTTGCAAAACTTTTATCCGGT
AGCTCTCTAGGTCACACTAGGCACAAGGGGTTGAATTGGAGCTTATAGGTTGCTTAAATTTT
TAGAGAAGCCCAATTCACCCCCCTCTTAGGCATCTTGATCCTTTCAGGTAGATTTTCGAAGC
TTCAACCACCTGAACGATGCTTTCATGATCTTGATGAAAAAGAAAGTTAAGCTGAGGGAAAT
CAGAGACTTCAGACCGATAAGCCTCATCCATAGCTTTGGAAAACTTATCACGAAATGCATGA
CAGGAAGGCTAGCCCCTAAGCTAGACACGTTGGTACTATAGAACTAGAGCGCCTTCATCAAG
GGTAGATGCTTGCATGACAACTTCAGGGCAGTCTACCAAGCGTGCCATCAAGTTCACAAAAA
GAAGATCAGTTGCATCATTCTAAAAATTGATATCGCGAAATCCTTCGACTCGGTGTGCTGGA
CCTTTTTGTTAGACCTACTGCAACACATGGGCTTTGGTTTGCGTTGGAGGAACTGGATATCT
GCTATCCTAGCCACGACAAGCACCAAAATTCTGCTGAATGGAAACCCAGGAAGACAGATTTG
CCATGCACGTGGGCTCAGGCAGGGCGCCCCTATCTCCGATGCTATTTGTGTTGGTCATGGAG
GTCCTGAACCGCCTTCTTCTTGGCTGGAATCTAGAGATCTGCTCACGCCGATGACAGGGTTA
TCTTCCCCGCGGGCCAGTCTGTACGCTAATGATCTGGTTATGTTCGTCAGACTAGTTGACGG
TGATCTTCGGGCGGTAAGGGCGGCGCTGCAGATCTTTGGTCAGGCATCTGGGTTGATTGCAA
ACCTAGACAAGAGTGTTGCCACACCCCTTCACTGTTCATCAGAGGAAATCACGCGGGTTCAG
CAGCTTCTCTCCTATCGGATTGAGGAGTTCCCCACGCGCTACCTTGGAATCCCCTTGTCGGT
CTACAAGCTAAGGCGGTCTGAGGAACAACCTTTGATTGACAAGGTGGCAGCTAGGATCCCGG
AATGGAAAGGAAACTTACTCAATGAAGCCGACAGGACTGCTTTGGTCAAAGCCACACTCAGC
CATCCCAGTGCACACGTCGATTGCGATGTGTCTCTCCCCTTAGGCCTTAAACATGATTGACA
AGCTGAGGAAGGCATTCCTTTGGACAGGCTCCAATGCCGTAGCCGGTGGCCGGTGCAAGGTG
TCTTGGTCCAGAGTCTGCATGCCAAAGCACTTGGGTGGCTTGGGGGTTTCCGACTTGCGTCG
TGTTGGAATTGCTCTCAGGGTTCGTTGGGTTTGCGGTATTGAATGAAGATAATTTTTATATA
AAAATTATAGATTTCGATGAGATCTATATTTTTTTATTTTGTTTTTTTCCATTTGAAGTCAT
TAAGAGCAACTCCAACAATTTGCTAAAAGTACTTGACAACTTAGGATTTTTGCCAAAACCAT
AAAAAACAGTCTCCAACAAGTTGGCAAAACTACTTGGCAATTTTGTGAGCTTGGCAAAATTT
CCCCTTCACTTGGCAAATATGCCAAGTCCTCCATCACTTGCCATTATGTGTATCTCAATTTG
CCAACTAGTTTTGCCAACTTGTTGGAGGCTTAATTTTGTGATTTTGTCAAAAATCCTATGAT
GCCAAGTTCTTTTGCCAAGTCCAAATAACAAATTGTTGGAGAATGCTTCTTTTTTCACTTGG
CATTTGGTTTTGAGACTTGACAAAACTATAGATTTTCCAAGTGAGTTTTAGCAAACTATTGG
AGTTGCTCTAAGATACTAAAAAAAGTAACAACATATTTACAGGTATTTTTGACTTTTTACAC
TCGCAATTTGACACCATTAGAGCCTAATTAAACAGTAGTGGCTTGGAGGGCAAAAAAATATT
TGAAGCAATGACACTGAAGTCCGCTAAACTTTTCTAGTGACTCATAAGAAATTACAAATTTG
CTTGGCCTTATTTAGTTCCTAAAAAATTTTGCAAATTTTTCAGATTCTCTGTCACATCAAAT
CTTGCGACACATGCATGAAACATTAAATATAGATAAAATAAATAACTAATTGTAGAATTTAA
CTGTAATTTACGAGACAAATTTTTTGAGCCTAATTAGTCTATAATTAGACAATATTTGTTTA
AATACAAATAAAAGTGCTATAGTATTTATTTTGCAAAATTTTTTGAACTAAACAAGGCCTTA
AAAAAAGAAAGGGTCACAGCCTTGTTTCAAATTTCGTTTTGGCGCCGGCGTGCCGCGCGCCG
GCGACATATCCCTCCGTCTTCTGCCGTCCTCCTCTGCGCCACGTCAGGGATCCGCGTGAAAA
CCGCATCGCGACCGTCCGTGCCAAGAAGCACCAACGGCCCAGGCCGGTTGAAGCCAGCGATC
CGCGGCACCTGCCCCTCCACCAATCAGCGCTCACCTCTCCCGTCCTATAATAACACACCGCC
CCCAGCGTCCTCTCCCAACCAACAACAACAGCAAACACATCTCCTCGCTCGCATTTCTCCCC
AACCCAATCAATCCCCCTCGCCCCCGAACCCCAGCTCGCACCGCATCGATG
SEQ ID NO: 23
Sequence Length: 3094
Sequence Type: DNA
Organism: Sorghum sp.
TGCATCCTATTAACTTCACTTCTTTGATCTTCAGTCATTTGTGTACACGTCGTGCACTGTCT
CTTTGGCTTTTATATTCTTTGGGATCGATGACTTATGTAATCGATTTGGATTCACTTTGGTC
ACCTATTTTTTTAAGATGACAGAAATAAGAAGTTAACAATATTTCTATTATATATCAAAACA
TTTATATTGATGATACATATTTATACACATGTGAGCATCAGTATTTACCTTAGTCAATTAGG
TTTGTCCTGTCTTTAAATGTCGCGGAATAATCGTCCTTTTCAAAATCCATTAGCAGATATGT
GACTAAAAAATAGAAGAAGACAGTCATCAAAAATGATAATAAAATAAAAAAACTATAAATGT
CTATATAGTTAATAAGAGGGTGTTTGGTTGGGTGTGTTAAAGTTTAACATGTATTGTAGTAT
TTTATTTTATTTAGCAATTAGTGGCTTAAAAGATTCGTCTCACAAATTACTCTTTATCTGTG
GTTTTTAGTTTTGTAAATAGTCTATATTTAGTATCCCATGCATGTGTCCAAACATTTGATGT
GATAAGTATTAAAAAACAGACACAACCAAACAGATTCTAACAACCACTGTTACTAAATCTGA
ATTTTTATTTTATTGTTGGTGTTCATGTCCACAAGTTTATAAAGGCCTTGCTTAAATCTAAA
AAGTTTTTGAATTTTGACACTGTAGCATTTTCATTTTTATTTGACAAACATTGTCCAATTAT
GGAGTAACTAGGCTTAAAAGATTTGTCTCATGATTTACAGGCAAACTGTGCAATTAGTTTTT
GTTTTCATCTATATTTAATGCTTCATGCATGTGCCGCAAGATTTGATGTGACGAAGAATTTT
GAAAAGTTTTTGATTTTTTTGGATGAACTAAACAATAATCAAGATAAGTCTGTAAAATTTGC
ATCAAATATTTTCTCTCATATTGTATCTAAGGTACAATCTAATTACTCACGTATGGTACCCT
ATGCTAACAGGGTCACAAATATGGAAGGAAATCATGAACACTTAGGCCTTGTTTGGATCTAA
AAAGTTTTAGATTTTGACACTGTAGTACTTTCATTTTATTTGACAATTTTTGTCTAATTATA
GAGTAAATATGCTTAAAAGATTCGTCTCACGATTTACAGGCAAACTGTGCAATTAGTTTTTG
TTTTCATCTATGGGTGTGTTTGGTTCGTTTTCTATACAAGCCTACCTAGCAAAACTAAGCCA
AACTACCTTTAGTCAGTTCTAACTAGGCCAATTCGTAGTTGTTTGGTTGTGTACATTGTACT
AGCCTGGCTAGCATTGGGTGTGTTTGGTTGTCTATCTTGTTTTGCTCAAAATTACCTCTTCT
CTCTTCTAGTAAGGTTATCGCCTCTCACATATTTTATCAAACACCACCACAGCTAACTAGTC
ACCATCGGTGAAGAAGACTAGTAGCGAAATAGAACGGGAGTGAAGACCAGGAGGTGATGGGA
ATGAATCACGGAGCCAACCGGAGCTTAGCTCCAGAAGAAACACCAACCTGGTGTTTCTACTT
GGGCCTGGCCCTGCCTGTACGGCAAGCCAGATATAGCTTGGCTCCAGGGGCTAGCCAGGCCA
AACACCCCAAACCTGGCCCAATCAAGCAAAAAGCCAGATTTGGAGGCCAACCAAATACACTC
TATATTTAATACTTCATACATGTGTCGCAAGATTCGATGTGATGGAAAATTTTGAAAAGTTT
TTGATTTTTAGGATGAACTAAACAAGGCCTTAGCTTGGTTTAGATCTAAAATTTTTTTGTAT
TTTGAGACAGTAGCAATTTTATTTGTATTTGGTAATTATTGTCCAATCATAAACTAACTGGA
TTAAAAAGATTCGTCTCGTAAATTAAAGATAAATTGTGCAAGTAATTATTTTTTCATCTATA
TTTAATGCTCCATGCATGTGCTGAAAGATTCGATGTGATAAAAAATTTTAAAAATTTTTAGA
TTTTGGGTGAACTAAACAAGGTCTTAGGCCATGTTTAGTCGGTGAGGTGAAAATTTTCACGA
CAATGTATCACTTTCGTTTGTTTGTGGTAATTATTGTCCAACCATGGACTAACTAGACTCAA
AAGATTCGTCTCGTACATTTCGACCAAACCGTGCAATTAATTTTTATTTTTATCTACATTTA
ATACTTCATGCATGTGTCTAAAGATTCGATGTGACGGGGAATCTTGAAAAATTTTGGGTTTT
TGAGTGGAAGTAAACAAGGCCTTATTGATGAAAGAAGGGCGCACATAAACAGCCTGTTCGCT
TGAGTTTATCAGTCGAATATATCAGTTAGGGCACTCACAATTTAAGACTCTATCACAAAGTC
TAAGACAAATAATTACATATTATTTATGGTATTTTACTGATGTGGCAGCATATTTATTGAAG
AAAAAGGTAGAAAAAATAAGACTTCAAATCTTATTTAAACTCTAAGTCCATATTATTCGAGG
TAATAAATAACTTTAGACTCTATGATAGAGTCTGCATTGTGAATGCCCTTATTTAACTATAT
TTTTCTCTTATAATAAATCAGTCAACGATACTTTCTGTCATGATTTAGTCAAACGAACATCG
CAGAAGTACCGGTGCACTAAACCATCCCTTTTTAGGCGTAGAGATTTTATATTAAAAATAGG
CTCAATTAAATAGCCAAACTCTAATTTAAATTAATCTCCAAAATATCGGAAACAAACGGCAC
GGAACGGAAATTTTTCCGAACCGCTTGATCCAGCTTGAAACAGCACGCGCGGCGCGGACGCC
TCGCGCCCATCTATTTCGTTCCACGCATCTCTATCCCTACCCGTCGAAAATTCAACGCTCCA
ACTCTCCGCCGTCCATCCTCCCACGGCAGCCCAGATCCAACGCCTGTAGCTTGCGCCAACTC
ATCGATCCGCGCTCCACCCATCTCCACCAATCCCCTTCCATCGTGCTCCTCTACAAAAGCTC
CCCTCCCCATCAATCAATCCCCCATTTCACGCCAAGAAAAGCCTCCTCCTGAGTCTCGAACC
AACCGCATCGTCCCCCGTCTCCTTCCCCCTTCGTCCCCGACATCCCCGACCCGATG
SEQ ID NO: 24
Sequence Length: 3003
Sequence Type: DNA
Organism: Sorghum sp.
CTAGCCCATAACTTTTATATTTACATCAGTAGAAATTAGCCAATGTGCATGTTGCCTTCGCT
AATTTATTGTGTTTTCATAAATGCAGGTTGAAAGTATAGTTTTGAGCATCATTTCAATGCTT
TCTAGCCCGAACGACGAGTCTCCAGCGAATATTGAAGCTGCTGTAAGTGCACATGACATTAT
TCTCTCTTTTTTGTATAGGAAAACAAACTGCAGAAGTTCATTTGTGCTGCTTCGAGTGCAAC
GTTAAAAGTGGTCACTTATATTGGCTTTGCAATTGCATTAGTCAGTGTTGCTGATGGATAAA
TAGTTTAATACAGTATTGGGTCCTTTGAAATACACTACTTGAGTAATTATTTGCTTATTAAC
CCGTTAAATATTTATTGTGATCATTTCTCCTTGATTAGCACATAGCGCTTCTGCTATTGTCA
AGGTAGTTGACTTTATGCACTTCTGAAGTTTTCTAATCATTTAGTCTAAGCCACACCATATC
TGAAAACTTGCTGGCTTTTGGTAAAACACCGAAATGTTGTTATCAGATACCATTGGTTTCTG
AAGTTGTACGTGAATGTCTTGCAGAAGGATTGGAGAGAAAAGCGGGATGAGTTCAAGAAAAA
GGTAAGGCAATGTGTCCGCAGATCTCAGGAAATGCTCTGAAGGAGGAACATATGGGGAGTTG
AACGAGTGCTGCAACCGGTCTGCTGCAATTCACAGCCAATTACCTCGTGCCAGCATTCTTTT
GCTTTTCCCCTGTATATTTTCCGTTCAGTGTCATTCGTATGGTGGTGTTGGGTCTCCTTAGA
CAAACTCGGGACTGTTGCTTATCCTAAAAATTCGATTGTATTGTGTGGCATGAAATGATCGG
TGTCGAAGAATATTTTGAACATACTGCCACCTAATCATAATTTTCATGGAGAACATCATAAG
AAAGATGTGATGGCTGCCAAATGTGTCTTTAGCTCATTTCCTTGCTGAGTATTCAGATTCCC
TCTGCGTACCTGGGCAACTTAGGATTTTAATATTTGTACACTCCCTCGTTGAGTATTATTTA
GATTCTTTTGCTATATATTTAGACATGTTATGTCTAGATATATAATTGATTTGATGAGCTAA
GAAAAAGTCAAAGCGACTTATAATTCGGAACGATAGGAGTAGTTAATTTAAGTTCTGGATGG
CTCTGCACTGTGCTGGGCCTGGGCTACAGGTACCATTAGGATCTCAAATGATCAAGACTAAA
TCATGAATCAACACCATGGATCCTCCAATGATTAGCACTAGTATTAGGAACTATTTGAATCT
CCTTCTCTAAAGCTCTAAAAACTTTAAAGCACTTTAGCTCAGTTTCGAGATCTAAAGTTGTA
GCGTGAGATGGGCTAAAGTTTAGAGCTATCTTTTGACCTCCTATCTTTAACTCAAGTTTAAA
GCTCTAATTTAGAGATGAGGATCCAAACAGGCACGCACTTGCATACATGTTGAGCCCATGGA
ATAGTGTCTAATTACTTACATGCACGACATGTCTAAAGTAATACTATTTCAGGCATTTTGCT
CCTGCGGTTGTCTTCGTAACCTTGCTTTTGTTACTGTCTACTGCATATATATAATTTTGTAC
CTTCAATATATATATCCTTTTGATTTTTACATATAGTGATGGAATGTTCAATTAGATTTATA
ATAATATAAATTTTCGCAGCAACACGAGGTGTCATCTAGTTCATCAGAAAAAAGACATCTAA
ATTTTACATCTTCTATACTAGTATAGATGGATATTCAACTATTCTTTTTTTCCCTCAACTTC
AACTCTTGTAGTGTTCGAACCCACAGGTAAATTACTTCGGTGGACTAGAGTTTAAAGCTCTA
AAGTTTAGAGAAGGGGATCCTAAAGTCTCCTGAGTCGAATGGATTAAAATTATGTTTGTATC
CCTACATCTAAACTTTAGAGCTCTAGAAATTTTAGAGCACTTTAGATTAGTTTTAAGATCTA
AAGCTCTAATATAAGGTGGACTAAAGTTTAGAACTAATTTTAGACCACGAGTTTGAAGCTTT
AGCTAAGTTTAGAGAAGAGGATCCAAACAACCCAATTCAGTTTGAGAGCAAGAGAAGTCTTG
TAAACCAAAAACGCAACCTCAAATGTAGAAGTATGTTTTCTTTGATTTCTATGTCTAAGATA
TTTGTGCCTTTATGTATATTTGAGAACTATCTTCATCCATGAAATAATGTAATTTTAGATTT
TTGAGGAGTCAAATATTGTGAATTTTAAATAAAGGTATATAAAAATACTAACATATATAAAT
ACTAATAACATTCATAATACAAATGTTAACGTTATTTATTGTAAATTTGATCAAATTTGAAC
AATTTTTTTATAGCAGCCGACACAAAATGAGGCCTTGTTTAGTTTCAAAAAAATTTGAGAAA
TCGACACTGTAGCACTTTCGTTTGTATTTGACAAAAATTGTTCAATTATGGACTAACTAGAC
TCAAAAGATTCGTCTCGTAAATTCCGACCAAACTGTGTAATTAATTTTTATTTTCGTCTATA
TTTAATACTCCATGCATGCATCTAAAAATTCGATTTGACGGAGAATCTAAAAAATTTTACAA
ATTTTTTTGGGAACTACAAGGCCTGATATAACTTGATGTCCGGCAATCCCCCCGCGTGAGGC
CTTGTTTGGATGTTGTCGGATTCACCTCAATCCACGTGTGTTGAAGTGGATTGGGGTGAACC
CACCCCGATACATGTGGATTAATGTGAATTCGACTACATTCAAACAAGCCCTGACACAATTC
TCACCGCCAGTCTGCAGAAAGCTTGAGACTGAGATCATGCCATGTCACCGATCCGCGCCCCC
CTCCTGCTGACCAATCGCGGCCCGCCGTGCACCTCTTTAAATTTCAAGCACGCTCCTTATTC
GCGTCTCACTACGCAACCGCCCGAACGACTCTTCCAGTCTCCTCGCGAGTTTCCTTCAACTT
CCCGCCATCTTCGATCCGTGCGTAATG
SEQ ID NO: 25
Sequence Length: 3045
Sequence Type: DNA
Organism: Sorghum sp.
TGAACTCTGTCATCAAAACATAACGAGGTGTGCTGGGCTGGGCAACTCTGACGAACGACAGG
TGAAGGCGATCCGGATCGAGCCGGGCCTGCGCGTGGGCAGCAGCACCAAGGGTGGTATCATC
GACTCGGACGGGGAGGTGCTTGCGAGGGGCGATGATGGGTCCCACTCCCGCGCCGGCGACGA
GCCGGGGCACCTGATGGCGTACGGCCCGCCCATCCAGCTGACGGTGGACCAGGGGCTGGCCA
CCATCTTCTCCCCGAGATGACGATTCTTCTCCCTGTTCTCCACTACACTACTGTCTGCGAGT
AATTTCCTTCGTTTGTAGAATCATGTGCTTCTACTGTATATGTAAATATACTCACACCCCAC
TCCAGTGGATGAGCAATGAGCCGAGAGGGACGGATCCTGGATCTCCGTCGATAAATTGTTTT
CTTTTCACGGCCCCAGCCCACATGGCTGCCGCTTTTAGTGCTGGCGCGAGAAATGAGAAGTG
GGCAGTTCATAATCTTTTTTTTTATTGTTGGCCGTGTTGTTGATTGTCACTTGTACAAACAG
TTGGACTGTTTTCCTGCAAACTTGTGCTTCCATCTAAAAAGACCTTGACACTCTAGTTTAAG
TCGAACTATCTTAATTAATTTTGATCAAGTCTATATAAAGAACCAATGTTTATATCGAGAAA
TAAGTATCACTAAATTTATCATTAAACATATTCTTAGAAATACCTATTTAATGTCATGCGTA
TTGACAATCTTCTATAAATTTGGTCAAATGGTAAATAACTTTGATGACTTGAAATGATTTTT
TTAAAAAAGGGATACTACTACCTCCGCCCATAATTTTTTTGGACTAACTTGGAAAATATTAT
TAATAATTGTATATGTAATGATACATATTGCACGTCATAAATAGTAGTAAATTTTTAATTAT
ATCTTCTCGCCTTCTGAGTGTTGGTTGTCTATCTGGCTATCTATGTTGTAGTGTGCTATAGT
TGGAGTTCGTGGCAGAGGATGAGACCATCAACATCGTTTCCAACCTAAACGCCTTCGACATG
ATCAGCATGCATATGCTGATCAACACTAATTCCTATCTCTTTCAACTCTCGATGCCCCCACC
TATTATTAGCCTCATACCGCTACCACCGTCTTTTGTATAAGCTTTGTCTATGCACACTTATA
TCCAATTCAATCGATCTGTGTTGTGGTGAACTGGGGTACAACTAAACTAAGGGCGTGAGCTA
CGGAAAAACTGTTACGAGCTGTGTGATGTAAAAATGTTGTAAGCTATTTGGTTGAAACAATT
ACAAAACCTACCCACTATCTTTATTTATCTTGAAATAACTATAAAGCATCCTGTATTTTTCA
CTCATTTGTGAAAATTAAAAACTGAAAGCCAAAATCAGCACCCAACTAAGTTAAGAAAATTG
TACTACTCGAAAGGTGAGTATGTTTCTAGAAAATCAGCTTCAGATTCCACCTATTTTGTTGG
TGTTCTGTTTTTAGAGGTAGAAATATTTTTCAAAAGTTGGACTAAACACAACATAAATTCTA
CTCTAGTCTCTTCCAAAACATATGAAATGATTTAAATCTTATACGAGTTTCCAACGAGCCCG
TACGGGGATTTTGGACCCTTCTTCCGACGACAGATTCCCCACTACCGTTTGGCTCGCCGTCG
CGATCAAGAAGCCTAGCAGTGCACCGTCCTTCACATATTTTTTTTTCTTTTGATTTCAAAAA
AGGAAGCGCCGTTTCCCGAACGAAGAAAAAGATAAGGTATGGAACGAGTGACCGCGCGAGGC
AGCGCGGGTGGAGTGGGCCCCAGGGCAGGGTAGCCGCCAAGGCAGGGCCGTCCGTAGGCGCT
ACGAAAGCTGGAGGAGTTCCTGTTCGCATGATGACAATCGCACGGCCACGGCAAACCCTAGC
CGCCGGGCAGGTCGGTCCCCGCGCGGGGGGCGGCGGCGCGGGGGGCGCGCTATATAAACAGA
GCCCTTCATCCGATGCCTCCAACCCATCTGGCGACCTCGATCCCCTCCCCTGTTGGTTCTGT
CTGTTGACTTCCCCCCATCGAGGTAAAGTACTCGCTCGATTCCTCTTCCGTCCTCCGATCCG
GGCGGGGTGCTTGATTTGTTATCATTCACGGTTCTGATTCAATTGTTTCTATCAAGTTTTGT
CCGAATTCTTTGATGCTCGATTCATTATTAGTCTTCAAATTTCTCTGAATTGTTCCCTAGCT
TTTATCCTCCACGCATATGTACTAGTATACTAGCAGAATTGTTCCATAGCTTTTGTCCTCCA
TGCATATACTAGTAGTACCCAAAATCTTGTGCTGGCCGATCGCTCTTGTCCCGCAGCAATCA
ATCGTTTTTCTTTCTTTTACTTTTCTGATAATAAAGCAGATAGATCAAATCAAATAGTTATG
ATACACATAATATATATCATGGCATCATCCACACTTGATTAAATCCAAAACTGGTATACACA
TAATATATATCATGGCATCATCCACACTTGATTAAATCCAAAACTGGTACTGGAGATGCGAC
TAGTGTGCCCATTGTCTAATGGAAAAGACAGAGGGTCTCGTCTCCTATCTCATCGGAAGGGG
CCGGGCCTTCTGATATAGGTTCGAATCCTATTGGGTGCTTCTATTCTAGTTTCTGCATCTCC
AGTTTAATTTGATCCACTGCCAGGTCAGATTGCCACCACTCACTCACATAACCTGCTTATAT
CTGTTACTGTTTTTGTTGCTGTATGTTTCTTTATAGTATATTCATTTGGCAATTGTATTGAA
TAATCAGGTCGGTTGCTATGAATTACTATGGATGAATACTGACTTCAGGGTCTCTGTTTTGT
CTCTGTTTTCCTGATCACTTTATTCTAAATAAAAGAACAATTAATCTAGCAGTCTGCTTATG
TATATATGCTTCTAATTTACTGCTAAAAAATCAATCTATCAACTAGTATTTTTGTGTGACTG
CGTTCTCTATGTATCCTTCTGCTGATGTTTGTGAATACAGCTAGCTAGTCAGCTGGTCCCGT
TGCCATG
SEQ ID NO: 26
Sequence Length: 3248
Sequence Type: DNA
Organism: Sorghum sp.
AGTCTCCTCTCTCTAGTTCCTCCATGCACCGCTTTAGTGTGACCACGTGCTGCCAAAAAAAC
ACAAAGAAACTGCTCCTTCACACACGTAACGGAATAGGACACATCGTCACGTGGTCGCCGCT
GATTTGTTGTATCTAGACACAATGATATATTTAAGGTCATGTTTGACAAGGTTTTTGTGCTC
GGAACATTAAAGGGTCGGTTTAGTTACTTGGAATGGACCCATGAACCATTCTAGATTTTAGA
CTATCAAGATTGAATAAATTAGTAAATTATTTCATCTGAGAATCATTACTCAATCCAAAGGA
ACTGAACAAGCCTTAAGGAGAAACTAGTTTTTTTACATGGCTCCTCTATCATTCATTAGAAA
ATGGTTTTTCCTCCTAAGATGTTTGGTAGGGCTCATCCAGCTCCTCTACTGAAGCTGCTTAT
ACAGTAAAAGCTATGTATTTAGAAAACTTAAAACGTCTTCTAATTTAGAAGGAAGAGAGTAT
CAAGCAATATTATATGAGCAAAAGGATACTTAATGGGAGAACAAGACCAGACCAAATACTAG
CAGCACTACGAGTGCAACAAACTCCTACATGACAAGGAGCTGAACTCTCCATGGCAAGTGAG
GTCTGTCAACCCTCTTTATGGCTGCGCCATCCCTCTTCACCATCGTTGTTCCACATGTTGTT
GAACACTAGATGAGGAGGGATGCATCATACATGCCCTAATGTTGCTACACAACACTTCCACC
TCTACGTCTTGCTATCATATCACTTTCCATATCTCTTGTCATCTTCGCCACTACATGCTGCT
CCTCTTCGCGATCCACAACCATGGGGGTAAGTTGGAGGAGATTTGTGCCGACCTGCTGCTCC
ATGGCATGAGCATCGGCGCGTGTCGCGTGGAGGCTGAACAACATCTGGTGTTGTGCATCAAG
CTAGGAAGAGAAAGAATTCACTCGTGAAGCAGTGCCAAACGTTTAGATCAAGGTGCATAGAG
GAGTTACCTAATTCTGGGTCAGATCTAGAGTAGGAGGAGCTGCATTAGGAGCATTACCAAAC
TAACCCTAAGAATTCTTCTCTTAAATCTACTAGTTTTGATCTTATTCTCATCTTATAATTTT
TAGAAAATATAAATAAGACTTTGTTTTCGTGGGGAAAAAATCAGTGGAAATGGTCTTCATGG
GGCTGAGCACCTACCGTAGACTTATGACACATACAATGTAAAATCATTCGATGCATTAAAAA
AGTAGAGTTTTTTGTTTCAACTATTGCTCCTAAATTTATGCAGCAACCACATGCTTTGGATG
ATTTTATATAGAGACGTGTCACCTATCCGCAATAGGTGCTTATGGTTGTCCATGAGAACTGT
CCCTACTAAATTTCTTTATAAATCAACTTTTTTAAGATGGTGAGAGTACAATATTTTAACCG
GCATGGGGTGAGTCATTTTTCCCTTTTGAGTAGCATGGGTGAGTCTAATTTAAAACAACTTA
AATTTTGTAACTTATAGAACACTCCTTTGCCTATTTGGTCAGCTGTGTTGTTACTTTTAAAT
CACTCAATCATATTGCTATTTCAAGCTATGCTGTTATAGCTTTAGGTGGACCGTCTAAAAAA
GGTGTAAAAGGTGAAGAAATAAATTAGTGATGACTCGAAGGTTACGAAGCTGTAAATCTAAT
CATTGCCGTATATCCTCGCAATAGAACTATAAAAGTATTGAAATTGGGCCTGTTTAGATTTA
AAATTTTTTTACCTAAAGAGAAAATTTTTTGTGGAATTGGGGTCTAAGAACTAAACGGGGCT
AAAAAATTTTGGGGCCAAAATTTTGGGCTGCAATTGTGCACGCACTCCTATAAAAACCCACC
AATGACAACTTGAGCTGCATGTTGTTATTGGTAGGCTTCCATAGGAGTGCGTGCACAGTTAC
AGTCCAAATTTTGGTCCAAAATTTTTTGGTCTCATTTAGTTCTCAGGCTCCAATTCCATAAA
AATTTTTTTCTTTGGATAAAAAAAATTCAAATCTAAACAGGCCCGGTAATTTGTAAAAACAA
CGCCACACCCCTATCTACCTAACAGGAACCTCAGAATGAGCCACGACGTTCAAAATTCTGGT
TTAACAGAGACATATAGGCCTTGTTTAGGTCCTAAAAAAATTTGCAAAATTTTTCAGATTCC
CCGTCACATCGAATCTTTAGACACATGTATGAAGTATTAAATATAGACAAAAATAAAAACTA
ATTACACAGTTTGGTCGAAATTGACGAGACGAATCTTTTGAGCCTAGTTAGTCCATAATTGG
ACAATATTTGTCAAATACAAACGAAAAAGCTACAGTGTCAATTTTGCAAAATATTTTGGAAC
TAAACAACTTTCCAAAATTTTTGCAAAATTGCTACAGTATCTCTTTCGTTTGTATGTGACAA
ATATTGTCCAATCATAGACTAACTGGGCTCAAAAGATTCGTCTCGTAAATTTCGACCAAACT
GTATAATTAGTTTTTATTTCCGTCTATATTTAATACTTTATGCATGTGTCTAAAGATTTGAT
GTGATGAGAAATCTTGAAAAATTTTAGATTTTAGGGTAGAAGTAAACAAGACCATAGCCTTA
AGTACTGTACAAGGCTAGTTACGCTTACGGTACATTCAGACGAGCTTTAAGCTGGGATATTG
GGGAGGGACGGCATGGTACTGCACTGACCAGTGACCACCAGCCTTAACTTGCAAGCAAAACC
AAAGCCTTTTTGTTGCTCCATATAAAGTTTAACTCCTGTCACATCGAATATTTAGATATATA
CATATAGTATTAAATATAAACTATTTATAAAACTAAAACACAGCTAAAGAGTAATTTGTAAG
ATAAATCTTTTAAATTTAATTAATTTATAATTAGACATTAATTATTAAATAAAAAAATACAA
TAGCAGCTGTTGAACTTTAACGACCCGACAAAGACCCCTCGAGGAAACACAGTGAAGCGGAG
AAGTCACAGGCTCACAGCACTGCAGGGAAAGCTGCTTGAGCCTTAGCGCTGCGCAGAACTCC
GGCCGGCACGAGGAGGACGAGAGGAGGGATAAGGAGAGGCCAAGAAAATCCAGGACAAAAGC
CCTCGAGCTCACGTCGATCCGCCTCCCGCATTGCCTTGAAGCCTCTCGATTCGGGCGGACCC
GAGCTGCCCCGCGGAGGGCTCGATCTGGGCCCAGATCGAGCAGCATTTCGGTCCTGTGCATG
CTCAAGCCCCGGCGGTGAGGCATG
SEQ ID NO: 27
Sequence Length: 3523
Sequence Type: DNA
Organism: Sorghum sp.
CTCGCTGGCACGGGTCAAGACTTTAACATATCTAAACTATATTTGAACATAAAACGAACTTG
TATCACATGATGTGTGAGCTCAATCGTTCTTGAACATGCTGATAATCGAGAGCGTGACATTT
CGAAATGTTTAAATCAACAACCTCCATAAACGGTAACTTATATTGTGTCACCTTGCTAGCTA
GTTTCACTCTATATAGACGTGAAACAAAATGGAGTTTTAGCTGCGGATCGAATTTATTCTGT
GCAAAGCTACTTTCACAAAGAACGATCTAAATTTAAGCTACAGGTTTGTACTTGCCAACACT
TAGCGTACGTACCCTAAGTTGTTCCTCGATTCCTGGGGCTGTCATCGCTCTTGTTAGTCTTT
ATAACTTTATTGATTCTTGATCATGATGATCCTAACCAGTGAGGTTCTGTTTGGCAGCTTTG
TAGCTTTATTCATATATGCACAAGAATATAATTGTAATACGGTGGTAAATTTAGGCTCTGTT
TAGCAGGGCTTCTTCAGCGGCTTAAGGAGCTGTTTGGAGTTATTTTCTACCAAACAGAAGTA
AACTGAAATGACTCCACCGATGAAGCTCCTTAAAAACATGATCTAAGAGCTTTTGCGGTGCA
AAGGTGCCAAAAATAGTGGCTTCTCTCGGCTTCACCTCATCCTAAGTGGTGTTCTGTGAGAG
TATTTTAAGGAATGAGCTGTTTTGTCGAACGATTTACCAAAATGGCTCTAACTGTTTATGGA
GCTTAAGCCTCTAAAAATAGCTTTACTAGTGAAGTGGAGCCGTGTCGAACGGGTCTTAGCTT
GTTCTTCATAAGGCATGCACTATAATTTACATTGGTATTTGGTAAGTACGAAACCGTGCTTG
CAAGTTGCAAAGAGGATGTGATGTGAAGCCAGACGTTGTCGGTGACGGTGCTGACTGCTGAC
GGGCCGGGCTCCACGGAAACAAACTCGCTACTCGCCGCACCGGACGTACGTACAGGTCGGCA
GCTTGCTCGGCCCCGGCCGCGCGCGTCTCCGTGTCCTCCGCGACTGTGCACGTTTCGTCGGG
AGCGGCGTGCCCACGCCCACCCCCCGTCCACCAGCCAGCAACCGACGGCACTGGTGACACGC
GGCTGGTCCGCTCGGTCCGCCCCGCGGCTCCAGATCACGGCAAGCGCGCCCGCCGCCCGCTG
CTGCGCTGCGCTGCACGTCCCGCCCTGACGCCACGCCACGCCAAGCGCGACACGACACGACA
CGACACGACCCGACCCCCGCCAACGAAACGCCGAAACGCGGCAACGCGTGACGGGCGCGCAT
GGTCGATGCTCTACCCGCGCGTCCGCCCCACGCCAATCTCCCGGCGGGTCCCTCGTGGGACG
GGGAACGCGATGCGGCTGCAGGCTGCGACCGCGACCGCGACCGCGACCGCGCCCACGTGAAG
GCAGGCAGGCAGCCCCGGAGCGGGCGCGGCGGTGGGCCAACGACGCGTTGCCGTCGCGAATC
TTCTTCTGGCCACGGCCAAGGGCCAATCGCCCGCTCCGCTCCGCTCCGCACTCCGCCTCCGC
TAGGGAATATGGAACCCGATCCCACGGCCCTCTGGGTCTGGTCGACGGGTCCTCTCGCCGTG
GCAGCTGCTTCCCGGACCGGAGGATCGCTGAGCGCGGACGCCACTGCCATTGCCGTCCGACT
ATAGTTGTTAATTACCATAAAATAATTTGTTAACGATAAAACCCGTGTCAGGCACCGTCGTC
TGGACGCTGCTATGGGATAACCATTCGCGTACGTCGGTTGTATGGGTGGGATCCTCTGCGGC
ACGCCATTCTGGTGCTGCTAGTGGAATAGACAAAAAAAGGGCCGACGGTGTTTGCTCGTGGC
AGGCCACACAGAGTGACAACCAGAGTGGTTGCCGCAAAAACAACCAATCACACAAAAAGTGT
TGTACCGGTGGAGGACAGCCATTAATCAGCAGGCCGGCTTCGCGGCCAAAAGAAACGGAGAA
GAGGAAAAAGGGGGGCAAGCAAAGAAGAAACCACGGACGGAGCGAGCTCCGAGCGTCCTCAT
CCTCCCGTCTATAAATTCCCTTCCTTTTCTTCCTCCATATATAGGGGGCGCCATCCAAGCCA
AGAAGAGGGAAGAGCACCAAGGACTTCCCGGCGCCCGTTCAGGATCCACATCCTTCCCGAGC
GAGTTCTTGGTTGACCTCTTCCTCTTCGACCACCTCCTAGGGTATGCATGCACTGCACCCCC
GTTCCCCCTTTCTCCGTTTCCCTTTTCTCTGAAGAAGAAATCTGTGATTATTGTGTCCTGGT
TTACGAGATTAGTTGTTTTGCTGAGTATGTGCTAGGCTACTGCGCTGAATTTGTGTGTCGAT
CTTGCTTTTTTCTTTTAATCAAGGTCAGCCCTGTCAATGAACAAAAGGTCGTTATTCCCCCC
CCAGAAGTTTGCGATCATGCTTGATTTTTTGTTAGATATCGTTTTTCTTGTCTGGATCTTAG
TATGACTGTTGTTCGTGAGGCTGTTAAGTAATCGTAATCAGACTGGGTACGGTTTGCTGGCC
CTGAATTCCAACAGTCAGCTTGCTCTGGTTTCAGAGGATTTATGTTCGGCAAAATTTTGATC
ATGGCTGTACAGAAAGAATTAATCTTGATGGAAATAATTTAGATGAAATCCTTCATGACATG
AAAGCATGTCATCTTATGCCCCCTTGCTTTGCTTATTCTACAGTTATGTGAAGCCAAAGATA
GTGACCTAAGCAGATCAGTACTACCAAGGATGCATTTTTTAGCTGTTCGTACTTTGTAGTAT
AAAGACCAAGGGGTGCTCATTAGGTTTGTATGTATGTTAGAGCATAGGAATTGAAAGGGTTG
GACTTGATGCTTCTAGTCAGCTCAATTTCTGTTTTGGACTTGGGGTGGTTGATTTGATTATA
ACTAACTCCTTATTTCTAATGTGGATGGCATCCATCTATCCCTGGCATCTTCTCTACAATTG
AGATGCCTTCATTTAAAGCTTAGGACCACTTAATTGAAAGTTATCTGGACTGTAGGAATAAA
TAGTTGCTGAGAGGAATGATGAGGTAAATTACAATGGGCCCATGTCATAGACACTTGCACAG
ACAATGATATAAAGTCATTGAGATCTTATAGAGATGGTCACATGGTGGGGTTTGTGACTGAA
TTCTTCAACATAACCCATTTCTGCTAGCTTTATTTTTTACCCCTTAGTTTTAGGAAAAGCTA
TATCATATAGTCTAGATATGCTTGGCTCGGTACAAATTGCTCCTAGATTCTTGTATGGAAGA
ATGTCGTCAGCTGTGTTCAGTATTGACCTCTACTCTCTATTGTTTCATGGTGTGCACCCCTA
TGACTCTAGTAGAAACTTAGCCTGTGTTTTTAGTAGTGTTTGATCACAGAAAATGTAGATGT
TTGAAAGTGTGTTGTGGTTGCCTTTGTCTGCACTGAATTTTCTCTAATATCTATATCATTCC
TTTGTACAGCTCTTGGTGTAGCTTGCCACTCTCACCAATCAAGTTTTCATG
SEQ ID NO: 28
Sequence Length: 4012
Sequence Type: DNA
Organism: Sorghum sp.
GCCACCACAGAACGTACCAAGGATGTCACTCTTTCGTGGTGAAGAACAAAGAGATTTACAAG
CTAAGCCACCAAAAGGTAACTACTGTGCTTGCGTGATAAATAACCGGCTAGTTTTCATGCAA
ACTAGCAAAGAAACCATCAAAGCTCTCACTCGAGAGGGATCCCGTCAAGGCAAGGACATTGC
CGGGTCGCCCTAGGTCAGCCGGCTTAGAGCGCCATCCTTGGTTGTGGATCAAGGGATGAACA
ACATGGAGATTTGAGAAGAGAGTAAAAGGGTTTGAGTAGATTGGTTTGTCTATTGATTGGAT
AGTAGGAACTCAATTGGCCATGATCCATTTGTGTATATAGAGGGGTTGGTTTTATCCCAGTA
GAAATTTTTGGACTGAAAACTAGAGGACTCGGCTTAGCCGACTGGAGGACTCTGTTAGAAAT
TTCGGATGAAAAGCTTCCGAAATTCATAATTAATTCATCCGAACTCCAAGCAAGACGATCTA
TATATGTTTTTCGATCAGCTCAACGAGAAAAACACAATAGTGAAGTATATTCTTGCATTTGA
AAAAGTTAGACAAGTCAGCTTAGCCGATATAAGAGTTGTCTAAGGCGGCTTTAGCTGGTTCA
ACATCTGAAAGGTCATTTTCTACACTTAATGTGGTGATCCAAATATGCTTTCTGACCATCTT
TACGACAGATGTTCATTATGACGTGATGTCTATTACACACTCTAGATAATTTTGAGTGTCAA
CAATAGGTCTCACATATATGTACTCTATTGGAGGTCATATGTACTATGTTGAGCGTACACTC
ATCTTCCACTCGACCGTTATTCATCTTCCCTAAAAAAAATCAACCATTATTCATCTCATCTC
AGGCACATAAGGACACAGAGAGAAGATATTATAGCCATTATATCAAACCTGACCACTTGTGT
TAGCGAATGATTTGTCACCAATAGTAATTTTCTCGAGAGGAAGCTGCGGCCATTATATATAT
CGAACGCGTTAGGAGCTCTGCGGCAAGTTTCGGTTGGGGGCCTCTCGTTGTATGTCTAGAAT
GTGAAGAGTCTTTTTAGTTTTTTATGCCCTACTTTACAGTTTGTCAAACCTGAAAGCTGTTT
GGGCTACTTGCGCATGTAATTCTCCCTCTCCTTAATAATATATGACAGCTATGTTTCAGATC
TTTTAAAAAAAAGTTTCGGTTGGGTACGAGGCGAGCAGTTCTGTTGGGACCTTTCTTCATTT
CCTAGTCAATTGAAAATTTACATTTCGTTCTAATTTCTAATTGCATTTTTATCTGCTGACAC
ATACTCTATGCCTCGCTAGAGAAGTGATACCAACAAGATCCAGACTACAACTGTTCATAGGC
CATTCATTTGTTTGTAATAATTATTGTCTAACCATAGACTAACTAGGCTCAAAAGATTCGTC
TCGTAAATTTCGATCAAAATGTGCAATTAGTTTTTATTTTCATCTATATTTAGTACTCATGC
ATGCGTTTAAAGATTCGATGTAACGGAAAATCTTAAAATTTTTAGATTTTGGGATGGAAGTA
AACAAAGCTCTAAGATAATAGAAAAAGCCCGCACCCCCACCCAAAAAAAATATCAACCCATC
ATAACATCGGGTCAGATTCAAACCAAAAATTTGAGATTTTTTTTGGAGAAACTACCAAAAAT
TCGAGATGTTCATGAGTTCATGGGAGCCCAGTTTTTGTCGGGCCGTATTTGTTGGTTGGGAC
TTTTTTTCGGCATTGCAATTTGGGTAGAGCCAGCACAAGTTTCATAGGCACCAGCCCACCAA
GATCACTAGTGGGCCTAAAACTACAGTACTTGAGAACCCTCAATTGATTCCCACTTAATTTC
ACCTAAGCCCACAAGGGGAATCGAGTGGGCCGAATCCTGATCCTATTGTCGGTTCATCCAAG
CAGGCAAGCGCACGCCTCCTCCTCCTGCTATAACCAACCGGGCGGTCGACCAGCGGGAGGAC
CCAGAAACAGAGAGCGCGCGGTTCTACGTCCGAGTCGTCGCATCGCCCTGTTCCCTCGATTC
GCCGGCGGCGCCACCTACCAAGGTGATGCCTCCTCCCTCCTCCCTTCTCTTCCCCGATCAAT
TCCGTGTTTCCGAGCTTAGAATTTGGAGGACCTGATGATGAGCTCCTCCATCTCTTGATTGA
TCTGTGGGCGGTCGGATTCTGCGGTCGTAATCTCGCCCCAAATCGAGCAGATCTCGGGGCTG
TTTCGAGGAATATAATCGAGCAGGTCTCGGGGCTGTTTCGGGGAATATGTGCGTTGGATTGT
TAAGGTGGAGAATGTTTGCTCCGATCGGGTTTTGGTGGTGTCGGGGAGGGTGGCGATGGGTT
GGCGCCTTGGCGGATCGATTTTGGGGAACCTCCCTCAAATCAAGCACCACCACCTGGGTTTG
TGATTCGATACAGTTTCATGATTTGGTTACCGTGTTTTGGTATCCTTGATTCCTCGTTCCAT
CTAGACGTATGTATATGATAAGGTGTATCTTCTGACTCGTAGAACGATCGACACCACAACTA
AATTGTATTGCAATTTAGGAACTCCTATGCAGATTTTACATGTAGAGTTGCATATCGAATGG
TGGGTATCTGTAAATGATATCTTCACCTGCTGAAATAACTGAGAATTCCTGGGAGTTGAAAC
CTGTTGTTAATAAGCAGAGAACACAGTTTTGGTTATGGTTATCTGTAGCTATCATATAAGAG
GCAAGTTGCGTGTATGGTTAAGTCACCTCGGGCTACATTATTTGTGACTCGAGGCTAGCCCC
ATTTCCATTACTCATACAACGGAAAGAGCAGCCCCATTTCCATTACTCGCAGAACAGAAATA
CAGATGTTTTTTACTAGAACAGCATTCTAGATAAGGGAAACAAGACGATGACATGCTATCAG
CCTCCACTAAAGTGTTACTCTGCTTTGGACCACCACTATAGCAGGGAGATAGCAAGCTAGCA
ACTAGTCAATCCAGAACGTCACCTCAGGCTACTGTGTTGGTAAAAGTTGTACTTCAGTTCTG
TCATGTGCCTTTCTAGCCTATTCTGACCAACAAAAGAGGAAAATTTTATTGGATCTTTTGAA
CCCTTATCCTAGAATATTTCATTCAAATAACTCTGAACTGTGTGGTTTATTTCCCTGTCTTT
GTTCTGATCTGCCCTTGGTTATCATCCCAATAGCACCTCCATCAGTTAGGTATGGAAAACAT
CGTTTGGCTTCAGTGTCCATTCAGCCTATTTATTTCTTACCTCATGATAGGTCCACAAACTT
TAAAAATACATTTCTAGGTCCCTAAACTTGTTAAGTGATGCTCCATGCCAGGTTGCCAGCCA
CGTGATCATTTTCTGCTGATGTGGCATGCTGGAGTGGCATGTATTTATTTATTTCTGACCCT
CGCATTTATTTTTCCCTTTGAAAATAGATCATCCCTTCTCCTTCGGCTCTTTCATGCTCCTC
CCTTTCCTGCTGCTGCAAAAGGTCGCATGGCTCCACGAGTTGCATGTCGCAGCCCATTGTCT
ATTTTCAATAGAAAAATAAATTTGAAGTGTTATTAAATATAAAAATATATGCCACGCTAGCA
TGTCGCATCAACAAGAAATGTCCAGGTGGCTGCTATGGTCCTGTGGTGTACCACTTAACAAG
TTTAGGGACCCAGAACAAACTTAATGAACCTATATGACACAACCTTAAGTTTAAGGGCAGCT
GGAGCATTTAACTTTATAGGTATTATCTTGTTAGATTTGTCTTCTTGTGTATGGAGTATTTT
AGTCAATATGAGATTTTGCATTTTGTCGTGAATTGCTGTTCCTGTATCACCCTGGATATTGG
ATGATTGAGTTGAGTTGTACATTTAATTTAAGTTCTTTTATTCCTTTATGACTGCATACAGT
GATTGATTGGAATGGTATTATGGTTTGCAGCTCATACACCCAGATTGACTAGTCAAACCCAG
TGATCTCTTTGGGGACTAATCAAACTCAAGAACTAAGTTTCATG
SEQ ID NO: 29
Sequence Length: 2740
Sequence Type: DNA
Organism: Sorghum sp.
TTGGGAAGAGGATGCTGAGTGAATAAAAACGACATGCATATGCATGTTTCAGATGAAAAACA
TCATCGTTTCCAAGGATAGAAGTCCAGTATCTTCATCTCATGCATGTTTAGATGGAGAGTAA
CTTTTTACCAAGGCCAGTAGAAACATACACCTTCGTTACTCGTTAGTGGTGTACTGGTGTTC
TAAGATCAGCGCCAACGCACAGTGGCGGACCCAGGAATTGGGAGCAAGGTATGCCTATGGTA
AAAAAAATTTGGATGAACAACACAAAATATTTAGATCTGCTTAGATAAAAGATACATATAGT
TCAAATGCATTGCATATCTGTAAATTTTATTTTGCAATTGAAAATGACATTTAATAGAACCA
ACAAAGGGAAAAGGAAAGGGATTTAAGTTTTATTACTCCAAGCAAAGGGGGCGTCGTGGCCG
ATTGGCCGCGCGGGGACGCCCGATGCCGGGGAGCGTAGCCGCCGAAGCGGCAGCGCGGGGAC
GCCGGCAAATCAAGCATCCGCACAGGACGCAGCGCCTACCTGCGCGTCTGTGGCGAATCACG
AGCGGCGGCGCGCGGTGAAAGCGGCGGCGAGCGGTTGAGAGTCGCGGAAGTGCCTGCCCGCG
CGTCAGTGCGCGGCTGCCCTACCCCTACGGACTAGGCCTGATGGTTTTAGGATTTTGGGGGA
GTGGAAAAGTGAGTGGGAAGGTTAGATGAGTCATGGACCGTTTGGATTCGCGTGGCTTATGG
GGCTGCTTTTGAGTTAGAGGTGAATTGCTGAATAAAATGACCTAGAAACACAGAAAACGTAG
TTTTAACACCTTATGCATATTATATATGTATATATCTCAAATATCTATTAAATTTTTTTCCA
AAAATGTAGGGTATATCCGGGAATACCCGAGCACAACTGTAGGTCCGTCTATGCCAACGCAC
AAACTCAACTTGTAGGCCTAGCTTGCTAGCTATATTTGGATGTCACGCTGTTCTAAATTCAT
ATGCCTTAAAATTGATATAAGTCAAAGGCTACTATTTCCTCAAAAGAGAGAAAATGACATGT
GCGTACGTGAGACGGGAATTAGAGGTTGTGTCCGCTTTAGCTTCTTTCTGACAAATGCTGTA
ACGTCTTTGTTTGCAACTGTGCGTGCAGCCGTGAGCTTCTTTAGCTTTGGTTCTGACATAAT
GCCACAGGGCGTCTATAGGCGTTGTTTAAATACATCAAAAACCCAAAACTTTACAAGATTTT
CCATCGCATCGAATTTTACAGCATATGCATAAAACATTAAATATAGATAAAAAATAATTAAT
TAAACAGTTTACCTGTAAATCACGAAACGAGTTTTTAAGCCTAGTTACTTCACGATTGAATA
ATGTTTGTCAAATAAAAACGAAAATGCTACAGCCATAATGTTGAAGCTGGCGTGAGGGGTAC
CAAGCATGTCCTTGAGTAAAAAGAAGGCCCCGGTGAGGAAAAAAAAAGTTCAATCCTAGTTG
GCAAAAATAATGGTTCTATGATTCAATATCTATATGTCATGTTAATTGAAAGAACAGTGGTT
CTAGGATCATGTGCTATATCCTGTTTGTTTGAATTTATAATGATGCTGAAAAATATTGTTGC
GCTGATAAGTTCGAGTGAACAGATATTAACTTTTGTTGCGTGGGTGAAGGCCATGCCATGGC
CTAAAGATCAAAGAGACGCCATCACGGTGCTGCACTTTTCGGCTCCCTCCTGCTTCCACATG
CCGCGCGTCGTCTAGAAATCCCTGATTCAGCAGCACACCTGTGCGCCTAGCCGCCCACGCGT
ACACTGATAAACAGTTTTTTTCTAGTCCGCCCACACGCGCGCTCCGAGCCGCAGATCCTAGC
AAGCGCCGCGCATCCGACGGCCACGACAGCGCGGTGCCGTCCGCCGCCCCCACCGCAGCTTG
TCCACCTCCTGACCCATGAGCGGAAACCACGGTCCACGGACCACGGCTGCGTTCCAGTCCAG
GTGGAGGCTGTGCAACCCCGGTTTTCGCTCGCTGCGCCGTGGTTTGCTGCCCAAGGTGGCCG
GAGGTGGCGAAACCGCACCCGGATCCTTCCCATCGTTTCTCATCTCTTCCTCCTTTAGAGCT
TAGTATATAATCAGGGCTCTTGTCTCCTGGCTCCTCACAGGTTCGTTTCGGTTTGGATTGAT
TGGTTTGATCAGTCGTGGGGTGAGGGTCTTGGAGTCGATTGATCTGGGATACTGTTAGAGGA
TTTGGGGAGGGGGCAATGGCGACCGCGGGGAAGGTGATCAAGTGCAAAGGTCCGTGATTTCT
CCTCTGTTTCTTGATCTAATTAATTTTGGTTTATGGTTCGTGAAATCGTGAGTACTTTTGGG
GAAAGCTTCCTAGGGAGTTTTTTTTCCCCGATGAACAGTGCCGCAGTGGCGCTGATCTTGTA
TGTTGTCCTGCAATCGCGGTGAACTTGTTCTTTTTCTATCCTTTAACCCCCATGAAAATGCT
ATTTATCTTTCTTACATCTTCCAGTTCCAGCACTGCTATTACCGTCCATCCGACAGTCTGGC
TGGACTGACACTACTTATGGAGCATTGCTTTCTTTGAATTTAACTAACTGGTTGAGTACTGG
CTCTGTTTCTCGGACGGAAGACATTTGCTAATCCACCATGTCCATTCGAATTTTGCCGGTGT
TTAGCAAGGGCGGAAAGTTTGCGTCTTGATGGTTAGCTTGACTATGTGATTGCTTTCTTGGA
CCCGTGCAGCTG
SEQ ID NO: 30
Sequence Length: 1743
Sequence Type: DNA
Organism: Sorghum sp.
GACGGCGACGAGGACGGCGCGGGAGGTGGCGCGGCTGGGGACAGAGGCGGGCAAAGGCTTGG
ATCGACGGCGAGGGGGGTGGCGCGGCCGCACCGGCGACGAGGACGGGCGAGCGCGGCGGCGG
AATCGCGGGCGGGTGAGCGCGGCGGCGGCGGGGGCGGCTGAGTGTGGGGACGAGTGTGTGTG
AGAGAGAGAAGGAGTGGGGGGGAATTGGATAAGGCCAGTTAGGGCCTTTACCGAGTGCTGGA
TAGTAAGGCACTCGGTACATTTTTATTTTTATTTCAAAATTTCAAACAGCCCACGCGATCTA
CACGAAATTAAAAGTATGAATTCAACTTATCCCGAGCGCACAGCCAACCCTCGGTACAAAAT
GGCCACGTCACCGAGGGTTACCCATTACCGCGCTCGGGCTACAAACATTTTAGAGGCGCCAA
GGTGCACCCTCGGTAAAAATTTTGTACCGAGCGCCGCTGTATGCAACCCTCGGTAACTAGCC
ATTTTGTACCGAGGGTTAGCCAGGCTCTCGGTACAATTTGAAATCATACTTTGGATTGAAAT
GTTTTTGAATTTTTAATTTCGTAAATCACGTCTGCAAAATTTTGGATTCAAGTGTTTTTGAA
TTTTGAAAGACCAAACTCTACCGAGGGCTCCGCATACCCCTCGGTGTAAATATTTCACCGGA
GGCAACCCATTAGCGCTCGGTGGAATGACCATATTCTACCGAGCGACAGACGGTGCTCTCGG
TGGAGTTGACGACGTTGTGGGAGTTTACTCGACTGAGAGAGTGGTGCGTCAATTTTACCGAG
ATGTTTTTTCTACCGAGGAGCTTTGCTCGCTAAAACACAGTTTTAGCGAGGGTTTTATCTTA
CCGAGGGTTAGACGCTCGGTAGAAGGGAATGGTACCGAGCGTATGTGTTCACCGAACGCTAC
TCGTGAAGTGCACCGAGGGTCTTATTTCACCGAGCTTAACCGTCGGTACAAGAGTGATGTAC
CGAGGGCCCGTTTTCCTGCTCTCGGTACATCTTTATGCTCTCGGTGGAGATGCACTGTGCCG
TAGTGACTCGTGATTACGGATTCCTCTAAGTTGGGTACTTGTAATATCGATACCTGACAGTT
TGCTATTGTTGTTTTATAGCTTTATTAATAAGATAAAAATGCAACTATACATCGTAGCTCTC
TATTTACGAAATGGTACCACTAGCTAGTGACGTGTCCACTAGACGACATGGAAAACCATAAA
CAAGACAAGAAACCGCTGAAGGCAGAAACCGGCGGGGCCAAGGCTGGCTCGCGGAGGCCGGG
AAAACGGAAAGCGGCGGCGGACACCTCCCCGCGGTTTCTAACCGCGACTAAAAAATCCGAGC
CTTTCTACCCCCACCTTGTGCCGCTACAGTCCAGGCATTCTCGCTTAGTCCTAGACCACTAT
ATATACAGTACTCGTCCCCGCTTTCTTCCTCGCCAACTTCTCATCATCAGCCAAGTGTAAAG
GGTGCGAAGAAACAGCAGCAAAAGGATTCCATTCTCGTGTTCTTGGAGTGGTCCATCGAGCT
TCGTCAGGGAGAGCTATTGAGAGAGAGAGAGAGAGAGAGAGAGAGAGAGAGCAAGCAATGGC
GACCGCAGGGAAAGTGATCAAGTGCAAAGGTCCGTCGTCTTCTACCTCTGTTTTTCGTGATG
GCTAACTGGCCTCTAGCAGCCTAATCATGGAATTGATTTGGTTCTTTGATTATTCGCTGCCT
GCAGCCG
SEQ ID NO: 31
Sequence Length: 2712
Sequence Type: DNA
Organism: Sorghum sp.
CAGTTGAGCTGGGACCCGTATTCTGGTGACTGTGGTTAATTTGCTCTGTCTTTTACGTTTTT
TGTTGCTCTGATCTGGGTATCCTTTTTATGGTACCCAACACTTTGCTACGGATTTGTGCACT
CCAAACCCTAATCAAGACTCAACTCCAATACGACTCACGAACAAAAGCACTCGCCTACAAAC
AAATCTACATGTATGCCACGATTTGTTAACGGGTTAGGATCGAGTTAGACCTAAAAACTATA
TGCTCCACATGATTATAGGAAAAATTTTAAAAATTTCGTATTTTTAATTTACGTAAAAATCT
GGGATGTTACGATCAACATTTCTACGAGGTTGAGTTAGTAATCTTTGGAACGACGTTCGCGT
CTCCCCGTCTAAGGTCCAAGGTATGTACGATCCTTTCTTCTCTTTCTTTCATTTTAGGATTT
CTTTTTCTTTTTTATTTTTTTGTTCTCAGTGTATTTTATTATGTTTATATTATCAATATTTA
TTTTATTGTTTATTCTGCATAATTATTTCTTCCATTTATTTATTCTTAGTTTATTTTTTATT
CTTTCTTCTATTCTATTTTTTCTTTCTCATTTAACGACACCATATGGGACCTAGAAAGGTAA
GTATTATAATATCATATCAATGGATTGATAGTAAAATAGATTTTCAGTATATTTATTTGGAA
GTTATAAATATCAATAATATTTTTTATATAGTGGGTTAAATTTAAGAAAGTTTAACTCAATC
TAAATCTAAAATTATCTTTTATTTAAGGATGGAGATGGAGAGATAATATGTGTATCCATATT
CTCTTTTTTCCAAAGATTCTGGCACCAGGACTCCTTGTCTAATTAGGTTCTTGGGCTTGATA
GAATCCAAGCGAAGGACGCCATATACATGCTACTTGGTGCTCTCGTTCATCTGAACAATGTA
TAATTAAATTAGCATTATTAACAATTAAACAACTCTGTTTTGTTTTTCTTAGGCATCCACAA
AGATGCTTGCAATTGAACTCCATCATCAAGTACCAAACGGACGTGATTGTTTTTTCTTGGGC
ATCCACAAAGCTGCTTGCAATTGAACTCCTTCGTCGAGTACCAAATGGACACAATTGTACAT
AGCCTCGTCAGCATCTATAGCCCTACATACCAGACGTACGGATACACATCGACATTGATTCT
GAGTCATATTAGGAAGGACATAAATGCGTTGGAGGCAGATTCGATTAATTGGTTTGAAGTAG
GAGAGACGAGGACAATAGCCATACCAGTATGTCCTTCTGGCATATGCGTCTAGATGACGCTA
TACATTTTCACCCGTTGCAACACATGGGCACTTTTGCTAGTAGCAAGATAATAACCAACCTC
TATAATATAGCCGTATAAGAGATCCAATATATTATTGTAGTTGTGTTGCTGATATTATCCAC
ATCCTTCTATGTGTAGCGCCTTTAATTTATTTATATTAGAGTTTTTATGAATTTGTTCAATT
CTAAAGTTATACTCCCTTCGTTTCAAACTATAAGTCATTCCAAGAATCTTAGAGAGTCAAAA
CATTTTTAAGTTTGACCAAAAATATAGAGAGAAATATAAAGATTTATGTCATAAAATAGGTA
CACTATAAAAATATAACTAACAAAGAATCTAATGATACTTGGTCGATACCAAAAATGTTATT
ATTTTATTATATAAATTTGGTCAAACTTAAAAAACTTTGACCCTCTAAGATACTTTGACGCC
ATATACATGCTACTTGGTGCTTTCTCTCAGCTCGCCTTTGTGAGCGAGTCTTGCCTTGCCAA
GCTGAACGAGCCAAATTGGCTCATGGCACCTAGCAAGCATTTTCTTTGCCACCGCAAGGCTC
AAGAAAACCTTGCTTGCCAACATCTATGTATTCCACTACTATAATTTCGATGGGTAAAGTCA
TTTTAAGTTTATACATTAGCATCACTAAAATCACTTGATTGTTCAAATTCCAAAGCATCAAG
CAAGCAAGGTCTTCAATTGCTAGCTCCTCCGACATGCTTCAGGTAAGCCATGTTAGATTTAG
CATCTGGGGCAGTGATTATTTGTATGTTTTAACATCCTTTCCTTTGCATTTGCTATTTCACA
CACAGGTCTATGTGGAGTATCAGGGATATTCCTTAGTTCCATGCCCCAGATATATTTAGGGG
CTGTTTAGTTCAAATAAAATTGCAATTTTTTTTTAAATTTCTCGTTTCATCGAATCTTACAG
CACATGTATGAAACATTAAATATATAAAAAACTAATTTTACAGTTTATCCATAATTTATAAG
ATGAATCTTGTAAAACCAAATTAGTTTATAATAAGATAATAAATGTTAAACATAAATAAAAG
TACTGCAATAGCCAATTTACAACATTTTCTGCAGAAAAAAAAAATCGTCCCACCGGCCACCC
ACCGGAATGCGGACCAACCCACCCGTCCACGCGCCACCTCAAAGGCCAACGGCGTCCAAACC
ATAAACGACCAGCAAAAGCTCAATGACACACGGGCCTCCGTCGCCTCCACCAACCCCCGCCT
TCTTCAAAATTTTTATTCGCCGAAACCCCTTGCCACCTCGCCGCCTCCGCCCAACAAACAAG
CGCGACGACCTCTACCTCGCCGCCGCCGCCGCCGTCACCGCCGCCATATAAGTGGACCCTCG
TCTCGTCCTCCTCCCAGACTCTCCACCCTCCGCCTCCGAACACATG
SEQ ID NO: 32
Sequence Length: 3282
Sequence Type: DNA
Organism: Sorghum sp.
AAAGTTAAATCTGGAAATTTTGTAGTAAATAAAGAATAGTTAAATCTAAGAGACCATACTCG
AGGCATATAAATATTATTATTATGATCTGCAGCAAGATGTACCTCATCAAGTCATCAGAATA
GTTAATATATCATAACACGATAACACAACGATCTCTCTCTTATTTTTTTAAAAAAAGTCAAT
ATTGGTCTATTTGACTCGCTCATTGTAAAACCAAAATTTCAACCATGATTTTTTTTTTTGAC
TGTCAACGGGGGTGGGGAGTCGCTCCCCACCTGAGTATTTTGTATTTCATATCGGCCCTGAA
TGGCCTAATGTCATTGAACTTTTACATCTCCATCGGACTGATGAAGTTTAGATACGTACAGA
AAAGTTCAGATCGAAATCTAGTGAAATAAGACACTCATCCTTCCCCTTGTCTCGCCGACTCG
AAGGCTAGTGCCCATGCTTGCACAATATGGCGCTTGGATGGTTGGAGTCTGAACCCCCATTG
GACTGAAACCGCCGAGCATTGCTGCAGTAGTTGATTCAATCCGGTGGCTTGATTCCTGAAGA
CCTTCTCGTTTCTGGACTTCCACAATTGCCAACAACATAGGGCTATGAAGGTAGAGAAGCCT
TCACTTGGGATCATATCCGGGCGCGGCCAGGAAGTGACATTGTCTGGTTGGAGGGGAGCATC
GATACCCAGACAATTCCAAAGTTGGGTTGCCAAACCACAGGAGCAGAAGATGTGTGCCTGGG
TTTCTTCATGCTGCCCGCAGACTTCACAAGTTGAGTGATCAATGACATGCTTCCGCATTAGG
AGTGCCCTGGTATGGAGTCTATCTTGTGAGAGCAACCACATGAACAGTTGTACTCTCGGTGG
TGCAAAGGACTTCCAAATGAAGGTAGCACTTGCAGATGCCGGCTGTCCCTTAGCCTTTAGCA
TTCTGTAGATGGCCCCACTATCAAGCCTTGAATCTTGTGTACAGAACATTGACTCCCGCTTG
TCCGGCACATCAGAAAGTTCAACCTGGTGGATCATTTCGTTGAGCTGTAGCAGTTCATTACG
CGCTTGGGGGGTGATCCGTGGCACCATGTACGGGCCGATTCCATTCTTGATAACCTCGCTGA
CCGTGGCATTCTTGAATGTGCAATGGCTCAGAAGCAGTGGGAAGGCATCCCCAAGTGCATCC
TCCCCCAGCCATACATCATTCCAGAAGGAGGTTGACTTCCCATTACCGATATGCACAGATGT
GATAGCCTCGTAGAGAGGTAGTAGGAAGTGTATTCCAGTGATCACCATGGATGTCACCGTGA
AGAGTTGCAAGAGGCGCGCTCTTGTACCCACTGAGACCAGGCAGATGAGGAGGGACAGTGTA
GTCGATGCATCAGCTTCAGCAACAGGGAGATGTTCTGGATGCCGATGTCCCTGACGCCTATT
CCACCCAAATCCTTAGGATTGCACACCGCTGTCCAGGCCACCAGGCAAGCTGCAGGCGATGC
TTTGCCATCCTTGGCCCCAGACCACAGGAAAGCTCGTCTAGTTTTGTCCATCTTCGAAATCA
TACCCGGTGGCAACTGCAGTGAGCTCATTAGATATACTAACTGCGAGTCCAAAATCGAGTTG
ATGAGCACTGTTCTCCCCATTTTGTTCAGGAAACTTGCTTGCCAAGCACTTAGACGTCGACC
AGCTTTGTCGATCTGAGAGGTGAAGGCAGACAGTGGTAGTTTGTTGATGGACAAGGAAAGGC
CCAGGTATGGCTGTGGGAAGGATTCTCTTTTGCAGCCGATCGCCTGCACACATAAAGAAACA
ATCGCTTCATCCATATGGATTGGTACAAGAGTACTCTTGTCATAGTTGATCTGAAAACCAGA
CAATGCCGCGAAGGTGTCCAGTGTTTGGCGGACAGCTGCAGCACCAGCGAGGTCGCCCCTTA
GAACTATTAGCGTATCATCGGCATATTGGAGAACTGCACAAGGGCGATTGGCTTCAGTCGGG
TGCCTGATGGCTGCATCTTACCGGATCAGCCTCTGCAGAGTTTCAGCCACTAGTAAGAATAA
GTATGGAGAGAGGGGATCCCCTTGACGTAGACCCCGTTTACATTGGATCCAAGGGCCTGCAC
ATCCGTTCACCAAAACAGCAGTCAGCGCAGAGTTTAGGATGGAGCTGATCCACATCAACCAT
TTTTCTGGGAAGCCCCTTGCTCTAAGTACCCTCTGCAATCCATCCCAATTTACAGTGTCAAA
AGCTTTCGCGAAGTCCAGTTTTAACACGATTGCCGACATTCTCCTTTTGTGACAGACTTGAA
CTAACTCCATTGCAAACACAAAAGTGTCAGAGATGGCTCTTTCCTTGATGAAACCTGTTTGA
TTTATGTCAATCAAATTTGGGATTTCTGCTTGGAGCCTCATAGTGAGAACTTTCAACCATGA
TTGTTATCTCTCTTGCTTATTGAACGACTTGTAATAATGATTATAGAGCCTGCTTGTGTATA
GAAAGATTATGTTTTTGAGGAATTGTATAGATAGATTATAGATATAGTAAAAGAAATTCAAG
AAATCAGGCAGAGGCAGCTGCGGCAGCCGCCGCCACCGCCGCCGCAGATTGAAGAAGCCACC
ACAGCTTTTCAACAGAGAAGAATTTCTAATGATGGTGTGATAGTGGTACTTTCCAGTAATTA
TTAAGGCCTTGTTTAGTTCCAAAAAAATTTGCAAAATGGACACTGTAGCACTTTCGTTTGTA
TTTGACAAATATTGTCCAATCATGGACTAATTAGACTCAAAAGATTCGTCTCGTCAATTTCG
ACCAAACTGTGTAATTAGTTTTTATTTTCGTCTATATTTAATACTCCATGCATGCGTCTAAA
GATTCGATGTGACGGAGAATCTGAAAAATTTTGCAAAATTTTCTGAGAACTAAACAAGGCCT
AAGCAAGCTCCTAAATTTGAACGAAAGGAAACCAGCAGAGGAGAGAGAGCGCACACAGACAG
TACCACCAGGGAACAAGAAATCGAATATGCTGCCGCAGCCGCCGTGGAAACCGAGGGCGGAC
CTTCCCCTCCTCCAAGCAAAGCCTTAGGGCCAGGCCCACCGACGGGCGGGCTCCACCCGCTC
AAACATCCGTCACCGCCAGGTGGCCCCAGTCCATCCCTCCGCGGGGCGTCCCTCTGATCTTC
TACAAATAGTCCCGGCCGGCCGCGGAGGCGAGGCAAAAGCGCAAACGCCACCAGCAGCCCAC
CACAGATAGCGAGCGAGCGAGCGGGCGAGCGCTTCTAGGGCTTTTTTTTTTCGAGATG
SEQ ID NO: 33
Sequence Length: 4383
Sequence Type: DNA
Organism: Sorghum sp.
ACGATTTCCTGAATACGTTTATAGGATATATGATTGTGCGCGCATATATGTCCATAAATAAA
CATATATTAAATATCTGGTTCTACTGGGTATGTGCAATTTTAGCACCACCCCCAGCCCCTCC
CCTTCTGATCAAATAACAGGAAAGAATGTCCCCCACCATCGTCGGAAGCATTATCCTCTTTG
TTACGCCATAATTATAAGATCTTGTTTAGTTTTCTGGGTAAAAAAGATACAATAACACTTTT
ATTTGTATTTAGTCATTATTATTTAATCATAAACTAACTAGGCTCAAAAAATTCGTCTCGCA
AATTATAAACTGTGCAATTAATTATTTTTTGTTTATATTTAATGTTTCATATACATGTTGCA
AGATTTGATGTGATTGAGAATTTAAAAAAAAATAGATTTCGGACGTAACTAAACAAAGCCTA
GGTAGAAAACCTAAAACAAACACAAACTTCGTAGCAACCTTTCCAACCAAACTTCAAAAACA
CCCTTAAAAAAAAAGAACTCCGTTTCAGAAACGCCCACTGCAGAACGGAGCTGGTTCGTTCC
TCCGGCCACCAACGACGACAACAGACCTGATCTGACCTAACCTACCCCACCATGCATGTTGT
GATGATGACCCAAATGGAAATCGCCGCGCCCATTTGCAGATTAAAAAAATGAGGGTCATCAA
TCACAGCGGCGTGGTGTTGCAAAACCAGCACCCCCGTCGCCTGATTTCGGAACCGGACTTGG
AAACCACCCGCCGGCGGCGGCGGCCGGAGCAGCAAATGGGAGAACATGCCACCGAGATGCCA
TCTCATTCACCCGCCTTTCCTTTTCCTGCCGTGGGTGGTCAGCACATTGAACCGAAAAGGAA
ACCGGAGGTGGGTGATCAGGTTTCAGATCATGCATGGTCCATGGCTTGTTTCACAAGCACAG
GGGAAGAGAGACTTATCCGGCAATGTATATATCCTCCCTGCCGGGTTGCCGCTATATATATA
AATTGCTCCCTTCCTGCTCCCCCAACCTCCGCATTGCGTTGCGTTCTACTGCATCTGTTCGT
TCCAGTTCTCTGTTTTGTTCTGTTCTGTCGACGCCATTCCTGTGCTGCCGTCCTGCCAAGGT
ATGCATCTGACGACGACTCGTCTCGTCCGTGTTGCCTCTACGATGCAATGCCGGCCACTGTG
TTCCAAAATTGAGCATGCGTTGGTTGGGGGTAGTACCGTAGTAGTACGTCTGTTAGCGGAAA
ATGTGCATGTAATGCAAGCTAGATCTGTTTGCTGGTGTTATTTAGTTTTTTTTTTTTTTTTT
TTTGTTTTTGAGGAAAGCTGGTGTTATTTAGGTCTGGTTTATCTAAAAAGTTTTTGAATTTT
TATACTGTAGCACTTTCATTTTTATTTGACAAACATTGTCCAATCATAAAGTAACTACTGAT
TTACAGATAAACTGTGTAATTTTTTTTTTACTTTCATCTATATTTAATGCTTCATATATGTG
CTGCAAGATTCGATGTGACGGGAAATTTTGAAAAAGTTTTTGGTTTTTGAGGTGAACTAAAC
AAGGCCTTAGTTTATATCCACTGCACATTTTATTTTTTTATTATTTTTGTCTTTTGCATGCA
TCCTGATGAAATCCGCCGAAAAGTTGAAAAATGTTTTTGAGACAAGTAATTGTCAACGTGAC
GGGATCGACGATCTTTAATGGTACACACAAATCATGCATTGTCATCGTCCCCGCTAAGGGCA
CTCACAATACAGACTCTATCATGGAGTCTAAAGTTATTTATTACCTCGAACAATGTGGATTT
GGAGTCTAAATAAGACTTAGAGTCTTATTTTTTCTATCTCTTTCTTCAATAAATATGGTGCC
ACATCAGCAAAATATCATAAATAATATATAATTAATTGTCTTGGACTCTATGATAGAGTCTT
GCATTAGGAGTGCCCTAAGCGAAGCGTTTGGATCCCATAAGCTGTTGGGAGACTTGCCGTGA
GCCAGTAACATGACAAGAGCTCTCCTGTAGTCCTCTCGTGTCTTTCTTTTCGTGCAAGAAGA
AGAAGAAGAAGAAGAAGCGCCGCCTCCTTTTTCTGTAATTCTGTTCCGCTACCTTCTCGTCA
CCAGTCACCACTTCGCTTGATTTTTTCCATTCCATGTCAGCACCGCATCGGCACACTTCTTG
TTTAGGCAGCTCTGTTCTTGTTTGTAACGTACGGCCCCCACCTCGTCGCGGCGGCACGATCG
ATGCGAGTGCCGTCTGGCGGCTCATCACTCACCGGGCGCCCAGCTGCATCGATGCAGTATCT
TCGCAAGAACCGATGCCTTTCTGTTCTTGAAACGATGTCTGCGCCGGGGCAACTTTTCTTGT
TGCCGCTTGTCGCTTGCGCTGCTGCTGACTGGACGGCAGCTCTGGAGGGAGGGTGTATTTTG
GATGCATTTTTAGGGATGGTTGTGCGTGCCTGATTTCGTATACAATTCGCCACTTGTTTGCA
CCACGTTCTTGGTGTTTCCCCCCCGATGTTTCTGCACACGGGCTTTCCTCTGGCTTCTGAGA
GACCCATGTTTTACAGGCTTTTTCTAGAAGGAGGGCAAACCTACTGATCCAATGGGAGGCAT
TAGGGAGGGGCAAAAATATCTCGCTGCTTTTTTAATTTATAATTTAATCCGGACAATCTCAT
TTGCGTTTGCGTCTGATGGTGATGGCAATGGTTATCGTATCTTGGGTGCCGGATCACCGCCT
TTTTGCTGATCACCGAATCAGCTGGGAGATTTCGGTGGTGAAATTAGGAACTCAATACAGTA
GAAAGAAGCTTTTTTTTTTGGGTTCCTTTCTTTTGTTTTGTCACAGTTTCGTGCTCTGCTTT
CTCTCCCAGCTAGTAGTCCTTCCTTGCGTTCACTGCACCTACACAGGTCATCGCAGCCTGCA
CTGTACACGAGTCTGCATAAAAAAAGTTCCAAGCTTTTTCGAAACCGGCCATTGGTCTGGTA
GTGGTAGGCCAGCATATGCTAATGGGATGCTTTTGCCGCACCATTGAGAGTCCATGACACTA
TCAGTGACACCACCAGTATTTGGAAATTCTATGGTAGTAATTTGGCATTATCCATTGCTTAA
AATTCCCAGCTTTGTCAGCTTGAAGGTGGGCCCTACCATCTGCACCACAGCTAGCTACCACC
TCGGCACCTCACGCCTGGGCTTAGAGCAGCTGCTGCCCCCTCTATTTATTGGTTCCTCTTCC
GTCCCGGGGAAAGCCTCCTCCATTGGACTGCTCTCCCTGTTGACCATTGGGGTATGCTCGCT
CTTCTGTTTATCTCCGTACTAAACCACTGTCCTCTGGTAAATCCTGGGTGGGGTTTTTGCTG
GGATTTTGAGCTAATCTGGCCGCGGTAGAAAAGATCGTGTCTTGATGAGCGCAATCACTCGC
CTTAATTGTCTCCTTGCCTCGCCATTTCTTCCGGTTTTCATGCGTTTCCGTGACTCGTTGGG
TGCGTCATCTCCTGAATCTTGCCAGGGCTCTGCTGACATGTTCGGAGTTGGGTTTATAGATT
TCTCTGATCTAAATCGTTGCTGTGCTGCGCACAGAGCTTCCCCCGTCCTTTTCTGGGAGTTT
TGAGCTTAGGATTTTACTTGGTGGTGGTGGTAAACTTGGATTCACACATGGATGCAGTAGAA
GTTCTAGGCTCTGTAGTTTGCTTGAGATCTTGCTGTGATTGCTTGCCGTGCTCATCTCTTTT
GCTTTCCGAGAAATGTATTTGTCGTTTTGGTGGATTATTAGCGCGAAAAAACCTTTCTTTTG
TTTTTGGTTCTTTTACTACGAAAAGTCATCTTGTTGGATTTTTCTATATTTCCCCTTTTGAT
GATGATGATGTCCTTACTCTAGGAATTTGTGATGTCCATGTCCATTCTTGGCTTCTTGCGGT
TGGCTGTGCTTATTCGGAAGCCAAATCCTCTTATTTTTACTGGTTTTTGGCTGCCTCTTAGT
GGGGTTTAGGGTCTGGATGGCATCAATACTCAGCAAATTAACTCAATCATGTTGGTTCCTTT
CTGCTTTGAAAATATTATCATATAACTAAGTGCTTGTGCGGAATCAGTACTTGCTTTTGTTT
GGTGGAGGATCAATACTGAATACTTGCTTTGTTTGGGTGGGGATAAGTGCCTGCTTTATCAT
GACTATTTTTCTATATGATTCTATCTGGTTAAGTGTTTCTGTTGAGATAAATCAAATTGTAT
AGCTGCATACTACATTTTTTTTTTCAAATTCAGGTTCCTCTTGCATTACCTACTTTTTCAGA
CAGTCTTCTAAGTGCTAGCTCTTTATTTATTGTTCTTGTACAAGTGGTGCTGCTGAATCTTA
ACTGTATAGCTCGAATTGCAGTATTGAGTATCATTGAGCCATG
SEQ ID NO: 34
Sequence Length: 3100
Sequence Type: DNA
Organism: Sorghum sp.
ACAATAAGAAGCACTTCCTACTAGACATGTCAACAGGAAACCCACCTCTTCAACCGACAATC
ATCCCTTACTTTATACTCTCTCTCTCGTACCAACTTTTAGAGTTGTCCTAAGTCAAGCTTTT
GTAATTAAGTTTAGCTAATTTTATAGAAAAAACTATTATCATTTATAACAATAAATAAATAT
AATGTGAAGAAAATATATTTCATCGTTTAATGATACTAATTTTGTGTCATAAAGTTTTTCTT
ACTTAATATAAACTTAATTAAACTTATAAAGTAACTTACAACAACCCTAAAAGTTGATTTAT
TTTAGAGATGAAGGTGGTACTTGATAGATGGGTTTCGTGTCATAAGCCATTGCATTAAATGT
TGCCGACTATTGAGACCACGTTACTTGTCACAAAATGTGCAAGACCATTTTTAGCATGCATC
TATTTGCCTTTTCTCATACAGTCATACCATGCTCTATGTGTACTCGCTCCATCAAAAAAAGT
GATGCTACGAGATACAAAAGATTAACTAGATCTGCATAAAATATATTCAAATATTTATATTT
GGAGAACAGATTTGCATAAAATCTAGTCCCCCCCCCTTCTCCAAAAGAGTTCATAATTTGTA
TTGCAAGAAGACAGATAATTGAAATTTTCATCAAATCTATTAAAAATACTTAGATTTATAAT
ACCAAATAGTATTATTAGATTGATAATGCTATATATACTTAAAGACATAAATGTTGCTGTTA
CTTTTTATAATTTTTTATCAAACTTTTTTTTTTACTTAGTCAAAACCTAGAAGTGTACTCAG
TATTCCTTTTAGAACTAGGGAGTGAAGTCCATCTTTTGGACCTCCTCTATTCAAAACCACCC
GTGAAATTTTGGAAGGTTGATTGTGATTGCTTTTACGTTTTTATATAATACATACTTTCTTT
GTTCTAAGATTATAAGACGTTTTGTCTAGATACATTAAATTTACTATATATTCAGGTGTACT
TCCTCCGTTCTAGTAATGATATTTAGGCCTAGATCACCCTAAAATTTAAAAACTTTTCAAGA
TTTTCCATCACATCAAATCTTGCGGCACATGTATGAAGCACTAAATATATAAGAAAACAAAA
ACTAATTGCACAGTTTGTCTGTAATTTACGAGACGAATCTTTTGAGTCTACTTAGTCTATAG
TTGGATAATAATTATCAAATACAAACGAAACTGCTACAGTATTGAAATCTAAAAAAAATCGG
AACTAAACAAGACCTTAAGTCTATAATAAAGCTATGTATTAAAAAATCTAAAGTATTTTTAT
AATTTAGAATGAAGTATATATCTATATTATGTGTGTATGATTGAAGAAGTAGGGCATGTTTA
CTGTCACATTTGCTTTTGCCTTTGTGTTCTTTTTTCTCCAATATATATAATCTCCTTTGCCC
TGGGCTTTTTCCTGCCCTTTGCGTCTTCTAGCACGTTCTCCCATCTCCCTCCTCTGCACGTC
CCCATCCTGAAGCCTGATTACCACCCGTCCAAGAAACAAGCACAGGGACAAAAGCCACTGAA
GCAACCATCAACAAATCAGCATCCTCCAGCTCCAGGCTCCAGGCTTCCAATCCAATCGAGTA
TCCAATCCGGTTGCTTCTTCTCCGACGCAACCCCAGGCCCGCGGCAACCGCCAGTAGGCAGT
ACCTCCCTCTTTCGCAACCGCCGCCACGCGCAGGTAACGACCGGTGCCATTCTCGTCCTTCC
TTCCCGAGTCCCGACCTTTCCTCCCGGCTTCTCCGCTTGTTCGGCTGCTCTCGCCTGGTGCT
GGTAGATTGGGTGCTTCAGCTTCGTCTGATCAGGCGGCGTTAGAGGATCCCCGCGGGTGTAT
TCCGTTGTGGGTTTCTGACTATCGATTGGTAGTTTTGTCAATCATCTGCAGTAGCCGAGTAC
AATGGATTCTACTGATAGGGATGTTTAGTACTCTGCACGTTGCGCTTCAATCATCTGCACGT
TGCGCTTCGATCTTCGTTTTCCGGCGCATCCAGTCCGTGTGGAAAGCAACGGAGGGTTGATA
TTATGTAGTTGGGTGTAACAGGAGCATCTGCTGTTGTTTTGGGTTATTTGCACAGTTCCTGC
ATAAAATTCAAGGTACATGCTATTAAAAGGATATACTCGCTCCATTGTAAATTGTAGCTCGC
TTAGCTTTTTCGTAAGTCAAATTTCTCTAACTTTGGCTAAGTTTATTGTTGAGTTGCATCTT
GCATGCACCGACCAGTTCAACTCAAAAGCTTAAGTTGTTAATTAAAGGTGAACAATTTACTT
ATATACTTCAACATTCCCCCTCACACCTCAAATCTGGAAATATGAGTCGTTTGTAGTTAATT
TATTTAATTATTATGCCTGTTAAGATTTGAACTCGTCGAGACCTGCGGCTCTGATAATTGTG
CATTTTTATTTGGATCTGAACTGAGGGAATATCTGTAAATGATATCGGTGAATGGTGAAATT
TTCTGTTGGTAGGGTGTCCAACTGTTACACAACCAACCGAACCCTGGGTCTGCACAAAAGGG
GTCTTGCTGAGTGTGTTCCTGGGGCCTCCGGCCTTTGGTCTCTTTTGTCGGCCCTGTTTTGT
GATCCTCTTCTTGATGCAATGTCTCAGGGGAGGTCTTTCCCCTAGGGATCAAGTTTCCTTTT
TAACGTAAAGGATGCATAACAGGTGGAAATGCACTAGCCATTCCAAATGAGTGAAAATGGGT
GAAGGGTTGGATATATGATATTTTCACCAAAGCACTAGTCACCCATGGTAAAAATCCCAATT
TGCCCGAAATGTTTCTTCTTTTTGTGCAATTCCAAGTGGTGGAGCTAGGGCCCGGGCTGCCC
TAGCTGCAGCACTGGCTCCAAGCCAAAAGGCAATGCAAATCCTCTATAAATTCTACAAATTC
GAAAAACATCCAAACAACTTGATCTTGTATTAATACTTCTGGCTCTGGCAACACCCCATTCC
ACTTGCTGTTACACACCCAATATTCATACTGGGGCCCTACTACCCTAGATACCATGAGACAC
TTATTATTCATTCTATTATTCAATTTGTTTGTTTGTTTTCCTTTCTATTCTTAATTATCATG
SEQ ID NO: 35
Sequence Length: 3097
Sequence Type: DNA
Organism: Sorghum sp.
GACGCGGCCCGTGGCGCCACCGATGAGCGCGACGGCGGTGTGGCCGCATCGGCGGAACCAGG
AGAGCGCGACCAGGCCGAGGAGGTTGCCGAGATGCAGGCTCTCGGCGGTGGGGTCGAAGCCG
CAGTAAACCTTGAGCTCCCCGGGCCGCGCTGCGGCGAGCGCCTCCGAGGTGGTGGCCTCGAC
GAGCCCTCGCTTCATGAGCACGTCCACGACGCCGGCCGACGCAGCGGTGGAGGTGGGAGCGG
AGGCAGTGGCGTTGGTGGAGAGGCGGCGGCGAAGCGGGTGGGTGTGGGGGAGGAGGAGGCAA
CGGTGCGGGCGCAGGAAGGACCGCGAGGAGGCGGCCGCGGCCATGGCGGCGGCGGCGGCCAT
CGTGGGGAAGGAGGACGAGGAAGGATTTCGGCCTTGCGCTGGTGATAGGGCCCTACCTATGC
TAGGGCTGGGCATAACAATTACCAATTTAACATAATAATTAACTTGAATTCTTTCAGTTCCT
ACTCTCGATTAACCAAAAATTAAATTATTGTACTCGCTTATTTAAATTTTGTAAGAATATAT
ATTTGATTTATGTGTGATATGCCATGTTTTGAACTGGTGTGTATTTATGTTTCTCCAAGAGT
ACCCAATTTTGTTTTCCAACTCCTAAAAAGATATTAGAAGGAATAAATAAGATCATCGCCAA
CAGTTTCTATAAATCCACTCATAAAATAAGAATACAATTTATATCAGGAATCTTATACTATT
TCTAAATTATTTTAACCACTTTTATAAATTGTTTATCTCTTGTATATTTGCACCAAGAATCT
TTTACTACTCTTTATTCTTTTCATGTCCTTACAACTTTAGATTGTCCACACCGTAAGCGCAT
TTAGATCACCGTGAGGGGATACACATGGTTTCCAGTTCGCATAACTTTACGCGGAACGGATG
GTTTTTTTTCCAAATGCTAAAAGATTAGAAGTTGTTGGAGATTATTTTCTTTCTATCCTTGT
GAAAATCGTGCATTGGGAGAGTGTTTTAGATACACTGGAGATGCTTTTATATTGCTATATAA
TGCTTTCTTACCTGTTACTATTATCTAAAAGAAGAGGAATTGTTGCCAAATTTATTTTAGAA
ACACCTTTTTTTTTTGTTGTCTTAGTCTTTGCTAAAAGAATGTTCTGTTAGTTCAGCGAGGA
CCGAAACTAAACCAAAATAACCAAAATGCCAAAATACTTGTTTTTTCAGAACTGAATCCATT
TTCTAGTAAGGCCTCCTTTGGAATGGAGAAAAAATATAGGAATTTTGAAGGATCTAAATCCT
ATAGGGGGAAAATATTCTATGACACCCTTTAGAACAAAGGATCTAAATTTGGAAGCCCTGTC
TTTTTTGTTTTTGGTATTCTTTTGAAAGATTCTCTCAATTATGTCTCTGATAGTTTAATTTA
GAATCTAGATCCAGTTGGCGCCAAGGCCACAAATTACGTGCATAGATGTGCACTGTTGGCGA
TGTGGCGCCAAGCTTGGCGCCAGTGACGATAGGGCTAAGTCCTACGTTCAGATTTTAAAATC
AAACTACCAGAAATATAATTGTGAGAATCTTAAAAAAAAACCTTAAAAAAAGACGGCTCCCG
GCCCCTGCCCCGAGCCCCAACGTCATCCTCCCTGACGGTGTCTTCTACACCCCTACTCGCAC
GAAGCTGGGGGCATTTTTAGGAGGCTCCACACGCCTGAATGAAGGAGCTGGGGAAGCCATTT
TTTTAGCTCCCGTGTCCCAGCTGCAGAGAACGTGATTTTTGGGCGAAGCTGCGAGGAGCGGA
GCTCTCCGACCCGTTTGGTAGGCAAAAGTTGCGAAGCTGCGCGTGAAGCGCTTCCAAATTCT
GTACCAAACAGGGCCGTACTATCTTGGCTAAGGGAAGGAATTGGATAATAAAGAATGCATGT
TTCGCGAGGAAGCTAAATCTGTGTATCATCTGTTTTTTTTTTAATGTTGTGTAGCAACTGAG
CTTTGGTGTTTAATGTATGATATTTTTATAGATACAGATTGGTAAGAATCAGTTGCAGACGG
TGGATTTCTAATAATAAAAATGCAGTGATCAACACATGCAATCTTACCATTTTATAGAGCTT
ATGGAAAACACGAAATTTTGTTTGTTTTCAGGGAGAGTGTTGTCGGGATGTGAAGGTGGTGA
TGCGAACTGCAGGGGAATACGTTGGGAGGATGATGGCGAGTGTTATATATAGTAGACAGAAA
AATTAATGCCCCCAAGACGAAAGAATTGCTAAGAAGGCTATGGGGGATACCCAAGTCAGCAA
CCAGAATTGGCTGGGGGTATGTACCAGTAGCTACAGTACACTGGTGACAAGGCGAGAGCATA
CAAGCTGGCTAGGCTCGGCAACAACACGCGAGCTGTTCCAACCGTGCGACAATGGCACGGAG
CAGGCGGCGGGAGAACGACGCTTTGCCGCACCCTCTCCCCATGAAAACTAGCGGTCAGTGTG
AGCTGCAGCGCCAAAACCCCCAAAGGCTCACCCCCGGGACGGACCAGGAGTGGACGACGACC
ACAGCCCAGAAATCCTGCTGCCATCCCGTTCACGAACACGACCGCCTCACAAAAATTGGGAG
GCCGGGGAGCGGGAGCGCGCACCTGACCCAAACTACCCCCCAACCACGATCGACCGACCAAC
CAACCAAGGCGACGTCGTGCTCGTGCAACGGCGAGCACCGCCAGCACCGCACCGACAAGCCC
GGTCGCGCAACGGAAACAGCCGGCGTGGAACGAGTGTGGGGGGGGGGGCGAAGAACGAAAAC
TGAAGCGAAGCCAGCCACGGGCACGCACAACTCGACAAGTCGTAACAGGGGCGGGTCGGGCA
CGGGAAATGGGCCACACAATGATCGCGTCGCGCAGGAGGGGAGGGAGAGCGGCCATCGACAG
CCATTCGTCGGGCGTGCTATCCGAAATCTGATCCCTCCACGAACCCCGACCTCACGATCTCC
GTTCGCGGCCCCGCGCACCCCCCCAATCCGCCCCCAACTCACTTCGTATATACGCGCCCGCT
TGTTGGCTAGCATCGTCATCTTGTCTTGATCCTCTCTTGCTGCCTGGTCCGTGGCCATG
SEQ ID NO: 36
Sequence Length: 3281
Sequence Type: DNA
Organism: Sorghum sp.
CCCGACAGACTTCTTGAGTCATTTGGAACTCGTGCACGTCGATCAGAAGTCTTTGGGACAAT
GATGTGTGCATGTGGGCCACTTAATTCAGGAGGTTCTGCCACAGAAATCATAAAGCCATGTT
GTTTTTTTATCTATAGTATAAGAAGGGACCTACGTATCACGAGGTTGGAGCGGGTTTGCGGG
CACGGGTACAAGTTTTCTATGCCCACAAGTTTTTATCTGTTGGGTTCAACTACAAACCCGCA
TCCACACATAGGCGGATCCACCACTAGGGCGAGCCAGGGCGGCCGCCCTAGTTCCTCTTAGG
ACTCATCTGACACTCTATGGAAATTTTAGACATTAGTGCGAAATAAAAATAGGCTCCTCGAG
TATCCGACGGAGGTTAAAGATAAACTTAGACGTCTTGGACTATCATGTGTGGTTCAGCCCAT
ATAGAAAACCACAACCCACAAAGTGCAATAAGAATACGATCGGCCTTTCTCATGTTTCTCGC
TATTCTCGACTTCTGACTTTCTGGTCGTCTTTCTCGCTAATTCACAAACTTGCGGAACCTAA
GAACCTGCGCCGGGCTGCAGGTATCTTTATTTTTTATTTTTCCTTTTCACATAGTTAATTGT
TTAAAATTTGATTAAGGATGAACCATCGAATGATTTTGTGAAGTCAGTTTAGAACTATTAGT
ATTAGTATGTATGGCTTCTCTTAATTTAAGGAACAAACTTCTCTAGTTTTGGTGTGGAAAAT
ATATGCTAAAAACTTTAATATTGGTCAAATTATTATGTTGCCAAAGCAACTTATGGAATTTG
TTAGCTTATATTACCATTGGTTTGCCAAAATTTTATGGTATAAGTCCGCCCTAGGTCATTTC
TCGAGCTGGATTCGCCACTCCACACCAGCGGGCACAAAGTTGTATCCGTACCCTCACTCTAC
CGGGTTTTCACCCACAGGCACGCGAGTAATCTGTACTCGTTGCCATCTTTACAATAGACCAT
GTCCCGGCAAGCCATGGCCCTACTAGGCGACACCCTTGTGAGCCATACTCCTGGCGCCGCTC
CAGCTGGCTGTGCTCTAGTGAGCCGCGCCCTAGAAGCCATCCACCACGCGAGCCGCGACCCT
GTAAGTGCAATCAAGCCTATTGTGGGTTTTGGCGTTGATGACCACCGAATTAGGGAACTAAT
GAGATTTGCTGAGATAACAAGCAGGGAATATAGCAAAGAGGTTGTTGAATACCATGGAGGAT
CCCCCATTTCTAAAGGATGGTTTTCCTAGCTCCAAAGGAGGTTTAATTCTTTTTCGGTTTGA
ATTTGAGTATAGGAAAAGCCGTACTATAAAGAGGGACTCCAAGGTTGTTGATCAAATTGTGA
ACCAAAGGCTCAAAAGCTCATCAACATCCTCAGACCCAAGCCTAGCCAGCATATCCTTCTCT
CTACACTTTGGTGTTTTCAGGCTGGTTCAGGCAAGGGCGGCACTGCCGCCCTACCCTGTGAC
AGTTGGAGAGCTGGGTATAAATACCCTTTCAGACCGTCTCAAACGGCAACCTGCTCATCTTC
CTCGCTCCCAACCGTTGCAAACTGACCAGAGCTCACTTCTCTCTCCCTCCATTGTTGCTCCT
CAATCCCCCAAGCCAATCCTTGATTCCAACCATCAAAACTTGAGGGAAAAGGCAGCAAACTT
CGATTGGAGAGCAGATCCATTGATTCCCAGCGTCAAAAAGAGCTTTTGGTTCACGTTTGGCC
GGCAACCTTGAGTTTGTTACTCTTGGAGCTTGCTCCTAGCCGGCTAGGCGTCGCCCTAGAGC
TTGCCAACTTGTGTGGCAGCCAAGGGAAGGTTTGTAAAGTTACCCTTGCAGCTAATACATTA
TTCACTCTTTGCAAGGGGTAAAATCCTTGCTTTGAGAACGAGGAGAAGGTAAGCCTGTGTGG
CTAAGCCGGTCCTAGTGTGGGCGCCTCAACAACGTGGAGTAGACCAAGCCTTGTTGTGGCAA
CAGCTGAACCACGGTAAAAATCGTGTGTCTTGTGTGCTTTCACTTGTGTATAAGTTTGTGTT
AGGATTTGAGGCCGATCTACTTGGTGGGGAGGCTCCAGCACTTTCTAGCCACATACTTGTGC
TCTAACATCTTGCAGGAAGCTTGGAAATTTAGTCGATCTAAATTTCTGTGGGTAAACTTTGA
ATCATTTCAATTAAGCTTCTACCTGTTTTTCTGTAGAGGCCGGCAGTGCCGCCCTGGGAGGG
CCTCACTGCCGCCCTTGCCTGTGTGACTGATAAACTTTGCTGAAAAAGTTTGAACAGGCCTA
TTCACCCCCCCTCTAGGCCACTTCCTGTACGTCCAGAGATCCTACAGACCCTAGCTGGCGGC
ACTCGGCAAGCCACGCTCCCGGTGGTCGCACCCCTGGTTCCCGAGCCCTGGCAACGGCACCC
ATAGCGATCCGCGCCCCTAGTGACCATTCCTCGGCCAGCCGTGCCCCTGGTCATCTCGGAAG
GATTCTCTTTTGGGTCTTGTTGTTAGAGAAGATTTAAAATGATATAGAACCTTTTATTTATA
GCACTATCCAAACTTCAAATAAATCTTACATTTTGGGTAAGGGTTGTTGGAGACAGTCTTAG
CAAACATACAAGAGTAACTTTAGTTTCTTTGAGAGGGGTCAAAGTGGTGAAGAACCAACATC
TTCACCAACATCGTTGTAGTTGATACTACGAACAAGTACTGCAATAATGCAAATATTATGTT
AAGCCGACGATGGTATTAACAGATAATAAACAGGTAAAAAAAATGTAGTAGGTCCTGCACAG
AGAAGGAGAAAAGAAGGGGCGAATTTTGTTGTAAAAAAATGTTATAAAAAAAAGGGGAGGAA
TTTCAAAAAAGATAAGCTCCAGAGTGCAGAAACCCAAACCCAACCCGGAACCTAACCACCGC
CGCCGTTGAATGGTCGGGTAGAAAACGTAACCATGGTTAACTCCGGGACCCTTTAAAAGCCG
AGCCGGGCTCGTCCCATCCGCCCCACACCGCTTCACTCGTCTCCTTTCGATACATACATACA
CCCCCGCGCACGTACGTCGTTCGTCCCGTACGTGCTCGTCTCTCCTTCTTGTGTGTGTCTCC
ACTCCCTTGCCTTGTGCAATCTTTGCACGCAGCAAACGCCATGATGATGCTCTCCGTGCATC
AATGGCAGGCTTTGGTCCCAAGAAGCTAGGTTGCTAATGCGCGGTCGTCGCTGCTGTTGTGT
CGTTCTCTGTTTGATTGCAGGCAGCTGGTTCACGCACGACCTCGAGCGAGAAAGATG
SEQ ID NO: 37
Sequence Length: 3037
Sequence Type: DNA
Organism: Sorghum sp.
CTAACCGTTTGGCCGTGGAACGATGTGGAGGTGCTTACTTTTCGACATCGTCCTTCTATTAG
ATGAAGCAAGAAGTCATTCAACTAACCAACAAATTTTCATCGGGCAGTGGCGCAAACGCGGT
GCGACGGATTGATGGCGCGCGGCCACTCCCTCCCGATCTCTTTCACTCCACTACTCACGCAC
ACACTGTCTCAACCTCTTTCACTCGCAGATCCAGTGACGGCGGCCACTCCATCTCCCTCTCA
TATATTTATTTGTATTTTTCAAGCATACATATATAACATAACACATGCAGTTAACCTCAAAA
GTGATTTTGAATTTTGTGATTTTTCTATATTTTCTTTTGATTTTTGTAACTCCTTTAGAAAT
GGTACAAAACTTGTACTCCCTCCGTCTCAAGATATAAGGCGCGATTTGACATGGCGCGGTCT
TGAAGATCATACTTTAACTATTAATTTATACTATTATATATAATTTATGACAACAACAAAAG
TATCATTAGAAAGTATTTGTAAAGGCAAATCGAATGTTACCATGATTATACTATACTTTTTT
TATATTATTAGTAGACTAATTATTGGTTAGGGATAACAAAGTTTGAATTTTAAAATATGTGC
ATGCCTTATATCCTGAGACGGAGAGAGTAGCCGGTAGTGTAACCTGTAGAGATGCATGGTAC
GGAACTCCTGTAATACCAAATCGTCCGCCCGTGGAACGACAAGCAGTAATCGTTTTCCCGTA
TACACCAATTATTTTTTCGGCGTCGTCCTTCAATCGGACGAATCAAGAAGTCCAAGTAGAAG
ATAATTACTCCAGTTTTAGTCTCTGGTACAAGACTTCCTTGCTAATCGTTTCCCCGTGGAAC
GATTATTGCATGTGATCATTTTCCCGTACATCCTTTCATTTAGAGAAATTAAAATCTACTTA
ACAAACTATGCTATTTAGCTTGGAGTTTGATATTCCAAAACGTTCCAAAATTCACATATATG
ACTATTTCAAAAGACTCTGCTGCGGTACTCCCAAATAACTGTGAGCCGTTCATTGATTTCGA
TCGGACGGTTACGATAAACTGTTACCTCCTAAGAGGTAATAGATGAAATGTATATTTAATAA
ACATGACAACTAGGTCCCATATATTTGAAACAAATGATAAAAAAAAGAAATATACGAAGATA
AATTATTGATGAGTTGCTACCGTCGTTACCTCTAACTGTGTCTAGCTAGAGGGTAATTAACT
TCAAGAGTTCACCTAAAATGAGATGTCAAATTACATGATTTAGCCATTATCTAAAATAAAAA
ATAACTTAAAAAGTAAATCACTCCAACAAGCTCTTCTATTTTTTTTCATTCACTCCATAATA
AAAACTTGCACGTGATCTCGTTTTCTTCGATAACGGATCCGTCCAGTCGCACGGCAAAATCA
ATCACGACGCCCGTCCCTGCCATAGTCCGTCGCGGGGCACCGGCCGGGTCACGTCCTGGCCA
CCCGTGTGGCCACCTGCCAAGGACAAGGCCTAATCTACGTACGTGATGGTAGGTGCTTGGGC
CAGCCGTTGGCCAAGGCAGGCAGGGAGGATGTTCGGTTGTAGCCGGATGCAACCATTGGGTA
GCGTTTGGATCCACGGAGTCACGGAGATTTTAAGAATCTGGATAGAAAAAACTTATAAATTC
TAAAAGTCTCATTCAAACATCCAAAGATTTTAGAAGATTCTAACACACAAGCATATAACTAA
AAAGCATTAGAGATTATTTTTATCTAGAATCTGGCCGGGTATGACTACGCGTTTGTCAGGTC
ACGTCGTGTGGCACTTGACCGAAGGGCAACACCCTTTTTTTACAAAGAATAGATTTTATTAA
TTTCATCATAACTATCACACCGAGTTGATATAATAAAAGTGATTTTTTTTTGTTTTTGCCTA
AACAGTAATCACCAATCATAAGAGAAGGAAGCTTGAGACTTTGATGACAATAAATTCTAAGA
CTATGTTACCACCTATGTGTCCAGAAGAAAACGTTGTAACTGCTTGCATCATTGTTGAGACG
CCTCAACACTATAGCCTATGTGTAGTGCTTTTGGAGGTAATCCATATACATAGCCAATGAAT
TACAAATGCCTGCAAGAGAGAGTAAATAAGTTTTTTTAATACTAAATCATTTCTGCATAACC
AAAGCGACCAACAAATAGATGTGACGAGGTTTGAGATCCTTCTGAAACCCTCAAAGTTGCTG
GTAGGAGAGATAAAAAAGACATGAGAGAGGTTGATAGACATTCTATGAGGGACTTATTTTTA
CACAAAATTATCCAAATCAGTCTATACAACACACGATAGCAAGTCTGTTGGAGTTGTTCTAA
ATAGACTACCATTATGCTTCTGTAGCCAGAGTACAACAAAACTAGTGGCTAATTACAAACTA
ATTAAATTTAAAGTACTTCGGCATTCCGAGTCCATGCATGCATTATATTAGTACTAAAAAGC
ATAGACTAGACTATCACTTTATTTTGACCACTGTTTTTACTCTATTTGTGCGGGTTATCTGG
GAAGAACTTCTCAGATAAATTGTGTCATGTTTGGATCATCTTCTATAAACTTTAGAGCTCTA
AAAAATTTTAGAGCACTTTAGCTCAGTTTTGAAATTTGAAGCTATAATATGACGTGGGCTAA
AGTTTAGAGCTAACTTTAAACCACCTATTTGAAAACTTTAGCCTTAAAGTTTAGAGCGCTAA
AGTTTAGAAGAGGGGATTCAAACAGGCTCTAATATAAGTGTGTCGGCCTTGTATACAAGGCA
TTCAATGACGTTTTGTTACGCAAAATTCGTGGGCTGTGATTTAAATTTCCATCCATGTTGAC
TAACAGGCTACTTTCATTGACTATCTAGAGCGCCACTCCACGTACACACTGTTATGTGTCAT
AAGGTTTTCGAGCGTTCGCTAGATTTCACTCTCTAATTAAGGACGGAGCTTGATCTATAAAT
AGATGCATGCACAAACATAGTAGCCACACAACACAACATACATACATAGACGACGATCATG
SEQ ID NO: 38
Sequence Length: 3003
Sequence Type: DNA
Organism: Sorghum sp.
AATTGAGCGTAGATAAAGAAACACAACCAAAACAACCTCTAGGGGGGATGGAGCGGTGTAGC
CCCTGTTACCACCATATGGAGATTCAAGCCCCCAACATCAGCGATACCTCCTCTATCATGGC
TGTTCATGGCCTACATGGAGTGCCATGCTAGGACGATGAGGTCATCTGCAATAGGATGGCAG
CTGCCAAAGATGGCAAGCTTTGGTGTCAGTGTGCAGCCTACCACCACTCTGCGCCTCAAGTC
TATTGTGATTGATCCACAACAACCAATCCCTCCCCCAATGTGGTAGGAGGTTTGGAAGAGGA
TTTAGTGGTATAAGATGTAGCCGACGAACCCAACACAAATGAATCTCCAATGCAGGTACAAA
AGCTCAAAAACCCACTGCCTGGTGCACAGCCTTTTCCAATGTCGTTGCTTCATATGCTTAGC
CACAGACCATCAAATTGTGTAGTTTCATGACACCTGGAAGTGCATCTTATGTTTCTTCTTTG
GCCATCACAAAGCAAGCTCTTGTTCATCTAAAGACATGCAATTTCCATTGCAACCCACAGAC
TCCACAATTATCTCCTCTTCCCGATAGCATTGATGCCACCGCCACTTCCGAGCATCCACAAC
CATATGTCTTTCCCTGAGCTACCTTCTCCCCTGCTACCAACGTGCCCTCCCATCCAACAGTA
ATCATGACCTACGCCTTGAGGTCACACATTGAATGGGCCAACACTTGGCTTTGTTGCGGTAG
TGGCTGCCCCCACCATGGTTCTCCTCCACATTCTTAACAGTCGAGTCTCCCCGCAACATGTT
GCATTGGAGTCCACTGCTCTCACTCACTGTTGACGATTAGCATGATGTTGTCTGTGGCTACC
TAGGAGATGGACTAGCATGGCATGGTCTACAAGGTCCGTAGAAAGGGCTGGCTTGCTGCACG
TACCTTGGGTGAAGAGCAAATGAAACTTGACGAGCCTTCTCTACCACACCACTAGCTGGATC
TATATTACTTGATTTCTACAAGGTATAGTAAGATGAGGTGCTTCCTACTAACCCTTGCCTTC
CTAGATTGGACCCCATGGTTAATGAGTTCTACTCTATGGCTTGATCCTCCATGCAAATGTCC
TCCTCTCTTTGATGAGGATCCTACATGCTCTTCCCAATGATGCTACACCTTTGGCTACCCAT
GACAACACATCCACCAATATTGATCAAGTTGTCCTCCTCTAAGGTAAGCAAAAGTTTGCAAC
CAACAAAAAAAGTTTGTAGGCAATAAGCTCCAATGATTTCACCAAGCTAGTGAAGGCACAAC
ATTATAGTAGCTGCCTTGCTACCAATCCCTTAGCACCATTTGACCCGCGAGTTGAGCTTCAT
CAATCCTAAAGAATTTGTGGGTGAAGCGTGACCGCAATATTCTTAGTTTCTTGTAAGGAACC
TTTTCCGCTCGAGGCTATCACAACAATCCAGTCAATAGTCCGCTTCAACAACAATGAGAAGT
ACTCTAAGACTACTATTGAGGCCTTGTGTCACTAAGGATGAAGCTTAGGTTTCCGATGAGTT
TCTAGTTGCAGCCATATGTGTTCTTTGGTTAGTTTTCTGTCGGCACACAGTAGATTAGCTTT
TAGTCATTTAATTTAGGTCTTGAGTTCTGTTTAGTTTTTGATGTAGGCTTTGGAGTTGTTTC
TTCTGGGTCGTAATTGTTCTAAGACGAAGCATCAGAGCCGAGTAAGAGCAGATCCTTTATAG
TTGTTAGTTGACAGTCTTAGTATTTGGCTACTTTATTTTTTATAACCCCCCCCCCCACCCCA
ATAGCAGTATGCTAATTTGGGTTTGATGCAGCTGTGCCTCTAGCACAAAAACTCTTCATCTT
ATTAATTTATGCGGACTTTGTCTTACCTTTCAAAAAAAGTGTTTTGGTGACCATATAGGTTT
TTTCTTTTTAAAATAGCACAATTTAAATTAGATAAAGAATAGCATATCCCTGCTCAATGTTC
TAGACCTTAGTGCTTTGACTATGATGGCCAAACGGGCGAAGCCCATTTAAGCATGGGTCTGC
TAGGCATAAATAACTTAATATGACGTGCATGCATGCTTTGTATATACTCTTAACACAATTCA
TAAAACTCTTGCTATTCATAGTACCATGGTGTTGTTTCTTATGAATCACAGTTCAGTTAATT
CTTGTATAAGTTGTTTCTATGACAACAGCCCTAGAATATATGTATGCGCGGTTTTCAAAAGT
TAGTTTTCGTGCCAAGCTTTATCACCATACATGATGCTGTGATAAACGATAGATGGTTATGA
TATACAATATGGAAGTATGGAACTAGCCTAATAGTTGACTTTATATAACCCTAAAACATCAC
TTATTCATGATCAAAGGGAATACAACTCAAGTATTATCACTTTGTGATAGAAATAGAATGCT
TTTTTGACGCTGGCAGGTATATGGGTGCTTGGAGAATAATATGATTAGAGCATGGTTTATTA
GAGGAGGTGCTTATGCATAGAAAAAAGATCATTTAATTGTCGCTATTCCCTCTATTCCAAAT
TATAAGACATTTTGACTTTTCTAGATTCATAGCTTTTGCTATGCACGTAGATATATATTATG
TCTAAATACAAAGCAAATACGTACTATGAATCTAGAAATTTCAAACCATCTTATAATTTGGG
ACTGATGAAGTATGCCATATGCAACAACCTTCTTATAAAGTGGGTAAAAACTAAATTTTGCT
CTTGGTGTGTGTGTGCGTAATAGACAAATCAATTGTCCGTTTCGTGTAACCCATTGATCAAC
CATTAGTCCAGCTTAAAACATCTCACTAATGGTAACTAATGCATCATGAATTTCACCCTATA
GCATTAATATATCCTAGCAGCTATAAAACGGCGAGACTGAGGTGAACCTTCGCAACAGGGCT
TGGGGTGTGCGAGAGAAAGAAGTTAGAAGAGGTAGCAATTAGCATAGGTACGGTCTCTATTG
CATTGGGCCGGGCCTGCTCTTGCGATG
SEQ ID NO: 39
Sequence Length: 1550
Sequence Type: DNA
Organism: Sorghum sp.
TTTCGTATTTGCATGTTCATGGACGAAGGTGGCACATGTCGATAAGTTTAGGGGGCACTTTT
TCGCGTTTGCAAGTTCATGGACCAAAGTGGCACAGACGGACAAGTTCAGATGTCACTTTTTC
GGGTTTGCAAGTTCATGGACCAAAGTTGCACATGCTGACAAGTTAAAGGGCCGCTAGTGTAT
TTATAATGAACTAACTAGGTTTAAAATTCGTCTCATGATTTTCAACCAAACTGTGTAATTAG
TTTATTTTTTATCTACATTTAATATTCCATGCATGTGTTCAAAGATTCGATGAGATGAATGA
AAAGAATTTCGAGTTGGGAACTAAACAGGGCCTAAGCACTCCAGCTCCAGACAGTTACACAG
CCTCTGCCTCTGTAGTACGTGTATCGGTGTATGGGCTTCCGTCGCTACCAACCGCCACCGCC
GGTACGTTCAACTCTCATGGTAATGGCGGATCGAACGACTTTCGTCTCAAACCACTCCCTCA
CCAGACACCAGCGGCACTGCATGCGTTTAGCCGTTTACCGTGGTCAGTGGTCCGTGGACGTG
GACACCCCTGCCCTGGCTGCACCTGCAGCATGCAAGTACGCACGTACCACTCGAATCCAATG
CCATGGACGGGAAGGCCGGCAAAGTGGTGGCCAGGCCGGCCAGCCGCCGCGTCCCTTCTCCC
CCGGGCCACGGGTGCAAGGCAAGGGCAACCGAGCCGGCAGCCGGACGAACCTTCCGCGAATC
CCAGACCTGCGCGCACGTCCTTGGCACATGCCACGGCCGGATCTCGCCGCGGGCTGGCACTA
GCAGACGGGCAGCACGAGCGGGGCACGGCAAGCAGCGGTGCCCTTGCCCTTGCCCCTGCCCC
TGCGTGCCTGCCCACCTCCAGCCGGCGCCTCACTGTAAAGCAGCGAGCGCCACTGTGCGCGA
CCGGAGCACGCAGAGGAAAGCAGCGCAAAAAGCTGCACGCGCGCTTTCCCCCGCCCGGCACT
CGGCACCCGGCTCCCGGCACGGCAGGCACCCCACCACAACTTGCCAGCCTAGGCTACCACCC
CTTTCGCGGATGCCGCCGGGGTCGGGGTGGACGCGTCCGCGCGCGGCGCGTGGGGCAAGTAA
AGGCGCCCCGCGCCCGCGCGGCCCCCACCGGCGTGGACGTACGCGCGGGCAGGCAGCCTCCT
CCTCCACTGGATCCAGGGTGCGGCCAGCCGTGTCGTGTCCCAAATCTACCCGCATTCACTCT
GCCAGCCCACCCGAGCGCCGGAGCCGCCCGCCACTCGCCCGTTGGTTCACCACCTGCTGCCT
GCCTGCCTGCCTGGCTAGCTGGCTCCCACAGTGCCACGGCTAAGGAACCGCCCCCCGGCGCT
CCCGTATAAATACCACCCCACTCCATTGCCGTCCCAACCCACTCACACACCAGATCGAAACA
TCTCTCAAGTGTTCAAGTTCCTTCCCAGTTCCCACACACTACACAGACCCCACTGTGTCACT
AGCTAGAGCTCCGGCAGCCACAAACACGAGCTAGTAGAGCTGCCAACAAGAACAAGAAGATG
SEQ ID NO: 40
Sequence Length: 3125
Sequence Type: DNA
Organism: Sorghum sp.
TAAACTCGTTCGCTTGTCTTATCAGTCGTACTTTTTCTGCCAGCCAGCAGTGTTTTTCGCTC
ACAATAAATCAGCCAACAGTAATTCAGACATGACTCACGTAGGCTTTTTGCTAATGTGGCAA
GTAATTCAATGAAGAGAGATAACAAAAAATTAGAAACCATTTCTAAGATAGAAACCATGCTT
CACGTGCAACGAAGGAGTTAGCCGAAAACCATCTGAGATTACGTTGGGGAGGGGGGAGGAGC
GCATGGTTTCCATCAGTGATTGGGGATACCATGCACGCTGCCGGTGAGAGATAGCGCGGCCA
CTGCAACACTTGCTTGTGCGGTTGCTGGTGGGAGCTTGTGCGCCACCGCCACTGGGACCTCA
CAAAGGTTGGTGGTGGAGCTCATGCGGACACTGCCACACATGCTCACGCACCGCCGCCACTC
AGAGCTTGCGCACGTGCCGCGACACGCTCATGTGCCACCACCGATATCTCGTGCAGCGTGCT
GGGACCTCGTGTGATCGCTAGAACCTTACGCTGTCGTCGGGCAAGGTGGTGGTGGTGGTGGA
GGAGGAGGAGGAGGAGATGGTGCCTGGTGTAGGCGGAGCGGACATGTCTCCATAGTTTCTCA
AAACATATTCCTAAACCTACTTGTTTTTCCTAAAAAATAGAGAAAGATTGAAATATGAGTTG
GATTAGTAATTAATGCTACATATTCAACAACCACTCGAAGTGAGTTATGTAATTCTATATGT
TGGGCTTGCTTAGATCAGTTAGAGCTAATTAGCCCAAATTAGCTGGGATTGGTGAGTTAATT
AGTTGGCTAACAACTAGTTGGAGGCTTGGTTAGAGGTTTGTTTGAATGGGCTAGAGTTAAAT
TTGACTACTAGCTAACAATTAGCTCTATGCATCCAAACATATCCTTATTTGGCTAGACTATT
CAATATTTTAGGTTGACCTAAGAAAACTACTCTTTCTAGTCAACTTTTAGCTTGACTATTCA
AAATGGATATATAAACGTGCACTACAAGAATAGTGTTGATCCGTGACAGATTTTTTTATCAT
AGATTTAGACTTGGGCTTGCTTGAAGCACATCCTTGGGCCCAGTTTCTGTGACCATATTGAA
GAACCGTCACAAATTTGCATGAAGTGCTGACGTGGCCATGACCGTGTGCCTAAAATTGCCTG
AAATCCAAATCCGTCATGGATGGGCATTCGTGACGGATTTCGAGCCTTTTGTAGTTTTGTGA
CAGATTGAATCCGTCAGTACCTTTGGACAATGTATAGAATATTAAATATAGACAAAAATAAA
AACTAATTATACAATTTGTTTCTAATTTGGGAGACGATTTAGTTAGTTCGTGATTGGACAAT
AATTGTCAAATACAAATGAAAGTGTTACAGTAGTCAAAGCAAAAAAAATTTGCGAACCTAAA
AAGGCCTTAGTAAAGTAAAGTAATACTAACATGTGGGATCTTCTAAATGATTGTGTTATATA
CAATGAAGTATCACTTAAAATTGAATTGAATTGAAAATGGCGCGTATATATATAAAAGCAAC
TCCAACAGAGATGATAAAAATAGATGGCTAAACTTATGATTTAGCCAACCTCTAAAATAGAA
ACCCCAACAGAAAAGGAGGAAACTCCAAAAGCCTTTCCAAATGGGTGAGGCGAATGGCTAGT
GGCACATACAAGGTATATCTAGCAAGCAAGAACTCTAGGGTACATAACTTGCTATTTTAGAA
AATTAGATACAATAGTTGTTGGACTCTCTTTTTCAAAATACTCAAATATAGATTACACAACA
TGAATAGCTCTTTTTGTTGGAGTTGTTCTAAGTGTACTCAACTTTACAAGATTAAATTAACT
TAACGAAAATCAATACTATGCGATTCAATATTTAACAATGTATTTGAATTCTTAATAAAGTA
TACAATTCTTTTAAGAAATAAAATAAGATAAAACCATGATTTTTTTTGGATTGATCAGTTAT
ATTACTCGATCCTGTTAGGTAGAAAGATCATACTACCTGTTATTCCGATCGTCGAAAATAAC
CTTGGTCAAGCTATGCCTACTTAAAAAATGAGCAAGGAGTAATTGAGTACTAATTAAATACA
TACAGTAAAATAATACCACAATAATAATAATAATAATAATAATAATAATAATAATAATAATA
ATAATAATAATAATAATAATAATAATAATAATAATAATAATAATAATAATAATAATAATAAT
AATAAAATACTTGTGAAAACCCACGACCGTTGCCACTGCCACGGTCGTCGCTGAACTAACCC
GGCCGCTGGCACGGGCCAAAAAGTCCAGGCGTCCACAGCTCATCATCATTTTATTTCTCGCT
GAATTTGTGCACAAAATTTATCAAATCCTTGGCACGGTTGCAAACCCAAGCCAAAAAAAAAA
ATCGACAACCAGTCGGAATTTTCTCCGACAATAGTCCTACTATTCAGCTCACGCCTGGAACA
CATCACTGTCATGGCTTTGTAGCTAGTGAACTGAATTTTCTGAGCGCACACAACGGCATCAC
CGGCCGGTCACACTCACAATCACAACCACTTCGGCCGGCTCTTTGGTTGCGTTTGCGCGAGC
ACATCTCCCAAATCTCTACGCACTTGACACTCACGCTAAACCTACCTAATTAACGCATAGAT
TAATCATGTCATCACCAACAACGCCACCAGAAAAAATGGACCCTACTTACACTACTACCTAC
TTATCATCTTAAAATCACTGTCCATGCATTATTATTAGCATGCATATATAGGAGATTAGCAG
TATAGCTTTTTCTTAGTGCCATGCATCTTTCATGCTACCTTTTTTCTTCCCAAAATTTCAAT
CCATTGTTAAATAAAATGCAAAAAAAAAGAAAAGAAAAGAAAACAGTTAGTAATTAATTGAC
TAATTGGTAAGCTAGTGCGTGATTTGGTGTGGTGGTTGGTGAGCTCTCCGGCCCCATATAAC
CCCCCTCCCTGCTCCTCCTTCCTCCTCGCAGCAGCAGCACACGCCAACACTTGCCAAGCTCT
CGCGTCGCTCAGCGCTAGCTCCTAGCTAGTATCTTCTTCCACCGGGCACCGGCCGGCCAGCC
GTCGTCAGCTAGCTAGCTAGCCATG
SEQ ID NO: 41
Sequence Length: 3036
Sequence Type: DNA
Organism: Sorghum sp.
TCCAAACTTATTACAGTAGCACACTCATAATTAAAATTTGTTACAGGTTTAGAACTAAAGTT
CAAAACAGCAAATGAAGGATCATCCATGCAATGCGAATAACTCCATGGCTCTCGATTCTAGT
AGTTTATGTGTTCTTATAGTTCTATCATTGTTCTTTTTCATTGTCATATGGTCTTTTCTTTC
GTACCTTTTTCTATATGCTTATTATTTGACCTCTCGACTTGAGCTGTAATTTTTCTATAATA
TTTAATACAATTACAAATTTTAAAACTCCTTTTAATATAAGACAAATATTCAAGCACTTATA
ACTAACGTGAAGAAGCTATTTTATAGTACCATAGGCAGGAATAGATACAAGGGTATTTCTAG
GTTAAATTTTTGCAATGACAATGGTGGTTAGTTATTAGTATGTAGATGTTTTAAAATTTTAT
TTTAGGCCTTGAGATTTAACATGGAGACTTAATAAGCAATGTTAGAGCAACTCCAAGAGACT
CTTCATATTCTTTCTAGTTTATAGGAATAGAGATTTTGGTGACAAAAGTACTTCCCAATATC
ATCTTTAATTGGATCCCCCAATATAGACATGCTCTATTCCAAAATTCTCCCTAGTTAAAGAT
AGAGAGCGAGGATGACTCTCTAGAGTGCGCACAAGATACAAAAAAACAGTTGGAGGGTACAA
CAATATATAAAAACAATTGTAACTCAAATGACTCTCCAAATAATAGCTCAAAGAGTAAATTT
TAGAAAGACTCTTCGGAGATGCTCTTACAATCAACTAATACTTCAATTTATATAGCCACCCA
TAAGGACATTGGTATAACTTGCAATTATTCTACTACTTCCTCTTATCATAAATGTAAGGTTA
TCTAAATTTGTTTTAGGACATATTTCTAACTTTGATTACAAACGGTTGATTGAGTATTTGTA
TTAGGGCTCAAACAAAGCCTCCTTCAACTAACAAGAATGGTTCAAAGGCTAAAATTCCCATG
TCATAATATAAAGGATATAAATTCCTTCTTATCGATACCTTGGGATTTTTTAAATGTTTGTG
TGGGAGACATAGATTGTATATGATCCATAGTTCTCCATAATATTGGTACTCATACAAAGTGA
GTGGGCACAATTTGCTATCTATAGTACATGAGATAAGACAACAAATTATGACAACTCATGGT
GAATTAATATCCTAATTTTATTATTTCTGTTTACTTCATGGTGTATTATTGTTTTGATAGTA
CTCATGAGATATTTTGTGGTGTTTATCTACGACTCATGTATTATGTATGGCACATCCCATGG
TTCCTATAATATTTCTTAGATGTTCGATGGTGTCCTTTTTATCATTATCATATTATTCATGA
GATGCTTCATGGTGTACATCTCACTGTTTTCATTAGCCATTGGATTTTCCATGTTGTATTTT
TACTACTCAAATAATGTTCATTAGATGTTCCATGTTATATTTTGATTGTTCTACTATCATTC
AGAAGATTTTATTCGTGGTGCATAGTTTATATTTTATATTTTCATTAAAATTGAGCCACAGA
CTTCTATTTAAAGTCATGGCTGAAAGTACGGACTCTCTATAGATACTAATGGGCCGTGGATT
CGATCAAGGATACAGAATTAGGCCAGTAATTCCCAGGCATGGGCTGCTAGGACACTGGCCCG
TTGGAGAGGCCACCGAACAGCGCAAGAAGCACCACCGCGTGGCCGGGCCAAATTGTAGCAGA
GCAGAACCACACTTCTTGGACTTGGCACTTGGGCCTCTCTACCCGCAAACTCTCTACCACGA
CTCTGCTGCCCTGACTTCGTTCGATTGTCGACTTGTCGTTGAGTCGAAGCGATGTTGCACGT
GGGCGAGCGGGTGTCATTTGTTTCCAGGTGGACCCGAAAAGACTTCGAGCTGGCTTCACTAT
ACGAAACCGCCCGAAGGCCGATCGTCCCACAGGAATCAGGCCCACAGGAACCCAACGACCAT
GTTTCTTCTCCATCTCACCAAACATCGATGAGGCAGCCACCGGCACCAGCAGACAATTGAAG
CAGTGGAGATTTCAAGACTTCAAGTTACAAAACAAAATCTATATAAAAAATAGTAGTAGTAG
TAGTAGTATCTCTCGGGGTCATTTCTATCCGACGACAACCAAAAACAAAAGAAAGTCTTATC
TGCTCTCTCTCTCTCTCTCTGTCCCACAAGTTTCCATCGTTTTGAATGCCGAATGGGGCTTG
TGTTGGATCACATTCTAGCTAGTCATGTGCTCTTGTGTACTAGTGACAAGGCTTGGAAAATA
CAAGGGATCGGTCAACAATATGATCCACTCCAAAGTTGTCAACAAGCCTTTAAATCAGCAGG
AGCACCTGGTATCCTCCCTCCAGGGGGTTGGTGTGGTGAACACTGACCCTCAAATTTTTGGG
CAAGTGCCTGTGCTCCCGAAGAGCACACAAGTAAGCTCAAACCAATCACCATTAACTTTTTT
TTTTCTTACTGAAAGATGCCTAGGCATCTTATTCCAGAGGCCTTGTTTAGTTTAATCTAAAA
ACAAAAAACTTTTTAAGATTTTTCGTCACATCGAATCTTACGGCACATGCATGAAACATTAT
ATATAGTCGAAAATAAAAACTAATTACACAGTTTGACTGTAAATCGTGGGATAAATCTTTTG
AGTCTAGTTAGTTCATGATTTGACAATAATTGATAAATAAAAACAAAAATGTTACGGTATCC
AAAATCAAAAATTTGGAAACTAAATAATGCCTGAATTATATTAAAGGAAAAAACCTTTTTAG
CGGCGTGAAAAACACTCAATTTTTATATCTAACAATTATTTATAAAAATAAAATACACTGAG
CTGAAAGAAAGTGGAAAGACGAGTAAAGGGGCAAGTCAGCGGGCCCCAGCCCCACTCACCTA
CCGCCAACCGCCCCCGAGATCTCCCTCTTCTTCTCCACTCCCGTTTCCCGCCGTATAAATTC
GGCGAACACCGCACCACCATTTTCCACCAACCCCGGCGCCCGCCGAGTAGCCCAGCCATG
SEQ ID NO: 42
Sequence Length: 3064
Sequence Type: DNA
Organism: Sorghum sp.
AGTCATCAAACAGGCTTCACATTAACTGATTGTTTATATAAAGTTTGATGTGAGCCATCAAG
AGAAACTTCGATAAGGATATAAACGTGAGGTATGATAGGTTTTCTGTTTACTACTTCAATTG
TCATCTGCAGTCAGTCCCAACTTGATGTCATTTTAAAAGTTCCATGTGGAAGGCATGCGGAA
GATTACGGCCTTGTTTAGTTTACCCTGAAAACCAAAAAGTTTTCAAGATTTCTCGTCACATC
GAATCTTGTGACACATGCATAAAACATTAAATATAGACGAAAACAAAAACTAATTACACAGT
TTAGCTGTAAATCACGAGACGAATCTTTTGATCATAGTTAGTCCATGATTGGATAATATTTG
TCACAAACAAACGAAAGTGCTACAGTACCGAAAACTTTTCACTTTTCGGAACTAAACAAGGC
CGGATGTCACACTTTCTTTTTCAGGGCAGAGATATGATGACAGTAGTGGTTTCTAGACCATA
AATAAGTCATACAAAGAGATCCATTGATTGTTATTATCTGCTACCAATAGGAGATAAAAGCA
AGTTCATATAAAACATTGAATCTCTTTTATAACAACAGAAAAACAGTTTATGTCTATGATGC
CTCCTCTTCCGTACTGTATGGTACGAGAATAAAGTAGAAAAGATATGTTTCTGCAATCAATA
AAACTCCTCTGGACTTGTGCCAAAGAAAAACTTCATAAATGTCTATGTGAACCAAACCACTC
ATCTTTTTTAAAAAGAATGATTTGGTTCAAATCTAAAATTACACTCTTTTTTTGGAACGAGG
AAGTACATCCATACAAAATTTCTCAAAATTTCAAGTACAAACCCAGTCATGTTTATATATTA
TGAGATTAAAAAGGCAAACTTTGCCTGAAATCGATGTGAACATGTTCGTTCTAGTTCTGTGC
AACGCATTCATTTGCATCTGAAATTCCACATGGGCCTTGTTTAGTTCACCCCAAAACCAAAA
AATTTTCAAGATTTTCCGTCACATCGAATCTTACGGCACATGCATAAAGTATTAAATATAAA
TAAAAACAAAAACTAATTACACAGTTTAACTGTAAATCACGAGACGAATCTTTTGATTCTAT
TTAGTTCATGATTGGACAATATTTGCCACAAACAAACGAAAGTGGTACAGTAGCGAAATCCA
AAAACTTTTTGCATCTAAACAAGGCCATGAATGTGGAGGACACAACGTCACCTATGGATGGT
CGCGAAAATTTTGAAATCATCTCTGTAGCTGATTTGCACGAACGATCAACTCATGAACATCA
CCTTCGCCCCGTCGCCGCCGGTGGCTTGCCGCCATTGGACCGACGGCCGGCGGCCGAGGCCT
GCCACTAGTGCCGTGCCGTGCCCGGCGGCCAATGATGCGGCATTGTGGCACGTCACCGAATG
TTGGCTAGATGCTTTTTGGCCAGCAATTACTTTTTTTTTCCTCAAATGTGACTAAGAATCAC
TTTGCTGGAACAATTTTTTTCCGTGGCTACAAAGTGCAAGGATGATGGATCGCTAGATGCTT
CTTGTGTTCGTGGCTGTTGATTTTTTTTAGGAAACGTGGCCGTTGATTTTTGCACGCGCCAT
TACCCTAACGTGCGTTTTTTTTTTCTTTCCTTTCTTGCGCATACACTTTAATTTGCTCATGA
TTAATTACTGGGTAATCTCGGATCAAGAAATATAGGTGTGGTTAGCCACGAACTCAATTTGA
AACTAGAAACAAAGCATTGGCGTTATGTTTTTTTTTTTTGAAGAGAGCATTGGCGTTATGTA
GGAACTCCCTTTTGAATTAATGGCTAAAGTTAGGTCTGGTTTAATTTCCAAAAATTTTCAAC
ACATCAAATCTTTGGACGAATGCATGGAACATTATATATAGATAAAAAAACTAATTGTACTG
TAATTTGCGAGACGAATCTTTTGAGCCTAGTTAGTCTATGATTGGACAATAATTACCAAATA
CAATAAACGAAAGTGTTACAATAGCCAAAGCTAAAAATTTTCGCGAAGTAAACAAAGCCTTA
GATATGCTTGCCTGTCATACTATATTTATAAACAAATATAAAGTAATTCACTTAACTGTCCG
GTTCAAACTACAGTTCATTCTATTTTTTTTAAGTCAAGCTTCTCTAATTTTGACAAATTCTA
TAGAAAAGTGCACAGATTCTACTGCATGAAATTAATTTCATAAATTCTCTGCAAAACATGCT
TCCTGTTGCATACTTGAACTTGTAGATACTAATACATTATCCTAAAAACTTAGTCAAAATTA
GAGAAGCTTGGCTTAGCATAGAGCTAAAGTCTACTATAATTTGAAATTCAGGATTATATATA
CATATATCAGTAATAAAACTAAGCTAAATTTTGCTATTGAATTATTGGTCGTTTATGATATT
CACTTATTAGGGCCAACATTTTTCGCTTGACAACTTACACAAGTTAGATACGGGCATACGGC
GTTACATTAGTTACACAAGTTAGACACGGCATTGCCTGGGGCCTGGCTACACACATTCGCTT
CACAACTTACACATGTGCAATTTTTCTAACACGACCTCTCTAACAAATGTACTTCATCTGTC
CCTAAATGTTTGTCACCATAAATTATATGCCGATAACTTTAACTTAATTTATAAAAAAAATA
TATAATATTTTTATCTCTAAATAAATTTATTAAAAATCTAGATTCAAATATCTATCTAATGA
TACTAATTATGTATCATAAATATTATTATTTTTAATTTATATTTAATTAAATTTATTTTTTA
AAAAATGAAAACCGTATACATCTAGGGATGAAGAGAGTACACAATGTAGTGGTACCAGACAA
GCAGTCAGTCCAAGCATCTCCACACAAACTGTTGTTCAAACACGCAGTCTCACTTGCTCACC
TACTCCAAGTCAATGTGGTAAGTACACTTTTACCTATTAACCTATTTATTAGATTATATAAG
CACCACCACACATGCATTTTTACCAACACAAGCCAGCCAAGCTAGCACACAACATGCAAGCC
AAACTTGACCGAAGCCGCCACTGATG
SEQ ID NO: 43
Sequence Length: 3021
Sequence Type: DNA
Organism: Sorghum sp.
TTTTAAGCAACCATAATTAATCGTCATAATCCGTTGAGCAAAACACTTTGATTATTATAACC
CATAATCCTGATTATGGTAATCTTATATATAATCCATATTATAATAATCAAACCATGATCTA
AACAAGCCCTAAGTTTTTGAAACCGATATAAAAGGTCTCCCCTGCTCCCTAAGTTCTCTCCA
TTTGACATATTCTTTTTTTCCATTTTTAAAGCCTACGTATACATTACGAAACAAAATCACAT
AGCAATTCTTTGTTTCCTTTTCTTTTGAGACCGTACACATTAGGAAACAAATACTACAAAAT
TAGAGAGCCACAGCCACATGCTTTGAGCTCAGTCCGTCGGTGCAGATTGGAGACTCGAGTCC
TGGACACGGACTCGGAGGCGATGCCGACGGCATCCCGTGTGTCCGCCATTCACACAATTCAC
AGCATGGTGGTCCGGCGGCCGTCCAGCCTCCAGCGAGCGAAGAACGACCAGTCACCAGCACC
TGCTTGGAGCGCGAGCGCACCCTCATTTTTCGGTTGCCGCCTCCGTCCTTAGTGGCGGAGCC
AGAAAGTTTTAGGGTGGCTACCTTTTAGTCAGACTTCACAAAAACATAGGGGTGATCGTTTT
TTTGGGAGCCAGGCTTGTCTTAGCTCCGGTGGGTATTTTTCTCACCTTCCATCTTTCCGTGC
TCGCTCACCCTTCAAAGTCACCGATGACTTGATCCGGTGGTCCGCATCTGCACCTCCTCTGA
TGGCTCGCATCTGCTGTTCCGGCGCTCCTCCCATCGGCGAACTCGCTAGACAGGGCCTCCCG
CGGTGGCTTGCTTTTGGCCGACCTCTCTCATTGGCTCGCCACGGCTACAGGCACGCAAGTTG
AGTCGTGCGTGCTCCTAACCCCATGGACGTTGCCGTCTTCCCTGGCAGACACCGTCGTATTC
CCTCGACAAATGGAGGCGAGTGGACTCACCACTCGGTGGAGGAGGCAGAGAGGAGGGGTGTG
GAGGAGGGGAGGCGCGTACGGTATTTTCTAGCTTGGTTTTGCTGTGGCATTACCAAGGTCGG
AGGATATCCCGAGGACGACGATGACAGGACGGTCATTGGCAACTCCGCTTGCGCATTGGACT
AGGGTCTTGTTTACTTTCACCGAAAAATCTAAAATTTTTCAAGATTCCCCATCACATCGAAT
CTTTAGACGCATGCATGTAGTATTAAATATAGATAAAAATAAAAACTAATTGCGCAGTTTGG
TCAAAATTTACGAGACAAATCTTTTAAGCCTAATTAGTCTATGATTGGACAATAATTTTCAT
AAACAAACGAAAGTGCTACGGTGCCGCGAAATTTTTTTCCTCAAGAAGTAAACACGGCCTAG
ATGTACTCCACGTGCAGTAGAAGTGCAGCAGCACCATCCACCGTCATCAATTGCCAAGCTCT
CCTGGCTATGTGTGCTTTGCTGCACACCTGTAGATTAGTATTAGATGTGTTAGGGATTTGAA
TTTGGTGAAGTATTGGTTTAGATTGGATGCATATACTAATAATTAGGATTGAATTGGATAGG
ACTGATTTTAATTAGATTTGATTTGAATTCAAATGTCTCAAAGGAAGGTGCTCACCATGTAT
TCAATGAATTGTCACAAAGAAGAGGTAACTCTATCTAAGTCAAACCAGCCAACCAAACAAAC
GACATGCTAGCTAGGCTTAAATAGTAGAGTGAACCAAACAAGTCTATCTTGACTTGTTGAAG
GCAGGCTTAGGCTAGCCTGGCTTATTTCCTAGTCAGGCTAGAAGTAAGCCAGTTAACCAAAC
AAACCCTTACGAGCCGAGGCGATTTTCATTATACATAAATGTTTATCTAGAAATATATAATA
TTTTTCTATAATACAAAGAAAATTTCCCTTTGTTGTGTATGGGCGAGCCCGCCCAGCCAGCC
AAGGTCGCCGGGCAGTGGCTCCGCCGGTGTCCATCCTCACTGTCGAGGGAGATGCCACAACC
AGAACTCGGGACTGTTTGGTTTCCAAATTAAATTTGAGTCAGTAAAAATTTTAGTTACTTTA
GCAGTTAAATTTTTAAATACACTAATTTTAAAAAGAGTTAAAATAGTTTAATCCTATTAGTC
ATCAAAAATAACTAAAGTAATTTTAATTAACACCTTATTTAGATGTGAAAATTTTTTGGATT
TCACTACTATATACTTTCATTTGTATTTGATAAATATTGATTCATCTTGCGATTTACAGGTA
AACTGTATAATTGATTATTTTTTCATCTACATTTAGTGTTTCATGCATGTGACATAAGATTC
AATATGACAGAGAACCTTGAAAACATTTTGGATTTTGGTGAGAACTAAACAAGGCCTAACTA
AAATTTAGTAAAGAGAACTAAGGCCTTGTTTACTTTCACCCCAAAACCCAAATATTTTCAAG
ATTTTCCGTCATATCGAATCTTTAAATGCATGCATAGAGTATTAAATATAGACGAAAATAAA
AACTAATTGCACAGTTTGGTCGAAATTTATGAAACAAATCTTTTGAGTCTAGTTAGTCCATG
GTTGGACAATAATTACCACAAACAAACAAAATGCTACAGTGTCACGAAATTTTTTTCTTCGT
AGACTAATCACGGCCCAAATAACCCTCGTTCCTTCCTTCATCAACCCCTGTCGCAACCCAAC
CGCAAGAAGGGAGGGGCGCGACCCGTCGGCTCGTCTGAGCCCCGAACCCCCTTTGACTATGG
GCCCGCCTGGCCGCCTGGCCGCCCGTAGCACCAACCAACCAAACCAAAAGCCATACCAACCA
CCGCGATCGCAATTCGCAAACCAAACAAAAAATTAACAAAAAATTCGTGTACCCAAATCGGA
CCCGTCTCGTCTCGCCTCCCCTCCCAAACCGCTATAAAATCCCTTCCCATTCCCCTCCGCCT
GTTCCATCGCCTCTTCTGGCAGACGGCCAACAACAAACAAAACAGAGAGAGCCACACACCCC
ACCTACCCCCACCCCGGCGGCCGGGCTCCACGCTCCTCCAGCATG
SEQ ID NO: 44
Sequence Length: 2100
Sequence Type: DNA
Organism: Sorghum sp.
TGGGAGCGGCATTGTTGACGAGACTTTGACTTCACAATACACGAGTGGGACGTGTGGCCTGG
GCACACCGACTACCGACATAGTGATATCAAGCCGCGTGGTCACACCGTCATACTCAATCCAC
ACCTGCAGGAGCTTCCCACTGGCCAGGGCGATGTCGACGGACTTGGTGGACTTGGAGGTCAC
AATCGAGGTCACCCCGTAGTTCACCATGGATGGAGGCGGAGGCAGAGGTGTTCCGTTGCTGT
TGCAGTTCGCCTCATAGCGCGTCGCCGGCAAGCTCCGGGTATCGTTGTAGTACGACAGTTCG
CCTCGCCATGAGCTGGAAGACGTTGTGTCCTCTCGCTGTTGCCCATGCTTGAAGGAGCTTTT
CCTTAAGGAGATCGCCCTGTATGATGAGCTCATGACCCAACCGAAGTAGAGCAGCAGCACCA
TGGCGGAGCTCACCATATGTTCGAGAGAATACTTCAAAGGGGGTGAACTCACCCTCATCCTA
GCCATGCTCGGCAGCGCACGCAACCAAATAAATTAAATGGGTCTTGACTTGCTAGGGACAGG
CTTAGACTAGTTTGGCTTAATTTTGCTAGCTAGGCTTGTCTAGGGGATAAACCAAATACACT
CATATTTTCTTGAATGGAATAATAAAATAGAGATATTTGTAGCCATTAAGGCATACTATTGC
ACAGTAACCAATTCTCTAAAAAAACTATTGCACAGTAACTTGTTTCGGCAAGTGGAAATAGT
TGTGTATCTGAAACCAACAATTGGCTGGGGCCGAGCCCACTGAGGAAATTTCTAGAAAAGAA
GGCCAATTTCGTGCCGGCAAGAAAAAATACAGGTACGTAAAATAGTGGCTCTTATTATGAGG
TCTTGCATCGATGTTCCTAAAATGAAAAATAAACGTGTTGGATGGTGTTTGTGTGTGAATGG
CGTCCATCCATCCATGAGATCAGAACGACAAGTCAAGCACGGCATATAGGAGCTAGCTTATT
AGTGTGGCTTTGCTGAGACGAATGAAAGCAACGGCCGGCGCGCATATTTTCAATGCGTGTAG
CTTTCAAGCTCGAAGATAAAGACATGACAAATGAAAGGCCGGCATCCGTGCAATTCAGGAAA
TTCGTCAACCAAGCTAGCCAGTGAACTTGCAGATAGATGCGTGTCTGTTCGCTTGATTGATA
AGTCATGACTAAAAATAATATTTGTTATTTTTTTATTAAAAAACAACTGTTAAATGGTTAGC
AAATTCGGTAGATAAGCTTAAGCGAACAGACATGTATATATGCTTCAATAATTGACACTAGT
TAGCTAGAGAGATGGTACATCATAAAAGAAACCAAAGTTTAATTTCCACTGATTATTAGTTA
GCTACTCCTATGTTCTTTTAGCTCAGCTATTAATGTTATACTCACTCCGTTCAAAATTGGAA
GACGTTTTCACTTCTGTTATGTATCTAGACGTAATGGTGCATATTTTAAAGGCATATTATCA
TAATTTGAAATGGGAAGAGTATTTTAATTTGGCACATCAACTCCTGCAGTTCCAATCAATTT
AATTTTGGTTTTGCACTTTTGCAGCATCTAATACGGTTGTCCCTTACTACTGAAATAGTATA
AGATATTCTATTGGGGAAAAAAACATTGGATCATCTGATACACCTCTTTGATTGCTAGATAC
TATTAATCCTCTTTCCTATGAACTTGATCGAAGTTAGGACAGTTTGACTTTGAACAAACTTA
ATTGGATCTTTTATATTTAAGGACATAAGGAGTATTACTTACAAATAATTAAAGTAGAATTC
GATTACCAGTTAAATTGAAATCGAAATATATACTCCAAGAATAATTCTGGAGACAAGTGGAC
ATTGGATCGGAGGCCAGGAGGACTTGTTCCGGAGAGAGCCTATGGCGTGCTGACACGGCGCG
TTGCGTGCCTGTGTTGGCTCAATAATTGGACGAAGCCGAATCCTCCATCCACTGCTATAAAA
ACCGGTGTAGGGGCTTCATTGTGCTCAAGCTCAACCAAAGCGACTTTGTACAACGCCCTTTG
ATAGATATTTGTTCTTGAGCTTCTTCGTTTTTGCACCAAAAGACAAGCAGGATG
SEQ ID NO: 45
Sequence Length: 3003
Sequence Type: DNA
Organism: Sorghum sp.
AGGGCCCATGGCCTGCGCACCTGCTGCCCCTCCCATAGGGCCAGCACTGCACCCGCTTCTTC
CCTTCTGCGTGTCTGGATCTGTGCACTGTGGATACTGGATCCGCCACCAGGGCTTCTCGTGC
GCCATCGCAGGGAGGGCGGCCCCACCATAGAGATAGGCGCCACCAGCGGGGGAAGGTGCCAT
CGGATCTACCGTAGGACGTCAAGGGTACAGTAGATCCACCACCGCCAGTGTGGAGGGGAGCC
GCTGCAGTCACTATGGGGGTGGAGGGGGCCACCACCATCGCTGTCAGGGTAGGAGAGGACGA
CCACAACTACCGTCAGGTGTGGGAGGAGGCGATGTTGCGGGAGTGAGGCGTCACCGCATGCT
GCCGCACTAGCCATCGTGGGTGAAGGAGAGAGGGAGCGCCGGGATGTGTTGTAGCTGGTGTA
TGGAGGGGAACGGGGACGCCGTCGTCACCGATGGAATTAAGGGTGCTCCACCGCTGCCATAT
CTACATGTCTGGTGCTCCGGATGTGCTGCTCGGGCTGCCCGTGCGCCACCATTAGGTGGGGA
AGAACCGCCCCGACCACTATGGGAGGAAGATGCGCCGGAGTAGCCACCAGGATGAGGGAAGG
GGCGAGGATCTCGCCATCTATTGAGGAGGCCGGGAGGGGTACTGCCGTTGCCAAGATAGGAG
AGAGAAAATAGCTAGAGAGAAATGAAAGACGACTAGGGTTAATGAGGGGTAGCATGGTCCAT
TGTTTTGAAATAGCTGGTGTATCCCAACACACTACCAGCTAAAGTTAGCTGTAACAATTAGC
TAGCTATATTTTAGCTAAGACATGGCATGCCCTGACTATCTAACAGCCTTTTGCGATTCCTT
TGATTTGGTGTATATATCATTTTTTGTAGAGTGACATGCATGAGTATAACTAAGAGGACACA
TGGATAATAACATATATATAATTTCAGCCACTCCGGTGTAGTATACAAGTGTAGAAGCGTGC
GTCGTTTTATATTTGAAAAACAAAAAATGCGCAGTATTGAGGAACAACGACGACCTAGGAGC
AAATTAATGCACAACAGTGTCTCTTAATGAAGAGACGGGATAGCTTGGTACGAGATTGGTAC
TGAGAGCGTTGTGGATGCATATGTTAATTAGCAGTAGCTACACAGGCACACAGCCCGCTTGC
AGTGGTTGGGACGTCAGTGTCATCAATGTCGGGTCGGTGGCAGGAGAGGATGGCGAAAACAT
CCAAGCAGAAAAGGACATCGCCGTTGGAACAAGGGACGAGTGCACCGCTCCGGCAAGCCGTA
CCGTACGCCTCCGACCCTGACCCCGCCACGGCGCGTTCGCTAGCTGCTGACTGTGAGCCTGA
CGCCTGAGCCTCAACACGGTCGGGTCGCCACCACACTGTGCATCATCCGTTCATCCACGACT
GTGCTTATTGCACAGCCACACACAACACTGCCCAGCCTATAGGGCAGCGACGTATGTACGTG
TCCCTTTTAACCTAGCTATAGTGATAAAACTGTGAATTTTCTAGCTAGATGAACTTTTGGGA
TGGTTTTTCAACCACGCACCGACGCAAGCTATGCGTAATTCAACAAGTTAAAGGTCTGTAGG
ATACTATTATTTACTTACAGGTCTGATTGACTGGTTTACCATCACCTTGGACCTGCAGGCAA
AAGCACGATGTCGACAACTGCCGTGTCGGTCACGCAGGAAATCCAAAGTTCTACGACGTGTA
TACGTACGGCGTGCGTAGCGTCACTCTCATACTCTCACTCACTACACAATCTGATGTCCTGC
AGTGGCCTGCAATGTAACCATGCATCGCCAATCATGTGTCTCACAGTGCCGGTCCTGTGTGT
CCTCTCCCTTTGGCGATGAGCTTCACGAGCTGATGCAGTGCCCCGCTTCCATGCATAGGTCT
TGTTTAGTTGCAAAAAATTTTGCAAAATTTTTTAGATTCTTCATCACATCAAATCTTTAGAC
GCATGCATGAAGTATTAAATATAGACGAAAATAAAAACTAATTGCACAGTTTGGTCGAAATT
GACGAGACGAATCTTTTGAGTCTAGTTAGTACATGATTGAATAATATTTGTCAAATACAAAC
GAAAGTGATACTATTCCTATTTTGCAAAAATTTTTGGAACTAAACAAGGCCATACATGATGT
CCACCGGTAGACATGCATGGCACACCAATCAGCTCGCCGTAGTACTATAGGATGATGATCTG
AGAGTTCCAGGACCATGCATGTGCTTGTGCAGCAGCGCGCGACAGGTGAAGATGCATGACGA
TGGCTAGCTAGCTCTTTGTCATGCATCCATCGTCCACACACCATAAAAATATCTTTGCTACC
TCTCAAAGCAAGATGTTCACTGTCCTGGGGATGAATCTTCACACATACAGTATACATAGCTG
GCTCGCTGGTCAACAGCGCGCGCGCGGCAGTTTGCGTCGTCAACCACAAGCTAACAAATACC
TACCTGTCGTCCCGTGTATCATCAAAAAAGTTAGCAGCAAACGTACACGTCGTCGGGTGTGT
GATATGCGCGCGGTGACTCGCATGGCAGGCAGCAGCGTGTATAGAGAGACTAGAGAGTATGT
TGGAACAAGAAATGGATGGAAGAATCCATGAGAAAGTAAAAGTGAAAGTTTTTCCTAAAAAA
AAAATTAAAAGTACTGAAAGTTACGTGCTACTGCTATCCGTTGAATAACATTAACACGGGGC
TTACCTGTTACCTACCCGTTGATACGGCGGAGGGCAAACGTGTTATTAGCTGGGCAGACAGC
CCATCCACGCGTCAAAACTTGGTTGGCTCTCGCGCGCTATAAATCCGACCCATGACCACACC
CCGTCATCCACACCACAGACACACAACAGAGACTGCACTCAGGCACTACCAACAGCTGCTCC
AGAAAGAGAAACAGAGAGAGCAACACAGAGCAGCAGAGAGCTAGCTAGCAGGCGAGCTTGCT
TGTTGCAGGAGCAGCGAAGCAGCCATG
SEQ ID NO: 46
Sequence Length: 3102
Sequence Type: DNA
Organism: Sorghum sp.
ATTAATCCAATTGATTATGTCATAACATTTTTAGATTTAAAATCATTTTGAAAATGACAATT
TTACAAAAAAGACCTCTAAGCTTTTCTGTAATTATTTGGAGGTAAGAACTACAACATTTTAG
TTCTTGGTTTTGAAAAATATTTACAGGGTCAGTTTTATTTACAAAAAAATCCCTAAATTTTG
AAAAGCTTAATCATAGCTTTAGGTGGTTTTTGCGGTGGGACAGTAGAAACAAGCTATATGTT
TTTTCTAATGCTTGTTGTAATGTTTTTTTTTGTTTTTTGCTATGTTTGTACCATGGTTCGAG
TAAACCATCACTTAGCCTGCTTAAACCTCATTTGAACATTGCATGGTGGTCTGTCATTTCTG
TTTATTCGACCATTGACGCCCATTTAAATAGCTGTTTTGCCCATTTCGTTGAGTTCCTAGCC
AGAAAAATGGCTAGATGACTCATTGTAAATTGTTTATCGTATCAATAGCATGTTTACTTTGT
CTACATGATGACCCGTAGCATTAATGCTTACTTTTAATTTTACAATGGTAGAATGTGTATAG
AAATATATATAGCATATTTTTATGGTTACTGAATTATTTTGTACAATATATATTATAGGTAA
GACAAACATTTGTGGCGTATTTACATTGTTTAGATATATGTAATCTAAAAAGGCACATTTAG
TGTTACTTTGTAATCTATATACTTTTATAAAGCTGGACCCGACTAGATGTTTTCTCTCTGAA
TGCAAGGTGTTAATATCATCCCTCACTTTAGTTTCTATCACATTGGATGTTTGTATACTAAT
TTAGAGTATTATTAAATATACACTAGTTACAAAACTCATTACATAGATTGTAGCTAATTTAT
GAGACGAATCTATTAAGCCTAATTAGTGTATGATTTTACAATGTGGTGCTACAGTAAACATG
TGTTAACAATGGATTAATTAGGCTTAATAGATTTTTCTTGTGAATTAGCCACAACTTATGTA
ATTAGTTTTATAATTAGCTCATGTCTAATTCTACTAATTAGTACCCAACATATGACAAGGAC
TAAACTTTAGTCCCAAGATTCAAACACCCCATAAGTGTCCCACTAACTTGCAATCCTTTCAT
GCAAGCTATCCATGTCATGCATCCTTTTTTTCGAATTACACAATATCTCTTATTAATTATAA
AATATATTCACATCATCTTTATTATGTGCAATACTTTTAATCTCTAACCTATTAACGTAAAT
GTCATCCCTTATTAATTACAAAAAATATCCTCATCATCTCTATTATGTAGATGTAAAGTTAT
TTACATATGTTATTTGTTTCATCACCTATTCTTGTATAACGCAATCAACTATTTTATTGGTT
TTTTCTATTAGCTTATTGTTTTTTTACTAGATCCGATACAATAAAATAACATGCAAGTCAAA
TTCATAAATATACACACAATATACAGAATACCATATAGCATTGCTATATACAAAACAGATTA
TTTTTAAGAAAATCCCGCGGCAATGCGTGAATATGTGTCTAGTACCTTTCATAGTAAAAAAA
AATGCAGACGCATTTTTAGGCATGTTTCGGTAATTAATTGCAATTTAGAAGGCTTTTTAAAT
TTATCTTTAGAAATGACAAACGTGGGGAATTTAAATGCAACTTTAGCGAATCGTTTATGTAT
TTTTCGTAATGGTCAAGGCGGGCGGCTTTTCTTTTTAGAAATATAGAACAGGTCAAATGGTT
GAGGAATGAAGCGTCGTACGCCCGATGAAGACAATGAGGAGGCGGACTCGTTTTGTCTTGGA
TAGCCCATGAATTTGAGGTAGCTAGGCCACAAATTCAGGCTCTGGAACAGCTTGGAATGTCA
CTTTCCAGTTCAATGCGCAGCCAAACTAGCCGAGACCCATGAAACTGATTCCAAATTCAGCC
CAATTAATTCTATAGATCCAAACAGGCCGTGATCATCAGTGCATCATCAGGTGGTCCGGAGC
AGCCGTCCGTGTAGAATGTAGTAGAACATGTGAGAGGGACGAGAGCACTAGAGGCGAAGAGC
AAGCAGACGCAGCCGAAGCAAGCCCAAGCCCAAAACTGTTTGTCTTTTTTTCCCCTTCCATT
GTCGTCGTTCTTCACTTATCCTTTCACAAACCACGACGATCGAGCTGAATGGAACTGCTTCT
GCTTGAGAGAGACGGCGGATGCATGCGTCCGGCGGAGATTTGCAGCAGGAGGAAGGGGACGA
GTCAGTCATTCACCGACCGAGCAGCAGGACCACGACCACCTCGGCCGGATCCTGGCTCGATC
GTGGACGACGAGCTAGAGGGCGAGTCCGGCCACGGCGGCGACGTCCTGCTCGATCGGTTTGA
TCGGCGACGGGAGACCGGAGATAGATAGCGAGCGAGTCCGGCCACGGCCAGCGAAATTTTGG
AAACAGCTTGTGCGGCGCAGATTTGCATGAGGAAGGGGACGACGAGTGGGTGTGTCTTCATC
TTCGCCGACGACGGAGCAGAGCAGGACCACCTCGGATCCTAGAGATCGAGCGAGTCCGGCCA
TGGCGACGGCCAGGGGACGTCCTGCTCAACATATACCTAGGTTTAATCGGAGACAGCGAGCG
AGTCCGGGCACGGCCAGGGAAATTTTGGTTTGCAGTCAGTAGTAGTGACTTTCACCACTGCA
CTACTACCTGCGGCTAGCTTATCTATCTATCTATCTATCTACAAATAATTAAAGTGGTGGCA
CATCACATATAGTCCAACCATGGCGTGGCGTGGCGTGGCTCCATGGACATGTTGGCTGGCTG
AGACGATAAGGCGCGCCACGGGGACGCGACATGTGGCGGCGGACGCGATCAGGATAGGCCAG
GCTGGCCGGGTTGCCCGCCATGGGACAACGGTGGCCACTCCTCCCACATCCGCTTCATTCGT
CCGATCCGTCCTTGCCCCAACGACAGCCATCCGTCGCCATGGACGCACGCTCGCTGCCTCTT
CTATATATGCCCTCGGTGGGGGAGCCTACAGGACGACCCAAGCAGCAAGAAGCAGCAAAAAC
AGCAAGCAGCTCACTCTCAGCTCGCTCCCTCACTAGCTACTAGTACTACATAGCAGCAGCAA
TG
SEQ ID NO: 47
Sequence Length: 3003
Sequence Type: DNA
Organism: Sorghum sp.
GGAGGTAAGAACTACAACATTTTAGTTCTTGGTTTTGAAAAATATTTACAGGGTCAGTTTTA
TTTACAAAAAAATCCCTAAATTTTGAAAAGCTTAATCATAGCTTTAGGTGGTTTTTGCGGTG
GGACAGTAGAAACAAGCTATATGTTTTTTCTAATGCTTGTTGTAATGTTTTTTTTTGTTTTT
TGCTATGTTTGTACCATGGTTCGAGTAAACCATCACTTAGCCTGCTTAAACCTCATTTGAAC
ATTGCATGGTGGTCTGTCATTTCTGTTTATTCGACCATTGACGCCCATTTAAATAGCTGTTT
TGCCCATTTCGTTGAGTTCCTAGCCAGAAAAATGGCTAGATGACTCATTGTAAATTGTTTAT
CGTATCAATAGCATGTTTACTTTGTCTACATGATGACCCGTAGCATTAATGCTTACTTTTAA
TTTTACAATGGTAGAATGTGTATAGAAATATATATAGCATATTTTTATGGTTACTGAATTAT
TTTGTACAATATATATTATAGGTAAGACAAACATTTGTGGCGTATTTACATTGTTTAGATAT
ATGTAATCTAAAAAGGCACATTTAGTGTTACTTTGTAATCTATATACTTTTATAAAGCTGGA
CCCGACTAGATGTTTTCTCTCTGAATGCAAGGTGTTAATATCATCCCTCACTTTAGTTTCTA
TCACATTGGATGTTTGTATACTAATTTAGAGTATTATTAAATATACACTAGTTACAAAACTC
ATTACATAGATTGTAGCTAATTTATGAGACGAATCTATTAAGCCTAATTAGTGTATGATTTT
ACAATGTGGTGCTACAGTAAACATGTGTTAACAATGGATTAATTAGGCTTAATAGATTTTTC
TTGTGAATTAGCCACAACTTATGTAATTAGTTTTATAATTAGCTCATGTCTAATTCTACTAA
TTAGTACCCAACATATGACAAGGACTAAACTTTAGTCCCAAGATTCAAACACCCCATAAGTG
TCCCACTAACTTGCAATCCTTTCATGCAAGCTATCCATGTCATGCATCCTTTTTTTCGAATT
ACACAATATCTCTTATTAATTATAAAATATATTCACATCATCTTTATTATGTGCAATACTTT
TAATCTCTAACCTATTAACGTAAATGTCATCCCTTATTAATTACAAAAAATATCCTCATCAT
CTCTATTATGTAGATGTAAAGTTATTTACATATGTTATTTGTTTCATCACCTATTCTTGTAT
AACGCAATCAACTATTTTATTGGTTTTTTCTATTAGCTTATTGTTTTTTTACTAGATCCGAT
ACAATAAAATAACATGCAAGTCAAATTCATAAATATACACACAATATACAGAATACCATATA
GCATTGCTATATACAAAACAGATTATTTTTAAGAAAATCCCGCGGCAATGCGTGAATATGTG
TCTAGTACCTTTCATAGTAAAAAAAAATGCAGACGCATTTTTAGGCATGTTTCGGTAATTAA
TTGCAATTTAGAAGGCTTTTTAAATTTATCTTTAGAAATGACAAACGTGGGGAATTTAAATG
CAACTTTAGCGAATCGTTTATGTATTTTTCGTAATGGTCAAGGCGGGCGGCTTTTCTTTTTA
GAAATATAGAACAGGTCAAATGGTTGAGGAATGAAGCGTCGTACGCCCGATGAAGACAATGA
GGAGGCGGACTCGTTTTGTCTTGGATAGCCCATGAATTTGAGGTAGCTAGGCCACAAATTCA
GGCTCTGGAACAGCTTGGAATGTCACTTTCCAGTTCAATGCGCAGCCAAACTAGCCGAGACC
CATGAAACTGATTCCAAATTCAGCCCAATTAATTCTATAGATCCAAACAGGCCGTGATCATC
AGTGCATCATCAGGTGGTCCGGAGCAGCCGTCCGTGTAGAATGTAGTAGAACATGTGAGAGG
GACGAGAGCACTAGAGGCGAAGAGCAAGCAGACGCAGCCGAAGCAAGCCCAAGCCCAAAACT
GTTTGTCTTTTTTTCCCCTTCCATTGTCGTCGTTCTTCACTTATCCTTTCACAAACCACGAC
GATCGAGCTGAATGGAACTGCTTCTGCTTGAGAGAGACGGCGGATGCATGCGTCCGGCGGAG
ATTTGCAGCAGGAGGAAGGGGACGAGTCAGTCATTCACCGACCGAGCAGCAGGACCACGACC
ACCTCGGCCGGATCCTGGCTCGATCGTGGACGACGAGCTAGAGGGCGAGTCCGGCCACGGCG
GCGACGTCCTGCTCGATCGGTTTGATCGGCGACGGGAGACCGGAGATAGATAGCGAGCGAGT
CCGGCCACGGCCAGCGAAATTTTGGAAACAGCTTGTGCGGCGCAGATTTGCATGAGGAAGGG
GACGACGAGTGGGTGTGTCTTCATCTTCGCCGACGACGGAGCAGAGCAGGACCACCTCGGAT
CCTAGAGATCGAGCGAGTCCGGCCATGGCGACGGCCAGGGGACGTCCTGCTCAACATATACC
TAGGTTTAATCGGAGACAGCGAGCGAGTCCGGGCACGGCCAGGGAAATTTTGGTTTGCAGTC
AGTAGTAGTGACTTTCACCACTGCACTACTACCTGCGGCTAGCTTATCTATCTATCTATCTA
TCTACAAATAATTAAAGTGGTGGCACATCACATATAGTCCAACCATGGCGTGGCGTGGCGTG
GCTCCATGGACATGTTGGCTGGCTGAGACGATAAGGCGCGCCACGGGGACGCGACATGTGGC
GGCGGACGCGATCAGGATAGGCCAGGCTGGCCGGGTTGCCCGCCATGGGACAACGGTGGCCA
CTCCTCCCACATCCGCTTCATTCGTCCGATCCGTCCTTGCCCCAACGACAGCCATCCGTCGC
CATGGACGCACGCTCGCTGCCTCTTCTATATATGCCCTCGGTGGGGGAGCCTACAGGACGAC
CCAAGCAGCAAGAAGCAGCAAAAACAGCAAGCAGCTCACTCTCAGCTCGCTCCCTCACTAGC
TACTAGTACTACATAGCAGCAGCAATG
SEQ ID NO: 48
Sequence Length: 3003
Sequence Type: DNA
Organism: Sorghum sp.
ATTAGGCGGACCAACGCCCCCGCTTTGCTCACGTTAGCTCAATGATTCCCCCAATGAGTAAG
GGAGTAGAGGGTTTTGGTGATGATCAAGGGTTATGATAAGGGATGAACGGACTGAGCTGGAT
TGAGGAAATAGACCTCTTAACAGGATCATCTCTGAAAGGCAGCCCTATTAAGAAGACAAACT
ATAAAAATCCATTTTCAGAGGGTGTCCAAACTGTTCATCTTCAGGTGATTCAAAAGATCTAC
CTCTTAACATAAAAATTGTGGTCTTCTAAAATTATTTTTATTGTAGTGAGTTAATAGTGTAT
TCAACAGAAAACAGTTAAATGGAGATATGAGGAGTTAAACCTCAATCCTTTAGAAAGCTTAT
CATAATGCTCTAACAATGGAGCTACATCCCCAACTTATATTTACTTGTTCATTATAAATATT
TTTATAGTTAAGTATTTTTCTTTTTTTTAGAATTAGACCATACAACACACGCACACTCATCC
CATTGCGACCAATTTTAGAATTTGTATAGCAAAAACTAGGCTCCAACGTATGTGCTATCCGA
CTTTGTAAAATAGGAAACTGGTTATCCCTTGTTTTTTTTGGTCATATATAGCCAACACGGGC
ACTTGCTATGTGGTTTTGTAAAATATAGTAAGACTGTTGTGAACCTTTTTTTGAAGAGTTTT
TTATTTTACAAAATAGATAAATAACAGAATTTCGCTAATAGTTTTTGCCAAACTTTTGGAGT
TGCTCTTGTAACCATTAGTCCACACGCCCTTTTGCAGCATTTTCTTTTTTACTTGAACTCCT
AACGTGAATTTATATAGTGAGTTGATCATATATATTTTTACAGGCACTCCTAACGTCAATTT
TATATACAAAAAAAAATAGTTACTGAATGGAGATGCAAGTATAGTTTAACCCCAACCTTTGT
AAGGCTCATAATGCTCTACCAATTGAGGCACATCCTAAATTTTATTTACTTATTCACTATAT
ATATTTTTATAGTTGACTATTTCTTTTTTACTTGCACTCGGTACTGTACTGATAAAATATAG
TTATTAAATGGAGATGTGAGGAAGTGAACCCCGAACCTGCTACATCCGCAAGTACCTAAAAT
CAATTATACATTCCCTATTTTAGGCATTTTTGCAGTATACACAATTAACTTTCTAATCAAAT
ACTTTTTTTAGTGCACTCACATTACTCATAGGGGTCTACAGTTGAACAAGTGGCTTCAATTT
TGCTCCCTAGAATATTAAAGAATATGAAAACAGGTTGAGATGGTCATGAAATAGAAGTAGCT
AGCTTTTTCGATATTCAAAAGAAAAGATCTGATAATGTAGTAACCAAGAAAATAATTTAATC
AGATATGCAAGGAATTGAACCCCAATAGAAAGCTTATCACAATGGTCCACTGATTAAGCCAC
ATCCCAATTTTACTTATTTTTGCACTGTATGAGTAATTTTATATATTATTTTTTCTTATATA
TAACTCATAATCGACAGTGGAAAAATCTGGGTCAATTTTGTACAATAGATCATTAGTCTACT
TGAAAAGAGTTCAATATAGTGATTGAATATATAGAAATACTGACTTTGATAATGAAACATAA
AAAGTGTTGATACTGTATTAATATAGAAGATGATTGAAGAGGCCGAGTAACTAAACACCAAA
ACATAGAAAGCCTATCATAATGGCATAATGGTCTACATAGCTATATACCCTCTATTTTTCTT
ACTTACTTTTGCACATTAAATTTATAGCTAGTTATTTTTTTGTACTTGCATTCGTGAATCAT
AAGAATCGATAATGGATACGGTGATGTCAATTTTATACTTTAACTAGATCATTATATTTGTC
GACATTAAAAGAGACTGAGATGGTGATTAAACAGAAAGATTTCTTTCTTATAATTCAAATAA
AAGAGCTTATGATATACTCAATCCATCCAAAATTATAAGATGTTTCATTTTTTGACACCAAG
TTTGACCACACGTCTTATTTAAAAATTTATATAAAATATTACTTTTTTATCATGGCTTGGGT
TATTAATAAAAATTCTTCAAGAATGACTTAAATTTGAGTATGTTTGCACAAATTTTTTGAAT
AGACGAGTGGTCAAACTTAGAGTAAAAAAAAGTCAAACGTCTTGTAATTTGGGACGCAGAAA
GTATTAAAAAAATAAGTTATATAGAGATGCGAGGAATTGAACCCGGGCCCGGAACATTAAAA
AAGCTTATAATGTGGAAAAGGATCAGCTTGTTGGATTCCTTGTAATAGAACTTGTCCACCGG
GATTTAAGTTCATGACTTGACACGGGTGCTCGTATTTTTTTGGATTTATTTTAGGATTTAAC
GGCGCTATATTTTTATTGGTAGGCGACGTGTCCGTCGATAGCGAGGCGCCTGTGGTGACTTT
GTCAATCTCGAGATTTGTCGGTCTAACTCGGTTCTTTGAAGATAGTCATAAGGGTACGGTGT
ACGTACGTGCGTTCATAGAGATGAGAGTGCGCTTATGTACCCTGAACATCCGCGTTAACCGA
GTCTGAAAAAAAAAATGATGTGTAACCGCTCAATACGGCAGGATCAGGGCTCTCAATTGATG
CAGTGGTGACAATTATATCCCTGTGGATTTTGTTTCCTGTACACTTGGGGTCGCTAGCTAAC
CTATATATGTTTCCAAAAGATATGTCCTCAAGTAATAGTGAGACCTGCTAGCTACGCATTGC
TGCTACTGCATTCGTGGAAGAAATTAAACTGTGTTGAAGCAACAAGACAAGAAAGCAAAATC
CACAGGGATTATTGTCGCCACTCCCGCAATGGCTGCTAGCCTGCCACCGCATCATCCTGTTC
GTTTTCGACGCGGCAAACAGCAGCCATTCCTTCCTCATCCTTCCCCTGCCTTAGCCGCGCGC
CTGGTTATTTGAACCCCACTGCCGCCGGCCATGGCGCAGAAGGACGGCCGGCCGGCCTCACA
CAAGTGTCAGTCATCACAACCTAGCTA

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. An isolated nucleic acid whose nucleotide sequence comprises a sequence having at least 85% identity to at least one of SEQ ID NO: 1 to 48.

2. The isolated nucleic acid of claim 1, wherein the nucleic acid regulates gene expression when operably linked to a gene.

3. The isolated nucleic acid of claim 1, wherein the nucleic acid has a nucleotide sequence comprising a sequence having at least 85% identity to at least one of SEQ ID NO: 1, 5, 6, 10, 11, 43, and 45.

4. The isolated nucleic acid of claim 1, wherein the nucleic acid has a nucleotide sequence comprising a sequence having at least 85% identity to at least one of SEQ ID NO: 11 and 45.

5. A vector comprising a gene regulatory element whose nucleotide sequence has at least 85% identity to at least one of SEQ ID NO: 1 to 48.

6. The vector of claim 5, wherein the gene regulatory element has a nucleotide sequence having at least 85% identity to at least one of SEQ ID NO: 1, 5, 6, 10, 11, 43, and 45.

7. The vector of claim 5, wherein the gene regulatory element has a nucleotide sequence having at least 85% identity to at least one of SEQ ID NO: 11 and 45.

8. The vector of claim 5, further comprising a heterologous gene operably linked to the gene regulatory element.

9. The vector of claim 8, wherein the gene regulatory element regulates expression of the heterologous gene.

10. The vector of claim 8, wherein the heterologous gene encodes an enzyme polypeptide.

11. The vector of claim 10, wherein the enzyme polypeptide is a cell wall modifying enzyme polypeptide.

12. The vector of claim 11, wherein the cell wall modifying enzyme polypeptide is of an origin selected from the group consisting of archael, fungal, insect, animal, and plant.

13. The vector of claim 10, wherein the enzyme polypeptide is a lignocellulolytic enzyme polypeptide.

14. The vector of claim 8, wherein the heterologous gene encodes a polypeptide selected from the group consisting of stress resistance polypeptides, nutrient utilization polypeptides, mycotoxin reduction polypeptides, and male sterility polypeptides.

15. The vector of claim 8, wherein the heterologous gene encodes a polypeptide that confers resistance to at least one herbicide.

16. The vector of claim 15, wherein the heterologous gene encodes a polypeptide selected from the group consisting of phosphinothricin acetyltransferase, glyphosate-resistant enolpyruvoyl-shikimate-3-phosphate synthetase, dalapon dehalogenease, and bromoxynil nitrilase.

17. The vector of claim 8, wherein the heterologous gene encodes a polypeptide that confers resistance to infestation from at least one organism.

18. The vector of claim 17, wherein the polypeptide confers resistance to infestation from an organism selected from the group consisting of insects, bacteria, fungi, and nematodes.

19. The vector of claim 8, wherein the heterologous gene encodes a polypeptide that confers resistance to at least one virus.

20. The vector of claim 8, wherein the hetreologous gene encodes an RNA molecule that regulates a plant gene.

21. The vector of claim 8, wherein the heterologous gene encodes a polypeptide having therapeutic value.

22. The vector of claim 8, wherein the heterologous gene encodes a polypeptide selected from the group consisting of phosphomannose isomerase and anthranilate synthase.

23. The vector of claim 5, further comprising a selectable marker gene.

24. The vector of claim 23, wherein the selectable marker gene encodes aminoglycoside phosphotransferase, hygromycin phosphotransferase or neomycin phosophotransferase.

25. The vector of claim 5, wherein the vector is a binary vector.

26. The vector of claim 5, wherein the vector is an expression vector.

27. The vector of claim 5, wherein the vector is a plasmid.

28. A transgenic plant, the genome of which is augmented with:
a recombinant polynucleotide comprising a gene regulatory element that has at least 85% nucleotide sequence identity to at least one of SEQ ID NO: 1 to 48.

29. The transgenic plant of claim 28, wherein the gene regulatory element has at least 85% sequence identity to at least one of SEQ ID NO: 1, 5, 6, 10, 11, 43, and 45.

30. The transgenic plant of claim 29, wherein the gene regulatory element has at least 85% sequence identity to at least one of SEQ ID NO: 11 and 45.

31. The transgenic plant of claim 28, wherein the recombinant polynucleotide further comprises a heterologous gene operably linked to the gene regulatory element.

32. The transgenic plant of claim 31, wherein the gene regulatory element regulates expression of the heterologous gene.

33. The transgenic plant of claim 31, wherein the heterologous gene encodes an enzyme polypeptide.

34. The transgenic plant of claim 33, wherein the enzyme polypeptide is a cell wall modifying enzyme polypeptide.

35. The transgenic plant of claim 34, wherein the cell wall modifying enzyme polypeptide is of an origin selected from the group consisting of archael, fungal, insect, animal, and plant.

36. The transgenic plant of claim 33, wherein the enzyme polypeptide is a lignocellulolytic enzyme polypeptide.

37. The transgenic plant of claim 31, wherein the heterologous gene encodes a polypeptide selected from the group consisting of stress resistance polypeptides, nutrient utilization polypeptides, mycotoxin reduction polypeptides, and male sterility polypeptides.

38. The transgenic plant of claim 31, wherein the heterologous gene encodes a polypeptide that confers resistance to at least one herbicide.

39. The transgenic plant of claim 31, wherein the heterologous gene encodes a polypeptide selected from the group consisting of phosphinothricin acetyltransferase, glyphosate-resistant enolpyruvoyl-shikimate-3-phosphate synthetase, dalapon dehalogenease, and bromoxynil nitrilase.

40. The transgenic plant of claim 31, wherein the heterologous gene encodes a polypeptide that confers resistance to infestation from at least one organism.

41. The transgenic plant of claim 40, wherein the polypeptide confers resistance to infestation from an organism selected from the group consisting of insects, bacteria, fungi, and nematodes.

42. The transgenic plant of claim 31, wherein the heterologous gene encodes a polypeptide that confers resistance to at least one virus.

43. The transgenic plant of claim 31, wherein the hetreologous gene encodes an RNA molecule that regulates a plant gene.

44. The transgenic plant of claim 31, wherein the heterologous gene encodes a polypeptide having therapeutic value.

45. The transgenic plant of claim 31, wherein the heterologous gene encodes a polypeptide selected from the group consisting of phosphinothricin acetyltransferase, phosphomannose isomerase, glyophosphate resistant 5-enolpyruvoyl-shikimate-3-phosphate synthetase (EPSPS), aminoglycoside phosphotransferase, dalapon dehalogenease, bromoxynil resistant nitrilase, and anthranilate synthase.

46. The transgenic plant of claim 31, wherein the recombinant polynucleotide further comprises a gene terminator sequence operably linked to the heterologous gene.

47. The vector of claim 31, wherein the heterologous gene encodes a polypeptide selected from the group consisting of phosphomannose isomerase and anthranilate synthase.

48. The transgenic plant of claim 28, wherein recombinant polynucleotide further comprises a selectable marker gene.

49. The transgenic plant of claim 48, wherein the selectable marker gene encodes aminoglycoside phosphotransferase, hygromycin phosphotransferase or neomycin phosophotransferase.

50. The transgenic plant of claim 28, wherein the plant is fertile.

51. The transgenic plant of claim 28, wherein the plant is not fertile.

52. The transgenic plant of claim 28, wherein the plant is a monocotyledonous plant.

53. The transgenic plant of claim 52, wherein the monocotyledonous plant is selected from the group consisting of maize, sorghum, switchgrass, miscanthus, wheat, rice, rye, turfgrass, millet, and sugarcane.

54. The transgenic plant of claim 28, wherein the plant is a dicotyledonous plant.

55. The transgenic plant of claim 54, wherein the dicotyledonous plant is selected from the group consisting of tobacco, tomato, potato, soybean, canola, sunflower, alfalfa, cotton, and poplar.


SEQUENCE LISTING (79 sequences)

SEQ ID NO: 1 - DNA, 2765 bp, Sorghum sp.
gtacaccattgatccccagcatataaaactttaataaagtcggtagtacatgtatatgggctcactaaatccgtatcagcacgcgtgtgccactaccactagagatgtgtgctcagctggagtactctagtttattattattattatagttccaggtcatatgatcctggacccaaatcgcattaaatttgttgcaactgcatgcaaggtgttgctctttaaaagcaattatatatatatatatatatagtaataaaaaaaggggaaaaataaggatctggaagccggcccaggcgcaaaaggaccggtccagcgaggaatgggttgggcttgctgggtgcacctccacgctagtccagccgcacaaatgggcccgccgccatctcgctccatcggacgggtcagcttgctccacgtagcccatcggaagggaaggccctttcctttttttttttcccttgccagtgccaggtatgctgcttcatattatacccctgcgcccaatttggaatcttggccaatcgatcgataataacaaggacagatgattcgtgacccacgcttcttccttgattgtttggttgctttagttgaaggcatcatcatcaactagctgtgctgtgcagctcgtcggctctagctagatgccatgtgggttatgcatgagtttgtttcgtgttgcacattttagcctatatgtttgctggtttcatcgacttcaagttaatctttgaaagagagcatacaacgtaaaatatttttctagaaaccggagccattttgtgaagaaaaacttctcccaaatgtgacgccttactattacattacacctctttagtagagctttggccagtcctagctactaggccctatgtgtactatagcaatgaattatttgtatctcttttgtcaaaccgaactaagattgggcgaatagttttactagctctgacttctcgatctaaatatatattactattttgtttgaaattgtaagtcattttgacttttataaatttagagtttttactatatatctagacatagtatgtatgtatgtatgtatgtatgtatgtatgtatgtatgtatgtatctatgtgtatagctaaatctattatggatctagaaaggctaaaatgacttagaacttgaaatggatgaaataattcattacaaaatagttgcttcatcctattgcaaaccttgattggccttgtttggatccacatatattgatccataatgcacatatattagagttgattgaaatgaaacttagtttaatttcacttcaacatatgtggattgaggtacatacacatacatgtaaacaagatcttatataatttatcttacaactcttctctatgctttagatcgatataactcatcacaaaaatatgtacgcgtctttggtcattaattccttgcatctatttaatgaaagagaccaatcttatcttgtaaataaataaaggcattatttgatggagctccaaaaaagttaggtatttgtaccttcatgaaaagttgcatttagagttaaggacttaaggtacaacgagggcatttagataattcattttcctactgggtacaattatttaaatatctagatctagagcataggcatcaatcaccgctcgatgtactaaacaaatggcatcaaaagtttttcttaaaaaaatggcctcaaaagtaaccacacaaaaagtttcaggagtagttagtggtgacattcatagataaaatcatctcaatcacttctaattttcctagatatagcctaacataaacaagaacacaaaaaagatcgtttaaggaaaaaaagtacacctgcctatacaaataaaaaaaactgtaccatttaaaccattttgcaatcagaattcagaactaggcagaaactactcctttttttttgtactaatgttttttaaattaattttctcccacccggatgcgcatataaaaaccgccgaaacccttggctctcctcacttgaccaccgcaaccacttctcccctgtttcctctcgtgcattctccgtgggaagcgaggaggactcgcggccggcggcaggttctgctagatctccgggtaagtgttgaaagaaaagaaaaattgttggaggaattaattccgaatctttctccattgcgattttggtgtctagtctgaaacgcgcagttcatcccattcctgcccacagtagatcagatccgatttgctcctgcggcaggcattgcgattgctgggccctggttgccgattagttatcgacataaaccgcggccaagccccccaagaagttcttgcggggtgacaagaagaagataaacagtttggccatctcgcctcgcaaggataaccgcccgttaatgcatttttgtttcttgatttctgaataaacataggtattattcgatttctggataatagagtgccattaactggtccctgcatagcgtggtattggttacagaaactgtgccggttctgtagatttagatgaatcacagtgtcaagagagacaggattcacctgaatccttctgttaaattagaaaatagacatgatccaaggtcctgtgatttctcagtgagtagtcgtgtagagtttatttattttggtcctgtttcctttgctgaaaatgcagttaataccaagttttctgctgtttcctattaagatagatactctgttactgatttttaccctttggctcctccttgtgtttttgttccagaatctgtagcaaatg

SEQ ID NO: 2 - DNA, 2720 bp, Sorghum sp.
atccgccggatcgtcggccctcgggcgctcaaacacgccagagcaaatattcctgctcgagccgcgcaagtcgcacaatatttccatcatcctcaagtccctcacggtggggcgggatgaaatcgacgccctccgggatgggcacacagacagaactcagcactgggaggtcctggagaagctttcgcggctcaacatctctaaagaggaagagtccaccatcttgaagttctgtgggaaccccgacaggcttgccccaacggaggccttcctcctccgtctcctccttgatgtgccagggggttgtgaaggctgcagaacaggagctgaaggcactaagaagggagcaggaaagagtacttgagctggtccagaagacaacagagtacaaccatgctggtgccgccaaggaacggaacgcacatcccctccagctgttcatcgtagtgagggacttcctgggtatggttgatcaggcatgtgttgacatcaagaggaaagtgcaacagaagaaaccagcaccatcgtcatcgcagccaaacgcagcagctgctgcccccacggtggcggctgcggctgcggccacaacagcggtgacagcgtcagtgacaaaggaagcgaccgatggtcaagcagcaccaactcacaaaccacccgaagaggcagatagtaaaaggaagagggtcatgccaagatttccaaacctaccagcgcacttcatgaaggaagttcagattctgattcaagtagtgacgaggaatagattgaacgggtggctgtcaatgattgtttacattgttttgaattggttttgtagaggtataggatagctgcagactgtacataaaagcaattttttacattggttcttttgtccatttcttcaatcaagatccatatcgagagcaccgaatagaaatatagaattcagaaatttgtgaaaaaaaatgatgtgaagatctcaattgcttagaaatgatatctttgtttgaggggaaagcccctactgtcggtagctatataaagaaaatgtgaaggcattgtacaaatacgaaggataaaaagttaacaaaaaggagagaagacctggacatagagagacaattaggaaagatatcccagccatgtcagaagatatgttgggtctcttttttaaaaaaatgggtccattccgttgcttccaaatttttcagactactattataaaaaaatccccatgaatgaagaaaggtaactgtaaatatttttttccagcttgcgtcaatcatgatgagaaaaggtagaggcatgatccaggtgagaccaaccacatgccaacaggtctactgaattgacatccaaagaagagatgcagccttgtttagttcccaaaaagttttcaaaattttttaatttctcgtcacctcgaatcttgtggcacatgcatagagcattaaatatatataaaaataataacttattgcgcagtttgtctgtaatttacatatatatactcaaatgtgtgataggatggcagagagatgcggcggaacttatttatagatttactagcgcacgaaagaaatggatatcgaagatttcttcctaccgatattaaacattatcttgtcgtatcacggaaaggtctataaagtagaatttgtgagcctataacgtcgtgctctccgtgaccgtgtttgatttcatgaactttgttttctttaagttaaatggaactttatttggtatttaagttttattataatattattatcttactgtgcgatccatgtattttaaataaaaaatgttagctatcccgtagcaacgcacgggcacgctacctagtttaactagtaaaatcatcgtaaaaataactttgaaccctttcaattttcggattgatagtatagcctcaaggggcttttggtgcggttttgagagctattttctttaaaaaaagatttataattttttttaaatccgggagtcgtgaggagtcaaggagaccacgttctcctcacgttctcctcctcctcctctccttcacaccacagacagcccccgactgccacaagtctctctcctctcctctctctctctctctctctctcctcgcgacgactgggcgagaccgccgccgcccttcgccaggtgcccaggtctccgccgcttccttcgccggaggtcaccaggttcgcctgcccccttcctttcccttggtgctgggcgaaaccgatctcccaaaccgtatcttaggcttcccatttgtgtactcgcatccagatctgatctagttacacgtatagcatgcttgcacccggatctaatctagttagaagcgtagtgaacttgcattcggatctgatctacagttgcatccggatctggcgtaaaagagtttgctagttttcttttacgaattggtctaagctaactggatgcttgttgttggtgtgattccagtgaagagcagttggtctttcgtccggaagtagacttccaccacgcatattagcttactggaatatgatgttgcaaatttcagatgctataagtcatgaataaattgttttctttgtgtgaccttttttctgtacgaagaatgtgatttactgctcaattggcacgtgtatccgaatcacagtgttcctttcaatcaatcatatattgaaaataaaaaaaattaatcactgatggattctctttatatgcgaacctgcagcagttctgtagaaatg

SEQ ID NO: 3 - DNA, 2348 bp, Sorghum sp.
cgggcgcgcacgtactgctactgccgcgtgggctgggccccacgctgggtgatcggacgactcggagctcctccgcgtcatctatcgtgcggtccagcttggccgagcatctccagcgcattccagcctcaggaactcaggatcttcggcacttttctttccccctttttttttcaaaaaaacgcttgatttgattttttctctgtttccattttctctggctgtggaccactagttattgtgcctgatgtggaaggaattatttctcacaccatatatattatttattttcatttttttaatatataaaagtgtgtccttcgatgtcacgctttcaagcccatgacaactggagttcgggagatgcataactgtatgtgagatttctttttcatttttttttcgtttgtattgcatactacttacagttggcactggccaggacggtctggaagattctagagatgttggttaacaatcaagtgtcctccacattttcatggaaaaaaataaaccgcattggaaaaatataatatgggttcctaaaaaaaggaacacgagtgatgatatttatagtattttttcaacaaagtagccttacttataatttttgctaaaaatgatggctctgtgtcattcagaaataatactattgttttcataaaagcaaaagttttttcttatccctaatggtattgaaatattagagttcattaaaaatggtaaaaagggaatgatggatgaaggaagtttgtttttaactcccctacaacatctaaacctatttatggttaggggtctaaataaaataaaatgaactggaaattagctaagcaaagatagttctagcagacgaaacaactgcaacactctttcgccagcaaaacaagaacaagaacacaattaaccaaggcacaatcaaacaggcatctaccatgtttttgttggtagtaaaatgaacatgaaaccaacaaacacatgatggtgcctagcacacaaacacatccaccacactcttttcacatcaaaatgaattagactacaataaaaccaggtgtgacaaccaaaattatggccacagagcaaactttggataaagtgaatgcaaatattaacaggagaatagatggcacttggacaaaaaaaccggatcccatcatagatagaacaaatgaaaagtaacattattaaggccttgtttagttcccaccaaaatccaaaaagttttcaagattttccgtcacatcaaatcttgcggcacatgcataaaacactaaatatagacgaaaacaaaaactaattacacagtttttctgtaaatcgcgagacgaatcttttgactctagttagtctatggttggacaatatttgccacaaacaaacgaaaagtgctacagtagcgaaatccaaaaaaaattcgcatctaaacaaggcctaaattacagcatgatcaaatcacaccaaaaaatacaccaaccaaacaacatctaagtgagtgtggagcactaaacatatgacaccatctatttaaaaacaaaatcaacacaactgaaaaatagttacacgtgtacttaaaaaaatctctatcgcagagagagagagaaacaataaatatatactatgaaaacattatcatggttgacgacgatccttgctcgaaatacaggctgaatatttgaaaccggttttgagggagaaaaaaaaaacccgaaaatgtacacctcgtgtttggaaccgtatccccgtggggtcccttccatcccttcccccggctgcctccctcgtataaaactccaccaccccagtcctgggcggcgagcacgccgccatccaggtccagccgacctgcctcccgccgccgcgaccccacaccgcctccctttgccggcggccccgtcccgcggatcggttggcgtctccccttgctgctggtatgcaagccctgccttcctctgtctgttttgtttgttttttccccccttctttcgtgctgttcagcggtggatctcaccctttgccgtgggtggtgagcgctcgaacccgaccgaatccgctggtacgcgcgctccgatccgtctagttcgtcgcggattcattcgcttaaacgcgggcggaggtttgtagctgggagcggttgatttcccgaactttgtgttaaaaaaaattatggggagtttaagtgcgacagcaaactgcagcaggatttgtaagaatttcctgcggaatttgcccagtaggactgcccttgatgggctgtgtgtgctggacacagattctgctatagtgattattagtaggagtagccctcttattatgcttgaatccgtggcagaatcactgaccagatg

SEQ ID NO: 4 - DNA, 3552 bp, Sorghum sp.
ccgagggcagcctgcctgcactgcagaatgtgcttggtcacgaaaaggtgacttgcagacagacaggatcacaggaggtggactggcgaggcgtcgaggggagggggggcagagcgaagcaagcaggtgccgtagagcgacggcgagaaatgttggcacggtggaggcgttgccgtaaagcgaccaaaacgaagccaaaaaaaaggcggtccggaaaaggccgccgcccacggtccatcttttgccctgttccgtggttggccccggcgcggcaccgtccccccctggccggcccccattccactttcgtgtcatgtgctcattttttcttctcttccaatcctactgtcaagtagtagtaccaaccaaagcaccgacagcgcaaggcgtaggacgaaaaggaaaacaaggaaatggtgtgctgggatgattggaaaaggtgatatgagatgggtgatatggactcctagacaacggcagctagctttgccaacaaagtaaagttgctataaggcgtatgagattatatgtatggctctttttttttttgttaagcgtacgaagtgctcactgtacagagatttaggaagccaattgctccggattatgactctgtttctaagcaactgataaagaggttgtggtttttcatgtgacccaccccttgttgccatcttgtgagataaatgttgccaagaggagtgctatccaccaggcttcagtctggccgagtttacgacggacgctggcctgctcagtacgtcgctacagggcgaccatccggtttgtggtcagtgacagttccgccacacctcaacaattttaccaagcgagtgctgaagaaggtggacgcctcgctaattcgttaacctcccaagctgatgtcacctaccaacccggtgctacagctgtggagcaaacagggtggctcacagtctttctcggtgcctgattctaaacaagatgagatcctaatcatgcaacatatgggcttgaccaaaggtctatcagcacctagtctatcaggaaggaatgcctatgttgaactcttcgaggcttggcgaaatatgtctaacgtcaaagcactgcacatgctcttcccaaatgggaataggtcgcacaagcagtagcaaagacgcaaagccatttcttaggctgcatcactgctcttatcccgattgtctatcttttatgtaatatcaaaacttgagtctccaggacgatccaacctttgactttgtacattgtgaaaatctttttgccaaactattcactcttactttttttgttacacatagatgtacacatttactatttggctaatactttatctattttaaattataagatattttagtttttatagttacattacttttgttatgtatttagtctaaatacatagcaaaatcgatttatctagaaaaatcgaaacatcttatgattgagactcaagggagtatatgttacttttgctcttacggtgttatattcctaggaatcttgtggacaactttgactcgtgacatgtgggtaaaaggaaaagatttgtgctgcctttgatgggtaagaagattttaaccttgaggacggtgacattatatagcgcagaggaaggtgacattatctaactactttaataatactttttagtgtataaataatgtttttctcttataatatttcaacataagtattagcataaaacaaattttagcaaaacgaacaattaaagaagcagctaagattgaatcaaaagacaacttagaaaaaaaattatattaaaagaaaattcgaacttactgctccttctgacttcggtctgcatacacggaaaatgaaatttgaaacaaaaaagaaaagaaaacgaaaacgaaaacacgatgcatcaatcaatcatataaaaccgaaagagaaaagaaatgtgattctgtatggtgtgccccagcccaggtgggcaaattattccgcgttggcacggccgccctcccccaactttccagtgcggcaccggccacctttacacctcattcccaccgccgccaccaccaccacctccacctccacgctccaccgttgccaccccagcacgctctcgccgtcgccacgccctatatctcgcgcctctcgcctcccactctttctccatccgcccgctcgcctccctgctacgcttcgtgccgccgccgcaacctcctccttcccgtcccgttccaggtgagcgaatcgagggccccttgccgtactgctttaatgctgctgtttcttgatgctctagaggactggagtctgggtgataggatgcactggggtctgggggttcgttggttgtattatgttctggttggctgttagtgctggatccgtagtaggagtaaggttcgtcaaagttgctgggactttattggtgacctgtgggctgtggatgcttcgatcctgtgtttatgttaaggtggctactagtattacactagatctctttgtaaattttcggtattaatattagcctgtggtaatggatccatggttcttctgtgcaagctctttgtgattagaatttatagaaaggaaatttgtgatccacgtttagagtcgtttaatggatccattgtcctcatttgaaacttcggaaactatggccttttttattggttttataacgccatctggctatagaaatcttggcgaagtttgtgcttccgtagagcagtataggcttatagccaaatgttgcttcaagatttcttgttcaaaaatatttccacctgaaaggggaagagtctatgaagtttgtgtaccagacatcgatgcatcgaatgccttctgatcagaaaatgctaaattcttcagatcctcacttgtgaggaatcataactgggcacgatcagaagtactggctgcagcagcactactcagtaaaacctatggaaatcaagcagcatcagccgctgcagctgtggtagaagtgcagccaagcagagcaactttgttttcaataattgaccatgtgtttgattaaatcttcagggcctggtgtcattccttgtgctattgcacaaagcgtttgtatgtattggagtttagatgctccagagtccagaacagcctgacttttttttgggtatatttacaactaaaaactaattgtttaatatagttggaaaattataaattaatatttaattgcacaaagggttttatttttgtaagaatagtggtttctgattacacttaattgttttttgcttgatttttcacagttaccgttgcttgtgtctttggtttcgtgtttgataaatgttagtactcaatgggaatatattagcctctttttctgagtcaacagataatagcttgttaagatgtgactcatgtcttctgttgggagtaggcacatcagcctttttttctcttactgaagtaattagcacatataagtctacggtcttttcttttaggggaatattagtctacagccttggtaaatgcttctcctgataaattgttttcattcatgcagaattgcagattacaggtcatttaacataaatg

SEQ ID NO: 5 - DNA, 3351 bp, Sorghum sp.
acctccgaatggtgatgctgcaaccgtttcatgaaaaaaaaagtcagacttacatacgatttttttactcataataccatgtgctatcttccatcgtttgcttgtaaaccataccatccctcctcggtgcagaactttgcttggtttagcgctgttcatcgcaataaaccgctgaaataattgcggaagcatgaagcacgcttttgactcggaccgtttggaaaatggaaacttccccccgcccgtccagagtgggagctgctggctgggccgtcgcccgtcggcgaaatgaaatggaattattgccccgcgtaatggcttctccccgggccttccactagcggaagcatcaggcgggcgtgtggctcgacgcagttgcaggacgccaataatgcagccactgttggtgaaagcaccgggctttttgcccatgctggtgtggtgaggcgtcaccgcgtcaggcgtagtatttgcgtatcgccttatcatgcgcgcggtcgagtgctgaaagcaccgacgatgtatcaccccccttgttcaccggtgcatgcgcgttgctgttccggcgtcagaggaaggaagggaaggaaaagatgatggaatcgcgtcagatctcccggaaaagaacgtgcgaattattgccactttgagcagctggaaaaagtcaatcattggacttgttaactcccggggagtttggtttcgcgcttttgcggtttctaaacgagagtgattcggcggtggtgcgggcagtttcggctcacagataatattgccgtcgatcccacgctctcaaactatcgcgtttgataagtaccagggctccaattgctgcgctccttgttgccatcccgagtcacgaggcatgaatgaaacggtttaccactcgtcggtagtagtaggccttgatgtgagggtaaaattgacgtcgtagtagctagcttacctcgtagctagccgttttttagggcacaggcagcagacacgggtgctggaaatcgcgatcatccgatgaggatttgttctcatgagcaaatggtcgacgttgaacatgcctgtctcgtggcctctgctagctgactctgctctgctgtgaaagatctttgagctcagatcgcacttgtggtaataaagggtgaaaagagttatctcgaaagtggaacgtgatacacgggttcctccataaaccttcgaaaatatcgcatgtggaatctgctagaaaaaaaggacagtgcgaccttgtaccggtgcaggaacaagtacctgttacaccccgactacctggactgcctggagtcgaaagcgaataccgagactgcattactattactaattctcgtaggttttaggacacatataccaatcacagggatgctaggaatcgcgatcatctgatgaacatggtcggaggtgagtaatggctgacgtcgaacttgcctgtcttgtcgctttgctctgaatttgtaatacgtagatctttgagctcagatcgcacttgataaagggtgagcagatggaactcgagcgtggatggaatgtgatacgggctcctctgaaaagtcttcaaagacattccatgtgattctgctcgaaaaacgaacagtgcgatcctgttgtgctctccgcgagtatttttgactggccgggggcgaaagaaaataatacagtaataacaatcggcccagctcggtcgcgatcctagagcctttgggccaaaggaaacaaatctgtgggcctttcgacaagtctagaagtctcgaatcttattctcgggcggttggcgacggtgacgatgagtgggccggaccggtcgcgttagttggcgcctgctgcttctgccagcgacggctgggtccaccagcccacggagcacggccgccgcccgtgccccaccacccgcccatatcatccgacgaggacgtgcgccggattcgcaccgaaagcttcgcggtccggccgccgtccgcctgcgcagatttttgtttcatctggcggccgctgcgtctccattgacccggcagccggcacgggcgaggtcagagagagcgagctgttggccgggtcgtccctgcgctcgccagcgctgctgtcatgccgtgccgagacacggtggcaggcggcttcccaacgacgctgggcatccgcagccagcgcgtgcagcagcagaccagtccaaccaggcgggctcgtataaagaggttcccctgttccccaactttggctgccgctctcccatttgtctcgtctcgctctcacgctcgcgtcaccggagctctccagaagcgagccccaactgcccaagggcgagcgatccgatccccttcgcagcctcgtcaacgacgccgaggtatacccgtgtttcccccttgctctcgcaccgattttatccgaggaagcgatcggctgtagacagtcggttcgattggtccccgctcgagcttcccgtcggtgttgacttcggtttttcatgtcgattttgttgtttttgtcatgtttgtttcggctctagaattcggataggcggtctgatatggttcaaagtgatccagcatataataatgagcaaaactaccgcatatgggtagtttgagtgatccagcatataacgagcaaaaactgccggcaaaaactgccggtctgtggtgtttgattggattccctgtttgtgtgatggaacttaatgtccagttactcagtcagttcgtaggtgtatatatgattcagcaatcagcatgtgctaagatatgtgctatgctgatccataacagagtagcagaacccgcaaattgctttcagatcactttgtgaagaactggaggtgatctcaagcaggtgaaaccacactgtttctgtcgatgattcagatgaaatgtgactcaaatattcagttaataattttcctcaaccaaattggcccccgtcatcctcttacccaaataggagtgtggtttcagccaattcggtacatacgtgtgatgcttgctcaccataaaaaaatgaaacatgcatggtataatggtagcagagaaatttgtggccgatcaagtatcaagataatgaataatttgtattatcagctggcaccaaagtctggtcgtctctgcgaattgcttgaaagcttatacttgtataagtcaagtatccgcttttttaacgcaaagtaaagtatcagtttcttaaaaaatgaacagttgtgcacattagttttgactgatgattgccatattgcaatttgatatttttttgtggggtttacctgactccatggacactttagtgattaacaccgctgcagataaactcaaatacattctgaatatagcattctgctgcagaacactttgaggaatg

SEQ ID NO: 6 - DNA, 3273 bp, Sorghum sp.
ctggcatagatatattagatctaaaaattaagagtatctccataaaaagaactttttaaaatttgccctctaaaacataatttgagaagtcatttgaataaaattcgctttctatatctttgtaccttgaataacttttctatatcttgtgaacactctagagagccatccttgctctccattttttgctacgaaaaatccaaaatagattatgtttttggagggtattttttcacctgaaatatttattccaagaaatcagaaaaaaatataaaggggtattttggagttgctctaaatttatatggcggtgctatttaggtaggtcccaaaacttatcacatgagccatgcaaaatgtcatttaggttacgctagtctcggtgatggatatgaaagttttatttacattaaatagtctagcacatatgtattttttatgacatgataatgttttaatgaagagagagagagagagagagagagagaaaataggttttgagggacagaattttcttgacacgattaggtcagcagtgccatatttcaatgcactgttttcaaaacaaatccgctcacagggcatccatgaaacgaataatgaaacaacctccacaatgcatgagtttcatcttgacgtttcctaggctgggcaaagcatttaattactgcaaaatgattggatcacatgcaagatggtgaaacgatttagccctcagtgagaatttcatctcgtttcaccgcgtgggaaacaacgcccgcggggtttcaccatggtgaaactacttccttctctctcctcttcgtttcatgaaaaaaatacagttttgctgacatgacgcactaataaatgtgcatgatatcctggtgaaacccccactgagaccggccttactaactttctattagtctaagaatttaatgtctatgaaaccatagaacgaaactttgcattaaaagtaaatgtttcatctaagttttattttattgtatatgacatgtctttgaaacaacgcaatctccactgagactggtcttagacttgcaatttagtgggccagtgtgctcatcgctggaggtccgaaaactgtcatttgcataggtccaaaacttattacacgatccaaacttattccaaagaagtcaaagctctttaaattcatgcaaaaatataattccagctcgccttcggcatagctagttttagaaatcgttttttttttcacgggatggacttcctgaaggcataatttgctcgtttggattctaaatttagctctctttttttaaaagaaaaatgtaaatttaactttctatgtattcatttcgaacgtaaccagtgcaatgcaattcactccaatgcactgcgcaccgcggtaatttgtgcttggacatgtgttgggcccataatctgtgatcattgatccaagtgatgtttggccctttcaccaaccgagtgagcccagcagaaagtccacttcttcatcgggccatcagacaagcccaatcctccatgccgtgcgaaaagtccataccgtgcagggtgcacggttgcgggtagcgtcagccggcacccggcagcgacggacctagccggcggccgctaggcgccgggattggggcgaggctcgccaaactctacgctggcgccgacgtggccctgccactttgggcaccgaaagttccctgcgacggcgacggcgacggctaggccggaacccggtggagcctttcggtttcgggcgctttctctgccgtcccccggaatcatcaccaggccggaccgcggggcccagatacatacagagacaagtcctgcacgtctcaaccggcggggacgctccgacgtgtcgatcgagaacaacacgtgccgattcgcagtacaagcggcgtgggagccaaaggtacgggaccagtcccgcgcggcccgtcaggccgtcgtcacgctcgcagcaccggccggtttctacgggcggtgcagtggacgcactagtctcttcgaagacggcggcggcgtgtggtataaacccggggccgcccgtcacgccgccccgtccgtgcgtttccttttgtttcttgctttgcttcttttcgagtgcttgccgtccgtcactgctcgccgatcgagttttctccggattccagcagagaggccccgaacgaagcgatcatcgccgcacctctcttcgagcaggtgataatccctcttgctcttctgttccatctcaaattctctgcggaatcgtactagtttttatcccccccgacagatcgcctcgctcaccagtcgccaggcttccgattggtggtgtctggtaacttttttttgtggtttctgcgtctggcgctgctgattccccgtggtttcggttggtttgtggcgatttctttcaagggcaaaggaattccctgggatagtaaggcgtgctgtaggctatggtaggattatgctacggcgatggcgccgtgccgccgtcagtactgaaacactactagtataggtcacccgctgtcagtggcgttggtgagttttttttttttgtttttgtttttttttgggtaaaacagcgagctacgaattaactggttgctgaaatacgtgtctcgtatctcttcgtatggttactccgcgccaaacaaagaaattaggattgactttcagattgtagaggctgctgctatatatggagctcttcggttccctgttggtctctagttgcctgggcagggcaggggtagtagtagtagtagtaatttttatttttatttgaaatgcccgtcaagagttctgccaagcatatattgatagagcagggataatactaattttattgccgtagtgcacagcagcagcagcaaggccagattgtgtagagacagttcttgctgttttggcaaatacccccagaagttagttcctgtgaccagtggtgatatatgatttactttagataactcagctcatcgtggagcataatttgtctgacacagatctacatttatacgcgaatactgcgtatagaagtatcttaggctgttgtactcccgtagtagctgtgtagaagtcacaagtaaaggaaagacaattacagaaacagaaggcttttccttgaaccatgagcagaaagtgtgttttgattcccctgaccaggcttgatgactattaactagtactcaattactgcagttttctctgaatgtgtactgaacgctttctctgaatgtgtactgaacgctgaacattgatctctctgttacagaatagttgagaatg

SEQ ID NO: 7 - DNA, 3003 bp, Sorghum sp.
tatgtcactcttggcctcttgcgcagaatagaatcatctccttccgctcatgtggtattaactttgatcaacgaagaacaaaacttggttctcacttcatttcaaattttaggcgtagtataacactaagatccatgcgcatcttataaaaagaaaccaaatgagagtgagggtactgcaaaatctggagccaatctctccaaccccatcaggctgattactggaagaaaaaaatattttaaaattattataacaatggctacagcgaagaaaagacacaactgaagtaataaggtatatgaagatacaactgcaacgaaggagagtaaccaccaatctccagcacagagattggctggtacatttgctggacacaaaattgttatctatggacaaacagctagcaacaccgcagcaccttttatatcttacactgcccctatcctattcgcctattagaaggtgcccgactatctctcaattagcgataatataggattattcaaccatcatctcttcttaatatttgaatatcaggcactagttttgtgatgccaaccagcaggaaaccttgtgattcataggcgttcaatgttaacacaccagagtaggcttaactgtcgaaaggataaacttatatcgcacaaataaggaactcaaagctgaaatggcaatgtagatggctgaagccctggaacaacatgtgtttgcacatcttgctggccctgtcgaaactaagactgctgcagaaacaaccagggttgcaaaattttctgaagaatgcctggctcttcaattcaggcaatgttttattcaaataagaaaattcaggcaatgccgcgcaaccagggaatgtgatggttgaacttcagcccatagccataaccaaaagtacctccgccagaccaaactgaagaactagatcaaactcatgaatcctaacaaattacctggaataaagcaacctttgcagaatgatttgtcaggataaagcaacctttgagaggttcaaccttccctcatgaatcttgaactaccaatatgccacaattatgtccatgacagttgaaacatgacttttgtgttccaagcatcaaatgccacaactatgtccatggcactacctctatcaactatgtcataccactctgaaaatccatgcaccttctcatttggttatctccagcacacaaacctcctgctcatggcaccatcaatccattctccatcagaagtgcgcaagcaccgttgaaaatggcttactgacatgatttcccatgaagttgacaacatccttctactgttacttctccgatacatccaacaagcaatcctcatgcttaacaaaatcatcaccactccctctggcatcagcagtggcaccagaagccatgttggcacttgaattagcattcccaagaccacgtcgacgcaagagcatgagctcgacgagtttgacccttgcctcagagacatcattcgactcaacaagctttccacgatcatacatctccctcagaaacaccgtgtgctgctttcgtttcgccgagaggtagaatatcccagggtgatccaagaacacgtcccggacattgacctcgatcccaaaccacttcctgaactgactgaacttctccacctccaccatcttctccacggtcaggctgaggaactcgtgggcaatccctaccgccctcttctccatcttcctccttgccaacttcgacaccctcttgctccctccacccctcggactgacgacctgatacgggccggtgtaaggcagcagctgccactccttcacctttttccgatactccttggtcagcctgaaccccggcggaaactgcagcttgaaggcgtacctgtccggccgggtcttctccaccgccggggtgaattcctcagtcgccggatcggcgacgaggtggagaatgtgggtgttgggctcgtcgggattgggggcaaggcggaagaggtgcgggtgcccctcgacgacggagtcctcgaagtcgtcggggagggcgagctcgcgccagacgcggaagacagcgcggaggggcaaggagcggctgaccgacatggcgaggaggcggtggagcgtccgcgccgcgtcggcgggggagctggcgactgcgaggaggcccgcggcggcgggggtcagggacagcgagagcgggagcggggcgcggaggtggaagggtcatgaagtctgtgtattggacatcgatgcgcttgtgctggattccttgagctgtctgctgtctgatgcatcgaatgccttctgatcagaaaatgctaaattcttcagatcctcacttgtgaggaatcatagctggccgcgatcagaagtgctggctgcagcagcactactcagtaaaacctatagaaatcaagcagcatcagccgccacagctgtggtagaagtgcagccctgttttcagttgtgaggaaatcacaacttttgttttcaataattgaccatgtgtttgactaaatcttcagggactggtgtcatttcttgtgctattgcacaaagcgtttgcatgtattggagtttagattttccagagtccagaacagcctgactttttttttgttgtatatttacaactaaaaactaattgtttaatatagctggaaaattataaattaatatttaattccacaaagggttttatttttgtaagaatagtggtttctgattacacttaatttgtttttgcttgatttttcacagttaccattgcttgtgtctttggtttcgtgtttgataaatgttagtactcaatgggaatatattagcctgttaagatgtgactcatgtcttctgttgggagtaggtgcatcagcctttttttctcttactgaagtaattagcacatataagtctacggtcttttcttttaggggaatattagtctacagtcttggtaaacgcttctcctgataaattgtttcattcatgcagaattgcagattacaggtcatttaacataaatg

SEQ ID NO: 8 - DNA, 3324 bp, Sorghum sp.
agctcaaaggaaatgcatttgcagctgtctgtcccaatcaatccactagcagactcatattattgatggaggaaattaaattcagtctttgacgtagatgcaacaactgcacatgatacgttttgagaaaattaaaccagctttgaccaacacgaaatgagcgccttacgtttggcacgtactccggcacggcaagttagactctgtatgtagtggtagagccggcctccttacgttgggcacagttttagttgagcccggcatggcaggttagaccagagtgtgagccggccaccacaagttattatttataacatatatataggagcaagtgcacataacaaaataattagcatgttcgcttgagcttatcagccgaatctgtcaatcatttagcagtgtttttcttttttaaaaaatcagccaacaatacttctgtcatggcttccaaacaaacaagcgaatgtgagcaaactatatgaattgtcacgtcatatttatgttgagatgaagaagagaaataaatggcatgtaaaattatagccagtgatagacgagcacaaggccttctattcttaaatcagactttgaaagaaaaaaaaaggacttgaatgggagacacgagtaaggccattttttttgtaagaatgggttcttaaaaaaattttaaaaattttcaagattttcagtcacatagaatttttggacatattcatagagcactaatatagataaaaaaataactaattacacagtttgtctgtaatttgcgagataaatcttttgagcctagtaagtccatgatcacacaaaaattatcaaatacaaatgaaagtgctacagtagctaaacctaattttttttgaccgactaaacaaggcccaaaattgttaaatttactcaggtgacacggcattaacgatagtaggtagctaaattaatagtcatactctaacagctatagccgagaaggctaaacaactataaccgtctggctagctaatggtcgagtgaggcccgtatagatgtagttaaatagctaaaatttttggagaaataagcatttttttggaagaatatatttaaacatgggcttgtaaaacttggctgtaaagatttggaatttaggatcttggagccccaaaactgtataaacttgcttagggacccgtgtcttgtgtgttgcagaccaaaaaatttagaaagcatctaaacacctatttgaatgtaaagtttacagccaaaagttttaggatgtaaagatttgggatctaaaagtagtcattaggaaataacacgttagagagagagagtagatcttcttattggtttctcatgcactaatcgaaccaatcactggaccacttgaaccaaactttatcacattgaactttgtcagttcagttcgaacgcaggactggagctgcccttaaggccaattgctcaagattcattcaacaattgaaacatctcccatgattaaatcagtataaggttgctatggtcttgcttgacaaagttttttttttgagggaatttcaactaaatttttgagtgaaactatcaaatactgattttaaaaattttttataaaaggaagcgcagagataaaaggccatctatgctacaaaagtacccaaaaatgtaatcctaaagtatgaattgcattttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggataggtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcagatg

SEQ ID NO: 9 - DNA, 3704 bp, Sorghum sp.
tcgggggtactatcgcgccgacttgcatgcaaagctcaatgacagcaggcgcatggccataataatccctcccgccatccacaaggaccggtggatcatgaggccgccgatgccacacaatcttctgatgaactaggtcatccactccggccttgtcccacctgtgcatccctttccagccctcaaacttgaatactccacacattctggccttgtgccctgattgcccaatatgcacttcagagcaatgcttacaaactttggatgggtacaccaagaggagttttgtgacacctaatctcaggctttcccatgcatctagtgttctctgaccaatatatctgagttcatctggcaagattggagaagattgctgattgttggttttgagtgtggtaggtatttgttcacttttgtatagaatctcatcaggtatgtttgcacctgcatgatggcaaagctcaagtacagctgggacacgagtgaagtcgaaccgctggttatgctttatctcagtctcaaacatgtttttctggtggaaagcctgaacagggacaaggatgtcattcaagttgctcggatcccattcatgtggtcggtccttgatcatacgcttgaaaccatagcatgttttcatttgatgacctgtggctccaatatgaacttcaggacaaaaccttcagtgcaaatcaatgaagaaagtgttaaacagactcataaaatgcagaaatattcaattcaggacaagaatcaataaactgtaatcaggcataaatatcacagagaacgcttagctacaagaatgattggattgtgtttataaaaacctaaaatggtgaagatacagtagaaaaacagtaacctatccattcatagtatctctactttattctccatttcttcaatccttttgatacttgcctaattcaagatgctatagtttctttttttttttaaaaaaaaagcacttccagcaaatcatactgggtttactatatgtaatactgccacactgggtttactatcgttttacagttaggacaagaacagactaatttcccatttattttccttacttgcatgactgaacaggaacaactttgagcagtctggagacaccctcatagacaacctcccttgccctgaccacctcctctgcgaccgggatcatccgcttgattgggtattcctttgtccggttcttaatcctcttaagaatcattggccgcagctgcttccaatccaccctcgaagtggagtagcgcctctggttcagagtcgtgccacacagctccgagcagatgtagctcccggctcttcgccacagagtcgccattgtagcacatatacaacaagcctacaagttcataattcgaataagcgggtcaccagcaaacaatgcgttctaaattccccccgtttaacagttgattagaactgagaaatcatactggatcagaagaacttactagtcggggagatcgatctggcgaaccgcgcccgacccctgatccggcacttccacgactctactaacagcgcctcccgctcgccgatagcgaaacagggggcgttccctcgtcggcgccggagagcagccgtggaagtgccgacgcctgagcgcggcgagacacagcagcgcctggagcgagaggatgcggagtgcggtggaccgataggacgggacctgccccgggggagaggcgacgacgccggggccggacgcgcagagatgactgggccggcggcagcgcggccgcaggcggagccgccgtgcgggaaaggtggatgcgcggcgtggacggcgtctcactctctccatgctttgcctgggcctcaattgggccatgaatctgggctcttggcccataaatatctcccagcttcagcctttttttttcagcccaaacatgccgattctttaaccgcaccggatatctctctcttcgcaccgccttaccggggctataaaaggagagccccgcacacgggctcctctcattgatcgccgccatcaccatttaatccccaaggaaatacctcatcagacgctaatcttctcctcatcaaggtaaaaaaaaaatctcatctcattgtcagttcttcgccaagtcaggggttagttagtgcggtcggcggattcatggttcgtttcgtcggccgcagtgttaggggttatggttcgtggggtgacgtttgatctaagtgggtttctagtcaaaatcatgtctagttcatttgagttggcaaattatctcaaaattgcgtgtgctggtttctaccgattttggcgaaaaataggtatactttgttagttctagattagattctaatttgcctttgactgggtaaatacttttatatggaactgattagttacatttggatttgtatgagattgataaatacttgtggattgctgttaattagattctcttgtgtttatagaactctgatctgattattttcagtagtctagtctggccctcagcagccggggtacgggaacataggtacgttcctcgttccgggaacttcgttccgaaacgtggaacgggaacatcatggaacatcgttcccaaatgcggtggaacacgaatcatatataggagcaatcataggaacgtcagtcccaagcgactatggaacttcgtggtgtgtgtgcatactgcgccgtgcccgtctatgagcggtcggcgaaaaatctagatggtgattgactcattcgcctacacatgcaatgagccaaaaggttggagttggacagccctaaatgaaacaaaaatacaatagtaaactatatcacgctagaaatcgaactcatttggttcaaaacaacatataaaactatctgagccaaccactacgtcccaaacacgttcctttagaaagtggaacgcgttccgcaacttaaaggaacgagacgaagctatataccccctacgttccatagtttatgagaatgtcgtaccgcgtaccacgttcccgtaccacgtaccctatgttccgggaacttggctgctaaggtctggcccttttgagattcctattaattgactgtgattgggattattgtcggtctggccttttgggattcctaattgtttgtatgtttaatatcgtttatttagtaatttattcagttttgatgtttttattgttcatctgttaattaatactgtacctatacatgtcatatgtagaactctttagtagtgaaaagatttatttgtacattggtggtttatgatgaatctgtttagtttccactgttgtcatcctacctatttgttaacccaggtttgtttctatatgtagcctatgcatgtgattaatagctctagctgaggtgcagcttgtgtggatccaatcatttcattttttctgaacagccagtaatcgccgaggctagctataataatctctcagattttcttgaaatggctagggcctctccctcttagttggtagtctctccttttatctgttctctcgccgggcttcagtcggctacaactgctttgcagttctgtactgtaatttgcttactgtaattacgagccgttttgggctaaaataaagtttttacaggtggggaacgcctccccccgttgacccttaaaaaaaaaatctctcagatatcattgcacattctgtaagatatgaattcgtcatgtttccataccttctgtccatttcagatg

SEQ ID NO: 10 - DNA, 3123 bp, Sorghum sp.
caacaaatataatctgtttggctattccaagtggttcttttttttctgggtaaagaagaacttgtcattttgaattctcgggttcaggaatttttctataacttaaatgattccataaaagctttcttttattcttttagttactgatttttttgttggatttcactgcacctaacagtgtttggaagggactagggaaacgtggaatggcgagataaattgtttctatactttgatgacttaggccttgtttagttcacgaaaagttttggttttgactactgtagcattttcgtttgtatttgacaaatactatttaattatggactaattaggttcaaaagattcgtttcgcaaattacaggcaaactgcgtaattagtttttattttcgtctgtatttaatgctccatgcatgtgttaaagattctatgtaatgaggaatcttaaaaactttttgttcttgggagaaactaaacaaggccttacgttttctaggatggtgagtttcggttcatggatcttgctgtctatatttatctatacacgttgttgtacttataattgaaaaaatattgtgtaagttcttgcatcgcattcactgccactgcaatgcaccagatggtgccacagccttgcattctgatgtcgagcggaccatggatgatactaattggtttggtaatgatgaattcagtccttcgtcatcaagtaaacgactctactttcagtgcatcaccagaagaagcacatatataccagattcctcaattttaaagacctttgttccagagcagttcctagaaactcagttacactttctagccatctcagaaaaaaaaaagtgatccactagaggagactcccttagggtccattcgtttagctctggttccggatgaattcatttcagatgatcaaaaataacataaatttacacaacattcttgactggaatcattccaggcatccattccataagaaacgaacagagccttaggatatggcaacactaagtagatgtcgcgcttcaaaccggggccgaccaggggcttcaacgatccctggaattcaacgttctaaccggttgcatcgtgataaacttagcttctggccatctccagagacagtgagttgatgcttgatgctagacgaggggaaaaaaagcagaaaatcagccatactaaatcaactaatgatttcaaagagaggtacctaatgctcaaaaaggaagagattgggcgatttgcgactaaagaagagaataaaatagattttttatagcgttaagaagtgtgtcacagctcttacaggaatgcttgatctacaaatggaatagatgataatggcagcggatatggacggatcggggttgtttagttcctaattttttaaaaagtttttcgtcacatcgaatcttataacacatgtgtgagtattaaatatagataaaaaataactaattacataatttatttgtagtttgctagataaaatttttgagcctagggttagtttatgattgaataataattatcacgaaagtactacagtagttaaatttaaaattgttcgcaaactaaacaaggccatggtgtgtttttattttactctctaaaaatctgcacaaaggttttctgactcatgggccacacgtctcagtgtcggtaaacacggacggaatcacgggagaaggcattaacagcgtcgggtctaacggccacaaaccagcgacgaacgaaacagacgttctgacgtctccgtgtccactccgtcactggttccttctggagagctctgacctcctccgtctctatctacggccggctcgccttccgttccgcgttcgcgttggactctttgcgctggcgtgttcctggaattgcgtggcggagacgaggcggatttctctcgcacggaacggaaccgccacgggcccaaaggcacggtgattccttctccaccaacataaatagccaggacccctcctcgcctttccccaatctcatctcgcattgtgttgttcggagcaaggagaacccagccccccatcgctctcaatcccaatcgatcttcttctcgtgagcctcgtcaatccatcacccgcttctaaggtacggctccccctctaatcttctcttcccatctcagattggcgagtttatgtgattagattagatgcttctcatctagattgcgagtttctgttcgtagatggctggcttgtaagcggttcctaggtgggtttctgttcgtagatgactggcttgtaagcggttcctaggtgggatcgttctgatgatttctttggctgctgcgtagagatagatctggtcctgcttttcttaattcttggtgcagattttgtgacctggttctatgttcttgttcctgctttgtagctcaaatagttgtcttaactagctgggcttattatttgatttgtacctgcatgtattatcaccaaatacaattactgtgaaggagtcaatataccctgctctgtaccttttacctgacgagccatactatcattttgattcgtgtcatatgcatgccagatacggaaattatatgctgctacttgcgttattatcatgctgatttgtttcatatgcacgcctagatagatggaaattatatgctactgctgagcgttattatcatgctgattcgtttcatatgcatgcctagatagttggaagttttgttgtttgctgagtgttactatcatgttgatttgtaatcatatgcatgcctagatagatgaagatacatgaatgttattcgtttcagatagatggaatatgctgctactgagcgttactatcatgttgatttgtttcatatacacgcctagatagatgaagagatggatgttgatttgtttcatatgcatgcctaatagatgaagatatatgctgctactgatgattacttactacttcgtgcccatgcatgctctttggtttacttggatggtgacatgctgatgcagttttgctggttctatagtacctatgtgcttagcatgtatatctgtttcttgttgctgactgtttctttccctccttagtctaccgccgtatacttatcatgttgcttgttttttcttctacagatg

SEQ ID NO: 11 - DNA, 3003 bp, Sorghum sp.
gtttttgtaagagaactaaataggagctgaatatatatagaaatatactagtatttttttaacgtaatggtgacaagatctctgtcttcaattaagatatattagtatttattatgtgtataataactactgtaaattgagcaaaaattatatttttagaataaatatatttgaaattatgaatatagatatttatttcataaattaattaaacttgaattcttttactcatccgaagggaattgagatttacgttttttctttacgaagggagtaacatcccgatgagaaaaagataaatggatatcgggtaagagtttccgagaaacaccgcataagcgtttggctggcattattttatagaagagggatcaaacttatttttagggtgtttggaactgcatgctttaaaactttacttcataaacttcactatagatatactattatttttacggtgtttggaactgcatgctttaaaacaaaattagtttaaagcacctctacaattttactctttttctcaaacaaagtacgttaaaatccttttgctcaaaaatatagaagggaactaattccaaacaccttaattctatttctatactcgtatattagaaaaaaaaaattcttccaagcggcaggccacatccatcagcgtcattgagcatagagatatttggcgtcgcgtcgaccgatcaactaccgccatccaacagaaagagaaaaaaacgattctaaggccttgtttagttcgcaaaattttttatttttggctactgtagcatttcgttttatttgacaaacattgtccaatcacggagtaactaggctcaaaagattcatctcacaaattacaagtaaactgtgcaattagtttttattttcatctatatttaatgctccatgcatacgaccaaagattcgatgtgacggagaatcttgaaattttttacgaactaaacaaggcctaaggaataaaaaaaaaggaaaaattgtgcaaactcttcgtcagtgctgatgacagaagcagctgcccttactctagcaaccacggtgctagaagctatgtacatgattgattccactattttaacagataatcaatagttagtactctttctaaacgggtcttagtttgatcatcatcctgatggagaattaaatcctacattcaaattaccagctccaagattcatggtacaactatagcgattcgcaagattaccagaatcatatggctgatcaactagctagataggctctgagtgaattagtttgcaatcaaatctctcttaatagtgcttgttgtcattctgctcatgagcaaaagtgtcctttactttcgacactctcaaatataactattaactctataatggtcctaaccgtaacacgctgttaatcatataggccttgttcagttggcaaaaattttgggttttaacactgtagcatttttgtttttatttgataaacattgtcagatgaactgtgtaattagtttttatttttatgtatatttaatgcaccatacatctgccgtaaaatttgatgggatggaaaatcttgaaaatttttgaaactaaacaaggccatagtttcattgtaaaaaaaaaaacagctaagcaagatggccgagagagccgttgacgcagagcattgaacggcatctctctcggctgctctcgaatgcgctgcctgccggcatcccggaaattgcgtggcggagcggagccgaggcgggctggtctcacacggcacgaaaccgtcccggcacacggcaccacgatttttccttcccctccccctgcccttctttttcctcataaatagccaccccctcctcgcctctttccccccaactcgtcttcgtccctcgtgttgttcggcgtccacggacacagcccgatcccaatccctcttctccgagcctcgtcgatcgcccccttccctcgcttcaaggtacggcgatcgtcctcccgctttcgcttctcccctcccctcctctcgattatgggttattggggctgcgagtcatctttctggcgatttattatggtctcgatctggtggtaactgtggcgatttattatgggagccctcgatctagaagtcgagtactctctctggtaactgtagcgatttgttatgggggctctcgatctagaagccgagtactctctggtaactgtgggacccttgtagggttgggttgttatgattatttgggcttgtgattaggttgtatctgatgcagaatgatgtattgatcgtcctattagattagatggaaacaagtagggtgactctgatttatttatccttgatctcgtttgatgtccctagctaggcctgtgcgtctggttcgtcatactagttttgttgtttttggtgctggttctgatgcccgtccagatcaagtcatatgaaccagctgctgtcttattaaatttggatctgcctgttttaacatatatgttcatatagaattgatatgagctagtatgaactagctgcttgtcttattaaatttggatctgcatgtgttatatgatggatgaaatatgtgcttaagatatatgctgcggttttctgccgaggctgtagcttttgtctgattaaagtgcatcatgcttattcgttgaactctgtggctgtcttaataagaattcatgtttgcctgatgttggagaaaacatacataagaattcatgtttgcctgatgttcgagaaaatatgcatcgacctacttagctattacttgatgcgcatgctttgtcctgttttgtttgatatgcatgcttagaaagattaaaatatatgtggctgctgtttgattcgataattctttagcatctacctgatgagcatgcatgctcttgttattcactgctactgttccttgattctgtgccacctacatgttacatgtttatggttgcttctttttctacttggtgtactactatatgcttacccttttgtttggtttctctgcagatg

SEQ ID NO: 12 - DNA, 1121 bp, Sorghum sp.
aaaactgacgatgttgcccctggtagcttcatgttcatggggtttcctactcttcctgcagtttacacctcagtacctcactgtccagccagcataacaggctaacagcactcaaaaatgcatgaagcccctcgtttttgaggaaacataatgcccctcgttgtcgttgttcgatcagatatcagaagatttgggaccctaattagtggcagtccagtcatttgtttgcacaatgagtagtcatcaagattgacgaaacaagtatttcttcaagagatcatcagcatggcagcacccggccccctgtttgtgctcattggtgtggtggcgtggccatcctaatggcgtcctgtcatggatgaccatgacggcagcgtggtgctggttatgatgacggcacttgatttggaatgcatggtgaaaacgaagtgcggtcattttttactaccaaatatcgtggttggtgtcctactatcctgactcctgagaggaacaacattcgacatagacctttggacgagtacacgaaagaaaacccaaacaggcaaggatgcaactcatgtgtcagacagaatgtgatcttttccccaacaaggatatgcgacaatacagatttctcgtaaacagtcattttccacataaacgaaaaaaaagaacccagcaccacaaaaacgtggaattcttgcactttttaaccctgtcgcagcaaaaagctaatgacaagattgccaggcaaataattccagcactgctgccagattgccaccatagcatagcagacagattggagtagacgatcatcatctccagcggccctatatagtagccatcgcaggagtattgatttttgtccgggtcgctttccgtccgacgtgtgtagtgtagcgcaatccatcggtcgctgtctgctttctgaagaacgttcccgttgacgccgctacgctgccttgtcctcttttcttcccttctaccctgccgcacgcccccttcttccgtgtgcagtggtgcaggccttttcggccctcaggcttcttcccttcctttccctgcttcggaactcccgaggctgcataacctgattcagaggcagagcgagagagcgtgaggaagggagggagatg

SEQ ID NO: 13 - DNA, 3003 bp, Sorghum sp.
gttaattcccccgctttgtcactgggttattaaaaatgtcgttataatgtatggttgtctacattgttgcttcttgataattaagatgctgtcgtcatgtcatcactcgatgagtagttgtcggagctttgtgcctctggagctttgttgcaaccttcaaggccagtcaagcttcagggtattactaccccaagcatgctccggtaagcgtcaaacttacattcgtcttgggcatgcaccagagtggcaggtctaccctcttcttgcttgaatgcttgaccattttgccacgcaaaacaagcggatggtcttcccatcctcgcgcaaaacaagcggtacgttccattagcggctgagtttggtttgggtttagagaatcgcaccggaaaacggtaatcatgtgccggctttcccttttgtaacggaataacatttccaggcaacgatacgcacaacacaccgtcacagcgttggtgttcggtagagtatctagttctcttcataagaagaaaatttcctatttgacagctgagaaaagttgtgctcttttatttgacatccaaactcaatctttcttatttgtttactctttcaattcttctttcctatataacattcccgtcaattagttggtgtaacagtgttaaatcccatgtaaaaagattattttgcccctagtcagttttttggtcatgatgttgcactctcaagatggatattgcaggttgtcttcctcatacgttggttgttgctgtctttcgtgacaattttttttaaaaaaatgaacatttttttgttgtacaaattaatagatctcattaattaaaagcaccagcatcaccttataatatgattcaattttgtgtataaaaaagacaaaaacatgagatggcaacaacatctaggtttcacatgtaacatgagatgaccaatgagatgtgtctgagcctaactgctttgtccccaacgcgtcgctatgctgctacgcccctgtgtcgggcgaccgcacccttgtgttggcagcctccacactccgcctctccatgaccaattagggatttggttgagaaccagtcgattcccctcttctatgagtacttattttggtctttgcagaagcatccatgagggcaacacatggtgaagtctatggacatgacagaaatatcatatatgaatgaaagaaaaattgagagagtggacagttaagaaatattttgagtcgagtgtcgaataaggaagcattacttttttcagtgtagaatagggaattttcttcttcatcatctgcacaagtttatatgacacactattacgctttttctagtcagagtaacctcccgatgctagctcgccgttgcgctggctctagtatctgggatgtcccctagcgatggtggccacacgagtccacatcaatcatcaagctccacacgctgtcggcaagggaactctcgccgccgcgggtcatgaggccttgtttagttcgcaaaaattttcaaaattctccgtcacatcgaatttttggtcgcatgcataaaacattaaatataaacaaaaataaaaactaactgtatagtttatctgtaatttgtgagataaatcttttgatcctagttactctgtgattggacaatgtttgtcaaataaaaacgaaatgctacagtagcaaaaaaacaaaatttttttaccaatctgaattcatctcaccgccgccacctaggcaccctcagctccgccatcactagggccttgtttagttcccaaaaaattttgcaaaatttttcagatttcccgtcacatcgaatctttagacacatgcatgaagtattaaatgtaataaaaataaaaactaattacacagtttagtcgaaattgacgagacgaatcttttaagcctagtttatcaaatacaaacgaaaaagctacaatatcgattttgtaaaaaatattttggaactaaacaaggcctaggatctcttgactccactgccaccaggagaccctcggctcgaccgccaccatggaactctcgactctgccgccactagggtgaaggcacgagtctttattttcaacggttttggtcaaatccattcgtaaaaatagtaggttcactggatatccgaacagcaagtttggatctgggttgaaaaaattgacaggcctgcaatttataagcgtttcggcccaattaccgggcatcaaacaggccaaactctatatatttgtttggccaacggcccaatggccagcgcagcgtcaggcagccagcactcccgctcccccatttcaaaatttgaaaatatcagcccggcaccgacgaagcggaaccacccgtccaaatcgacaccaacgtcgagcgtctcacctcactcacctataaacgggccccaccatattcctggcccacagacaggtaagatgtcgctgcttgtgctgtcttcccgcaccgaacgttaggtgacccgagaatagatcaccaactggtcttttttagagccacacaccttgggagtacacacagtcgtggacacgcacaaaattggagtacaagaaggtctgtggggcccatttaacagacaaataataggcagcggtgggtcgcgggctagtcgcaactcaggtaccaataaaacgcgagtagttttgaaatattttgctcctaagcccctgtaaggtttttctttttatgtcagcttgatccagagtcccagattagccgccttccgaccgttgaggagcccgaaccgtggaattaatcaaaactctaggggctgaaacgcaaaaaaccgtctcctcgctttcgcttcgccggcaatccatcgtggcccaggctgtcgttccgttctataaagcgagccgagtgggaggagccggaaccctagccgagcaccgcagagacaggcgtcttcgtactcgcctatctccgcgactcaaagcttctcccccttctcccatttcccaccgccgccgccgttccacccttccgacgacaccatg

SEQ ID NO: 14 - DNA, 3162 bp, Sorghum sp.
aaaactagcaagtaaaggacaaaacatattatattagttattaatatcgagaggggataaaagactacaaattttaaactattcgcagatgaggttttatgggacacaataagatgggtaaatattaaaactaaaagaaaaaatatattatatacagttaacttttaaaggggcaaaaaacagagaataaagaaatgcaaaacggaaaaatgaaaacaaaattgcagttgcaagggtttgaactagcgacctcttggttaaagagcaaaagtcttaccattgggttagcaagccatgtagataattaaagttgcaaattcttatatgtagattaatacattgttaattaagtaaaaggcttacccacagggtatgccgtggcccacctgacataccccatgggtccgcccctgctagggcaagtaggcacctagcggcgcataggcaccgacatgtgatccttgccttgtcgtcgcttgatgcatgatgctccagcaatggctgcccccatgcgcgacccgttggcgtatgacgcacgggctgccaccacgtgtgtggaggtgagcccatcctcatcctctttgttcctttcccctttttatttgcatctctctctctctctcacacacacatgaatctagtagcggtgtcaagggggtcaagtagatggcgatgggttggagagaggagtcaacaccactactacaaaataacctaaccacgacctttttgaaaccccctcgaaggcgggcacaatttggaactgccttagttaataacctaaaatccattatccgaggcgtgatgtaatataaccgccttggttaatgcctattaaccaaggcggacattataacgcaaccgccttagtaaatcattaactgaggcggttatattacattgcccgcctcctaaaatctagcccaacccactacaaattctcagcccaatatcagagacgaggcccgatatgagcgagtagtacataaacataagttagggtttcctagctaaccgatcccttcactccctccctccccagccacctcactccccaaccgagagcggttgctctcctagccatcctttggtggcgtcgtggggggtgtggccctccccactccggcggcagcgcatgggaacacgaccccctcctctagcggtgacgtggcgcgggtggtgcggcctctcctccgatggcgtcctgagaccctacttcggctcctcaccagccccatagccctggccaccccgacactctccagcagcggcgttgcacgagaaccgacgtgaggctatgaggatggcggcgtcggagcccgtgcgtcagcagcagggtggcgacaatggtggactgggcttggtgggcccgtcgatgatttaaatgggctcgtcgatgggctttttttaattattttttttctgatttatttatcgagacgggtaagcaatcgcctccgttaatgcttgattaaccgtgaccttttgttggagacatttatcttgcccgtatcggaaaatcctttttgcccgcttcagataaggatggagatgaccttacgagtggcccgtggtttttctctagtataactaaatgaactaaataaaaaaataagggaagaagttgtttatttagttcattaaattagactagagaaaaaccacacgtactggagcagcgtccccatagcttcagaaaaagaattttgtagtagtgcacaatgcagattcaaatagcgatggcatgagcaacacagattctcgacaaatagcaagaagcaccgacacacgactctctagcatcactctctggatgtttgatcgaatgagaaaataggatcaagatcaatgtggttgcaaaagatattcgatttctcaccggtctatagcggaaacaaccatcaacgattgcagacctaacttatgagctgtcttgcatggactatcagaaatcgaacaaaaagaatggagctgcgtgtgagaaagacaagcggaatttagttatttcactttgttttctttttatcatgtcacatatgggcagctagtgatgccttcgcatcacagcacttgaagtgagattctattttgtttttgttaccatgggacctgattttcttttggctcccacactctaggggcttgtttagttcctaaaatattttgcaaattttttcacatttctcgtcacatcgaatcttgtgacacatgcatgaagcactaaatatagataaaagaaataactaattacacagtttacttgtaatttgcgagacgaatctttaagcctagttagtctatgattaaatagtatttgttaaatacaaacgaaagtaacactatttatattttgtaattttttttaaagtaaacaaggcctagaatcagacacttggccgttacggttgcaactgaccggccattccataggggccgagtcagcaggtccaagcgcccaagggtaaccctgtactttcccgcgacggtacgatacaaagtttcaaatttcaaaatttgaaacggctggccaacagaacccgccggcggccgctcccctccattcccctgacgtcgtcccataggctccccagcctcacacatactacaaatctcacccgcatcaatgctccagggggctcaaatatttgtgcccatcagttggtcccacatgtccgtgtcacaacatccacgaccgggtaaatgtcgccgagaccccgagcgcgccggctccgcgggacccgcccgccacagctcattcccaccgttgccggccgccgatcacgcaagcctcagagccgttcgaatccaaacggtcgttaacccctcgttgcctccgccccgcccaccacccagagactgatccgtgggccacaccatcacaccgtcagtcccgaaccagacggcggctaggtctaccgcgccgcgccacaccatcacgggccggccgcggccgcctctccactctgcctataaaagccgccgcggggctgggcggcatttatcgttcacctcggcgtcttcacaaacgccggcgcttccactctcgatcgatcgatcctcgaccattccccattccgtcctcccccgatcgatcctcgaccattccccttcccgtcctcccccgatcgacgagcggttgtctgagagaagaggaggaagatg

SEQ ID NO: 15 - DNA, 3131 bp, Sorghum sp.
gccactttaccagactgcttcaacaaatttgagcagccaaattatgagttgtgcttgcaactgaacgtctggacctgttcaagcttttgagcaaaatgtctattctaaatgcgattcaaatttaaaaggctttgattcaacactcaaggagccttaatttgaatgtttgagaagccactaatcctctgtcagtctgcaatatgttttactccttccattctgaattataagacgtttgacttttttgactctaaatttgaccacttgtcttattaaaaaatttacacaagcataatcaaatttaagttattattgaagaacctttattaataaaccaggccacgataaaagaaatgatatcttagacaattttttgaataaaacgaatagttaaacttggtgttaaaaaaaatcaaatatcttgtaatttgaaatgaattgcgtactatattattgtcatgagtctgtttctttgccgtataactcgtataaaagagcagatttgttgttccctttttgaattctagtagctttgatgttctgctatctcaatttttattctcacctctcgtgctcgcgtctcccagagatccatggtagcagtttagccacgtaagaccttgtttggatgttgtcggatttacttcaatccatgtgtgttggtgtggattaagatggaatttagttcaagttctactccaatccacgtcgacacatgtggattggattgatgtgaatccgactacatctaaacaaagtgtgagcaggactgttgaccgatcgctatgttacaccattcaggaccggcgctgccccaagtcatgtacgataacaataacaagcatttcccctgactaatcaacgaagaatcggggcgaggacaagagtggttagcgttgctgttgaccatcctacctggcagcaatgttcaactcgaagctagtgtacccatatatagcgtgatagcaatgatgtactggcatcgaagcagtcaataaaataagaccccactgtttttgttaaccatgattggatcgatgtatcgctaggggctgttaggatcacttggctatctaaacgggtactgcgtccttagctttcctccaccgttgaacctaggtatcgtattgccgtgtggcaagcgaaaaaatagcggttcttttctcttgtctcatgataaagttttagtacgtgtttagcttgtactttcagctagtccgtccccagccatgcatgcattatagttggtgccactgccaccctctctgtgttccctacctgaacatgagctgatcagcaacatgctggtaattggtccttgtcattcctctgactaagcaacagctacgtctgttgacacgcagggtccaggctttgcttgcttgcattacctaccggccaagcgtcgctatccgctgtctaaaataaatagccgccataatcacacatctaatcattagggcacttacaagactctatcacagagtccaagagaattaattacatactatttatgatattttgctgatgtagcagcatatttattgaagaaagtggtaaaaaaataagactccaagtcttatttagactctaagtccacattgttcaagataataaataactttagactttatgatagagtctgcattgtgagtgcccttaggacagtcccaatggaagaaaccacgacagtttctatagcataggatactgtaccaagaaactatcttttccattgcattattttgtactagtgtctagataaaaatgttcccaatcattcactttatttctctctcacatctttggatcctctgtgcatttggtttactctttcttactctatcatcctctgctgccggcgtccaaatagcgaacgagcttcgtctctatcggataactagctagcagctccgtcacctggaaggaaggattgtttgcctgctcatcattcaaggctgccgggctagcaggcggcagagccttttctactactttgctgaggcacagcagaatgccgccaagccgctcacattgtggcttaatggaggtttgttgctactgctcgcacctcatccatggcgtgagcatgtgcggaccggcgagccctcgtccatggcgtgagcatgcacagaccaacgagccagcaagctccatcgcgtcgtgcggcacgatcgggagattgggctgcggccggagctccatggcggcgcatgtgctgccaggctagagttccacgccgctggcccttggcgacgggagcgacaacacaatttggaataggagatccgaacggagttgaatggaaacgaccagtttctccccaacgcagatcacgtagtttcctcacgcatcggtgtgagagacgacttcgtttcctccataggaaacggtttctctaatttttcttctctctccctaataaatctattttcacatcaccaatttgcttagttggcaagttaattaataacgataaaaactaccatcaacacatcattgggactgcccttacatgctcaagacgagaagagagcaccacaggctacaacaggctacagctcgggtaagcttgtctttttgggcccgccgttcggttacgacaggtcgacagcctgccacgtgggcccacgcccaaacctggacccaaaagtcgccaacgccaataccaacgccaacagaaagagcccaaccaacaaatcgacacaaacttccctttttttaaaaaaaaaacacaaaagaaatccaggaaacgggccctctagccgtccgatcaacaaacgcacggtggagatggaccagctccaccgcctcaacgcgtcggcgcctgggcccctaccgcggcggccgggtctctctccagtctccactctccacccaccgggcacgggccgccacagcacaagaaggtccacaaccccctcctccagctcgaaggctctcggtggaagtcgcacgggggcaacagcatagagcagcatttcaaatccgtcctcacctatagacaagaccgcaagcccacagcacccgagagaggtcgagaccgtgcgccgctccccgcccgccttttccccgcccgcgtccgacctcgaccccagccccggcgagccagcaggcaggcgtcagccatg

SEQ ID NO: 16 - DNA, 2680 bp, Sorghum sp.
agctatctgatagcaaggctgttgtggacctctttttttttaagaagttatctattttacaaaatagctaaacattagaatttgactattaattttagccaagctcttggagatgctctaaggtgttaagcctggcttgattaggaagccaagggggtttggaccctggtttatgggaaacggctagggcgagcattttcttggatctaggttagggctatgcgaccaccatctctgctcatcactgcttgtgctatcacctactcccttcgtcccaaaaaaagtgacgcttttgactttcaaacatcgtgtttgaccgtttatcttattcaaaaaatttatgtaaattataaaataaataaatcattagtaagtatctataatgataaaataattcttaacaaaatatataatatttatgtaaaaaatttgaataagacgaatagttaaataaaatgtctaaaattctaaaaccacattctttttaggatgaagggagtattatgccgttgtcaagctgccaagcacttgcgtcacttgtgttgccacccttcgtcccgtttgcacacaccgactaaagtctcatctgcaatggcttcgacttcgcggtgggtgcagactgccactacgcttcgaggaagccgtcgccggtgattgcatttgcatttgaagggatttttttttcataaacctaactttgtgattttttcatgtcagaggcattatgattttttattgtccacttaataccgttaaatgctagaaacgaacggaaagccatagaaggaacgaaagttggtttaaagaaagagagttcaaaaaaaagaactatacaaaaaagaggtattttttgagggccaagtaagggcatgtttagattggagatgaaaaatttttggatgtcacatcggatatgtcggaaggatgtcgagaggggtttttaaaaactaataaaaaaacaaattacatatctcgactggaaactgaaagacaaatctattaagtataattaatctgtcattagcacatgtgggttaatgtagcacttaaggctaatcatagactaactaggcttaaaagattcgtctcgcgattttcaaccaaactgtgtaattagtttattttttatctacatttaatattccatgcatgtgttcaaatatttgataggatgggtgaaaaaattttaggctgtaaactaaacagggcctaaatccttagcataacactcttggcacgatgtacagagaccaaaatccagtcgaatttcaaatttggataaacaaatactcctgacctgatgtacgcaaaccaataaggccttgtttagttccgaaaactgaaaagtttttggaactgtagcactttcggttatttgtgtcaaatattgtccaattatagactaaataggatcaaaagattcgtctcgcgatttacagataaactgtgcaattaatttttattttcgtctatatttagtgctctatgcatatgccacaagatttgatgtgacagagaatgttgaaaagtttttggttttcggagtgaactaaacaaggcctaaaataaaataaaaagatttgccatgtacgcaaaacgagacagtcagacagcccatcctgggccgacgccggcaaaccagaagcaaacaaacggcgagacgcgcccggggcagtagcgtcacaccgcaacaacctgttccgttccgcgccggggggggggggggggggtggggtggggtggggtggcgccggggcaacaccgtcatttccgctgacacggaagcggacacccgaaaaatttcaaaatccaagcgcccaacgggccgttttcgaacccgacgcagccgcccgtccgatgggaacgatcggacggcctccggcggtcgacggcggcgttggagggaacgcgactgggccgcctgatccggtgccctagccccccgcgcccactataaaatccgccccctttctggccactcgctcatttcatttaccacaccctcccccttcccctccccgctcccccctcatctggacggccgactcgcttcttcttctgtgaggtaatgcggcggaatccttgtgccatattacgattttgggttttgttttcgtgttccctccgggatttatgtctggtagtagcagatttggggacttttttttggtttcgttttgtgaggtttgaattttggggctagatttgggtggatgttgcggtgtccttcgctgctggtgcggctatgtttttttattagatctgcaccgctccaaattttgtttaggcgtttgattgtcagatcatcagtcatctttcgctgcttctggattctacatgttctcggttcttatattgggatttgagatttggctttgttcataggtgacgcgcttccgtgaggtattctcatagaatttcaggtagatctcaaggggctcctcacttcccttgtggtgctacagccagtattttaagttttctgcagtcctctctctttttttaactgcactttttcctttattcccggatctgattgatttcgtgccggagcttgttattcctccatagatctggttctccactccctttcggagtaatgtctccatcattttcacgctactaaccgcccttctgctcccctcccacctgcagctaccaaccttgagatcaagccatg

SEQ ID NO: 17 - DNA, 3081 bp, Sorghum sp.
ttatagtccaatagtcttttgcatcttcagacaaaagcctaagatcaacaaacatcactttgcatagcattatcatcgtcacagagataagtgataggatgttgtaacaaattttatgagtccttgatatatttcaagttctcatggtagaactacaaatatctaaaattaacatgagagctattcatagcaattcactttgctatctaagaaatcaatttcaaactatgacataattaattttttcgcacaaaaactgtaagcatatatgtgtgccatgaaagctaataggttacatgtttgattggcaaattggtaatggcaacgacaaattgcggagggggatcaatgacgagtacacttacaagactttgtttggttggacaacacgagattgagagtttgagtatatttagataacacctcgaggtgagaaattggcatcgcttgaacttatcagtcaaaatcagctatactttttcaatcatagaatggtgttgtttttttctcacagcgaattagcatcagtcacagaaatgagaaacatcaagtaacgtgaagtgatcatgttgttaatcatcgcaggggaaaagcactgaaccaaataacatgttagtgttcctgctttttgtttcaagcccaattatggcttaccctcctttgaagcccctttatatttcattaagatgatttaaaaatatcaactaagctataaaaaaactagttgccacacggaattgcaattgcctacttttgtacgtacttttatgacccccccttattgtgacattagcattttgaaagataccaaaataattttgacaataaaacttgacaaaaattgcatgcatttcaactttgatcaaactctgacaaacactattttaaaaagtacgtaagtgcatagataaaattacaaactcactaataattcttctaccaattctctagatgttttccctttttttaactctgttatttgaactccaaccagcacaattaaaaatagggaaagcagttgttcggggtgtaaaagaaaggacaaaatcacaaacttagacacaaaaagttaggccttgtttagttcccaaaaaattttgcaaaatttttcagatttcccgtcacatcgaatctttagacgtatgcattaagtattaaatatagacgaaaataaaaactaattacacagtttggtcggaattgacgagacgaatcttttgagtctagttagtctatgattggacaatatttgtcaaatacaaacaaaattggtactattcacattttgcaaaatattttggaactaaacaatcttcacaacaagggaaaggacgccattatcatctctcaaaaacttttatgaagctaaattgagatctagatctcctagatcatttatcctgaagtgatacttgcatagtttacttatctcaatagaagtgattctttctccaaaatcaaattagaaagttgaggctaaacttaactatcatgttgcttcaaattcgaaacaaacttttcattctccaaaaacatgggccatgaaacgtactctctccacataccaaaacaagtgcacgtattgcttatcgaagaaccaaccattttttaaaagtttaactaataaatatataaaaaacactatcaatatttatatctttaaataaatttataataaaattatattccactattaatccaataacattctacaactagagttgggccttgtttagttcactccgaaaaccaaaaacttttcaagattattcgtcatatcaaatcttcgagcacatacataaagcattagatatagacgaaaataaaaactaattgcacagtttgcctgtaaattacgagatgaatcttttgagtctagttagtctataattggataatatttgtcaaataaaaacgaaaattatatagtgccgaaatccgaatttttttcgaaactaaacaaggccttgcttgtttttgaaactaataaaacacacaattatccaggtcttgtttagatgcgaaaagattttggatttcgctactgtagcactttcgtttgtttgcggcaaacattatccaattatggactaattagaattaaaagattcatctcatataattagtttttatttttattcatatttaatgcttcatacatgtagcgaaagattcgcttgaaaatttttgtaaggccttgtttagtttcgaaaaaatttcggatttcgctactgtagcactttcgtttttatttgataaatattgtccaatcatgaactaactaggattaaaagattcgtctcgtgatttacagacaaattgtgtaattagtttttgtttttgtctatatttaatgcttcatgcatgtgccgtaagattcgatgtgacggagactctcgaaaactttttggatttcggttgaggccttgtttagttccgaaaaattttgggaaatggacactgtagcgctttcgtttgtatttgataaatattgtccaatcatggactaactagactcaaaagattcgtctcgtcaatttcgaccaaactgtgcaattagtttttattttcgtctatatttaatactccatgcatgcgtctaaaaattcgatgtgacggggaatgtgaaaaattttgcaaaattttctgggaagtaaacaaggcctgaacaaggccttggtttcgcgctcgtgctgtcatgcagtacccgcagaggcgcagagcaacagaggaattctcgctcacgtgacaatgacgtcacccgcgtgcgcgacgaaaaccatttccctccgtttcttcccgcgcacactttggccatgtcatcgatccgctccagaacgcatctcagccgtccaagccaagaagcaccaacgcctcgcgcgccttccacgccagcgatccgcggcatccacccttccaccaaccagcgcgcactacatttccgcttccgctataaagtaaccgccgccccacatcccttttctccaccgcaattcctccgcaacttcacaacacagatcatcgtcttccaatcgagcaaaccctccttcggtttagagaatccgagcggcggcatcgatg

SEQ ID NO: 18 - DNA, 3062 bp, Sorghum sp.
caaaagataacgcatatatttttactggcacaaaaagaatagagtggatggaaagacggtcatgcagagggtgtatagttcaccttttatttaaaaaaagaaaaagtctaaatagccccctcaactatacgcggtggactacttcaccctctgaactataaaaccgaattttctactccctgatctttccaaaactggttaaataacctcgcaagggttttagaccgtagttttactatagtgataatggttttgtcttttaaaaaaaaatattttcgttgaatctttgaaaaatcataataaattacaaaataaaaaatctagtctttttaggctccacatgagtagatctaatatgatatattttactacaatttttttgttgtaactttagagctatgaattattccaattaattaagcatagatctaaagctgcagtgaaaacttatactaaagtataccatattatatgtttactatgcatatctaggagtccaataatttattttataatttttaaatatttagcaaaaataaataaaaaagaaaaaaacaaaaccaccattaaaaccggctgagggggcttatctgactggttttggaaaggttaggggtctagaaaatctgattttatagttgagagggtgaagtagttcactatgtatagtcgagggggttatatagactattttcccccaaaaaattgggttttgattcatcattttgcaaaatagaactaaacattatgcatttttttaggaaaaaaatggttattctccattttggattttgacctcaagtggcttttacgagagcaataaattctacattttggatgaaactaaatatgaccctgaaaatttcagctttttacgttccattattccaaagtagttggtattttatattttatatttttattttaactaaacacccccatagattttcattggcacaaatgtttgcatccccttagggcctgtttagattggagatggaaattttttagatgtcacatcgaatgtgtcggaaggatgtcgggaagagtttttataaactaataaaaaaacaaattacatagctcgtctgaaaactgtaagacaaatctattaagcataattaatctgtcattagcacatgtgggttactataacacttaaggctaatcatggactaactaagcttaaaagattcgtctcgcgatttgtcggtgtttttcccccgggggggggtcacaccaacgagtaaatttgtatgcgtgctcccctttccggatggtgatgcaagaagacacagagatttatcctggttcgggcaagagaaggccctacgtccagcggggggagagagtttgtattatcttgcacctaagtgcttgtacaggggtgaatacaagcgtggtatgaagtgtgtagctctactatgtgtgtgtgttcttgtgttgtgatctctctcccttctattcctgagtctctccttttatagctccaaggagagacacagggtacatgcgtagatgttagagtagggatcgatagccacatggagcgctgacctactcgaggcttccgtacggcatggcctcgagccgtcccatcttgatagcctggtgatgattacgcgtgctcctgcgtgctgccctgcctgccgccttgtgctggttctgaggtcgcatgctcgtatggtatggtggcggatccggcggggcagctgtggtgttgtcagtcgacgcccaacttgtctctgggaagggaccttgttcggtcgagggtcgggcgccgcgtaatatgctgatatctggagcgctgaccttgggtgtcccgagggggtcccgattagacgttccatccttgtttcctgcgtcctgacacgccctgggtcgttcggtgggaagactgcaaaaagaacgatgggacagaagcttgttccctatcacgccttcccaaactgtgtaattactttatttttcatctacatttaatgtttcatgcatgtgtccaaatattcgatgggatggatgaaaaatttttaggttgggaactaaggccttgtttagttcctcaaaaaatttgcaaaattttttagattctccgttgtatcgaatctttagacgtatgtatggagtattaaatatagatgaaaataaaaactaattgcacagtttggtcggaattgataagacgaatcttttgagcgtagttagtccataattagacaatatttgtcaaatacaaacgaaagtgctactattcttattttacaaaattttttgaagtaaggccttatttagtttcgaaaagtgaaaagttttcagtactgtagcacttttgtttgtttgtgacaaatattatccaattatggactaattaggatcaaaagattcgtctcgtgattttcatgcatgtgccataaaattcgatgtgacggaaaatcttgaaaattttttgattttgagggtgaaccaaacaagcttagcgcactgactgttgggcctgaccgagaccgacgctccgacgccaaggccttgtttggttcaaaaagttttgcaaaatttttcagattctctgtcacatcgaatctttaaacatatgtataaagtattaaatacagacaaaaataaaaactaattacacagtttggtcgaaattgacgaaacgaatcttttaagcctagttagtctatgattggataatatttgtcaaatacaaacgaaaaaactacaatatcaattttgcaaaatattttggaactaaacgagacccaaaaccaaccgccagcgcgccgaaacgcacagttccctccggctcctcccggctacacacgtcagcaatccgcgtcaatacccatctctgccgttctgcgatggcaccaacgcatcgcgcccctccacgccaccgatccgcggcaccgacccctccgccaatcagagaccgctgctccattccataaataaaaccgcaccccacgcctctcctcgcagcaatcgaaattccccgtcctcaaatcgacctagctagcgaatccctccgtccccgcagcctcacccccacagcatcgatg

SEQ ID NO: 19 - DNA, 3076 bp, Sorghum sp.
accgatccgcggcaccgacccctccgccaatcagagaccgctgctccattccataaataaaaccgcaccccacgcctctcctcgcagcaatcgaaattccccgtcctcaaatcgacctagctagcgaatccctccgtccccgcagcctcacccccacagcatcgatggcgcccaaggcggagaagaagccggcggcgaagaagcccgcggaggaggagcccgcggccgagaaggccccggcggggaagaagcccaaggcggagaagcgcctccccgcgggcaagtctgccggcaaggagggcggcgacaagaagggcaagaagaaggccaagaagtcggtggagacctacaagatctacatcttcaaggtgctcaagcaggtgcaccccgacatcggcatctcctccaaggccatgtccatcatgaactccttcatcaacgacatcttcgagaagctcgccggcgaggccgccaagctcgcccgctacaacaagaagcccaccatcacctccagggagatccagacgtcggtgcgcctcgtcctcccaggcgagctcgccaagcacgccgtgtccgagggcaccaaggccgttaccaagttcacctcatcttagattggatggtgtaggtagatgtggctcggttcggtttatgtgatattgctacctgtagtagtagctggtgggggttcgaaatggttggatgttgatctatgtgtagatggattgtggtaagaattatggtggtgcttttggaaccctgtttcatgatccagaatagtcacagtgcttgttctatttttgatttgtcaggatggatgctcttaatgtgtagttatcatgttgctgacagtgaactgatgattccatgtgcaaagctttatgtcaagtctggagcaagcgtgttgtgcatttgatggttgctagctaaagtatctcagtgtgttgaggtgggaaatgttatccaagtgtcgtaaagttggatatcatattaaggtttgttgacacatttgccaggagggaatgaacatgcacaggcaatttaggcgtcatttcctctctggaagcttgatagtgtaggaagttgtgatctatggacaatgtcatggcaattgctgttctgctaatctgagttctgagcttctgagcttcaaattctgatatcaatggtaaatctacttgatatttagaatatttctgttgtcattgaggaacatgtagaaaagatatgctgtttttttgggttgcaagttggctagcactagaacccatgtataggctgggcatccctacttgtttggctcctgttatctcaggttcatattcgagcagcatgtttgttggtcattcttctgaatcccagctgcatggagccttccatttcttgcaagctatccttaaaaaaaacaagatagctggtaagttatcatcctgtccaagtccaaccttcagcattgatgttcattgttattcattttgcagagatgtctttccagccactgatgttcttctatcagctaaattccaggagtactattttttgtgttattcatcgtgactgtgtcatgcactgactaatgcttcatctggggtgttaggccatgtttagttcgggttggaaaaaatttcgtgacactgtagcattttcgtttgtttgtggtaaatattgtccaactacagactaactaggctcaaaagatttgtctcgtaaatttcgaccaaactgtgcaattagtttttattttcatctatatttaatacttcatgtatgtgtctaaagattcgatgtcacagggaatcttgaaaaattttgagtgttggggtggaagtaaacaaggccttacttggagttggagcatgatgagcaagcccaatgacatgatccaaatatttctaaattattattggtgcaatccttggattatgcgcttatccatattgccatattgttggttttgaactctggatgttacctgttttgatattgttgataaaatttctgtggttactttgttttttggttaagcttttaagtggttgattggaacttgtggtcattagttagaaatatagtgtgcctgttatgttgaagcatggtgaaatgtgtttacttctggaactgtgtaagttctggagtaagagtaattatggttcctacggcttacatttatatagttactacttgcaacggaatgattttatctggacctcaaatatatcttctgattttttttggccatcgcactgctttgtggaattgaagctgaaattgaagctttcaaggatagagagagtacgatgtacttactgttcgctaaatcgtttttgtggccaatgttgatttgttacaagagaaaaacgttgttctgtgcctagaaaagtatggttgattctggctaataagttcaaatgttattttagtaaatgctgagaatgtatattgcagtaatattattagtatattacacatttacactagacagttactggctgttttcttttatataaaagttgcataatggttgtataataaagtactattcccttcgttctaattgataagatattttatttttaagatttatattatttttattatgcatctaaatatagtttatatctaattgcatagcaaaagccaaaacagttaagcaaaaaaaactatatataatttttgaaatggaggaggtgtattctgtaggagtatcacattagacagttggaccaggccgaaaccaactgctaagagaaaggccgaccggcccaccccactctgcgcgctgaaagccagttccctccgtctcctcccgccctatgctctgaccacctcaactatccgcgccaaaacccatctccaccgtccatttgcgacaggatcaacacatcgcagccatccacgtcagccatccgcggcaccggcccttccaccaatcaccaccagctgctccgtcccgttaaattcgccgcacccctctcctctttctccatcgaaatcgaccgagcgaaagcgaatccctccccgccgcagcctcacatcgcacgccaccgcgaaaccccagcagccgcatccatccatg

SEQ ID NO: 20 - DNA, 3003 bp, Sorghum sp.
agattttgaattaggggttcaaattgaaaagggggcaatttgtaaaaatctgtattttcaaaattactttggattttgcattgaaacttcaaaaactcaaaacaccaaagttgtacaccttaacaagatctacaactttgcttttgaactcatccccaaattttgcttagtttttaagttacagaaaagggggtagaaactgaggttgaaattagggtttttcttaactatttccttacaactctccttaactagggattaaaccaccatcacaagcatcacttcacaaaataaacacactttatcttcctaagcacaatcatcaaaaataaacttattttaagttgatgcatcatgatgtgcttaacaaacatgttttgcaatgcttatgatgacatgatcaagttttaatattcgtaacatcagggatgttacatgaactctttggtacaacccttagactcatcatacaaaggctcttctgctgccttcttcaaggccttcatccctttcattaataacattgatggtttagtatgacgacgcaacattgccttcaagaattctacatcttccgcagtcgtatcatttggtaagacatgagttctggcaccatcatttcccccagcaaatccaccaacggtaacatttggcacaacatgttcactctggagtgtgtcgtggaaaaactgatcaacgggcatgtctagagcatcggtgtcgatgtttgctgcgtccccaactggataaggcaccgagctaccctctccatgctatgtccaaatcaagtagtcctttataaatcctcggtagactagatgagaaatgattacttcagtatcttcaaatagaacaatatttttgcagtcgtagcatggacaatatatgtgcttcgtctttgttctcaaagcatggttcttagcggcatcaacaaacctatgcacctcgggtatatatgatggatctagtcttgataagttatacatccataaggacctctccatcatgagctgtttaaaatttagtaaattaattgaaatattatctaggaatatttgttatcaaaaataaagaacacctaacaattaaatcaaattgaaaaaaataaaacaagtattaaaaataaagataaaaccctaagtaataattaaaaaagaacacctaacaactaaatcaaattgacaaggtagaaaaacaccgttgatggaagctaaatatatatctttatttcactaaaataaatttgagaaggaaacatggaaatggtgagaggagagacaacatcttaagcaacctcatacatgaaaaatgcttattgcataattaaatcatatttacataaaaaataacatgctaaacaatataatttaaaaaactaaaatgaaaacttatctttctctctcttcccaagcatgaaaacaccattcatggaaaccactacatatatatttcttggattcactaattagagaagaaaacatagaaaagaagagaggagagacatcatctaaccatcttaagcaacctcatttgagcaaatatagtccatacctagctattctactctctccctctcatggtgaaaccctagatccaattaaaaagtacctcaaaatgagcaagagggcacaaaaagaggcattggaggcatcaactaacctttcttagccacgtccttctaagaaatgaagatcaaaacctccccttgtattttgaaaaatatggacctccaagataggctgcaatggaaggtggctgcgagctacagttctgttcgaggaggaagaagaggggctgggggtatttataggaagaataagcacatgcggtttgcttaaggaaccgcttgtgtaaatctattaacacaggcggttcacatagcagaaccgcctgtgtaaatggactatttacacagacggttcagttatgtaaaccttctgtgctaatagatttgcacaggaggttcatataactgaaccgcctgtgtaaatgatccatttacacaggcggttttcttacaataaccgtctatagtgactcttttatacaaacggtttttaattctggccgtacaaatttacaacacagatgcattataagtaaaaccgtctgtgcaaagtttttgcccccgccgacttagagcatcgtactagtggaagaagccaatagactctaatgaatgtagtaatggatgtagtagattgcgattagattgagatttagattagaaatgtagtaactgcaaattgtggactaatgggaacgactttgtacaaatatcatagattagaagatttttacaatggcacggttacatcctaaacttctttctactccctccgttctatgatgtgaaaacatttataagaccaaattaatgcactataattaaaacatatattagtgaaagtgattttatcttataaaactatagacgacgttttatgcattttctacaaatttagtataaagaattagtgaagtatctaaaaattagctaagtatctaggacaaaactaaaaaatcgtaaacaaaatcttgaaggcgtgattcgaacaaaacagagagcccgcgcgcgcgtgggacgagttcctcaaaattcaaaactggtcgtcttctcgatctctcggctctttctgctcttatcctgtgtgtgtgtctcctatgggtctggacaggcatggagtggtcatgagaggacgacacgagcacgaccatgcagccgcgtctgtctgcggccggtctgcctgttcaccgagccccccaggcggtccaagttaccacgagttaccacgtcgcccgtccaggcagccgcgtgtccatccacccgcttctcacgtgtccctcccgtcctcttctgctctgcataaagcgtggagcctccgctccacccatgcttcgtactacctgtccctcgacgcgcgcgacccctccatttcttcatttcttcacgctactcaccgtgtagtttgttgcaagctgatttggtgcttcagcactatg

SEQ ID NO: 21 - DNA, 3069 bp, Sorghum sp.
gctgttgctatggcgagtgggtcacacgaagatgaatctcgtatcccttccctgcctttttggttctttgtgcttaaactcttgtatgtttgtacttaatcaaccgtagcatttctttggttctcgcggtgacaaccgcaccgctaagaaccttagtgatgtcttagtgcatttagtgcacctaggttgtggcgccctacaagtagtttgagcactcgtgtccttggtgtgtcactccctcttatgccctgtcttttgccgtggcatgagtattggaaggagtagaccacttcccttcttcttctctctttattccaccctctcttggctctctccaactacaatggcgtatgggttgagagagaccggaacttctcgtgctcatagtctttacgattcctgtcgatctctatgacacttggatcgaaagaccgtaagctgtggtgttgcttagaatgagttagagtctaagccatttattaaagccgtacaggtcgacacgatcgacccgggaagtaccggctaggctaagactcaagcttgtacttggtgaacaaccatttccgtttcttttagcccagggatcggacacccaccgagagggctaagccgttttcctttcggtgctctttcagtgtagttgtccttcagtgttttgtcgcctcttttcacagtctttctaggactagtggattgattgtttcgccttgtttgtcgtttacgaggtagttgcttcgggtagcgttgatcgaatcggaaccagttgaaggaaggacatgcagataggagaaagaccttggatgagtacaactacaagtgaactgaggatcttggaaatgtcactcgacaggtgccacgtcccacccaacctcgtagaatcctattagacatacaatatgtagatgctagtgctttacttttatgcaaatgaattgagataggttgcatggtagaaatgcttgtgagccattgccttgttgcaacctataaccctcgcacacccgctgttaggttagacgcttgcaaactacttgctactgcttctactacgcattatatctgtgatgtgatgcattgtggaggattggatgtgagtggatcaggcacgtggtgccgataactggttaagaaatggataatggatttggggagatcttggcgtgtgtcttgggtgtgtggtgagggtcgagtcgaccgagcaggatctacgacgagtcttgggacaagtcttgccggaggacgctacctgggcgttctccacgagagatacctgtggcgggtacatgtgacagggagaggtcccggagtggagtgtcttcgtgggacgaagcaccgggatgggaggtgctgtttagcacggggtaatcggatgtcccgtcgagcggggcatcggttggcccctcgtgaagatgtcctgttcggtcaccctaaggactgagatgtcctgagaaccggttcgtaggaagccttgcattcccactcgccttagccatggaacgggacggatgtacgaccagctagggcggtgccactactactaggttgttagcggaaagtgtagggaggtacgggcctgggacccacaccctcctaagacagcgtagtgaccttgggggcccggtactacgtctcacagtctcagcatgccggtggtactccggcatggccccagtcctgagtggtagggtggcatcgtgtttagttggaaggcagcccggtatcagcctagacgatgtacagcgtcgatgatggtgatcttgtgggtagtgcaaacctctgcagagtttctggttgatcgatcgatacatatgccgtttacggctatggacctttcctatgtttccgcttcacttgactagtgagaggagtcctttctaccttcccctgggtttgtgttggatccggcgttggccgatgaggcaaggcacgagcgggagtcgtacttgccgcctagagagtgagagtgtggtgagatgtgtgtgatgggatggatggatggatgtgtggaagagatggaataaaacttgatgaattattactatataatattgatgaacttacataggaaaaactacagccatatatataggcctcttgaatcacccttgcattccacttaccacaaagcttacgcaaaagcatagggtgggagccagtggccagtacaaatcgtactaaaaattgtttagcaggttttgaacgtggtccatgacgatgactacggagaatagaaggattaggtggtcttgttcctgcgctcaagtttggttcggagatgaaggctacgcccgctgataaactacgccgactctgatgattgcctgtgaaggaggagccttcaccgctgacgcgctacatcaactttgatatagacccgtgtgtgtttccgctagaaaaacgatgtaataggctggttgaccaagagttgtaaagtaaatgtgatgtaatcttgtttttcacgatgtatgactatgataacagctgatatatgataatgtgatggatcaatttttgaattatcacattataattcgaatctgaggatttttccctttgtggaaaaaatctaggtcgtttcagaggagggcattgtaatttgaaacggagggagtacattgcatatttgcatggtccaagatgcggaggttttcaaattccaactgcacaaatgtttacgtaactgagactgactagtaggtccaggagtgggcctggccagagctggaccgactccaaaatcaaccgccaaaagagcctggacgggcccaccgttgcgcgccgaaacccagttccatccgtctcctcgtagggcccacactccaacgacgtcagtaatccccggcaaaaacccatcgccaccgtctacttgcgatggcaccaacgcatcccacccgtccacgtcggcgatccgcggcacatgccgctccgccaatcagcgcccgctgctccgttctataaatacaccgcagccctccccttttcttcctcacagccaacgaaatctcccgtccccaaatcgaccgagcgaattcaccacagcctcaccgtcccgaatccgcaccaccgatg

SEQ ID NO: 22 - DNA, 3089 bp, Sorghum sp.
agagctctcttgcccatttgaacacctgagaaacttgttgtggagcaagagaacagcaagagcctagagaggattgagatttgagtgatttcttgagagaatccttctctagtaagttccaagagtcaagtgtgcatccaccactctctagagccttgttttggccaagtgagagttctttgcttgttactcttggtgatcgccattttctagacggttcggtggtgattggaggcacgaagaccgcccggagttcttgtgggtggctcgtgtcaagcttgtgagcggttttgggcgattcaccgcgacagagtgtcgaagaatcagcccgtagagagcacttggtccttgcgcggaccaagggggagcaaggcccttgcgcgggtgctccaacgaggactagtggagagtggcgactcttcgatacctcggcaaaacatcgccgagcactttcttccactactcctttacattctagcatttactttgtgtttttacattcttagaattgccttgctagaataggattggaactaggttgcaaaacttttatccggtagctctctaggtcacactaggcacaaggggttgaattggagcttataggttgcttaaatttttagagaagcccaattcacccccctcttaggcatcttgatcctttcaggtagattttcgaagcttcaaccacctgaacgatgctttcatgatcttgatgaaaaagaaagttaagctgagggaaatcagagacttcagaccgataagcctcatccatagctttggaaaacttatcacgaaatgcatgacaggaaggctagcccctaagctagacacgttggtactatagaactagagcgccttcatcaagggtagatgcttgcatgacaacttcagggcagtctaccaagcgtgccatcaagttcacaaaaagaagatcagttgcatcattctaaaaattgatatcgcgaaatccttcgactcggtgtgctggacctttttgttagacctactgcaacacatgggctttggtttgcgttggaggaactggatatctgctatcctagccacgacaagcaccaaaattctgctgaatggaaacccaggaagacagatttgccatgcacgtgggctcaggcagggcgcccctatctccgatgctatttgtgttggtcatggaggtcctgaaccgccttcttcttggctggaatctagagatctgctcacgccgatgacagggttatcttccccgcgggccagtctgtacgctaatgatctggttatgttcgtcagactagttgacggtgatcttcgggcggtaagggcggcgctgcagatctttggtcaggcatctgggttgattgcaaacctagacaagagtgttgccacaccccttcactgttcatcagaggaaatcacgcgggttcagcagcttctctcctatcggattgaggagttccccacgcgctaccttggaatccccttgtcggtctacaagctaaggcggtctgaggaacaacctttgattgacaaggtggcagctaggatcccggaatggaaaggaaacttactcaatgaagccgacaggactgctttggtcaaagccacactcagccatcccagtgcacacgtcgattgcgatgtgtctctccccttaggccttaaacatgattgacaagctgaggaaggcattcctttggacaggctccaatgccgtagccggtggccggtgcaaggtgtcttggtccagagtctgcatgccaaagcacttgggtggcttgggggtttccgacttgcgtcgtgttggaattgctctcagggttcgttgggtttgcggtattgaatgaagataatttttatataaaaattatagatttcgatgagatctatatttttttattttgtttttttccatttgaagtcattaagagcaactccaacaatttgctaaaagtacttgacaacttaggatttttgccaaaaccataaaaaacagtctccaacaagttggcaaaactacttggcaattttgtgagcttggcaaaatttccccttcacttggcaaatatgccaagtcctccatcacttgccattatgtgtatctcaatttgccaactagttttgccaacttgttggaggcttaattttgtgattttgtcaaaaatcctatgatgccaagttcttttgccaagtccaaataacaaattgttggagaatgcttcttttttcacttggcatttggttttgagacttgacaaaactatagattttccaagtgagttttagcaaactattggagttgctctaagatactaaaaaaagtaacaacatatttacaggtatttttgactttttacactcgcaatttgacaccattagagcctaattaaacagtagtggcttggagggcaaaaaaatatttgaagcaatgacactgaagtccgctaaacttttctagtgactcataagaaattacaaatttgcttggccttatttagttcctaaaaaattttgcaaatttttcagattctctgtcacatcaaatcttgcgacacatgcatgaaacattaaatatagataaaataaataactaattgtagaatttaactgtaatttacgagacaaattttttgagcctaattagtctataattagacaatatttgtttaaatacaaataaaagtgctatagtatttattttgcaaaattttttgaactaaacaaggccttaaaaaaagaaagggtcacagccttgtttcaaatttcgttttggcgccggcgtgccgcgcgccggcgacatatccctccgtcttctgccgtcctcctctgcgccacgtcagggatccgcgtgaaaaccgcatcgcgaccgtccgtgccaagaagcaccaacggcccaggccggttgaagccagcgatccgcggcacctgcccctccaccaatcagcgctcacctctcccgtcctataataacacaccgcccccagcgtcctctcccaaccaacaacaacagcaaacacatctcctcgctcgcatttctccccaacccaatcaatccccctcgcccccgaaccccagctcgcaccgcatcgatg

SEQ ID NO: 23 - DNA, 3094 bp, Sorghum sp.
tgcatcctattaacttcacttctttgatcttcagtcatttgtgtacacgtcgtgcactgtctctttggcttttatattctttgggatcgatgacttatgtaatcgatttggattcactttggtcacctatttttttaagatgacagaaataagaagttaacaatatttctattatatatcaaaacatttatattgatgatacatatttatacacatgtgagcatcagtatttaccttagtcaattaggtttgtcctgtctttaaatgtcgcggaataatcgtccttttcaaaatccattagcagatatgtgactaaaaaatagaagaagacagtcatcaaaaatgataataaaataaaaaaactataaatgtctatatagttaataagagggtgtttggttgggtgtgttaaagtttaacatgtattgtagtattttattttatttagcaattagtggcttaaaagattcgtctcacaaattactctttatctgtggtttttagttttgtaaatagtctatatttagtatcccatgcatgtgtccaaacatttgatgtgataagtattaaaaaacagacacaaccaaacagattctaacaaccactgttactaaatctgaatttttattttattgttggtgttcatgtccacaagtttataaaggccttgcttaaatctaaaaagtttttgaattttgacactgtagcattttcatttttatttgacaaacattgtccaattatggagtaactaggcttaaaagatttgtctcatgatttacaggcaaactgtgcaattagtttttgttttcatctatatttaatgcttcatgcatgtgccgcaagatttgatgtgacgaagaattttgaaaagtttttgatttttttggatgaactaaacaataatcaagataagtctgtaaaatttgcatcaaatattttctctcatattgtatctaaggtacaatctaattactcacgtatggtaccctatgctaacagggtcacaaatatggaaggaaatcatgaacacttaggccttgtttggatctaaaaagttttagattttgacactgtagtactttcattttatttgacaatttttgtctaattatagagtaaatatgcttaaaagattcgtctcacgatttacaggcaaactgtgcaattagtttttgttttcatctatgggtgtgtttggttcgttttctatacaagcctacctagcaaaactaagccaaactacctttagtcagttctaactaggccaattcgtagttgtttggttgtgtacattgtactagcctggctagcattgggtgtgtttggttgtctatcttgttttgctcaaaattacctcttctctcttctagtaaggttatcgcctctcacatattttatcaaacaccaccacagctaactagtcaccatcggtgaagaagactagtagcgaaatagaacgggagtgaagaccaggaggtgatgggaatgaatcacggagccaaccggagcttagctccagaagaaacaccaacctggtgtttctacttgggcctggccctgcctgtacggcaagccagatatagcttggctccaggggctagccaggccaaacaccccaaacctggcccaatcaagcaaaaagccagatttggaggccaaccaaatacactctatatttaatacttcatacatgtgtcgcaagattcgatgtgatggaaaattttgaaaagtttttgatttttaggatgaactaaacaaggccttagcttggtttagatctaaaatttttttgtattttgagacagtagcaattttatttgtatttggtaattattgtccaatcataaactaactggattaaaaagattcgtctcgtaaattaaagataaattgtgcaagtaattattttttcatctatatttaatgctccatgcatgtgctgaaagattcgatgtgataaaaaattttaaaaatttttagattttgggtgaactaaacaaggtcttaggccatgtttagtcggtgaggtgaaaattttcacgacaatgtatcactttcgtttgtttgtggtaattattgtccaaccatggactaactagactcaaaagattcgtctcgtacatttcgaccaaaccgtgcaattaatttttatttttatctacatttaatacttcatgcatgtgtctaaagattcgatgtgacggggaatcttgaaaaattttgggtttttgagtggaagtaaacaaggccttattgatgaaagaagggcgcacataaacagcctgttcgcttgagtttatcagtcgaatatatcagttagggcactcacaatttaagactctatcacaaagtctaagacaaataattacatattatttatggtattttactgatgtggcagcatatttattgaagaaaaaggtagaaaaaataagacttcaaatcttatttaaactctaagtccatattattcgaggtaataaataactttagactctatgatagagtctgcattgtgaatgcccttatttaactatatttttctcttataataaatcagtcaacgatactttctgtcatgatttagtcaaacgaacatcgcagaagtaccggtgcactaaaccatccctttttaggcgtagagattttatattaaaaataggctcaattaaatagccaaactctaatttaaattaatctccaaaatatcggaaacaaacggcacggaacggaaatttttccgaaccgcttgatccagcttgaaacagcacgcgcggcgcggacgcctcgcgcccatctatttcgttccacgcatctctatccctacccgtcgaaaattcaacgctccaactctccgccgtccatcctcccacggcagcccagatccaacgcctgtagcttgcgccaactcatcgatccgcgctccacccatctccaccaatccccttccatcgtgctcctctacaaaagctcccctccccatcaatcaatcccccatttcacgccaagaaaagcctcctcctgagtctcgaaccaaccgcatcgtcccccgtctccttcccccttcgtccccgacatccccgacccgatg

SEQ ID NO: 24 - DNA, 3003 bp, Sorghum sp.
ctagcccataacttttatatttacatcagtagaaattagccaatgtgcatgttgccttcgctaatttattgtgttttcataaatgcaggttgaaagtatagttttgagcatcatttcaatgctttctagcccgaacgacgagtctccagcgaatattgaagctgctgtaagtgcacatgacattattctctcttttttgtataggaaaacaaactgcagaagttcatttgtgctgcttcgagtgcaacgttaaaagtggtcacttatattggctttgcaattgcattagtcagtgttgctgatggataaatagtttaatacagtattgggtcctttgaaatacactacttgagtaattatttgcttattaacccgttaaatatttattgtgatcatttctccttgattagcacatagcgcttctgctattgtcaaggtagttgactttatgcacttctgaagttttctaatcatttagtctaagccacaccatatctgaaaacttgctggcttttggtaaaacaccgaaatgttgttatcagataccattggtttctgaagttgtacgtgaatgtcttgcagaaggattggagagaaaagcgggatgagttcaagaaaaaggtaaggcaatgtgtccgcagatctcaggaaatgctctgaaggaggaacatatggggagttgaacgagtgctgcaaccggtctgctgcaattcacagccaattacctcgtgccagcattcttttgcttttcccctgtatattttccgttcagtgtcattcgtatggtggtgttgggtctccttagacaaactcgggactgttgcttatcctaaaaattcgattgtattgtgtggcatgaaatgatcggtgtcgaagaatattttgaacatactgccacctaatcataattttcatggagaacatcataagaaagatgtgatggctgccaaatgtgtctttagctcatttccttgctgagtattcagattccctctgcgtacctgggcaacttaggattttaatatttgtacactccctcgttgagtattatttagattcttttgctatatatttagacatgttatgtctagatatataattgatttgatgagctaagaaaaagtcaaagcgacttataattcggaacgataggagtagttaatttaagttctggatggctctgcactgtgctgggcctgggctacaggtaccattaggatctcaaatgatcaagactaaatcatgaatcaacaccatggatcctccaatgattagcactagtattaggaactatttgaatctccttctctaaagctctaaaaactttaaagcactttagctcagtttcgagatctaaagttgtagcgtgagatgggctaaagtttagagctatcttttgacctcctatctttaactcaagtttaaagctctaatttagagatgaggatccaaacaggcacgcacttgcatacatgttgagcccatggaatagtgtctaattacttacatgcacgacatgtctaaagtaatactatttcaggcattttgctcctgcggttgtcttcgtaaccttgcttttgttactgtctactgcatatatataattttgtaccttcaatatatatatccttttgatttttacatatagtgatggaatgttcaattagatttataataatataaattttcgcagcaacacgaggtgtcatctagttcatcagaaaaaagacatctaaattttacatcttctatactagtatagatggatattcaactattctttttttccctcaacttcaactcttgtagtgttcgaacccacaggtaaattacttcggtggactagagtttaaagctctaaagtttagagaaggggatcctaaagtctcctgagtcgaatggattaaaattatgtttgtatccctacatctaaactttagagctctagaaattttagagcactttagattagttttaagatctaaagctctaatataaggtggactaaagtttagaactaattttagaccacgagtttgaagctttagctaagtttagagaagaggatccaaacaacccaattcagtttgagagcaagagaagtcttgtaaaccaaaaacgcaacctcaaatgtagaagtatgttttctttgatttctatgtctaagatatttgtgcctttatgtatatttgagaactatcttcatccatgaaataatgtaattttagatttttgaggagtcaaatattgtgaattttaaataaaggtatataaaaatactaacatatataaatactaataacattcataatacaaatgttaacgttatttattgtaaatttgatcaaatttgaacaatttttttatagcagccgacacaaaatgaggccttgtttagtttcaaaaaaatttgagaaatcgacactgtagcactttcgtttgtatttgacaaaaattgttcaattatggactaactagactcaaaagattcgtctcgtaaattccgaccaaactgtgtaattaatttttattttcgtctatatttaatactccatgcatgcatctaaaaattcgatttgacggagaatctaaaaaattttacaaatttttttgggaactacaaggcctgatataacttgatgtccggcaatccccccgcgtgaggccttgtttggatgttgtcggattcacctcaatccacgtgtgttgaagtggattggggtgaacccaccccgatacatgtggattaatgtgaattcgactacattcaaacaagccctgacacaattctcaccgccagtctgcagaaagcttgagactgagatcatgccatgtcaccgatccgcgcccccctcctgctgaccaatcgcggcccgccgtgcacctctttaaatttcaagcacgctccttattcgcgtctcactacgcaaccgcccgaacgactcttccagtctcctcgcgagtttccttcaacttcccgccatcttcgatccgtgcgtaatg

SEQ ID NO: 25 - DNA, 3045 bp, Sorghum sp.
tgaactctgtcatcaaaacataacgaggtgtgctgggctgggcaactctgacgaacgacaggtgaaggcgatccggatcgagccgggcctgcgcgtgggcagcagcaccaagggtggtatcatcgactcggacggggaggtgcttgcgaggggcgatgatgggtcccactcccgcgccggcgacgagccggggcacctgatggcgtacggcccgcccatccagctgacggtggaccaggggctggccaccatcttctccccgagatgacgattcttctccctgttctccactacactactgtctgcgagtaatttccttcgtttgtagaatcatgtgcttctactgtatatgtaaatatactcacaccccactccagtggatgagcaatgagccgagagggacggatcctggatctccgtcgataaattgttttcttttcacggccccagcccacatggctgccgcttttagtgctggcgcgagaaatgagaagtgggcagttcataatctttttttttattgttggccgtgttgttgattgtcacttgtacaaacagttggactgttttcctgcaaacttgtgcttccatctaaaaagaccttgacactctagtttaagtcgaactatcttaattaattttgatcaagtctatataaagaaccaatgtttatatcgagaaataagtatcactaaatttatcattaaacatattcttagaaatacctatttaatgtcatgcgtattgacaatcttctataaatttggtcaaatggtaaataactttgatgacttgaaatgatttttttaaaaaagggatactactacctccgcccataatttttttggactaacttggaaaatattattaataattgtatatgtaatgatacatattgcacgtcataaatagtagtaaatttttaattatatcttctcgccttctgagtgttggttgtctatctggctatctatgttgtagtgtgctatagttggagttcgtggcagaggatgagaccatcaacatcgtttccaacctaaacgccttcgacatgatcagcatgcatatgctgatcaacactaattcctatctctttcaactctcgatgcccccacctattattagcctcataccgctaccaccgtcttttgtataagctttgtctatgcacacttatatccaattcaatcgatctgtgttgtggtgaactggggtacaactaaactaagggcgtgagctacggaaaaactgttacgagctgtgtgatgtaaaaatgttgtaagctatttggttgaaacaattacaaaacctacccactatctttatttatcttgaaataactataaagcatcctgtatttttcactcatttgtgaaaattaaaaactgaaagccaaaatcagcacccaactaagttaagaaaattgtactactcgaaaggtgagtatgtttctagaaaatcagcttcagattccacctattttgttggtgttctgtttttagaggtagaaatatttttcaaaagttggactaaacacaacataaattctactctagtctcttccaaaacatatgaaatgatttaaatcttatacgagtttccaacgagcccgtacggggattttggacccttcttccgacgacagattccccactaccgtttggctcgccgtcgcgatcaagaagcctagcagtgcaccgtccttcacatatttttttttcttttgatttcaaaaaaggaagcgccgtttcccgaacgaagaaaaagataaggtatggaacgagtgaccgcgcgaggcagcgcgggtggagtgggccccagggcagggtagccgccaaggcagggccgtccgtaggcgctacgaaagctggaggagttcctgttcgcatgatgacaatcgcacggccacggcaaaccctagccgccgggcaggtcggtccccgcgcggggggcggcggcgcggggggcgcgctatataaacagagcccttcatccgatgcctccaacccatctggcgacctcgatcccctcccctgttggttctgtctgttgacttccccccatcgaggtaaagtactcgctcgattcctcttccgtcctccgatccgggcggggtgcttgatttgttatcattcacggttctgattcaattgtttctatcaagttttgtccgaattctttgatgctcgattcattattagtcttcaaatttctctgaattgttccctagcttttatcctccacgcatatgtactagtatactagcagaattgttccatagcttttgtcctccatgcatatactagtagtacccaaaatcttgtgctggccgatcgctcttgtcccgcagcaatcaatcgtttttctttcttttacttttctgataataaagcagatagatcaaatcaaatagttatgatacacataatatatatcatggcatcatccacacttgattaaatccaaaactggtatacacataatatatatcatggcatcatccacacttgattaaatccaaaactggtactggagatgcgactagtgtgcccattgtctaatggaaaagacagagggtctcgtctcctatctcatcggaaggggccgggccttctgatataggttcgaatcctattgggtgcttctattctagtttctgcatctccagtttaatttgatccactgccaggtcagattgccaccactcactcacataacctgcttatatctgttactgtttttgttgctgtatgtttctttatagtatattcatttggcaattgtattgaataatcaggtcggttgctatgaattactatggatgaatactgacttcagggtctctgttttgtctctgttttcctgatcactttattctaaataaaagaacaattaatctagcagtctgcttatgtatatatgcttctaatttactgctaaaaaatcaatctatcaactagtatttttgtgtgactgcgttctctatgtatccttctgctgatgtttgtgaatacagctagctagtcagctggtcccgttgccatg

SEQ ID NO: 26 - DNA, 3248 bp, Sorghum sp.
agtctcctctctctagttcctccatgcaccgctttagtgtgaccacgtgctgccaaaaaaacacaaagaaactgctccttcacacacgtaacggaataggacacatcgtcacgtggtcgccgctgatttgttgtatctagacacaatgatatatttaaggtcatgtttgacaaggtttttgtgctcggaacattaaagggtcggtttagttacttggaatggacccatgaaccattctagattttagactatcaagattgaataaattagtaaattatttcatctgagaatcattactcaatccaaaggaactgaacaagccttaaggagaaactagtttttttacatggctcctctatcattcattagaaaatggtttttcctcctaagatgtttggtagggctcatccagctcctctactgaagctgcttatacagtaaaagctatgtatttagaaaacttaaaacgtcttctaatttagaaggaagagagtatcaagcaatattatatgagcaaaaggatacttaatgggagaacaagaccagaccaaatactagcagcactacgagtgcaacaaactcctacatgacaaggagctgaactctccatggcaagtgaggtctgtcaaccctctttatggctgcgccatccctcttcaccatcgttgttccacatgttgttgaacactagatgaggagggatgcatcatacatgccctaatgttgctacacaacacttccacctctacgtcttgctatcatatcactttccatatctcttgtcatcttcgccactacatgctgctcctcttcgcgatccacaaccatgggggtaagttggaggagatttgtgccgacctgctgctccatggcatgagcatcggcgcgtgtcgcgtggaggctgaacaacatctggtgttgtgcatcaagctaggaagagaaagaattcactcgtgaagcagtgccaaacgtttagatcaaggtgcatagaggagttacctaattctgggtcagatctagagtaggaggagctgcattaggagcattaccaaactaaccctaagaattcttctcttaaatctactagttttgatcttattctcatcttataatttttagaaaatataaataagactttgttttcgtggggaaaaaatcagtggaaatggtcttcatggggctgagcacctaccgtagacttatgacacatacaatgtaaaatcattcgatgcattaaaaaagtagagttttttgtttcaactattgctcctaaatttatgcagcaaccacatgctttggatgattttatatagagacgtgtcacctatccgcaataggtgcttatggttgtccatgagaactgtccctactaaatttctttataaatcaacttttttaagatggtgagagtacaatattttaaccggcatggggtgagtcatttttcccttttgagtagcatgggtgagtctaatttaaaacaacttaaattttgtaacttatagaacactcctttgcctatttggtcagctgtgttgttacttttaaatcactcaatcatattgctatttcaagctatgctgttatagctttaggtggaccgtctaaaaaaggtgtaaaaggtgaagaaataaattagtgatgactcgaaggttacgaagctgtaaatctaatcattgccgtatatcctcgcaatagaactataaaagtattgaaattgggcctgtttagatttaaaatttttttacctaaagagaaaattttttgtggaattggggtctaagaactaaacggggctaaaaaattttggggccaaaattttgggctgcaattgtgcacgcactcctataaaaacccaccaatgacaacttgagctgcatgttgttattggtaggcttccataggagtgcgtgcacagttacagtccaaattttggtccaaaattttttggtctcatttagttctcaggctccaattccataaaaatttttttctttggataaaaaaaattcaaatctaaacaggcccggtaatttgtaaaaacaacgccacacccctatctacctaacaggaacctcagaatgagccacgacgttcaaaattctggtttaacagagacatataggccttgtttaggtcctaaaaaaatttgcaaaatttttcagattccccgtcacatcgaatctttagacacatgtatgaagtattaaatatagacaaaaataaaaactaattacacagtttggtcgaaattgacgagacgaatcttttgagcctagttagtccataattggacaatatttgtcaaatacaaacgaaaaagctacagtgtcaattttgcaaaatattttggaactaaacaactttccaaaatttttgcaaaattgctacagtatctctttcgtttgtatgtgacaaatattgtccaatcatagactaactgggctcaaaagattcgtctcgtaaatttcgaccaaactgtataattagtttttatttccgtctatatttaatactttatgcatgtgtctaaagatttgatgtgatgagaaatcttgaaaaattttagattttagggtagaagtaaacaagaccatagccttaagtactgtacaaggctagttacgcttacggtacattcagacgagctttaagctgggatattggggagggacggcatggtactgcactgaccagtgaccaccagccttaacttgcaagcaaaaccaaagcctttttgttgctccatataaagtttaactcctgtcacatcgaatatttagatatatacatatagtattaaatataaactatttataaaactaaaacacagctaaagagtaatttgtaagataaatcttttaaatttaattaatttataattagacattaattattaaataaaaaaatacaatagcagctgttgaactttaacgacccgacaaagacccctcgaggaaacacagtgaagcggagaagtcacaggctcacagcactgcagggaaagctgcttgagccttagcgctgcgcagaactccggccggcacgaggaggacgagaggagggataaggagaggccaagaaaatccaggacaaaagccctcgagctcacgtcgatccgcctcccgcattgccttgaagcctctcgattcgggcggacccgagctgccccgcggagggctcgatctgggcccagatcgagcagcatttcggtcctgtgcatgctcaagccccggcggtgaggcatg

SEQ ID NO: 27 - DNA, 3523 bp, Sorghum sp.
ctcgctggcacgggtcaagactttaacatatctaaactatatttgaacataaaacgaacttgtatcacatgatgtgtgagctcaatcgttcttgaacatgctgataatcgagagcgtgacatttcgaaatgtttaaatcaacaacctccataaacggtaacttatattgtgtcaccttgctagctagtttcactctatatagacgtgaaacaaaatggagttttagctgcggatcgaatttattctgtgcaaagctactttcacaaagaacgatctaaatttaagctacaggtttgtacttgccaacacttagcgtacgtaccctaagttgttcctcgattcctggggctgtcatcgctcttgttagtctttataactttattgattcttgatcatgatgatcctaaccagtgaggttctgtttggcagctttgtagctttattcatatatgcacaagaatataattgtaatacggtggtaaatttaggctctgtttagcagggcttcttcagcggcttaaggagctgtttggagttattttctaccaaacagaagtaaactgaaatgactccaccgatgaagctccttaaaaacatgatctaagagcttttgcggtgcaaaggtgccaaaaatagtggcttctctcggcttcacctcatcctaagtggtgttctgtgagagtattttaaggaatgagctgttttgtcgaacgatttaccaaaatggctctaactgtttatggagcttaagcctctaaaaatagctttactagtgaagtggagccgtgtcgaacgggtcttagcttgttcttcataaggcatgcactataatttacattggtatttggtaagtacgaaaccgtgcttgcaagttgcaaagaggatgtgatgtgaagccagacgttgtcggtgacggtgctgactgctgacgggccgggctccacggaaacaaactcgctactcgccgcaccggacgtacgtacaggtcggcagcttgctcggccccggccgcgcgcgtctccgtgtcctccgcgactgtgcacgtttcgtcgggagcggcgtgcccacgcccaccccccgtccaccagccagcaaccgacggcactggtgacacgcggctggtccgctcggtccgccccgcggctccagatcacggcaagcgcgcccgccgcccgctgctgcgctgcgctgcacgtcccgccctgacgccacgccacgccaagcgcgacacgacacgacacgacacgacccgacccccgccaacgaaacgccgaaacgcggcaacgcgtgacgggcgcgcatggtcgatgctctacccgcgcgtccgccccacgccaatctcccggcgggtccctcgtgggacggggaacgcgatgcggctgcaggctgcgaccgcgaccgcgaccgcgaccgcgcccacgtgaaggcaggcaggcagccccggagcgggcgcggcggtgggccaacgacgcgttgccgtcgcgaatcttcttctggccacggccaagggccaatcgcccgctccgctccgctccgcactccgcctccgctagggaatatggaacccgatcccacggccctctgggtctggtcgacgggtcctctcgccgtggcagctgcttcccggaccggaggatcgctgagcgcggacgccactgccattgccgtccgactatagttgttaattaccataaaataatttgttaacgataaaacccgtgtcaggcaccgtcgtctggacgctgctatgggataaccattcgcgtacgtcggttgtatgggtgggatcctctgcggcacgccattctggtgctgctagtggaatagacaaaaaaagggccgacggtgtttgctcgtggcaggccacacagagtgacaaccagagtggttgccgcaaaaacaaccaatcacacaaaaagtgttgtaccggtggaggacagccattaatcagcaggccggcttcgcggccaaaagaaacggagaagaggaaaaaggggggcaagcaaagaagaaaccacggacggagcgagctccgagcgtcctcatcctcccgtctataaattcccttccttttcttcctccatatatagggggcgccatccaagccaagaagagggaagagcaccaaggacttcccggcgcccgttcaggatccacatccttcccgagcgagttcttggttgacctcttcctcttcgaccacctcctagggtatgcatgcactgcacccccgttccccctttctccgtttcccttttctctgaagaagaaatctgtgattattgtgtcctggtttacgagattagttgttttgctgagtatgtgctaggctactgcgctgaatttgtgtgtcgatcttgcttttttcttttaatcaaggtcagccctgtcaatgaacaaaaggtcgttattccccccccagaagtttgcgatcatgcttgattttttgttagatatcgtttttcttgtctggatcttagtatgactgttgttcgtgaggctgttaagtaatcgtaatcagactgggtacggtttgctggccctgaattccaacagtcagcttgctctggtttcagaggatttatgttcggcaaaattttgatcatggctgtacagaaagaattaatcttgatggaaataatttagatgaaatccttcatgacatgaaagcatgtcatcttatgcccccttgctttgcttattctacagttatgtgaagccaaagatagtgacctaagcagatcagtactaccaaggatgcattttttagctgttcgtactttgtagtataaagaccaaggggtgctcattaggtttgtatgtatgttagagcataggaattgaaagggttggacttgatgcttctagtcagctcaatttctgttttggacttggggtggttgatttgattataactaactccttatttctaatgtggatggcatccatctatccctggcatcttctctacaattgagatgccttcatttaaagcttaggaccacttaattgaaagttatctggactgtaggaataaatagttgctgagaggaatgatgaggtaaattacaatgggcccatgtcatagacacttgcacagacaatgatataaagtcattgagatcttatagagatggtcacatggtggggtttgtgactgaattcttcaacataacccatttctgctagctttattttttaccccttagttttaggaaaagctatatcatatagtctagatatgcttggctcggtacaaattgctcctagattcttgtatggaagaatgtcgtcagctgtgttcagtattgacctctactctctattgtttcatggtgtgcacccctatgactctagtagaaacttagcctgtgtttttagtagtgtttgatcacagaaaatgtagatgtttgaaagtgtgttgtggttgcctttgtctgcactgaattttctctaatatctatatcattcctttgtacagctcttggtgtagcttgccactctcaccaatcaagttttcatg

SEQ ID NO: 28 - DNA, 4012 bp, Sorghum sp.
gccaccacagaacgtaccaaggatgtcactctttcgtggtgaagaacaaagagatttacaagctaagccaccaaaaggtaactactgtgcttgcgtgataaataaccggctagttttcatgcaaactagcaaagaaaccatcaaagctctcactcgagagggatcccgtcaaggcaaggacattgccgggtcgccctaggtcagccggcttagagcgccatccttggttgtggatcaagggatgaacaacatggagatttgagaagagagtaaaagggtttgagtagattggtttgtctattgattggatagtaggaactcaattggccatgatccatttgtgtatatagaggggttggttttatcccagtagaaatttttggactgaaaactagaggactcggcttagccgactggaggactctgttagaaatttcggatgaaaagcttccgaaattcataattaattcatccgaactccaagcaagacgatctatatatgtttttcgatcagctcaacgagaaaaacacaatagtgaagtatattcttgcatttgaaaaagttagacaagtcagcttagccgatataagagttgtctaaggcggctttagctggttcaacatctgaaaggtcattttctacacttaatgtggtgatccaaatatgctttctgaccatctttacgacagatgttcattatgacgtgatgtctattacacactctagataattttgagtgtcaacaataggtctcacatatatgtactctattggaggtcatatgtactatgttgagcgtacactcatcttccactcgaccgttattcatcttccctaaaaaaaatcaaccattattcatctcatctcaggcacataaggacacagagagaagatattatagccattatatcaaacctgaccacttgtgttagcgaatgatttgtcaccaatagtaattttctcgagaggaagctgcggccattatatatatcgaacgcgttaggagctctgcggcaagtttcggttgggggcctctcgttgtatgtctagaatgtgaagagtctttttagttttttatgccctactttacagtttgtcaaacctgaaagctgtttgggctacttgcgcatgtaattctccctctccttaataatatatgacagctatgtttcagatcttttaaaaaaaagtttcggttgggtacgaggcgagcagttctgttgggacctttcttcatttcctagtcaattgaaaatttacatttcgttctaatttctaattgcatttttatctgctgacacatactctatgcctcgctagagaagtgataccaacaagatccagactacaactgttcataggccattcatttgtttgtaataattattgtctaaccatagactaactaggctcaaaagattcgtctcgtaaatttcgatcaaaatgtgcaattagtttttattttcatctatatttagtactcatgcatgcgtttaaagattcgatgtaacggaaaatcttaaaatttttagattttgggatggaagtaaacaaagctctaagataatagaaaaagcccgcacccccacccaaaaaaaatatcaacccatcataacatcgggtcagattcaaaccaaaaatttgagattttttttggagaaactaccaaaaattcgagatgttcatgagttcatgggagcccagtttttgtcgggccgtatttgttggttgggactttttttcggcattgcaatttgggtagagccagcacaagtttcataggcaccagcccaccaagatcactagtgggcctaaaactacagtacttgagaaccctcaattgattcccacttaatttcacctaagcccacaaggggaatcgagtgggccgaatcctgatcctattgtcggttcatccaagcaggcaagcgcacgcctcctcctcctgctataaccaaccgggcggtcgaccagcgggaggacccagaaacagagagcgcgcggttctacgtccgagtcgtcgcatcgccctgttccctcgattcgccggcggcgccacctaccaaggtgatgcctcctccctcctcccttctcttccccgatcaattccgtgtttccgagcttagaatttggaggacctgatgatgagctcctccatctcttgattgatctgtgggcggtcggattctgcggtcgtaatctcgccccaaatcgagcagatctcggggctgtttcgaggaatataatcgagcaggtctcggggctgtttcggggaatatgtgcgttggattgttaaggtggagaatgtttgctccgatcgggttttggtggtgtcggggagggtggcgatgggttggcgccttggcggatcgattttggggaacctccctcaaatcaagcaccaccacctgggtttgtgattcgatacagtttcatgatttggttaccgtgttttggtatccttgattcctcgttccatctagacgtatgtatatgataaggtgtatcttctgactcgtagaacgatcgacaccacaactaaattgtattgcaatttaggaactcctatgcagattttacatgtagagttgcatatcgaatggtgggtatctgtaaatgatatcttcacctgctgaaataactgagaattcctgggagttgaaacctgttgttaataagcagagaacacagttttggttatggttatctgtagctatcatataagaggcaagttgcgtgtatggttaagtcacctcgggctacattatttgtgactcgaggctagccccatttccattactcatacaacggaaagagcagccccatttccattactcgcagaacagaaatacagatgttttttactagaacagcattctagataagggaaacaagacgatgacatgctatcagcctccactaaagtgttactctgctttggaccaccactatagcagggagatagcaagctagcaactagtcaatccagaacgtcacctcaggctactgtgttggtaaaagttgtacttcagttctgtcatgtgcctttctagcctattctgaccaacaaaagaggaaaattttattggatcttttgaacccttatcctagaatatttcattcaaataactctgaactgtgtggtttatttccctgtctttgttctgatctgcccttggttatcatcccaatagcacctccatcagttaggtatggaaaacatcgtttggcttcagtgtccattcagcctatttatttcttacctcatgataggtccacaaactttaaaaatacatttctaggtccctaaacttgttaagtgatgctccatgccaggttgccagccacgtgatcattttctgctgatgtggcatgctggagtggcatgtatttatttatttctgaccctcgcatttatttttccctttgaaaatagatcatcccttctccttcggctctttcatgctcctccctttcctgctgctgcaaaaggtcgcatggctccacgagttgcatgtcgcagcccattgtctattttcaatagaaaaataaatttgaagtgttattaaatataaaaatatatgccacgctagcatgtcgcatcaacaagaaatgtccaggtggctgctatggtcctgtggtgtaccacttaacaagtttagggacccagaacaaacttaatgaacctatatgacacaaccttaagtttaagggcagctggagcatttaactttataggtattatcttgttagatttgtcttcttgtgtatggagtattttagtcaatatgagattttgcattttgtcgtgaattgctgttcctgtatcaccctggatattggatgattgagttgagttgtacatttaatttaagttcttttattcctttatgactgcatacagtgattgattggaatggtattatggtttgcagctcatacacccagattgactagtcaaacccagtgatctctttggggactaatcaaactcaagaactaagtttcatg

SEQ ID NO: 29 - DNA, 2740 bp, Sorghum sp.
ttgggaagaggatgctgagtgaataaaaacgacatgcatatgcatgtttcagatgaaaaacatcatcgtttccaaggatagaagtccagtatcttcatctcatgcatgtttagatggagagtaactttttaccaaggccagtagaaacatacaccttcgttactcgttagtggtgtactggtgttctaagatcagcgccaacgcacagtggcggacccaggaattgggagcaaggtatgcctatggtaaaaaaaatttggatgaacaacacaaaatatttagatctgcttagataaaagatacatatagttcaaatgcattgcatatctgtaaattttattttgcaattgaaaatgacatttaatagaaccaacaaagggaaaaggaaagggatttaagttttattactccaagcaaagggggcgtcgtggccgattggccgcgcggggacgcccgatgccggggagcgtagccgccgaagcggcagcgcggggacgccggcaaatcaagcatccgcacaggacgcagcgcctacctgcgcgtctgtggcgaatcacgagcggcggcgcgcggtgaaagcggcggcgagcggttgagagtcgcggaagtgcctgcccgcgcgtcagtgcgcggctgccctacccctacggactaggcctgatggttttaggattttgggggagtggaaaagtgagtgggaaggttagatgagtcatggaccgtttggattcgcgtggcttatggggctgcttttgagttagaggtgaattgctgaataaaatgacctagaaacacagaaaacgtagttttaacaccttatgcatattatatatgtatatatctcaaatatctattaaatttttttccaaaaatgtagggtatatccgggaatacccgagcacaactgtaggtccgtctatgccaacgcacaaactcaacttgtaggcctagcttgctagctatatttggatgtcacgctgttctaaattcatatgccttaaaattgatataagtcaaaggctactatttcctcaaaagagagaaaatgacatgtgcgtacgtgagacgggaattagaggttgtgtccgctttagcttctttctgacaaatgctgtaacgtctttgtttgcaactgtgcgtgcagccgtgagcttctttagctttggttctgacataatgccacagggcgtctataggcgttgtttaaatacatcaaaaacccaaaactttacaagattttccatcgcatcgaattttacagcatatgcataaaacattaaatatagataaaaaataattaattaaacagtttacctgtaaatcacgaaacgagtttttaagcctagttacttcacgattgaataatgtttgtcaaataaaaacgaaaatgctacagccataatgttgaagctggcgtgaggggtaccaagcatgtccttgagtaaaaagaaggccccggtgaggaaaaaaaaagttcaatcctagttggcaaaaataatggttctatgattcaatatctatatgtcatgttaattgaaagaacagtggttctaggatcatgtgctatatcctgtttgtttgaatttataatgatgctgaaaaatattgttgcgctgataagttcgagtgaacagatattaacttttgttgcgtgggtgaaggccatgccatggcctaaagatcaaagagacgccatcacggtgctgcacttttcggctccctcctgcttccacatgccgcgcgtcgtctagaaatccctgattcagcagcacacctgtgcgcctagccgcccacgcgtacactgataaacagtttttttctagtccgcccacacgcgcgctccgagccgcagatcctagcaagcgccgcgcatccgacggccacgacagcgcggtgccgtccgccgcccccaccgcagcttgtccacctcctgacccatgagcggaaaccacggtccacggaccacggctgcgttccagtccaggtggaggctgtgcaaccccggttttcgctcgctgcgccgtggtttgctgcccaaggtggccggaggtggcgaaaccgcacccggatccttcccatcgtttctcatctcttcctcctttagagcttagtatataatcagggctcttgtctcctggctcctcacaggttcgtttcggtttggattgattggtttgatcagtcgtggggtgagggtcttggagtcgattgatctgggatactgttagaggatttggggagggggcaatggcgaccgcggggaaggtgatcaagtgcaaaggtccgtgatttctcctctgtttcttgatctaattaattttggtttatggttcgtgaaatcgtgagtacttttggggaaagcttcctagggagttttttttccccgatgaacagtgccgcagtggcgctgatcttgtatgttgtcctgcaatcgcggtgaacttgttctttttctatcctttaacccccatgaaaatgctatttatctttcttacatcttccagttccagcactgctattaccgtccatccgacagtctggctggactgacactacttatggagcattgctttctttgaatttaactaactggttgagtactggctctgtttctcggacggaagacatttgctaatccaccatgtccattcgaattttgccggtgtttagcaagggcggaaagtttgcgtcttgatggttagcttgactatgtgattgctttcttggacccgtgcagctg

SEQ ID NO: 30 - DNA, 1743 bp, Sorghum sp.
gacggcgacgaggacggcgcgggaggtggcgcggctggggacagaggcgggcaaaggcttggatcgacggcgaggggggtggcgcggccgcaccggcgacgaggacgggcgagcgcggcggcggaatcgcgggcgggtgagcgcggcggcggcgggggcggctgagtgtggggacgagtgtgtgtgagagagagaaggagtgggggggaattggataaggccagttagggcctttaccgagtgctggatagtaaggcactcggtacatttttatttttatttcaaaatttcaaacagcccacgcgatctacacgaaattaaaagtatgaattcaacttatcccgagcgcacagccaaccctcggtacaaaatggccacgtcaccgagggttacccattaccgcgctcgggctacaaacattttagaggcgccaaggtgcaccctcggtaaaaattttgtaccgagcgccgctgtatgcaaccctcggtaactagccattttgtaccgagggttagccaggctctcggtacaatttgaaatcatactttggattgaaatgtttttgaatttttaatttcgtaaatcacgtctgcaaaattttggattcaagtgtttttgaattttgaaagaccaaactctaccgagggctccgcatacccctcggtgtaaatatttcaccggaggcaacccattagcgctcggtggaatgaccatattctaccgagcgacagacggtgctctcggtggagttgacgacgttgtgggagtttactcgactgagagagtggtgcgtcaattttaccgagatgttttttctaccgaggagctttgctcgctaaaacacagttttagcgagggttttatcttaccgagggttagacgctcggtagaagggaatggtaccgagcgtatgtgttcaccgaacgctactcgtgaagtgcaccgagggtcttatttcaccgagcttaaccgtcggtacaagagtgatgtaccgagggcccgttttcctgctctcggtacatctttatgctctcggtggagatgcactgtgccgtagtgactcgtgattacggattcctctaagttgggtacttgtaatatcgatacctgacagtttgctattgttgttttatagctttattaataagataaaaatgcaactatacatcgtagctctctatttacgaaatggtaccactagctagtgacgtgtccactagacgacatggaaaaccataaacaagacaagaaaccgctgaaggcagaaaccggcggggccaaggctggctcgcggaggccgggaaaacggaaagcggcggcggacacctccccgcggtttctaaccgcgactaaaaaatccgagcctttctacccccaccttgtgccgctacagtccaggcattctcgcttagtcctagaccactatatatacagtactcgtccccgctttcttcctcgccaacttctcatcatcagccaagtgtaaagggtgcgaagaaacagcagcaaaaggattccattctcgtgttcttggagtggtccatcgagcttcgtcagggagagctattgagagagagagagagagagagagagagagagagcaagcaatggcgaccgcagggaaagtgatcaagtgcaaaggtccgtcgtcttctacctctgtttttcgtgatggctaactggcctctagcagcctaatcatggaattgatttggttctttgattattcgctgcctgcagccg

SEQ ID NO: 31 - DNA, 2712 bp, Sorghum sp.
cagttgagctgggacccgtattctggtgactgtggttaatttgctctgtcttttacgttttttgttgctctgatctgggtatcctttttatggtacccaacactttgctacggatttgtgcactccaaaccctaatcaagactcaactccaatacgactcacgaacaaaagcactcgcctacaaacaaatctacatgtatgccacgatttgttaacgggttaggatcgagttagacctaaaaactatatgctccacatgattataggaaaaattttaaaaatttcgtatttttaatttacgtaaaaatctgggatgttacgatcaacatttctacgaggttgagttagtaatctttggaacgacgttcgcgtctccccgtctaaggtccaaggtatgtacgatcctttcttctctttctttcattttaggatttctttttcttttttatttttttgttctcagtgtattttattatgtttatattatcaatatttattttattgtttattctgcataattatttcttccatttatttattcttagtttattttttattctttcttctattctattttttctttctcatttaacgacaccatatgggacctagaaaggtaagtattataatatcatatcaatggattgatagtaaaatagattttcagtatatttatttggaagttataaatatcaataatattttttatatagtgggttaaatttaagaaagtttaactcaatctaaatctaaaattatcttttatttaaggatggagatggagagataatatgtgtatccatattctcttttttccaaagattctggcaccaggactccttgtctaattaggttcttgggcttgatagaatccaagcgaaggacgccatatacatgctacttggtgctctcgttcatctgaacaatgtataattaaattagcattattaacaattaaacaactctgttttgtttttcttaggcatccacaaagatgcttgcaattgaactccatcatcaagtaccaaacggacgtgattgttttttcttgggcatccacaaagctgcttgcaattgaactccttcgtcgagtaccaaatggacacaattgtacatagcctcgtcagcatctatagccctacataccagacgtacggatacacatcgacattgattctgagtcatattaggaaggacataaatgcgttggaggcagattcgattaattggtttgaagtaggagagacgaggacaatagccataccagtatgtccttctggcatatgcgtctagatgacgctatacattttcacccgttgcaacacatgggcacttttgctagtagcaagataataaccaacctctataatatagccgtataagagatccaatatattattgtagttgtgttgctgatattatccacatccttctatgtgtagcgcctttaatttatttatattagagtttttatgaatttgttcaattctaaagttatactcccttcgtttcaaactataagtcattccaagaatcttagagagtcaaaacatttttaagtttgaccaaaaatatagagagaaatataaagatttatgtcataaaataggtacactataaaaatataactaacaaagaatctaatgatacttggtcgataccaaaaatgttattattttattatataaatttggtcaaacttaaaaaactttgaccctctaagatactttgacgccatatacatgctacttggtgctttctctcagctcgcctttgtgagcgagtcttgccttgccaagctgaacgagccaaattggctcatggcacctagcaagcattttctttgccaccgcaaggctcaagaaaaccttgcttgccaacatctatgtattccactactataatttcgatgggtaaagtcattttaagtttatacattagcatcactaaaatcacttgattgttcaaattccaaagcatcaagcaagcaaggtcttcaattgctagctcctccgacatgcttcaggtaagccatgttagatttagcatctggggcagtgattatttgtatgttttaacatcctttcctttgcatttgctatttcacacacaggtctatgtggagtatcagggatattccttagttccatgccccagatatatttaggggctgtttagttcaaataaaattgcaatttttttttaaatttctcgtttcatcgaatcttacagcacatgtatgaaacattaaatatataaaaaactaattttacagtttatccataatttataagatgaatcttgtaaaaccaaattagtttataataagataataaatgttaaacataaataaaagtactgcaatagccaatttacaacattttctgcagaaaaaaaaaatcgtcccaccggccacccaccggaatgcggaccaacccacccgtccacgcgccacctcaaaggccaacggcgtccaaaccataaacgaccagcaaaagctcaatgacacacgggcctccgtcgcctccaccaacccccgccttcttcaaaatttttattcgccgaaaccccttgccacctcgccgcctccgcccaacaaacaagcgcgacgacctctacctcgccgccgccgccgccgtcaccgccgccatataagtggaccctcgtctcgtcctcctcccagactctccaccctccgcctccgaacacatg

SEQ ID NO: 32 - DNA, 3282 bp, Sorghum sp.
aaagttaaatctggaaattttgtagtaaataaagaatagttaaatctaagagaccatactcgaggcatataaatattattattatgatctgcagcaagatgtacctcatcaagtcatcagaatagttaatatatcataacacgataacacaacgatctctctcttatttttttaaaaaaagtcaatattggtctatttgactcgctcattgtaaaaccaaaatttcaaccatgatttttttttttgactgtcaacgggggtggggagtcgctccccacctgagtattttgtatttcatatcggccctgaatggcctaatgtcattgaacttttacatctccatcggactgatgaagtttagatacgtacagaaaagttcagatcgaaatctagtgaaataagacactcatccttccccttgtctcgccgactcgaaggctagtgcccatgcttgcacaatatggcgcttggatggttggagtctgaacccccattggactgaaaccgccgagcattgctgcagtagttgattcaatccggtggcttgattcctgaagaccttctcgtttctggacttccacaattgccaacaacatagggctatgaaggtagagaagccttcacttgggatcatatccgggcgcggccaggaagtgacattgtctggttggaggggagcatcgatacccagacaattccaaagttgggttgccaaaccacaggagcagaagatgtgtgcctgggtttcttcatgctgcccgcagacttcacaagttgagtgatcaatgacatgcttccgcattaggagtgccctggtatggagtctatcttgtgagagcaaccacatgaacagttgtactctcggtggtgcaaaggacttccaaatgaaggtagcacttgcagatgccggctgtcccttagcctttagcattctgtagatggccccactatcaagccttgaatcttgtgtacagaacattgactcccgcttgtccggcacatcagaaagttcaacctggtggatcatttcgttgagctgtagcagttcattacgcgcttggggggtgatccgtggcaccatgtacgggccgattccattcttgataacctcgctgaccgtggcattcttgaatgtgcaatggctcagaagcagtgggaaggcatccccaagtgcatcctcccccagccatacatcattccagaaggaggttgacttcccattaccgatatgcacagatgtgatagcctcgtagagaggtagtaggaagtgtattccagtgatcaccatggatgtcaccgtgaagagttgcaagaggcgcgctcttgtacccactgagaccaggcagatgaggagggacagtgtagtcgatgcatcagcttcagcaacagggagatgttctggatgccgatgtccctgacgcctattccacccaaatccttaggattgcacaccgctgtccaggccaccaggcaagctgcaggcgatgctttgccatccttggccccagaccacaggaaagctcgtctagttttgtccatcttcgaaatcatacccggtggcaactgcagtgagctcattagatatactaactgcgagtccaaaatcgagttgatgagcactgttctccccattttgttcaggaaacttgcttgccaagcacttagacgtcgaccagctttgtcgatctgagaggtgaaggcagacagtggtagtttgttgatggacaaggaaaggcccaggtatggctgtgggaaggattctcttttgcagccgatcgcctgcacacataaagaaacaatcgcttcatccatatggattggtacaagagtactcttgtcatagttgatctgaaaaccagacaatgccgcgaaggtgtccagtgtttggcggacagctgcagcaccagcgaggtcgccccttagaactattagcgtatcatcggcatattggagaactgcacaagggcgattggcttcagtcgggtgcctgatggctgcatcttaccggatcagcctctgcagagtttcagccactagtaagaataagtatggagagaggggatccccttgacgtagaccccgtttacattggatccaagggcctgcacatccgttcaccaaaacagcagtcagcgcagagtttaggatggagctgatccacatcaaccatttttctgggaagccccttgctctaagtaccctctgcaatccatcccaatttacagtgtcaaaagctttcgcgaagtccagttttaacacgattgccgacattctccttttgtgacagacttgaactaactccattgcaaacacaaaagtgtcagagatggctctttccttgatgaaacctgtttgatttatgtcaatcaaatttgggatttctgcttggagcctcatagtgagaactttcaaccatgattgttatctctcttgcttattgaacgacttgtaataatgattatagagcctgcttgtgtatagaaagattatgtttttgaggaattgtatagatagattatagatatagtaaaagaaattcaagaaatcaggcagaggcagctgcggcagccgccgccaccgccgccgcagattgaagaagccaccacagcttttcaacagagaagaatttctaatgatggtgtgatagtggtactttccagtaattattaaggccttgtttagttccaaaaaaatttgcaaaatggacactgtagcactttcgtttgtatttgacaaatattgtccaatcatggactaattagactcaaaagattcgtctcgtcaatttcgaccaaactgtgtaattagtttttattttcgtctatatttaatactccatgcatgcgtctaaagattcgatgtgacggagaatctgaaaaattttgcaaaattttctgagaactaaacaaggcctaagcaagctcctaaatttgaacgaaaggaaaccagcagaggagagagagcgcacacagacagtaccaccagggaacaagaaatcgaatatgctgccgcagccgccgtggaaaccgagggcggaccttcccctcctccaagcaaagccttagggccaggcccaccgacgggcgggctccacccgctcaaacatccgtcaccgccaggtggccccagtccatccctccgcggggcgtccctctgatcttctacaaatagtcccggccggccgcggaggcgaggcaaaagcgcaaacgccaccagcagcccaccacagatagcgagcgagcgagcgggcgagcgcttctagggcttttttttttcgagatg

SEQ ID NO: 33 - DNA, 4383 bp, Sorghum sp.
acgatttcctgaatacgtttataggatatatgattgtgcgcgcatatatgtccataaataaacatatattaaatatctggttctactgggtatgtgcaattttagcaccacccccagcccctccccttctgatcaaataacaggaaagaatgtcccccaccatcgtcggaagcattatcctctttgttacgccataattataagatcttgtttagttttctgggtaaaaaagatacaataacacttttatttgtatttagtcattattatttaatcataaactaactaggctcaaaaaattcgtctcgcaaattataaactgtgcaattaattattttttgtttatatttaatgtttcatatacatgttgcaagatttgatgtgattgagaatttaaaaaaaaatagatttcggacgtaactaaacaaagcctaggtagaaaacctaaaacaaacacaaacttcgtagcaacctttccaaccaaacttcaaaaacacccttaaaaaaaaagaactccgtttcagaaacgcccactgcagaacggagctggttcgttcctccggccaccaacgacgacaacagacctgatctgacctaacctaccccaccatgcatgttgtgatgatgacccaaatggaaatcgccgcgcccatttgcagattaaaaaaatgagggtcatcaatcacagcggcgtggtgttgcaaaaccagcacccccgtcgcctgatttcggaaccggacttggaaaccacccgccggcggcggcggccggagcagcaaatgggagaacatgccaccgagatgccatctcattcacccgcctttccttttcctgccgtgggtggtcagcacattgaaccgaaaaggaaaccggaggtgggtgatcaggtttcagatcatgcatggtccatggcttgtttcacaagcacaggggaagagagacttatccggcaatgtatatatcctccctgccgggttgccgctatatatataaattgctcccttcctgctcccccaacctccgcattgcgttgcgttctactgcatctgttcgttccagttctctgttttgttctgttctgtcgacgccattcctgtgctgccgtcctgccaaggtatgcatctgacgacgactcgtctcgtccgtgttgcctctacgatgcaatgccggccactgtgttccaaaattgagcatgcgttggttgggggtagtaccgtagtagtacgtctgttagcggaaaatgtgcatgtaatgcaagctagatctgtttgctggtgttatttagttttttttttttttttttttgtttttgaggaaagctggtgttatttaggtctggtttatctaaaaagtttttgaatttttatactgtagcactttcatttttatttgacaaacattgtccaatcataaagtaactactgatttacagataaactgtgtaattttttttttactttcatctatatttaatgcttcatatatgtgctgcaagattcgatgtgacgggaaattttgaaaaagtttttggtttttgaggtgaactaaacaaggccttagtttatatccactgcacattttatttttttattatttttgtcttttgcatgcatcctgatgaaatccgccgaaaagttgaaaaatgtttttgagacaagtaattgtcaacgtgacgggatcgacgatctttaatggtacacacaaatcatgcattgtcatcgtccccgctaagggcactcacaatacagactctatcatggagtctaaagttatttattacctcgaacaatgtggatttggagtctaaataagacttagagtcttattttttctatctctttcttcaataaatatggtgccacatcagcaaaatatcataaataatatataattaattgtcttggactctatgatagagtcttgcattaggagtgccctaagcgaagcgtttggatcccataagctgttgggagacttgccgtgagccagtaacatgacaagagctctcctgtagtcctctcgtgtctttcttttcgtgcaagaagaagaagaagaagaagaagcgccgcctcctttttctgtaattctgttccgctaccttctcgtcaccagtcaccacttcgcttgattttttccattccatgtcagcaccgcatcggcacacttcttgtttaggcagctctgttcttgtttgtaacgtacggcccccacctcgtcgcggcggcacgatcgatgcgagtgccgtctggcggctcatcactcaccgggcgcccagctgcatcgatgcagtatcttcgcaagaaccgatgcctttctgttcttgaaacgatgtctgcgccggggcaacttttcttgttgccgcttgtcgcttgcgctgctgctgactggacggcagctctggagggagggtgtattttggatgcatttttagggatggttgtgcgtgcctgatttcgtatacaattcgccacttgtttgcaccacgttcttggtgtttcccccccgatgtttctgcacacgggctttcctctggcttctgagagacccatgttttacaggctttttctagaaggagggcaaacctactgatccaatgggaggcattagggaggggcaaaaatatctcgctgcttttttaatttataatttaatccggacaatctcatttgcgtttgcgtctgatggtgatggcaatggttatcgtatcttgggtgccggatcaccgcctttttgctgatcaccgaatcagctgggagatttcggtggtgaaattaggaactcaatacagtagaaagaagcttttttttttgggttcctttcttttgttttgtcacagtttcgtgctctgctttctctcccagctagtagtccttccttgcgttcactgcacctacacaggtcatcgcagcctgcactgtacacgagtctgcataaaaaaagttccaagctttttcgaaaccggccattggtctggtagtggtaggccagcatatgctaatgggatgcttttgccgcaccattgagagtccatgacactatcagtgacaccaccagtatttggaaattctatggtagtaatttggcattatccattgcttaaaattcccagctttgtcagcttgaaggtgggccctaccatctgcaccacagctagctaccacctcggcacctcacgcctgggcttagagcagctgctgccccctctatttattggttcctcttccgtcccggggaaagcctcctccattggactgctctccctgttgaccattggggtatgctcgctcttctgtttatctccgtactaaaccactgtcctctggtaaatcctgggtggggtttttgctgggattttgagctaatctggccgcggtagaaaagatcgtgtcttgatgagcgcaatcactcgccttaattgtctccttgcctcgccatttcttccggttttcatgcgtttccgtgactcgttgggtgcgtcatctcctgaatcttgccagggctctgctgacatgttcggagttgggtttatagatttctctgatctaaatcgttgctgtgctgcgcacagagcttcccccgtccttttctgggagttttgagcttaggattttacttggtggtggtggtaaacttggattcacacatggatgcagtagaagttctaggctctgtagtttgcttgagatcttgctgtgattgcttgccgtgctcatctcttttgctttccgagaaatgtatttgtcgttttggtggattattagcgcgaaaaaacctttcttttgtttttggttcttttactacgaaaagtcatcttgttggatttttctatatttccccttttgatgatgatgatgtccttactctaggaatttgtgatgtccatgtccattcttggcttcttgcggttggctgtgcttattcggaagccaaatcctcttatttttactggtttttggctgcctcttagtggggtttagggtctggatggcatcaatactcagcaaattaactcaatcatgttggttcctttctgctttgaaaatattatcatataactaagtgcttgtgcggaatcagtacttgcttttgtttggtggaggatcaatactgaatacttgctttgtttgggtggggataagtgcctgctttatcatgactatttttctatatgattctatctggttaagtgtttctgttgagataaatcaaattgtatagctgcatactacattttttttttcaaattcaggttcctcttgcattacctactttttcagacagtcttctaagtgctagctctttatttattgttcttgtacaagtggtgctgctgaatcttaactgtatagctcgaattgcagtattgagtatcattgagccatg

SEQ ID NO: 34 - DNA, 3100 bp, Sorghum sp.
acaataagaagcacttcctactagacatgtcaacaggaaacccacctcttcaaccgacaatcatcccttactttatactctctctctcgtaccaacttttagagttgtcctaagtcaagcttttgtaattaagtttagctaattttatagaaaaaactattatcatttataacaataaataaatataatgtgaagaaaatatatttcatcgtttaatgatactaattttgtgtcataaagtttttcttacttaatataaacttaattaaacttataaagtaacttacaacaaccctaaaagttgatttattttagagatgaaggtggtacttgatagatgggtttcgtgtcataagccattgcattaaatgttgccgactattgagaccacgttacttgtcacaaaatgtgcaagaccatttttagcatgcatctatttgccttttctcatacagtcataccatgctctatgtgtactcgctccatcaaaaaaagtgatgctacgagatacaaaagattaactagatctgcataaaatatattcaaatatttatatttggagaacagatttgcataaaatctagtcccccccccttctccaaaagagttcataatttgtattgcaagaagacagataattgaaattttcatcaaatctattaaaaatacttagatttataataccaaatagtattattagattgataatgctatatatacttaaagacataaatgttgctgttactttttataattttttatcaaacttttttttttacttagtcaaaacctagaagtgtactcagtattccttttagaactagggagtgaagtccatcttttggacctcctctattcaaaaccacccgtgaaattttggaaggttgattgtgattgcttttacgtttttatataatacatactttctttgttctaagattataagacgttttgtctagatacattaaatttactatatattcaggtgtacttcctccgttctagtaatgatatttaggcctagatcaccctaaaatttaaaaacttttcaagattttccatcacatcaaatcttgcggcacatgtatgaagcactaaatatataagaaaacaaaaactaattgcacagtttgtctgtaatttacgagacgaatcttttgagtctacttagtctatagttggataataattatcaaatacaaacgaaactgctacagtattgaaatctaaaaaaaatcggaactaaacaagaccttaagtctataataaagctatgtattaaaaaatctaaagtatttttataatttagaatgaagtatatatctatattatgtgtgtatgattgaagaagtagggcatgtttactgtcacatttgcttttgcctttgtgttcttttttctccaatatatataatctcctttgccctgggctttttcctgccctttgcgtcttctagcacgttctcccatctccctcctctgcacgtccccatcctgaagcctgattaccacccgtccaagaaacaagcacagggacaaaagccactgaagcaaccatcaacaaatcagcatcctccagctccaggctccaggcttccaatccaatcgagtatccaatccggttgcttcttctccgacgcaaccccaggcccgcggcaaccgccagtaggcagtacctccctctttcgcaaccgccgccacgcgcaggtaacgaccggtgccattctcgtccttccttcccgagtcccgacctttcctcccggcttctccgcttgttcggctgctctcgcctggtgctggtagattgggtgcttcagcttcgtctgatcaggcggcgttagaggatccccgcgggtgtattccgttgtgggtttctgactatcgattggtagttttgtcaatcatctgcagtagccgagtacaatggattctactgatagggatgtttagtactctgcacgttgcgcttcaatcatctgcacgttgcgcttcgatcttcgttttccggcgcatccagtccgtgtggaaagcaacggagggttgatattatgtagttgggtgtaacaggagcatctgctgttgttttgggttatttgcacagttcctgcataaaattcaaggtacatgctattaaaaggatatactcgctccattgtaaattgtagctcgcttagctttttcgtaagtcaaatttctctaactttggctaagtttattgttgagttgcatcttgcatgcaccgaccagttcaactcaaaagcttaagttgttaattaaaggtgaacaatttacttatatacttcaacattccccctcacacctcaaatctggaaatatgagtcgtttgtagttaatttatttaattattatgcctgttaagatttgaactcgtcgagacctgcggctctgataattgtgcatttttatttggatctgaactgagggaatatctgtaaatgatatcggtgaatggtgaaattttctgttggtagggtgtccaactgttacacaaccaaccgaaccctgggtctgcacaaaaggggtcttgctgagtgtgttcctggggcctccggcctttggtctcttttgtcggccctgttttgtgatcctcttcttgatgcaatgtctcaggggaggtctttcccctagggatcaagtttcctttttaacgtaaaggatgcataacaggtggaaatgcactagccattccaaatgagtgaaaatgggtgaagggttggatatatgatattttcaccaaagcactagtcacccatggtaaaaatcccaatttgcccgaaatgtttcttctttttgtgcaattccaagtggtggagctagggcccgggctgccctagctgcagcactggctccaagccaaaaggcaatgcaaatcctctataaattctacaaattcgaaaaacatccaaacaacttgatcttgtattaatacttctggctctggcaacaccccattccacttgctgttacacacccaatattcatactggggccctactaccctagataccatgagacacttattattcattctattattcaatttgtttgtttgttttcctttctattcttaattatcatg

SEQ ID NO: 35 - DNA, 3097 bp, Sorghum sp.
gacgcggcccgtggcgccaccgatgagcgcgacggcggtgtggccgcatcggcggaaccaggagagcgcgaccaggccgaggaggttgccgagatgcaggctctcggcggtggggtcgaagccgcagtaaaccttgagctccccgggccgcgctgcggcgagcgcctccgaggtggtggcctcgacgagccctcgcttcatgagcacgtccacgacgccggccgacgcagcggtggaggtgggagcggaggcagtggcgttggtggagaggcggcggcgaagcgggtgggtgtgggggaggaggaggcaacggtgcgggcgcaggaaggaccgcgaggaggcggccgcggccatggcggcggcggcggccatcgtggggaaggaggacgaggaaggatttcggccttgcgctggtgatagggccctacctatgctagggctgggcataacaattaccaatttaacataataattaacttgaattctttcagttcctactctcgattaaccaaaaattaaattattgtactcgcttatttaaattttgtaagaatatatatttgatttatgtgtgatatgccatgttttgaactggtgtgtatttatgtttctccaagagtacccaattttgttttccaactcctaaaaagatattagaaggaataaataagatcatcgccaacagtttctataaatccactcataaaataagaatacaatttatatcaggaatcttatactatttctaaattattttaaccacttttataaattgtttatctcttgtatatttgcaccaagaatcttttactactctttattcttttcatgtccttacaactttagattgtccacaccgtaagcgcatttagatcaccgtgaggggatacacatggtttccagttcgcataactttacgcggaacggatggttttttttccaaatgctaaaagattagaagttgttggagattattttctttctatccttgtgaaaatcgtgcattgggagagtgttttagatacactggagatgcttttatattgctatataatgctttcttacctgttactattatctaaaagaagaggaattgttgccaaatttattttagaaacaccttttttttttgttgtcttagtctttgctaaaagaatgttctgttagttcagcgaggaccgaaactaaaccaaaataaccaaaatgccaaaatacttgttttttcagaactgaatccattttctagtaaggcctcctttggaatggagaaaaaatataggaattttgaaggatctaaatcctatagggggaaaatattctatgacaccctttagaacaaaggatctaaatttggaagccctgtcttttttgtttttggtattcttttgaaagattctctcaattatgtctctgatagtttaatttagaatctagatccagttggcgccaaggccacaaattacgtgcatagatgtgcactgttggcgatgtggcgccaagcttggcgccagtgacgatagggctaagtcctacgttcagattttaaaatcaaactaccagaaatataattgtgagaatcttaaaaaaaaaccttaaaaaaagacggctcccggcccctgccccgagccccaacgtcatcctccctgacggtgtcttctacacccctactcgcacgaagctgggggcatttttaggaggctccacacgcctgaatgaaggagctggggaagccatttttttagctcccgtgtcccagctgcagagaacgtgatttttgggcgaagctgcgaggagcggagctctccgacccgtttggtaggcaaaagttgcgaagctgcgcgtgaagcgcttccaaattctgtaccaaacagggccgtactatcttggctaagggaaggaattggataataaagaatgcatgtttcgcgaggaagctaaatctgtgtatcatctgttttttttttaatgttgtgtagcaactgagctttggtgtttaatgtatgatatttttatagatacagattggtaagaatcagttgcagacggtggatttctaataataaaaatgcagtgatcaacacatgcaatcttaccattttatagagcttatggaaaacacgaaattttgtttgttttcagggagagtgttgtcgggatgtgaaggtggtgatgcgaactgcaggggaatacgttgggaggatgatggcgagtgttatatatagtagacagaaaaattaatgcccccaagacgaaagaattgctaagaaggctatgggggatacccaagtcagcaaccagaattggctgggggtatgtaccagtagctacagtacactggtgacaaggcgagagcatacaagctggctaggctcggcaacaacacgcgagctgttccaaccgtgcgacaatggcacggagcaggcggcgggagaacgacgctttgccgcaccctctccccatgaaaactagcggtcagtgtgagctgcagcgccaaaacccccaaaggctcacccccgggacggaccaggagtggacgacgaccacagcccagaaatcctgctgccatcccgttcacgaacacgaccgcctcacaaaaattgggaggccggggagcgggagcgcgcacctgacccaaactaccccccaaccacgatcgaccgaccaaccaaccaaggcgacgtcgtgctcgtgcaacggcgagcaccgccagcaccgcaccgacaagcccggtcgcgcaacggaaacagccggcgtggaacgagtgtgggggggggggcgaagaacgaaaactgaagcgaagccagccacgggcacgcacaactcgacaagtcgtaacaggggcgggtcgggcacgggaaatgggccacacaatgatcgcgtcgcgcaggaggggagggagagcggccatcgacagccattcgtcgggcgtgctatccgaaatctgatccctccacgaaccccgacctcacgatctccgttcgcggccccgcgcacccccccaatccgcccccaactcacttcgtatatacgcgcccgcttgttggctagcatcgtcatcttgtcttgatcctctcttgctgcctggtccgtggccatg

SEQ ID NO: 36 - DNA, 3281 bp, Sorghum sp.
cccgacagacttcttgagtcatttggaactcgtgcacgtcgatcagaagtctttgggacaatgatgtgtgcatgtgggccacttaattcaggaggttctgccacagaaatcataaagccatgttgtttttttatctatagtataagaagggacctacgtatcacgaggttggagcgggtttgcgggcacgggtacaagttttctatgcccacaagtttttatctgttgggttcaactacaaacccgcatccacacataggcggatccaccactagggcgagccagggcggccgccctagttcctcttaggactcatctgacactctatggaaattttagacattagtgcgaaataaaaataggctcctcgagtatccgacggaggttaaagataaacttagacgtcttggactatcatgtgtggttcagcccatatagaaaaccacaacccacaaagtgcaataagaatacgatcggcctttctcatgtttctcgctattctcgacttctgactttctggtcgtctttctcgctaattcacaaacttgcggaacctaagaacctgcgccgggctgcaggtatctttattttttatttttccttttcacatagttaattgtttaaaatttgattaaggatgaaccatcgaatgattttgtgaagtcagtttagaactattagtattagtatgtatggcttctcttaatttaaggaacaaacttctctagttttggtgtggaaaatatatgctaaaaactttaatattggtcaaattattatgttgccaaagcaacttatggaatttgttagcttatattaccattggtttgccaaaattttatggtataagtccgccctaggtcatttctcgagctggattcgccactccacaccagcgggcacaaagttgtatccgtaccctcactctaccgggttttcacccacaggcacgcgagtaatctgtactcgttgccatctttacaatagaccatgtcccggcaagccatggccctactaggcgacacccttgtgagccatactcctggcgccgctccagctggctgtgctctagtgagccgcgccctagaagccatccaccacgcgagccgcgaccctgtaagtgcaatcaagcctattgtgggttttggcgttgatgaccaccgaattagggaactaatgagatttgctgagataacaagcagggaatatagcaaagaggttgttgaataccatggaggatcccccatttctaaaggatggttttcctagctccaaaggaggtttaattctttttcggtttgaatttgagtataggaaaagccgtactataaagagggactccaaggttgttgatcaaattgtgaaccaaaggctcaaaagctcatcaacatcctcagacccaagcctagccagcatatccttctctctacactttggtgttttcaggctggttcaggcaagggcggcactgccgccctaccctgtgacagttggagagctgggtataaataccctttcagaccgtctcaaacggcaacctgctcatcttcctcgctcccaaccgttgcaaactgaccagagctcacttctctctccctccattgttgctcctcaatcccccaagccaatccttgattccaaccatcaaaacttgagggaaaaggcagcaaacttcgattggagagcagatccattgattcccagcgtcaaaaagagcttttggttcacgtttggccggcaaccttgagtttgttactcttggagcttgctcctagccggctaggcgtcgccctagagcttgccaacttgtgtggcagccaagggaaggtttgtaaagttacccttgcagctaatacattattcactctttgcaaggggtaaaatccttgctttgagaacgaggagaaggtaagcctgtgtggctaagccggtcctagtgtgggcgcctcaacaacgtggagtagaccaagccttgttgtggcaacagctgaaccacggtaaaaatcgtgtgtcttgtgtgctttcacttgtgtataagtttgtgttaggatttgaggccgatctacttggtggggaggctccagcactttctagccacatacttgtgctctaacatcttgcaggaagcttggaaatttagtcgatctaaatttctgtgggtaaactttgaatcatttcaattaagcttctacctgtttttctgtagaggccggcagtgccgccctgggagggcctcactgccgcccttgcctgtgtgactgataaactttgctgaaaaagtttgaacaggcctattcaccccccctctaggccacttcctgtacgtccagagatcctacagaccctagctggcggcactcggcaagccacgctcccggtggtcgcacccctggttcccgagccctggcaacggcacccatagcgatccgcgcccctagtgaccattcctcggccagccgtgcccctggtcatctcggaaggattctcttttgggtcttgttgttagagaagatttaaaatgatatagaaccttttatttatagcactatccaaacttcaaataaatcttacattttgggtaagggttgttggagacagtcttagcaaacatacaagagtaactttagtttctttgagaggggtcaaagtggtgaagaaccaacatcttcaccaacatcgttgtagttgatactacgaacaagtactgcaataatgcaaatattatgttaagccgacgatggtattaacagataataaacaggtaaaaaaaatgtagtaggtcctgcacagagaaggagaaaagaaggggcgaattttgttgtaaaaaaatgttataaaaaaaaggggaggaatttcaaaaaagataagctccagagtgcagaaacccaaacccaacccggaacctaaccaccgccgccgttgaatggtcgggtagaaaacgtaaccatggttaactccgggaccctttaaaagccgagccgggctcgtcccatccgccccacaccgcttcactcgtctcctttcgatacatacatacacccccgcgcacgtacgtcgttcgtcccgtacgtgctcgtctctccttcttgtgtgtgtctccactcccttgccttgtgcaatctttgcacgcagcaaacgccatgatgatgctctccgtgcatcaatggcaggctttggtcccaagaagctaggttgctaatgcgcggtcgtcgctgctgttgtgtcgttctctgtttgattgcaggcagctggttcacgcacgacctcgagcgagaaagatg

SEQ ID NO: 37 - DNA, 3037 bp, Sorghum sp.
ctaaccgtttggccgtggaacgatgtggaggtgcttacttttcgacatcgtccttctattagatgaagcaagaagtcattcaactaaccaacaaattttcatcgggcagtggcgcaaacgcggtgcgacggattgatggcgcgcggccactccctcccgatctctttcactccactactcacgcacacactgtctcaacctctttcactcgcagatccagtgacggcggccactccatctccctctcatatatttatttgtatttttcaagcatacatatataacataacacatgcagttaacctcaaaagtgattttgaattttgtgatttttctatattttcttttgatttttgtaactcctttagaaatggtacaaaacttgtactccctccgtctcaagatataaggcgcgatttgacatggcgcggtcttgaagatcatactttaactattaatttatactattatatataatttatgacaacaacaaaagtatcattagaaagtatttgtaaaggcaaatcgaatgttaccatgattatactatactttttttatattattagtagactaattattggttagggataacaaagtttgaattttaaaatatgtgcatgccttatatcctgagacggagagagtagccggtagtgtaacctgtagagatgcatggtacggaactcctgtaataccaaatcgtccgcccgtggaacgacaagcagtaatcgttttcccgtatacaccaattattttttcggcgtcgtccttcaatcggacgaatcaagaagtccaagtagaagataattactccagttttagtctctggtacaagacttccttgctaatcgtttccccgtggaacgattattgcatgtgatcattttcccgtacatcctttcatttagagaaattaaaatctacttaacaaactatgctatttagcttggagtttgatattccaaaacgttccaaaattcacatatatgactatttcaaaagactctgctgcggtactcccaaataactgtgagccgttcattgatttcgatcggacggttacgataaactgttacctcctaagaggtaatagatgaaatgtatatttaataaacatgacaactaggtcccatatatttgaaacaaatgataaaaaaaagaaatatacgaagataaattattgatgagttgctaccgtcgttacctctaactgtgtctagctagagggtaattaacttcaagagttcacctaaaatgagatgtcaaattacatgatttagccattatctaaaataaaaaataacttaaaaagtaaatcactccaacaagctcttctattttttttcattcactccataataaaaacttgcacgtgatctcgttttcttcgataacggatccgtccagtcgcacggcaaaatcaatcacgacgcccgtccctgccatagtccgtcgcggggcaccggccgggtcacgtcctggccacccgtgtggccacctgccaaggacaaggcctaatctacgtacgtgatggtaggtgcttgggccagccgttggccaaggcaggcagggaggatgttcggttgtagccggatgcaaccattgggtagcgtttggatccacggagtcacggagattttaagaatctggatagaaaaaacttataaattctaaaagtctcattcaaacatccaaagattttagaagattctaacacacaagcatataactaaaaagcattagagattatttttatctagaatctggccgggtatgactacgcgtttgtcaggtcacgtcgtgtggcacttgaccgaagggcaacaccctttttttacaaagaatagattttattaatttcatcataactatcacaccgagttgatataataaaagtgatttttttttgtttttgcctaaacagtaatcaccaatcataagagaaggaagcttgagactttgatgacaataaattctaagactatgttaccacctatgtgtccagaagaaaacgttgtaactgcttgcatcattgttgagacgcctcaacactatagcctatgtgtagtgcttttggaggtaatccatatacatagccaatgaattacaaatgcctgcaagagagagtaaataagtttttttaatactaaatcatttctgcataaccaaagcgaccaacaaatagatgtgacgaggtttgagatccttctgaaaccctcaaagttgctggtaggagagataaaaaagacatgagagaggttgatagacattctatgagggacttatttttacacaaaattatccaaatcagtctatacaacacacgatagcaagtctgttggagttgttctaaatagactaccattatgcttctgtagccagagtacaacaaaactagtggctaattacaaactaattaaatttaaagtacttcggcattccgagtccatgcatgcattatattagtactaaaaagcatagactagactatcactttattttgaccactgtttttactctatttgtgcgggttatctgggaagaacttctcagataaattgtgtcatgtttggatcatcttctataaactttagagctctaaaaaattttagagcactttagctcagttttgaaatttgaagctataatatgacgtgggctaaagtttagagctaactttaaaccacctatttgaaaactttagccttaaagtttagagcgctaaagtttagaagaggggattcaaacaggctctaatataagtgtgtcggccttgtatacaaggcattcaatgacgttttgttacgcaaaattcgtgggctgtgatttaaatttccatccatgttgactaacaggctactttcattgactatctagagcgccactccacgtacacactgttatgtgtcataaggttttcgagcgttcgctagatttcactctctaattaaggacggagcttgatctataaatagatgcatgcacaaacatagtagccacacaacacaacatacatacatagacgacgatcatg

SEQ ID NO: 38 - DNA, 3003 bp, Sorghum sp.
aattgagcgtagataaagaaacacaaccaaaacaacctctaggggggatggagcggtgtagcccctgttaccaccatatggagattcaagcccccaacatcagcgatacctcctctatcatggctgttcatggcctacatggagtgccatgctaggacgatgaggtcatctgcaataggatggcagctgccaaagatggcaagctttggtgtcagtgtgcagcctaccaccactctgcgcctcaagtctattgtgattgatccacaacaaccaatccctcccccaatgtggtaggaggtttggaagaggatttagtggtataagatgtagccgacgaacccaacacaaatgaatctccaatgcaggtacaaaagctcaaaaacccactgcctggtgcacagccttttccaatgtcgttgcttcatatgcttagccacagaccatcaaattgtgtagtttcatgacacctggaagtgcatcttatgtttcttctttggccatcacaaagcaagctcttgttcatctaaagacatgcaatttccattgcaacccacagactccacaattatctcctcttcccgatagcattgatgccaccgccacttccgagcatccacaaccatatgtctttccctgagctaccttctcccctgctaccaacgtgccctcccatccaacagtaatcatgacctacgccttgaggtcacacattgaatgggccaacacttggctttgttgcggtagtggctgcccccaccatggttctcctccacattcttaacagtcgagtctccccgcaacatgttgcattggagtccactgctctcactcactgttgacgattagcatgatgttgtctgtggctacctaggagatggactagcatggcatggtctacaaggtccgtagaaagggctggcttgctgcacgtaccttgggtgaagagcaaatgaaacttgacgagccttctctaccacaccactagctggatctatattacttgatttctacaaggtatagtaagatgaggtgcttcctactaacccttgccttcctagattggaccccatggttaatgagttctactctatggcttgatcctccatgcaaatgtcctcctctctttgatgaggatcctacatgctcttcccaatgatgctacacctttggctacccatgacaacacatccaccaatattgatcaagttgtcctcctctaaggtaagcaaaagtttgcaaccaacaaaaaaagtttgtaggcaataagctccaatgatttcaccaagctagtgaaggcacaacattatagtagctgccttgctaccaatcccttagcaccatttgacccgcgagttgagcttcatcaatcctaaagaatttgtgggtgaagcgtgaccgcaatattcttagtttcttgtaaggaaccttttccgctcgaggctatcacaacaatccagtcaatagtccgcttcaacaacaatgagaagtactctaagactactattgaggccttgtgtcactaaggatgaagcttaggtttccgatgagtttctagttgcagccatatgtgttctttggttagttttctgtcggcacacagtagattagcttttagtcatttaatttaggtcttgagttctgtttagtttttgatgtaggctttggagttgtttcttctgggtcgtaattgttctaagacgaagcatcagagccgagtaagagcagatcctttatagttgttagttgacagtcttagtatttggctactttattttttataacccccccccccaccccaatagcagtatgctaatttgggtttgatgcagctgtgcctctagcacaaaaactcttcatcttattaatttatgcggactttgtcttacctttcaaaaaaagtgttttggtgaccatataggttttttctttttaaaatagcacaatttaaattagataaagaatagcatatccctgctcaatgttctagaccttagtgctttgactatgatggccaaacgggcgaagcccatttaagcatgggtctgctaggcataaataacttaatatgacgtgcatgcatgctttgtatatactcttaacacaattcataaaactcttgctattcatagtaccatggtgttgtttcttatgaatcacagttcagttaattcttgtataagttgtttctatgacaacagccctagaatatatgtatgcgcggttttcaaaagttagttttcgtgccaagctttatcaccatacatgatgctgtgataaacgatagatggttatgatatacaatatggaagtatggaactagcctaatagttgactttatataaccctaaaacatcacttattcatgatcaaagggaatacaactcaagtattatcactttgtgatagaaatagaatgcttttttgacgctggcaggtatatgggtgcttggagaataatatgattagagcatggtttattagaggaggtgcttatgcatagaaaaaagatcatttaattgtcgctattccctctattccaaattataagacattttgacttttctagattcatagcttttgctatgcacgtagatatatattatgtctaaatacaaagcaaatacgtactatgaatctagaaatttcaaaccatcttataatttgggactgatgaagtatgccatatgcaacaaccttcttataaagtgggtaaaaactaaattttgctcttggtgtgtgtgtgcgtaatagacaaatcaattgtccgtttcgtgtaacccattgatcaaccattagtccagcttaaaacatctcactaatggtaactaatgcatcatgaatttcaccctatagcattaatatatcctagcagctataaaacggcgagactgaggtgaaccttcgcaacagggcttggggtgtgcgagagaaagaagttagaagaggtagcaattagcataggtacggtctctattgcattgggccgggcctgctcttgcgatg

SEQ ID NO: 39 - DNA, 1550 bp, Sorghum sp.
tttcgtatttgcatgttcatggacgaaggtggcacatgtcgataagtttagggggcactttttcgcgtttgcaagttcatggaccaaagtggcacagacggacaagttcagatgtcactttttcgggtttgcaagttcatggaccaaagttgcacatgctgacaagttaaagggccgctagtgtatttataatgaactaactaggtttaaaattcgtctcatgattttcaaccaaactgtgtaattagtttattttttatctacatttaatattccatgcatgtgttcaaagattcgatgagatgaatgaaaagaatttcgagttgggaactaaacagggcctaagcactccagctccagacagttacacagcctctgcctctgtagtacgtgtatcggtgtatgggcttccgtcgctaccaaccgccaccgccggtacgttcaactctcatggtaatggcggatcgaacgactttcgtctcaaaccactccctcaccagacaccagcggcactgcatgcgtttagccgtttaccgtggtcagtggtccgtggacgtggacacccctgccctggctgcacctgcagcatgcaagtacgcacgtaccactcgaatccaatgccatggacgggaaggccggcaaagtggtggccaggccggccagccgccgcgtcccttctcccccgggccacgggtgcaaggcaagggcaaccgagccggcagccggacgaaccttccgcgaatcccagacctgcgcgcacgtccttggcacatgccacggccggatctcgccgcgggctggcactagcagacgggcagcacgagcggggcacggcaagcagcggtgcccttgcccttgcccctgcccctgcgtgcctgcccacctccagccggcgcctcactgtaaagcagcgagcgccactgtgcgcgaccggagcacgcagaggaaagcagcgcaaaaagctgcacgcgcgctttcccccgcccggcactcggcacccggctcccggcacggcaggcaccccaccacaacttgccagcctaggctaccacccctttcgcggatgccgccggggtcggggtggacgcgtccgcgcgcggcgcgtggggcaagtaaaggcgccccgcgcccgcgcggcccccaccggcgtggacgtacgcgcgggcaggcagcctcctcctccactggatccagggtgcggccagccgtgtcgtgtcccaaatctacccgcattcactctgccagcccacccgagcgccggagccgcccgccactcgcccgttggttcaccacctgctgcctgcctgcctgcctggctagctggctcccacagtgccacggctaaggaaccgccccccggcgctcccgtataaataccaccccactccattgccgtcccaacccactcacacaccagatcgaaacatctctcaagtgttcaagttccttcccagttcccacacactacacagaccccactgtgtcactagctagagctccggcagccacaaacacgagctagtagagctgccaacaagaacaagaagatg

SEQ ID NO: 40 - DNA, 3125 bp, Sorghum sp.
taaactcgttcgcttgtcttatcagtcgtactttttctgccagccagcagtgtttttcgctcacaataaatcagccaacagtaattcagacatgactcacgtaggctttttgctaatgtggcaagtaattcaatgaagagagataacaaaaaattagaaaccatttctaagatagaaaccatgcttcacgtgcaacgaaggagttagccgaaaaccatctgagattacgttggggaggggggaggagcgcatggtttccatcagtgattggggataccatgcacgctgccggtgagagatagcgcggccactgcaacacttgcttgtgcggttgctggtgggagcttgtgcgccaccgccactgggacctcacaaaggttggtggtggagctcatgcggacactgccacacatgctcacgcaccgccgccactcagagcttgcgcacgtgccgcgacacgctcatgtgccaccaccgatatctcgtgcagcgtgctgggacctcgtgtgatcgctagaaccttacgctgtcgtcgggcaaggtggtggtggtggtggaggaggaggaggaggagatggtgcctggtgtaggcggagcggacatgtctccatagtttctcaaaacatattcctaaacctacttgtttttcctaaaaaatagagaaagattgaaatatgagttggattagtaattaatgctacatattcaacaaccactcgaagtgagttatgtaattctatatgttgggcttgcttagatcagttagagctaattagcccaaattagctgggattggtgagttaattagttggctaacaactagttggaggcttggttagaggtttgtttgaatgggctagagttaaatttgactactagctaacaattagctctatgcatccaaacatatccttatttggctagactattcaatattttaggttgacctaagaaaactactctttctagtcaacttttagcttgactattcaaaatggatatataaacgtgcactacaagaatagtgttgatccgtgacagatttttttatcatagatttagacttgggcttgcttgaagcacatccttgggcccagtttctgtgaccatattgaagaaccgtcacaaatttgcatgaagtgctgacgtggccatgaccgtgtgcctaaaattgcctgaaatccaaatccgtcatggatgggcattcgtgacggatttcgagccttttgtagttttgtgacagattgaatccgtcagtacctttggacaatgtatagaatattaaatatagacaaaaataaaaactaattatacaatttgtttctaatttgggagacgatttagttagttcgtgattggacaataattgtcaaatacaaatgaaagtgttacagtagtcaaagcaaaaaaaatttgcgaacctaaaaaggccttagtaaagtaaagtaatactaacatgtgggatcttctaaatgattgtgttatatacaatgaagtatcacttaaaattgaattgaattgaaaatggcgcgtatatatataaaagcaactccaacagagatgataaaaatagatggctaaacttatgatttagccaacctctaaaatagaaaccccaacagaaaaggaggaaactccaaaagcctttccaaatgggtgaggcgaatggctagtggcacatacaaggtatatctagcaagcaagaactctagggtacataacttgctattttagaaaattagatacaatagttgttggactctctttttcaaaatactcaaatatagattacacaacatgaatagctctttttgttggagttgttctaagtgtactcaactttacaagattaaattaacttaacgaaaatcaatactatgcgattcaatatttaacaatgtatttgaattcttaataaagtatacaattcttttaagaaataaaataagataaaaccatgattttttttggattgatcagttatattactcgatcctgttaggtagaaagatcatactacctgttattccgatcgtcgaaaataaccttggtcaagctatgcctacttaaaaaatgagcaaggagtaattgagtactaattaaatacatacagtaaaataataccacaataataataataataataataataataataataataataataataataataataataataataataataataataataataataataataataataataataataataaaatacttgtgaaaacccacgaccgttgccactgccacggtcgtcgctgaactaacccggccgctggcacgggccaaaaagtccaggcgtccacagctcatcatcattttatttctcgctgaatttgtgcacaaaatttatcaaatccttggcacggttgcaaacccaagccaaaaaaaaaaatcgacaaccagtcggaattttctccgacaatagtcctactattcagctcacgcctggaacacatcactgtcatggctttgtagctagtgaactgaattttctgagcgcacacaacggcatcaccggccggtcacactcacaatcacaaccacttcggccggctctttggttgcgtttgcgcgagcacatctcccaaatctctacgcacttgacactcacgctaaacctacctaattaacgcatagattaatcatgtcatcaccaacaacgccaccagaaaaaatggaccctacttacactactacctacttatcatcttaaaatcactgtccatgcattattattagcatgcatatataggagattagcagtatagctttttcttagtgccatgcatctttcatgctaccttttttcttcccaaaatttcaatccattgttaaataaaatgcaaaaaaaaagaaaagaaaagaaaacagttagtaattaattgactaattggtaagctagtgcgtgatttggtgtggtggttggtgagctctccggccccatataacccccctccctgctcctccttcctcctcgcagcagcagcacacgccaacacttgccaagctctcgcgtcgctcagcgctagctcctagctagtatcttcttccaccgggcaccggccggccagccgtcgtcagctagctagctagccatg

SEQ ID NO: 41 - DNA, 3036 bp, Sorghum sp.
tccaaacttattacagtagcacactcataattaaaatttgttacaggtttagaactaaagttcaaaacagcaaatgaaggatcatccatgcaatgcgaataactccatggctctcgattctagtagtttatgtgttcttatagttctatcattgttctttttcattgtcatatggtcttttctttcgtacctttttctatatgcttattatttgacctctcgacttgagctgtaatttttctataatatttaatacaattacaaattttaaaactccttttaatataagacaaatattcaagcacttataactaacgtgaagaagctattttatagtaccataggcaggaatagatacaagggtatttctaggttaaatttttgcaatgacaatggtggttagttattagtatgtagatgttttaaaattttattttaggccttgagatttaacatggagacttaataagcaatgttagagcaactccaagagactcttcatattctttctagtttataggaatagagattttggtgacaaaagtacttcccaatatcatctttaattggatcccccaatatagacatgctctattccaaaattctccctagttaaagatagagagcgaggatgactctctagagtgcgcacaagatacaaaaaaacagttggagggtacaacaatatataaaaacaattgtaactcaaatgactctccaaataatagctcaaagagtaaattttagaaagactcttcggagatgctcttacaatcaactaatacttcaatttatatagccacccataaggacattggtataacttgcaattattctactacttcctcttatcataaatgtaaggttatctaaatttgttttaggacatatttctaactttgattacaaacggttgattgagtatttgtattagggctcaaacaaagcctccttcaactaacaagaatggttcaaaggctaaaattcccatgtcataatataaaggatataaattccttcttatcgataccttgggattttttaaatgtttgtgtgggagacatagattgtatatgatccatagttctccataatattggtactcatacaaagtgagtgggcacaatttgctatctatagtacatgagataagacaacaaattatgacaactcatggtgaattaatatcctaattttattatttctgtttacttcatggtgtattattgttttgatagtactcatgagatattttgtggtgtttatctacgactcatgtattatgtatggcacatcccatggttcctataatatttcttagatgttcgatggtgtcctttttatcattatcatattattcatgagatgcttcatggtgtacatctcactgttttcattagccattggattttccatgttgtatttttactactcaaataatgttcattagatgttccatgttatattttgattgttctactatcattcagaagattttattcgtggtgcatagtttatattttatattttcattaaaattgagccacagacttctatttaaagtcatggctgaaagtacggactctctatagatactaatgggccgtggattcgatcaaggatacagaattaggccagtaattcccaggcatgggctgctaggacactggcccgttggagaggccaccgaacagcgcaagaagcaccaccgcgtggccgggccaaattgtagcagagcagaaccacacttcttggacttggcacttgggcctctctacccgcaaactctctaccacgactctgctgccctgacttcgttcgattgtcgacttgtcgttgagtcgaagcgatgttgcacgtgggcgagcgggtgtcatttgtttccaggtggacccgaaaagacttcgagctggcttcactatacgaaaccgcccgaaggccgatcgtcccacaggaatcaggcccacaggaacccaacgaccatgtttcttctccatctcaccaaacatcgatgaggcagccaccggcaccagcagacaattgaagcagtggagatttcaagacttcaagttacaaaacaaaatctatataaaaaatagtagtagtagtagtagtatctctcggggtcatttctatccgacgacaaccaaaaacaaaagaaagtcttatctgctctctctctctctctctgtcccacaagtttccatcgttttgaatgccgaatggggcttgtgttggatcacattctagctagtcatgtgctcttgtgtactagtgacaaggcttggaaaatacaagggatcggtcaacaatatgatccactccaaagttgtcaacaagcctttaaatcagcaggagcacctggtatcctccctccagggggttggtgtggtgaacactgaccctcaaatttttgggcaagtgcctgtgctcccgaagagcacacaagtaagctcaaaccaatcaccattaacttttttttttcttactgaaagatgcctaggcatcttattccagaggccttgtttagtttaatctaaaaacaaaaaactttttaagatttttcgtcacatcgaatcttacggcacatgcatgaaacattatatatagtcgaaaataaaaactaattacacagtttgactgtaaatcgtgggataaatcttttgagtctagttagttcatgatttgacaataattgataaataaaaacaaaaatgttacggtatccaaaatcaaaaatttggaaactaaataatgcctgaattatattaaaggaaaaaacctttttagcggcgtgaaaaacactcaatttttatatctaacaattatttataaaaataaaatacactgagctgaaagaaagtggaaagacgagtaaaggggcaagtcagcgggccccagccccactcacctaccgccaaccgcccccgagatctccctcttcttctccactcccgtttcccgccgtataaattcggcgaacaccgcaccaccattttccaccaaccccggcgcccgccgagtagcccagccatg

SEQ ID NO: 42 - DNA, 3064 bp, Sorghum sp.
agtcatcaaacaggcttcacattaactgattgtttatataaagtttgatgtgagccatcaagagaaacttcgataaggatataaacgtgaggtatgataggttttctgtttactacttcaattgtcatctgcagtcagtcccaacttgatgtcattttaaaagttccatgtggaaggcatgcggaagattacggccttgtttagtttaccctgaaaaccaaaaagttttcaagatttctcgtcacatcgaatcttgtgacacatgcataaaacattaaatatagacgaaaacaaaaactaattacacagtttagctgtaaatcacgagacgaatcttttgatcatagttagtccatgattggataatatttgtcacaaacaaacgaaagtgctacagtaccgaaaacttttcacttttcggaactaaacaaggccggatgtcacactttctttttcagggcagagatatgatgacagtagtggtttctagaccataaataagtcatacaaagagatccattgattgttattatctgctaccaataggagataaaagcaagttcatataaaacattgaatctcttttataacaacagaaaaacagtttatgtctatgatgcctcctcttccgtactgtatggtacgagaataaagtagaaaagatatgtttctgcaatcaataaaactcctctggacttgtgccaaagaaaaacttcataaatgtctatgtgaaccaaaccactcatcttttttaaaaagaatgatttggttcaaatctaaaattacactctttttttggaacgaggaagtacatccatacaaaatttctcaaaatttcaagtacaaacccagtcatgtttatatattatgagattaaaaaggcaaactttgcctgaaatcgatgtgaacatgttcgttctagttctgtgcaacgcattcatttgcatctgaaattccacatgggccttgtttagttcaccccaaaaccaaaaaattttcaagattttccgtcacatcgaatcttacggcacatgcataaagtattaaatataaataaaaacaaaaactaattacacagtttaactgtaaatcacgagacgaatcttttgattctatttagttcatgattggacaatatttgccacaaacaaacgaaagtggtacagtagcgaaatccaaaaactttttgcatctaaacaaggccatgaatgtggaggacacaacgtcacctatggatggtcgcgaaaattttgaaatcatctctgtagctgatttgcacgaacgatcaactcatgaacatcaccttcgccccgtcgccgccggtggcttgccgccattggaccgacggccggcggccgaggcctgccactagtgccgtgccgtgcccggcggccaatgatgcggcattgtggcacgtcaccgaatgttggctagatgctttttggccagcaattactttttttttcctcaaatgtgactaagaatcactttgctggaacaatttttttccgtggctacaaagtgcaaggatgatggatcgctagatgcttcttgtgttcgtggctgttgattttttttaggaaacgtggccgttgatttttgcacgcgccattaccctaacgtgcgttttttttttctttcctttcttgcgcatacactttaatttgctcatgattaattactgggtaatctcggatcaagaaatataggtgtggttagccacgaactcaatttgaaactagaaacaaagcattggcgttatgttttttttttttgaagagagcattggcgttatgtaggaactcccttttgaattaatggctaaagttaggtctggtttaatttccaaaaattttcaacacatcaaatctttggacgaatgcatggaacattatatatagataaaaaaactaattgtactgtaatttgcgagacgaatcttttgagcctagttagtctatgattggacaataattaccaaatacaataaacgaaagtgttacaatagccaaagctaaaaattttcgcgaagtaaacaaagccttagatatgcttgcctgtcatactatatttataaacaaatataaagtaattcacttaactgtccggttcaaactacagttcattctattttttttaagtcaagcttctctaattttgacaaattctatagaaaagtgcacagattctactgcatgaaattaatttcataaattctctgcaaaacatgcttcctgttgcatacttgaacttgtagatactaatacattatcctaaaaacttagtcaaaattagagaagcttggcttagcatagagctaaagtctactataatttgaaattcaggattatatatacatatatcagtaataaaactaagctaaattttgctattgaattattggtcgtttatgatattcacttattagggccaacatttttcgcttgacaacttacacaagttagatacgggcatacggcgttacattagttacacaagttagacacggcattgcctggggcctggctacacacattcgcttcacaacttacacatgtgcaatttttctaacacgacctctctaacaaatgtacttcatctgtccctaaatgtttgtcaccataaattatatgccgataactttaacttaatttataaaaaaaatatataatatttttatctctaaataaatttattaaaaatctagattcaaatatctatctaatgatactaattatgtatcataaatattattatttttaatttatatttaattaaatttattttttaaaaaatgaaaaccgtatacatctagggatgaagagagtacacaatgtagtggtaccagacaagcagtcagtccaagcatctccacacaaactgttgttcaaacacgcagtctcacttgctcacctactccaagtcaatgtggtaagtacacttttacctattaacctatttattagattatataagcaccaccacacatgcatttttaccaacacaagccagccaagctagcacacaacatgcaagccaaacttgaccgaagccgccactgatg

SEQ ID NO: 43 - DNA, 3021 bp, Sorghum sp.
ttttaagcaaccataattaatcgtcataatccgttgagcaaaacactttgattattataacccataatcctgattatggtaatcttatatataatccatattataataatcaaaccatgatctaaacaagccctaagtttttgaaaccgatataaaaggtctcccctgctccctaagttctctccatttgacatattctttttttccatttttaaagcctacgtatacattacgaaacaaaatcacatagcaattctttgtttccttttcttttgagaccgtacacattaggaaacaaatactacaaaattagagagccacagccacatgctttgagctcagtccgtcggtgcagattggagactcgagtcctggacacggactcggaggcgatgccgacggcatcccgtgtgtccgccattcacacaattcacagcatggtggtccggcggccgtccagcctccagcgagcgaagaacgaccagtcaccagcacctgcttggagcgcgagcgcaccctcatttttcggttgccgcctccgtccttagtggcggagccagaaagttttagggtggctaccttttagtcagacttcacaaaaacataggggtgatcgtttttttgggagccaggcttgtcttagctccggtgggtatttttctcaccttccatctttccgtgctcgctcacccttcaaagtcaccgatgacttgatccggtggtccgcatctgcacctcctctgatggctcgcatctgctgttccggcgctcctcccatcggcgaactcgctagacagggcctcccgcggtggcttgcttttggccgacctctctcattggctcgccacggctacaggcacgcaagttgagtcgtgcgtgctcctaaccccatggacgttgccgtcttccctggcagacaccgtcgtattccctcgacaaatggaggcgagtggactcaccactcggtggaggaggcagagaggaggggtgtggaggaggggaggcgcgtacggtattttctagcttggttttgctgtggcattaccaaggtcggaggatatcccgaggacgacgatgacaggacggtcattggcaactccgcttgcgcattggactagggtcttgtttactttcaccgaaaaatctaaaatttttcaagattccccatcacatcgaatctttagacgcatgcatgtagtattaaatatagataaaaataaaaactaattgcgcagtttggtcaaaatttacgagacaaatcttttaagcctaattagtctatgattggacaataattttcataaacaaacgaaagtgctacggtgccgcgaaatttttttcctcaagaagtaaacacggcctagatgtactccacgtgcagtagaagtgcagcagcaccatccaccgtcatcaattgccaagctctcctggctatgtgtgctttgctgcacacctgtagattagtattagatgtgttagggatttgaatttggtgaagtattggtttagattggatgcatatactaataattaggattgaattggataggactgattttaattagatttgatttgaattcaaatgtctcaaaggaaggtgctcaccatgtattcaatgaattgtcacaaagaagaggtaactctatctaagtcaaaccagccaaccaaacaaacgacatgctagctaggcttaaatagtagagtgaaccaaacaagtctatcttgacttgttgaaggcaggcttaggctagcctggcttatttcctagtcaggctagaagtaagccagttaaccaaacaaacccttacgagccgaggcgattttcattatacataaatgtttatctagaaatatataatatttttctataatacaaagaaaatttccctttgttgtgtatgggcgagcccgcccagccagccaaggtcgccgggcagtggctccgccggtgtccatcctcactgtcgagggagatgccacaaccagaactcgggactgtttggtttccaaattaaatttgagtcagtaaaaattttagttactttagcagttaaatttttaaatacactaattttaaaaagagttaaaatagtttaatcctattagtcatcaaaaataactaaagtaattttaattaacaccttatttagatgtgaaaattttttggatttcactactatatactttcatttgtatttgataaatattgattcatcttgcgatttacaggtaaactgtataattgattattttttcatctacatttagtgtttcatgcatgtgacataagattcaatatgacagagaaccttgaaaacattttggattttggtgagaactaaacaaggcctaactaaaatttagtaaagagaactaaggccttgtttactttcaccccaaaacccaaatattttcaagattttccgtcatatcgaatctttaaatgcatgcatagagtattaaatatagacgaaaataaaaactaattgcacagtttggtcgaaatttatgaaacaaatcttttgagtctagttagtccatggttggacaataattaccacaaacaaacaaaatgctacagtgtcacgaaatttttttcttcgtagactaatcacggcccaaataaccctcgttccttccttcatcaacccctgtcgcaacccaaccgcaagaagggaggggcgcgacccgtcggctcgtctgagccccgaaccccctttgactatgggcccgcctggccgcctggccgcccgtagcaccaaccaaccaaaccaaaagccataccaaccaccgcgatcgcaattcgcaaaccaaacaaaaaattaacaaaaaattcgtgtacccaaatcggacccgtctcgtctcgcctcccctcccaaaccgctataaaatcccttcccattcccctccgcctgttccatcgcctcttctggcagacggccaacaacaaacaaaacagagagagccacacaccccacctacccccaccccggcggccgggctccacgctcctccagcatg

SEQ ID NO: 44 - DNA, 2100 bp, Sorghum sp.
tgggagcggcattgttgacgagactttgacttcacaatacacgagtgggacgtgtggcctgggcacaccgactaccgacatagtgatatcaagccgcgtggtcacaccgtcatactcaatccacacctgcaggagcttcccactggccagggcgatgtcgacggacttggtggacttggaggtcacaatcgaggtcaccccgtagttcaccatggatggaggcggaggcagaggtgttccgttgctgttgcagttcgcctcatagcgcgtcgccggcaagctccgggtatcgttgtagtacgacagttcgcctcgccatgagctggaagacgttgtgtcctctcgctgttgcccatgcttgaaggagcttttccttaaggagatcgccctgtatgatgagctcatgacccaaccgaagtagagcagcagcaccatggcggagctcaccatatgttcgagagaatacttcaaagggggtgaactcaccctcatcctagccatgctcggcagcgcacgcaaccaaataaattaaatgggtcttgacttgctagggacaggcttagactagtttggcttaattttgctagctaggcttgtctaggggataaaccaaatacactcatattttcttgaatggaataataaaatagagatatttgtagccattaaggcatactattgcacagtaaccaattctctaaaaaaactattgcacagtaacttgtttcggcaagtggaaatagttgtgtatctgaaaccaacaattggctggggccgagcccactgaggaaatttctagaaaagaaggccaatttcgtgccggcaagaaaaaatacaggtacgtaaaatagtggctcttattatgaggtcttgcatcgatgttcctaaaatgaaaaataaacgtgttggatggtgtttgtgtgtgaatggcgtccatccatccatgagatcagaacgacaagtcaagcacggcatataggagctagcttattagtgtggctttgctgagacgaatgaaagcaacggccggcgcgcatattttcaatgcgtgtagctttcaagctcgaagataaagacatgacaaatgaaaggccggcatccgtgcaattcaggaaattcgtcaaccaagctagccagtgaacttgcagatagatgcgtgtctgttcgcttgattgataagtcatgactaaaaataatatttgttatttttttattaaaaaacaactgttaaatggttagcaaattcggtagataagcttaagcgaacagacatgtatatatgcttcaataattgacactagttagctagagagatggtacatcataaaagaaaccaaagtttaatttccactgattattagttagctactcctatgttcttttagctcagctattaatgttatactcactccgttcaaaattggaagacgttttcacttctgttatgtatctagacgtaatggtgcatattttaaaggcatattatcataatttgaaatgggaagagtattttaatttggcacatcaactcctgcagttccaatcaatttaattttggttttgcacttttgcagcatctaatacggttgtcccttactactgaaatagtataagatattctattggggaaaaaaacattggatcatctgatacacctctttgattgctagatactattaatcctctttcctatgaacttgatcgaagttaggacagtttgactttgaacaaacttaattggatcttttatatttaaggacataaggagtattacttacaaataattaaagtagaattcgattaccagttaaattgaaatcgaaatatatactccaagaataattctggagacaagtggacattggatcggaggccaggaggacttgttccggagagagcctatggcgtgctgacacggcgcgttgcgtgcctgtgttggctcaataattggacgaagccgaatcctccatccactgctataaaaaccggtgtaggggcttcattgtgctcaagctcaaccaaagcgactttgtacaacgccctttgatagatatttgttcttgagcttcttcgtttttgcaccaaaagacaagcaggatg

SEQ ID NO: 45 - DNA, 3003 bp, Sorghum sp.
agggcccatggcctgcgcacctgctgcccctcccatagggccagcactgcacccgcttcttcccttctgcgtgtctggatctgtgcactgtggatactggatccgccaccagggcttctcgtgcgccatcgcagggagggcggccccaccatagagataggcgccaccagcgggggaaggtgccatcggatctaccgtaggacgtcaagggtacagtagatccaccaccgccagtgtggaggggagccgctgcagtcactatgggggtggagggggccaccaccatcgctgtcagggtaggagaggacgaccacaactaccgtcaggtgtgggaggaggcgatgttgcgggagtgaggcgtcaccgcatgctgccgcactagccatcgtgggtgaaggagagagggagcgccgggatgtgttgtagctggtgtatggaggggaacggggacgccgtcgtcaccgatggaattaagggtgctccaccgctgccatatctacatgtctggtgctccggatgtgctgctcgggctgcccgtgcgccaccattaggtggggaagaaccgccccgaccactatgggaggaagatgcgccggagtagccaccaggatgagggaaggggcgaggatctcgccatctattgaggaggccgggaggggtactgccgttgccaagataggagagagaaaatagctagagagaaatgaaagacgactagggttaatgaggggtagcatggtccattgttttgaaatagctggtgtatcccaacacactaccagctaaagttagctgtaacaattagctagctatattttagctaagacatggcatgccctgactatctaacagccttttgcgattcctttgatttggtgtatatatcattttttgtagagtgacatgcatgagtataactaagaggacacatggataataacatatatataatttcagccactccggtgtagtatacaagtgtagaagcgtgcgtcgttttatatttgaaaaacaaaaaatgcgcagtattgaggaacaacgacgacctaggagcaaattaatgcacaacagtgtctcttaatgaagagacgggatagcttggtacgagattggtactgagagcgttgtggatgcatatgttaattagcagtagctacacaggcacacagcccgcttgcagtggttgggacgtcagtgtcatcaatgtcgggtcggtggcaggagaggatggcgaaaacatccaagcagaaaaggacatcgccgttggaacaagggacgagtgcaccgctccggcaagccgtaccgtacgcctccgaccctgaccccgccacggcgcgttcgctagctgctgactgtgagcctgacgcctgagcctcaacacggtcgggtcgccaccacactgtgcatcatccgttcatccacgactgtgcttattgcacagccacacacaacactgcccagcctatagggcagcgacgtatgtacgtgtcccttttaacctagctatagtgataaaactgtgaattttctagctagatgaacttttgggatggtttttcaaccacgcaccgacgcaagctatgcgtaattcaacaagttaaaggtctgtaggatactattatttacttacaggtctgattgactggtttaccatcaccttggacctgcaggcaaaagcacgatgtcgacaactgccgtgtcggtcacgcaggaaatccaaagttctacgacgtgtatacgtacggcgtgcgtagcgtcactctcatactctcactcactacacaatctgatgtcctgcagtggcctgcaatgtaaccatgcatcgccaatcatgtgtctcacagtgccggtcctgtgtgtcctctccctttggcgatgagcttcacgagctgatgcagtgccccgcttccatgcataggtcttgtttagttgcaaaaaattttgcaaaattttttagattcttcatcacatcaaatctttagacgcatgcatgaagtattaaatatagacgaaaataaaaactaattgcacagtttggtcgaaattgacgagacgaatcttttgagtctagttagtacatgattgaataatatttgtcaaatacaaacgaaagtgatactattcctattttgcaaaaatttttggaactaaacaaggccatacatgatgtccaccggtagacatgcatggcacaccaatcagctcgccgtagtactataggatgatgatctgagagttccaggaccatgcatgtgcttgtgcagcagcgcgcgacaggtgaagatgcatgacgatggctagctagctctttgtcatgcatccatcgtccacacaccataaaaatatctttgctacctctcaaagcaagatgttcactgtcctggggatgaatcttcacacatacagtatacatagctggctcgctggtcaacagcgcgcgcgcggcagtttgcgtcgtcaaccacaagctaacaaatacctacctgtcgtcccgtgtatcatcaaaaaagttagcagcaaacgtacacgtcgtcgggtgtgtgatatgcgcgcggtgactcgcatggcaggcagcagcgtgtatagagagactagagagtatgttggaacaagaaatggatggaagaatccatgagaaagtaaaagtgaaagtttttcctaaaaaaaaaattaaaagtactgaaagttacgtgctactgctatccgttgaataacattaacacggggcttacctgttacctacccgttgatacggcggagggcaaacgtgttattagctgggcagacagcccatccacgcgtcaaaacttggttggctctcgcgcgctataaatccgacccatgaccacaccccgtcatccacaccacagacacacaacagagactgcactcaggcactaccaacagctgctccagaaagagaaacagagagagcaacacagagcagcagagagctagctagcaggcgagcttgcttgttgcaggagcagcgaagcagccatg

SEQ ID NO: 46 - DNA, 3102 bp, Sorghum sp.
attaatccaattgattatgtcataacatttttagatttaaaatcattttgaaaatgacaattttacaaaaaagacctctaagcttttctgtaattatttggaggtaagaactacaacattttagttcttggttttgaaaaatatttacagggtcagttttatttacaaaaaaatccctaaattttgaaaagcttaatcatagctttaggtggtttttgcggtgggacagtagaaacaagctatatgttttttctaatgcttgttgtaatgtttttttttgttttttgctatgtttgtaccatggttcgagtaaaccatcacttagcctgcttaaacctcatttgaacattgcatggtggtctgtcatttctgtttattcgaccattgacgcccatttaaatagctgttttgcccatttcgttgagttcctagccagaaaaatggctagatgactcattgtaaattgtttatcgtatcaatagcatgtttactttgtctacatgatgacccgtagcattaatgcttacttttaattttacaatggtagaatgtgtatagaaatatatatagcatatttttatggttactgaattattttgtacaatatatattataggtaagacaaacatttgtggcgtatttacattgtttagatatatgtaatctaaaaaggcacatttagtgttactttgtaatctatatacttttataaagctggacccgactagatgttttctctctgaatgcaaggtgttaatatcatccctcactttagtttctatcacattggatgtttgtatactaatttagagtattattaaatatacactagttacaaaactcattacatagattgtagctaatttatgagacgaatctattaagcctaattagtgtatgattttacaatgtggtgctacagtaaacatgtgttaacaatggattaattaggcttaatagatttttcttgtgaattagccacaacttatgtaattagttttataattagctcatgtctaattctactaattagtacccaacatatgacaaggactaaactttagtcccaagattcaaacaccccataagtgtcccactaacttgcaatcctttcatgcaagctatccatgtcatgcatcctttttttcgaattacacaatatctcttattaattataaaatatattcacatcatctttattatgtgcaatacttttaatctctaacctattaacgtaaatgtcatcccttattaattacaaaaaatatcctcatcatctctattatgtagatgtaaagttatttacatatgttatttgtttcatcacctattcttgtataacgcaatcaactattttattggttttttctattagcttattgtttttttactagatccgatacaataaaataacatgcaagtcaaattcataaatatacacacaatatacagaataccatatagcattgctatatacaaaacagattatttttaagaaaatcccgcggcaatgcgtgaatatgtgtctagtacctttcatagtaaaaaaaaatgcagacgcatttttaggcatgtttcggtaattaattgcaatttagaaggctttttaaatttatctttagaaatgacaaacgtggggaatttaaatgcaactttagcgaatcgtttatgtatttttcgtaatggtcaaggcgggcggcttttctttttagaaatatagaacaggtcaaatggttgaggaatgaagcgtcgtacgcccgatgaagacaatgaggaggcggactcgttttgtcttggatagcccatgaatttgaggtagctaggccacaaattcaggctctggaacagcttggaatgtcactttccagttcaatgcgcagccaaactagccgagacccatgaaactgattccaaattcagcccaattaattctatagatccaaacaggccgtgatcatcagtgcatcatcaggtggtccggagcagccgtccgtgtagaatgtagtagaacatgtgagagggacgagagcactagaggcgaagagcaagcagacgcagccgaagcaagcccaagcccaaaactgtttgtctttttttccccttccattgtcgtcgttcttcacttatcctttcacaaaccacgacgatcgagctgaatggaactgcttctgcttgagagagacggcggatgcatgcgtccggcggagatttgcagcaggaggaaggggacgagtcagtcattcaccgaccgagcagcaggaccacgaccacctcggccggatcctggctcgatcgtggacgacgagctagagggcgagtccggccacggcggcgacgtcctgctcgatcggtttgatcggcgacgggagaccggagatagatagcgagcgagtccggccacggccagcgaaattttggaaacagcttgtgcggcgcagatttgcatgaggaaggggacgacgagtgggtgtgtcttcatcttcgccgacgacggagcagagcaggaccacctcggatcctagagatcgagcgagtccggccatggcgacggccaggggacgtcctgctcaacatatacctaggtttaatcggagacagcgagcgagtccgggcacggccagggaaattttggtttgcagtcagtagtagtgactttcaccactgcactactacctgcggctagcttatctatctatctatctatctacaaataattaaagtggtggcacatcacatatagtccaaccatggcgtggcgtggcgtggctccatggacatgttggctggctgagacgataaggcgcgccacggggacgcgacatgtggcggcggacgcgatcaggataggccaggctggccgggttgcccgccatgggacaacggtggccactcctcccacatccgcttcattcgtccgatccgtccttgccccaacgacagccatccgtcgccatggacgcacgctcgctgcctcttctatatatgccctcggtgggggagcctacaggacgacccaagcagcaagaagcagcaaaaacagcaagcagctcactctcagctcgctccctcactagctactagtactacatagcagcagcaatg

SEQ ID NO: 47 - DNA, 3003 bp, Sorghum sp.
ggaggtaagaactacaacattttagttcttggttttgaaaaatatttacagggtcagttttatttacaaaaaaatccctaaattttgaaaagcttaatcatagctttaggtggtttttgcggtgggacagtagaaacaagctatatgttttttctaatgcttgttgtaatgtttttttttgttttttgctatgtttgtaccatggttcgagtaaaccatcacttagcctgcttaaacctcatttgaacattgcatggtggtctgtcatttctgtttattcgaccattgacgcccatttaaatagctgttttgcccatttcgttgagttcctagccagaaaaatggctagatgactcattgtaaattgtttatcgtatcaatagcatgtttactttgtctacatgatgacccgtagcattaatgcttacttttaattttacaatggtagaatgtgtatagaaatatatatagcatatttttatggttactgaattattttgtacaatatatattataggtaagacaaacatttgtggcgtatttacattgtttagatatatgtaatctaaaaaggcacatttagtgttactttgtaatctatatacttttataaagctggacccgactagatgttttctctctgaatgcaaggtgttaatatcatccctcactttagtttctatcacattggatgtttgtatactaatttagagtattattaaatatacactagttacaaaactcattacatagattgtagctaatttatgagacgaatctattaagcctaattagtgtatgattttacaatgtggtgctacagtaaacatgtgttaacaatggattaattaggcttaatagatttttcttgtgaattagccacaacttatgtaattagttttataattagctcatgtctaattctactaattagtacccaacatatgacaaggactaaactttagtcccaagattcaaacaccccataagtgtcccactaacttgcaatcctttcatgcaagctatccatgtcatgcatcctttttttcgaattacacaatatctcttattaattataaaatatattcacatcatctttattatgtgcaatacttttaatctctaacctattaacgtaaatgtcatcccttattaattacaaaaaatatcctcatcatctctattatgtagatgtaaagttatttacatatgttatttgtttcatcacctattcttgtataacgcaatcaactattttattggttttttctattagcttattgtttttttactagatccgatacaataaaataacatgcaagtcaaattcataaatatacacacaatatacagaataccatatagcattgctatatacaaaacagattatttttaagaaaatcccgcggcaatgcgtgaatatgtgtctagtacctttcatagtaaaaaaaaatgcagacgcatttttaggcatgtttcggtaattaattgcaatttagaaggctttttaaatttatctttagaaatgacaaacgtggggaatttaaatgcaactttagcgaatcgtttatgtatttttcgtaatggtcaaggcgggcggcttttctttttagaaatatagaacaggtcaaatggttgaggaatgaagcgtcgtacgcccgatgaagacaatgaggaggcggactcgttttgtcttggatagcccatgaatttgaggtagctaggccacaaattcaggctctggaacagcttggaatgtcactttccagttcaatgcgcagccaaactagccgagacccatgaaactgattccaaattcagcccaattaattctatagatccaaacaggccgtgatcatcagtgcatcatcaggtggtccggagcagccgtccgtgtagaatgtagtagaacatgtgagagggacgagagcactagaggcgaagagcaagcagacgcagccgaagcaagcccaagcccaaaactgtttgtctttttttccccttccattgtcgtcgttcttcacttatcctttcacaaaccacgacgatcgagctgaatggaactgcttctgcttgagagagacggcggatgcatgcgtccggcggagatttgcagcaggaggaaggggacgagtcagtcattcaccgaccgagcagcaggaccacgaccacctcggccggatcctggctcgatcgtggacgacgagctagagggcgagtccggccacggcggcgacgtcctgctcgatcggtttgatcggcgacgggagaccggagatagatagcgagcgagtccggccacggccagcgaaattttggaaacagcttgtgcggcgcagatttgcatgaggaaggggacgacgagtgggtgtgtcttcatcttcgccgacgacggagcagagcaggaccacctcggatcctagagatcgagcgagtccggccatggcgacggccaggggacgtcctgctcaacatatacctaggtttaatcggagacagcgagcgagtccgggcacggccagggaaattttggtttgcagtcagtagtagtgactttcaccactgcactactacctgcggctagcttatctatctatctatctatctacaaataattaaagtggtggcacatcacatatagtccaaccatggcgtggcgtggcgtggctccatggacatgttggctggctgagacgataaggcgcgccacggggacgcgacatgtggcggcggacgcgatcaggataggccaggctggccgggttgcccgccatgggacaacggtggccactcctcccacatccgcttcattcgtccgatccgtccttgccccaacgacagccatccgtcgccatggacgcacgctcgctgcctcttctatatatgccctcggtgggggagcctacaggacgacccaagcagcaagaagcagcaaaaacagcaagcagctcactctcagctcgctccctcactagctactagtactacatagcagcagcaatg

SEQ ID NO: 48 - DNA, 3003 bp, Sorghum sp.
attaggcggaccaacgcccccgctttgctcacgttagctcaatgattcccccaatgagtaagggagtagagggttttggtgatgatcaagggttatgataagggatgaacggactgagctggattgaggaaatagacctcttaacaggatcatctctgaaaggcagccctattaagaagacaaactataaaaatccattttcagagggtgtccaaactgttcatcttcaggtgattcaaaagatctacctcttaacataaaaattgtggtcttctaaaattatttttattgtagtgagttaatagtgtattcaacagaaaacagttaaatggagatatgaggagttaaacctcaatcctttagaaagcttatcataatgctctaacaatggagctacatccccaacttatatttacttgttcattataaatatttttatagttaagtatttttcttttttttagaattagaccatacaacacacgcacactcatcccattgcgaccaattttagaatttgtatagcaaaaactaggctccaacgtatgtgctatccgactttgtaaaataggaaactggttatcccttgttttttttggtcatatatagccaacacgggcacttgctatgtggttttgtaaaatatagtaagactgttgtgaacctttttttgaagagttttttattttacaaaatagataaataacagaatttcgctaatagtttttgccaaacttttggagttgctcttgtaaccattagtccacacgcccttttgcagcattttcttttttacttgaactcctaacgtgaatttatatagtgagttgatcatatatatttttacaggcactcctaacgtcaattttatatacaaaaaaaaatagttactgaatggagatgcaagtatagtttaaccccaacctttgtaaggctcataatgctctaccaattgaggcacatcctaaattttatttacttattcactatatatatttttatagttgactatttcttttttacttgcactcggtactgtactgataaaatatagttattaaatggagatgtgaggaagtgaaccccgaacctgctacatccgcaagtacctaaaatcaattatacattccctattttaggcatttttgcagtatacacaattaactttctaatcaaatactttttttagtgcactcacattactcataggggtctacagttgaacaagtggcttcaattttgctccctagaatattaaagaatatgaaaacaggttgagatggtcatgaaatagaagtagctagctttttcgatattcaaaagaaaagatctgataatgtagtaaccaagaaaataatttaatcagatatgcaaggaattgaaccccaatagaaagcttatcacaatggtccactgattaagccacatcccaattttacttatttttgcactgtatgagtaattttatatattattttttcttatatataactcataatcgacagtggaaaaatctgggtcaattttgtacaatagatcattagtctacttgaaaagagttcaatatagtgattgaatatatagaaatactgactttgataatgaaacataaaaagtgttgatactgtattaatatagaagatgattgaagaggccgagtaactaaacaccaaaacatagaaagcctatcataatggcataatggtctacatagctatataccctctatttttcttacttacttttgcacattaaatttatagctagttatttttttgtacttgcattcgtgaatcataagaatcgataatggatacggtgatgtcaattttatactttaactagatcattatatttgtcgacattaaaagagactgagatggtgattaaacagaaagatttctttcttataattcaaataaaagagcttatgatatactcaatccatccaaaattataagatgtttcattttttgacaccaagtttgaccacacgtcttatttaaaaatttatataaaatattacttttttatcatggcttgggttattaataaaaattcttcaagaatgacttaaatttgagtatgtttgcacaaattttttgaatagacgagtggtcaaacttagagtaaaaaaaagtcaaacgtcttgtaatttgggacgcagaaagtattaaaaaaataagttatatagagatgcgaggaattgaacccgggcccggaacattaaaaaagcttataatgtggaaaaggatcagcttgttggattccttgtaatagaacttgtccaccgggatttaagttcatgacttgacacgggtgctcgtatttttttggatttattttaggatttaacggcgctatatttttattggtaggcgacgtgtccgtcgatagcgaggcgcctgtggtgactttgtcaatctcgagatttgtcggtctaactcggttctttgaagatagtcataagggtacggtgtacgtacgtgcgttcatagagatgagagtgcgcttatgtaccctgaacatccgcgttaaccgagtctgaaaaaaaaaatgatgtgtaaccgctcaatacggcaggatcagggctctcaattgatgcagtggtgacaattatatccctgtggattttgtttcctgtacacttggggtcgctagctaacctatatatgtttccaaaagatatgtcctcaagtaatagtgagacctgctagctacgcattgctgctactgcattcgtggaagaaattaaactgtgttgaagcaacaagacaagaaagcaaaatccacagggattattgtcgccactcccgcaatggctgctagcctgccaccgcatcatcctgttcgttttcgacgcggcaaacagcagccattccttcctcatccttcccctgccttagccgcgcgcctggttatttgaaccccactgccgccggccatggcgcagaaggacggccggccggcctcacacaagtgtcagtcatcacaacctagcta

SEQ ID NO: 49 - DNA, 33 bp, Artificial sequence
  Synthetic primer for amplifying gene regulatory element from Sorghum sp.
cgcggatccatggtagatctgagggtaaatttc

SEQ ID NO: 50 - DNA, 33 bp, Artificial sequence; identical to SEQ ID NO: 49
  Synthetic primer for amplifying gene regulatory element from Sorghum sp.

SEQ ID NO: 51 - DNA, 36 bp, Artificial sequence
  Synthetic primer for amplifying gene regulatory element from Sorghum sp.
gagaggcgcgccagcaaccacggtgctagaagctat

SEQ ID NO: 52 - DNA, 28 bp, Artificial sequence
  Synthetic primer for amplifying gene regulatory element from Sorghum sp.
gagaggatccctgcagagaaaccaaaca

SEQ ID NO: 53 - DNA, 30 bp, Artificial sequence
  Synthetic primer for amplifying gene regulatory element from Sorghum sp.
gagaaagctttcgcttcaaggtacggcgat

SEQ ID NO: 54 - DNA, 28 bp, Artificial sequence
  Synthetic primer for amplifying gene regulatory element from Sorghum sp.
gagaggatccctgcagagaaaccaaaca

SEQ ID NO: 55 - DNA, 36 bp, Artificial sequence
  Synthetic primer for amplifying gene regulatory element from Sorghum sp.
gagaggcgcgccctgtttggctattccaagtggttc

SEQ ID NO: 56 - DNA, 33 bp, Artificial sequence
  Synthetic primer for amplifying gene regulatory element from Sorghum sp.
gagaggatccctgtagaagaaaaaacaagcaac

SEQ ID NO: 57 - DNA, 34 bp, Artificial sequence
  Synthetic primer for amplifying gene regulatory element from Sorghum sp.
gagaaagcttgactcccttagggtccattcgttt

SEQ ID NO: 58 - DNA, 33 bp, Artificial sequence; identical to SEQ ID NO: 56
  Synthetic primer for amplifying gene regulatory element from Sorghum sp.

SEQ ID NO: 59 - DNA, 34 bp, Artificial sequence; identical to SEQ ID NO: 57
  Synthetic primer for amplifying gene regulatory element from Sorghum sp.

SEQ ID NO: 60 - DNA, 33 bp, Artificial sequence
  Synthetic primer for amplifying gene regulatory element from Sorghum sp.
gagaggatcccttagaagcgggtgatggattga

SEQ ID NO: 61 - DNA, 30 bp, Artificial sequence
  Synthetic primer for amplifying gene regulatory element from Sorghum sp.
gcgaagcttatttaatgctccatgcatgtg

SEQ ID NO: 62 - DNA, 33 bp, Artificial sequence; identical to SEQ ID NO: 60
  Synthetic primer for amplifying gene regulatory element from Sorghum sp.

SEQ ID NO: 63 - DNA, 33 bp, Artificial sequence
  Synthetic primer for amplifying gene regulatory element from Sorghum sp.
gagaggcgcgccagtcggtagtacatgtatatg

SEQ ID NO: 64 - DNA, 30 bp, Artificial sequence
  Synthetic primer for amplifying gene regulatory element from Sorghum sp.
gcgagttaacttgctacagattctggaaca

SEQ ID NO: 65 - DNA, 31 bp, Artificial sequence
  Synthetic primer for amplifying gene regulatory element from Sorghum sp.
gcgaagcttattgggcgaatagttttactag

SEQ ID NO: 66 - DNA, 30 bp, Artificial sequence; identical to SEQ ID NO: 64
  Synthetic primer for amplifying gene regulatory element from Sorghum sp.

SEQ ID NO: 67 - DNA, 33 bp, Artificial sequence
  Synthetic primer for amplifying gene regulatory element from Sorghum sp.
gagactagtagtgctgaaagcaccgacgatgta

SEQ ID NO: 68 - DNA, 29 bp, Artificial sequence
  Synthetic primer for amplifying gene regulatory element from Sorghum sp.
gagggatcctcctcaaagtgttctgcagc

SEQ ID NO: 69 - DNA, 30 bp, Artificial sequence
  Synthetic primer for amplifying gene regulatory element from Sorghum sp.
gagaagcttacacgattaggtcagcagtgc

SEQ ID NO: 70 - DNA, 29 bp, Artificial sequence
  Synthetic primer for amplifying gene regulatory element from Sorghum sp.
gagggatcctctcaactattctgtaacag

SEQ ID NO: 71 - DNA, 29 bp, Artificial sequence
  Synthetic primer for amplifying gene regulatory element from Sorghum sp.
gagaagctttactgagagcgttgtggatg

SEQ ID NO: 72 - DNA, 29 bp, Artificial sequence
  Synthetic primer for amplifying gene regulatory element from Sorghum sp.
gagggatccggctgcttcgctgctcctgc

SEQ ID NO: 73 - DNA, 30 bp, Artificial sequence
  Synthetic primer for amplifying gene regulatory element from Sorghum sp.
gagaagcttactaattgcgcagtttggtca

SEQ ID NO: 74 - DNA, 27 bp, Artificial sequence
  Synthetic primer for amplifying gene regulatory element from Sorghum sp.
gagggatccgctggaggagcgtggagc

SEQ ID NO: 75 - protein, 359 aa, Acidothermus cellulolyticus
  misc_feature (1)..(359): E1 amino acid sequence
AGGGYWHTSGREILDANNVPVRIAGINWFGFETCNYVVHGLWSRDYRSMLDQIKSLGYNTIRLPYSDDILKPGTMPNSINFYQMNQDLQGLTSLQVMDKIVAYAGQIGLRIILDRHRPDCSGQSALWYTSSVSEATWISDLQALAQRYKGNPTVVGFDLHNEPHDPACWGCGDPSIDWRLAAERAGNAVLSVNPNLLIFVEGVQSYNGDSYWWGGNLQGAGQYPVVLNVPNRLVYSAHDYATSVYPQTWFSDPTFPNNMPGIWNKNWGYLFNQNIAPVWLGEFGTTLQSTTDQTWLKTLVQYLRPTAQYGADSFQWTFWSWNPDSGDTGGILKDDWQTVDTVKDGYLAPIKSSIFDPVG

SEQ ID NO: 76 - protein, 1124 aa, Acidothermus cellulolyticus
  misc_feature (1)..(1124): gux1 amino acid sequence
MGAPGLRRRLRAGIVSAAALGSLVSGLVAVAPVAHAAVTLKAQYKNNDSAPSDNQIKPGLQLVNTGSSSVDLSTVTVRYWFTRDGGSSTLVYNCDWAAMGCGNIRASFGSVNPATPTADTYLQLSFTGGTLAAGGSTGEIQNRVNKSDWSNFDETNDYSYGTNTTFQDWTKVTVYVNGVLVWGTEPSGATASPSASATPSPSSSPTTSPSSSPSPSSSPTPTPSSSSPPPSSNDPYIQRFLTMYNKIHDPANGYFSPQGIPYHSVETLIVEAPDYGHETTSEAYSFWLWLEATYGAVTGNWTPFNNAWTTMETYMIPQHADQPNNASYNPNSPASYAPEEPLPSMYPVAIDSSVPVGHDPLAAELQSTYGTPDIYGMHWLADVDNIYGYGDSPGGGCELGPSAKGVSYINTFQRGSQESVWETVTQPTCDNGKYGGAHGYVDLFIQGSTPPQWKYTDAPDADARAVQAAYWAYTWASAQGKASAIAPTIAKAAKLGDYLRYSLFDKYFKQVGNCYPASSCPGATGRQSETYLIGWYYAWGGSSQGWAWRIGDGAAHFGYQNPLAAWAMSNVTPLIPLSPTAKSDWAASLQRQLEFYQWLQSAEGAIAGGATNSWNGNYGTPPAGDSTFYGMAYDWEPVYHDPPSNNWFGFQAWSMERVAEYYYVTGDPKAKALLDKWVAWVKPNVTTGASWSIPSNLSWSGQPDTWNPSNPGTNANLHVTITSSGQDVGVAAALAKTLEYYAAKSGDTASRDLAKGLLDSIWNNDQDSLGVSTPETRTDYSRFTQVYDPTTGDGLYIPSGWTGTMPNGDQIKPGATFLSIRSWYTKDPQWSKVQAYLNGGPAPTFNYHRFWAESDFAMANADFGMLFPSGSPSPTPSPTPTSSPSPTPSSSPTPSPSPSPTGDTTPPSVPTGLQVTGTTTSSVSLSWTASTDNVGVAHYNVYRNGTLVGQPTATSFTDTGLAAGTSYTYTVAAVDAAGNTSAQSSPVTATTASPSPSPSPSPTPTSSPSPTPSPTPSPTSTSGASCTATYVVNSDWGSGFTTTVTVTNTGTRATSGWTVTWSFAGNQTVTNYWNTALTQSGKSVTAKNLSYNNVIQPGQSTTFGFNGSYSGTNTAPTLSCTASZ

SEQ ID NO: 77 - protein, 683 aa, Acidothermus cellulolyticus
  misc_feature (1)..(683): XylE amino acid sequence
MGHHAMRRMVTSASVVGVATLAAATVLITGGIAHAASTLKQGAEANGRYFGVSASVNTLNNSAAANLVATQFDMLTPENEMKWDTVESSRGSFNFGPGDQIVAFATAHNMRVRGHNLVWHSQLPGWVSSLPLSQVQSAMESHITAEVTHYKGKIYAWDVVNEPFDDSGNLRTDVFYQAMGAGYIADALRTAHAADPNAKLYLNDYNIEGINAKSDAMYNLIKQLKSQGVPIDGVGFESHFIVGQVPSTLQQNMQRFADLGVDVAITELDDRMPTPPSQQNLNQQATDDANVVKACLAVARCVGITQWDVSDADSWVPGTFSGQGAATMFDSNLQPKPAFTAVLNALSASASVSPSPSPSPSPSPSPSPSPSPSPSPSPSPSPSPSSSPVSGGVKVQYKNNDSAPGDNQIKPGLQVVNTGSSSVDLSTVTVRYWFTRDGGSSTLVYNCDWAVMGCGNIRASFGSVNPATPTADTYLQLSFTGGTLPAGGSTGEIQSRVNKSDWSNFTETNDYSYGTNTTFQDWSKVTVYVNGRLVWGTEPSGTSPSPTPSPSPTPSPSPSPSPSPSPSPSPSPSPSPSSSPSSGCVASMRVDSSWPGGFTATVTVSNTGGVSTSGWQVGWSWPSGDSLVNAWNAVVSVTGTSVRAVNASYNGVIPAGGSTTFGFQANGTPGTPTFTCTTSADLZ

SEQ ID NO: 78 - protein, 1256 aa, Acidothermus cellulolyticus
  misc_feature (1)..(1256): aviIII amino acid sequence
MAATTQPYTWSNVAIGGGGFVDGIVFNEGAPGILYVRTDIGGMYRWDAANGRWIPLLDWVGWNNWGYNGVVSIAADPINTNKVWAAVGMYTNSWDPNDGAILRSSDQGATWQITPLPFKLGGNMPGRGMGERLAVDPNNDNILYFGAPSGKGLWRSTDSGATWSQMTNFPDVGTYIANPTDTTGYQSDIQGVVWVAFDKSSSSLGQASKTIFVGVADPNNPVFWSRDGGATWQAVPGAPTGFIPHKGVFDPVNHVLYIATSNTGGPYDGSSGDVWKFSVTSGTWTRISPVPSTDTANDYFGYSGLTIDRQHPNTIMVATQISWWPDTIIFRSTDGGATWTRIWDWTSYPNRSLRYVLDISAEPWLTFGVQPNPPVPSPKLGWMDEAMAIDPFNSDRMLYGTGATLYATNDLTKWDSGGQIHIAPMVKGLEETAVNDLISPPSGAPLISALGDLGGFTHADVTAVPSTIFTSPVFTTGTSVDYAELNPSIIVRAGSFDPSSQPNDRHVAFSTDGGKNWFQGSEPGGVTTGGTVAASADGSRFVWAPGDPGQPVVYAVGFGNSWAASQGVPANAQIRSDRVNPKTFYALSNGTFYRSTDGGVTFQPVAAGLPSSGAVGVMFHAVPGKEGDLWLAASSGLYHSTNGGSSWSAITGVSSAVNVGFGKSAPGSSYPAVFVVGTIGGVTGAYRSDDGGTTWVRINDDQHQYGNWGQAITGDPRIYGRVYIGTNGRGIVYGDIAGAPSGSPSPSVSPSASPSLSPSPSPSSSPSPSPSPSSSPSSSPSPSPSPSPSPSRSPSPSASPSPSSSPSPSSSPSSSPSPTPSSSPVSGGVKVQYKNNDSAPGDNQIKPGLQVVNTGSSSVDLSTVTVRYWFTRDGGSSTLVYNCDWAAIGCGNIRASFGSVNPATPTADTYLQLSFTGGTLAAGGSTGEIQNRVNKSDWSNFTETNDYSYGTNTVFQDWSKVTVYVNGRLVWGTEPSGTSPSPTPSPSPTPSPSPSPSPGGDVTPPSVPTGVVVTGVSGSSVSLAWNASTDNVGVAHYNVYRNGVLVGQPTVTSFTDTGLAAGTAYTYTVAAVDAAGNTSAPSTPVTATTTSPSPSPSPTPSPTPSPTPSPSPSPSLSPSPSPSPSPSPSPSLSPSPSTSPSPSPSPTPSPSSSGVGCRATYVVNSDWGSGFTATVTVTNTGSRATSGWTVAWSFGGNQTVTNYWNTLLTQSGASVTATNLSYNNVIQPGQSTTFGFNATYAGTNTPPTPTCTTNSD

SEQ ID NO: 79 - protein, 441 aa, Talaromyces emersonii
  misc_feature (1)..(441): cbhE amino acid sequence
MDPQQAGTATAENHPPLTWQECTAPGSCTTQNGAVVLDANWRWVHDVNGYTNCYTGNTWDPTYCPDDETCAQNCALDGADYEGTYGVTSSGSSLKLNFVTGSNVGSRLYLLQDDSTYQIFKLLNREFSFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYIQNSNVIPQPNSDISGVTGNSITTEFCTAQKQAFGDTDDFSQHGGLAKMGAAMQQGMVLVMSLDDYAAQMLWLDSDYPTDADPTTPGIARGTCPTDSGVPSDVESQSPNSYVTYSNIKFGPINSTFTASGD